SEQUENCE LISTING (161 sequences)

SEQ ID NO: 1 - DNA, 20 bp, synthetic construct
  misc_feature 1..20: note = Synthetic polynucleotide
  misc_feature 1..20: note = T1 Guide sequence
  source 1..20: mol_type = other DNA
ttccagggccgagatcttcg

SEQ ID NO: 2 - DNA, 20 bp, synthetic construct
  misc_feature 1..20: note = Synthetic polynucleotide
  misc_feature 1..20: note = T3 Guide sequence
  source 1..20: mol_type = other DNA
cgcctcgaagatctcggccc

SEQ ID NO: 3 - DNA, 20 bp, synthetic construct
  misc_feature 1..20: note = Synthetic polynucleotide
  misc_feature 1..20: note = T4 Guide sequence
  source 1..20: mol_type = other DNA
tcgaagatctcggccctgga

SEQ ID NO: 4 - DNA, 20 bp, synthetic construct
  misc_feature 1..20: note = Synthetic polynucleotide
  misc_feature 1..20: note = T7 Guide sequence
  source 1..20: mol_type = other DNA
ggccctggaaggttccccct

SEQ ID NO: 5 - DNA, 20 bp, synthetic construct
  misc_feature 1..20: note = Synthetic polynucleotide
  misc_feature 1..20: note = T9 Guide sequence
  source 1..20: mol_type = other DNA
tccagctgggcgaggctcct

SEQ ID NO: 6 - DNA, 20 bp, synthetic construct
  misc_feature 1..20: note = Synthetic polynucleotide
  misc_feature 1..20: note = T18 Guide sequence
  source 1..20: mol_type = other DNA
tcagacctgctgggggcccg

SEQ ID NO: 7 - DNA, 20 bp, synthetic construct
  misc_feature 1..20: note = Synthetic polynucleotide
  misc_feature 1..20: note = R1 Guide sequence
  source 1..20: mol_type = other DNA
gagccccgcctcgaagatct

SEQ ID NO: 8-14 - skipped (7 entries)

SEQ ID NO: 15 - DNA, 20 bp, synthetic construct
  misc_feature 1..20: note = Synthetic polynucleotide
  misc_feature 1..20: note = P1 Guide sequence
  source 1..20: mol_type = other DNA
attcccagggccggttaatg

SEQ ID NO: 16 - DNA, 20 bp, synthetic construct
  misc_feature 1..20: note = Synthetic polynucleotide
  misc_feature 1..20: note = P3 Guide sequence
  source 1..20: mol_type = other DNA
gtcccctccaccccacagtg

SEQ ID NO: 17 - DNA, 20 bp, synthetic construct
  misc_feature 1..20: note = Synthetic polynucleotide
  misc_feature 1..20: note = P4 Guide sequence
  source 1..20: mol_type = other DNA
accccacagtggggccacta

SEQ ID NO: 18 - DNA, 20 bp, synthetic construct
  misc_feature 1..20: note = Synthetic polynucleotide
  misc_feature 1..20: note = N1 Guide sequence
  source 1..20: mol_type = other DNA
cctctaaggtttgcttacga

SEQ ID NO: 19 - DNA, 20 bp, synthetic construct
  misc_feature 1..20: note = Synthetic polynucleotide
  misc_feature 1..20: note = N2 Guide sequence
  source 1..20: mol_type = other DNA
tataaggtggtcccagctcg

SEQ ID NO: 20 - DNA, 20 bp, synthetic construct
  misc_feature 1..20: note = Synthetic polynucleotide
  misc_feature 1..20: note = N3 Guide sequence
  source 1..20: mol_type = other DNA
ccatcgtaagcaaaccttag

SEQ ID NO: 21-26 - skipped (6 entries)

SEQ ID NO: 27 - DNA, 20 bp, synthetic construct
  misc_feature 1..20: note = Synthetic polynucleotide
  misc_feature 1..20: note = m T20 Guide sequence
  source 1..20: mol_type = other DNA
gactcctggggatgggccaa

SEQ ID NO: 28 - DNA, 20 bp, synthetic construct
  misc_feature 1..20: note = Synthetic polynucleotide
  misc_feature 1..20: note = m T22 Guide sequence
  source 1..20: mol_type = other DNA
ttggcccttggcccatcccc

SEQ ID NO: 29 - DNA, 20 bp, synthetic construct
  misc_feature 1..20: note = Synthetic polynucleotide
  misc_feature 1..20: note = m T23 Guide sequence
  source 1..20: mol_type = other DNA
ccagcttggcaagactcctg

SEQ ID NO: 30-32 - skipped (3 entries)

SEQ ID NO: 33 - DNA, 6679 bp, synthetic construct
  misc_feature 1..6679: note = Synthetic polynucleotide
  misc_feature 1..6679: note = 3008 pAAV_FoxP3_0_6kb_MND_GFP_WPRE3_pA
  source 1..6679: mol_type = other DNA
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgctcagaatctacccacttctcgccttctccactgccaccagcccattctgtgccagcatcatcacttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgacgcgtaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtgggtcgactgctttatttgtgaaatttgtgatgctattgctttatttgtaaccattataagctgcaataaacaagttaacaacaacaattgcattcattttatgtttcaggttcagggggagatgtgggaggttttttaaagcactagtgtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccacactgcccctagtcatggtggcaccctccggggcacggctgggccccttgccccacttacaggcaccgcggcgctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagacccc

SEQ ID NO: 34 - DNA, 6844 bp, synthetic construct
  misc_feature 1..6844: note = Synthetic polynucleotide
  misc_feature 1..6844: note = 3037 pAAV_FoxP3_0_6kb_FoxP3cDNA_WPRE3_pA
  source 1..6844: mol_type = other DNA
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgctcagaatctacccacttctcgccttctccactgccaccagcccattctgtgccagcatcatcacttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggaccttgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtgggtcgactgctttatttgtgaaatttgtgatgctattgctttatttgtaaccattataagctgcaataaacaagttaacaacaacaattgcattcattttatgtttcaggttcagggggagatgtgggaggttttttaaagcactagtgtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccacactgcccctagtcatggtggcaccctccggggcacggctgggccccttgccccacttacaggcaccgcggcgctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagacccc

SEQ ID NO: 35 - DNA, 7048 bp, synthetic construct
  misc_feature 1..7048: note = Synthetic polynucleotide
  misc_feature 1..7048: note = 3088 pAAV_FOXP3_08_MND_GFP 08_for T3
  source 1..7048: mol_type = other DNA
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgccttgcccactacatccaagctgctagcactgctcctgatccagcttcagattaagtctcagaatctacccacttctcgccttctccactgccaccagcccattctgtgccagcatcatcacttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatccccaggagcctcgcccagctggagggctgcacccaaagcctcagacctgctgggggcccggggcccagggggaaccttccaacgcgtaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtgggtcgactgctttatttgtgaaatttgtgatgctattgctttatttgtaaccattataagctgcaataaacaagttaacaacaacaattgcattcattttatgtttcaggttcagggggagatgtgggaggttttttaaagcactagtcgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccacactgcccctagtcatggtggcaccctccggggcacggctgggccccttgccccacttacaggcactcctccaggacaggccacatttcatgcaccaggtatggacggtgaatgggcagggaggagggagcaggtgggagaactgtggggaggggccccgagtcaggctgaaccggatcctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagacccc

SEQ ID NO: 36 - DNA, 7056 bp, synthetic construct
  misc_feature 1..7056: note = Synthetic polynucleotide
  misc_feature 1..7056: note = 3089 pAAV_FOXP3_08_MND_GFP08_for T9
  source 1..7056: mol_type = other DNA
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcatctcaggtaatgtcagctcggtccttccagctgctcaagctaaaacccatgtcactttgactctccctcttgcccactacatccaagctgctagcactgctcctgatccagcttcagattaagtctcagaatctacccacttctcgccttctccactgccaccagcccattctgtgccagcatcatcacttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatccccacgcgtaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtgggtcgactgctttatttgtgaaatttgtgatgctattgctttatttgtaaccattataagctgcaataaacaagttaacaacaacaattgcattcattttatgtttcaggttcagggggagatgtgggaggttttttaaagcactagtgcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccacactgcccctagtcatggtggcaccctccggggcacggctgggccccttgccccacttacaggcactcctccaggacaggccacatttcatgcaccaggtatggacggtgaatggatcctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagacccc

SEQ ID NO: 37 - protein, 90 aa, synthetic construct
  REGION 1..90: note = Synthetic polypeptide
MEMWHEGLEEASRLYFGERNVKGMFEVLEPLHAMMERGPQTLKETSFNQAYGRDLMEAQEWCRKYMKSGNVKDLTQAWDLYYHVFRRISK

SEQ ID NO: 38 - protein, 90 aa, synthetic construct
  REGION 1..90: note = Synthetic polypeptide
MEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 39 - protein, 90 aa, synthetic construct; identical to SEQ ID NO: 37
  REGION 1..90: note = Synthetic polypeptide
  REGION 1..90: note = misc_feature - naked FRB wild type polypeptide

SEQ ID NO: 40 - protein, 90 aa, synthetic construct; identical to SEQ ID NO: 38
  REGION 1..90: note = Synthetic polypeptide
  REGION 1..90: note = misc_feature - naked FRB mutant polypeptide

SEQ ID NO: 41 - DNA, 4187 bp, synthetic construct
  misc_feature 1..4187: note = Synthetic polynucleotide
  misc_feature 1..4187: note = DISC vector DNA
  source 1..4187: mol_type = other DNA
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgctagcaccggtgccgccaccatgcctctgggcctgctgtggctgggcctggccctgctgggcgccctgcacgcccaggccggcgtgcaggtggagacaatctccccaggcgacggacgcacattccctaagcggggccagacctgcgttgtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcttgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccccccacccccggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatctggtgctactaatttttctcttttgaagcaagctggagatgttgaagagaaccccggtccggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcatgtctttaggcgcattagtaagggcagcggcgccaccaacttcagcctgctgaagcaggccggcgacgtggaggagaaccccggccccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctctctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaactagtgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgga

SEQ ID NO: 42 - DNA, 393 bp, synthetic construct
  misc_feature 1..393: note = Synthetic polynucleotide
  misc_feature 1..393: note = uDISC DNA cytoplasmic tail only codon diverged
  source 1..393: mol_type = other DNA
ccagcagctctcggcaaagacacgattccgtggcttgggcatctgctcgttgggctgagcggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagcttgaatacagacgcttatctctcactgcaggaactgcaa

SEQ ID NO: 43 - protein, 131 aa, synthetic construct
  REGION 1..131: note = Synthetic polypeptide
  REGION 1..131: note = misc_feature - uDISC polypeptide cytoplasmic tail only
PAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSLNTDAYLSLQELQ

SEQ ID NO: 44 - protein, 108 aa, synthetic construct
  REGION 1..108: note = Synthetic polypeptide
  REGION 1..108: note = misc_feature - FKBP CISC domain
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGE

SEQ ID NO: 45 - protein, 245 aa, synthetic construct
  REGION 1..245: note = Synthetic polypeptide
  REGION 1..245: note = misc_feature - Entire uDISC polypeptide FRB truncated IL2Rbeta
MALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSLNTDAYLSLQELQ

SEQ ID NO: 46 - DNA, 3623 bp, synthetic construct
  misc_feature 1..3623: note = Synthetic polynucleotide
  misc_feature 1..3623: note = uDISC vector DNA
  source 1..3623: mol_type = other DNA
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgctagcaccggtgccgccaccatgcctctgggcctgctgtggctgggcctggccctgctgggcgccctgcacgcccaggccggcgtgcaggtggagacaatctccccaggcgacggacgcacattccctaagcggggccagacctgcgttgtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagcttgaatacagacgcttatctctcactgcaggaactgcaaggatctggtgctactaatttttctcttttgaagcaagctggagatgttgaagagaaccccggtccggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcatgtctttaggcgcattagtaagggcagcggcgccaccaacttcagcctgctgaagcaggccggcgacgtggaggagaaccccggccccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctctctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaactagtgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgga

SEQ ID NO: 47 - protein, 251 aa, synthetic construct
  REGION 1..251: note = Synthetic polypeptide
  REGION 1..251: note = misc_feature - IL2Rgamma_CISC polypeptide
MPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 48 - protein, 352 aa, synthetic construct
  REGION 1..352: note = Synthetic polypeptide
  REGION 1..352: note = misc_feature - IL2Rgamma_CISC polypeptide
MPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGGSQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 49 - protein, 349 aa, synthetic construct
  REGION 1..349: note = Synthetic polypeptide
  REGION 1..349: note = misc_feature - IL2Rgamma_CISC polypeptide
MPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 50 - protein, 251 aa, synthetic construct
  REGION 1..251: note = Synthetic polypeptide
  REGION 1..251: note = misc_feature - IL2Rgamma_CISC polypeptide
MPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 51 - protein, 429 aa, synthetic construct
  REGION 1..429: note = Synthetic polypeptide
  REGION 1..429: note = misc_feature - IL2Rbeta_CISC polypeptide
MALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 52 - protein, 543 aa, synthetic construct
  REGION 1..543: note = Synthetic polypeptide
  REGION 1..543: note = misc_feature - IL2Rbeta_CISC polypeptide
MALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKGGSKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 53 - protein, 541 aa, synthetic construct
  REGION 1..541: note = Synthetic polypeptide
  REGION 1..541: note = misc_feature - IL2Rbeta_CISC polypeptide
MALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 54 - protein, 379 aa, synthetic construct
  REGION 1..379: note = Synthetic polypeptide
  REGION 1..379: note = misc_feature - IL7Ralpha_CISC polypeptide
MALPVTALLLPLALLLHAARPILWHEMWHEGLEEASRLYFGERNVKGMFEVLEPLHAMMERGPQTLKETSWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 55 - protein, 345 aa, synthetic construct
  REGION 1..345: note = Synthetic polypeptide
  REGION 1..345: note = misc_feature - IL7Ralpha_CISC polypeptide
MALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKGEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 56 - protein, 4 aa, synthetic construct
  REGION 1..4: note = Synthetic polypeptide
  REGION 1..4: note = misc_feature - linker polypeptide
GGGS

SEQ ID NO: 57 - protein, 7 aa, synthetic construct
  REGION 1..7: note = Synthetic polypeptide
  REGION 1..7: note = misc_feature - linker polypeptide
GGGSGGG

SEQ ID NO: 58-59 - skipped (2 entries)

SEQ ID NO: 60 - protein, 4 aa, synthetic construct
  REGION 1..4: note = Synthetic polypeptide
  REGION 1..4: note = misc_feature - linker polypeptide
GGSP

SEQ ID NO: 61 - protein, 251 aa, synthetic construct
  REGION 1..251: note = Synthetic polypeptide
  REGION 1..251: note = misc_feature - IL2Rgamma_CISC polypeptide
MPLGLLWLGLALLGALHAQAGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 62 - protein, 443 aa, synthetic construct
  REGION 1..443: note = Synthetic polypeptide
  REGION 1..443: note = misc_feature - IL2Rbeta_CISC polypeptide
MPLGLLWLGLALLGALHAQAGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 63 - protein, 358 aa, synthetic construct
  REGION 1..358: note = Synthetic polypeptide
  REGION 1..358: note = misc_feature - IL2Ralpha_CISC polypeptide
MPLGLLWLGLALLGALHAQAGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 64 - protein, 358 aa, synthetic construct; identical to SEQ ID NO: 63
  REGION 1..358: note = Synthetic polypeptide
  REGION 1..358: note = misc_feature - IL7Ralpha_CISC polypeptide

SEQ ID NO: 65 - protein, 276 aa, synthetic construct
  REGION 1..276: note = Synthetic polypeptide
  REGION 1..276: note = misc_feature - MPL_CISC polypeptide
MPLGLLWLGLALLGALHAQAGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLGEETAWISLVTALHLVLGLSAVLGLLLLRWQFPAHYRRLRHALWPSLPDLHRVLGQYLRDTAALSPPKATVSDTCEEVEPSLLEILPKSSERTPLPLCSSQAQMDYRRLQPSCLGTMPLSVCPPMAESGSCCTTHIANHSYLPLSYWQQP

SEQ ID NO: 66 - DNA, 9405 bp, synthetic construct
  misc_feature 1..9405: note = Synthetic polynucleotide
  misc_feature 1..9405: note = CISC vector DNA
  source 1..9405: mol_type = other DNA
agcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatcgaattcccacggggttggacgcgtaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgctagcaccggtgccgccaccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcttgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccccccacccccggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatctggtgctactaatttttctcttttgaagcaagctggagatgttgaagagaaccctggtccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaactagtgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgca

SEQ ID NO: 67 - DNA, 10053 bp, synthetic construct
  misc_feature 1..10053: note = Synthetic polynucleotide
  misc_feature 1..10053: note = CISC vector DNA
  source 1..10053: mol_type = other DNA
agcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatcgaattcccacggggttggacgcgtaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgctagcaccggtgccgccaccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggcggtagtcagaaccttgtgataccatgggccccagaaaatctcacacttcataaactttccgaatcacaactcgaactcaactggaataaccggttcctgaatcactgtcttgaacacctggtacaatatcggaccgactgggatcactcatggacagaacaatctgtggactataggcacaaattctcactcccaagcgtagacggccaaaaaagatacacttttcgcgtacgatcccgctttaatcctctctgcggctctgctcagcactggagtgaatggtcccatcccattcattggggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagggaggttcaaaaccttttgagaaccttagactgatggcgcccatctctctgcaggtagttcacgttgagacccatagatgcaatataagctgggaaatctcacaagccagccattactttgaacggcatttggaattcgaggcccgaacactttcccccggtcatacgtgggaagaagctcctctcttgacgctgaagcagaagcaggagtggatttgtctggagactttgactcctgatactcagtatgagttccaagttcgggtgaaaccactccaaggcgagttcacgacgtggtctccgtggagtcaaccgttggcgttccgcacgaagcccgctgcccttggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcttgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccccccacccccggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatctggtgctactaatttttctcttttgaagcaagctggagatgttgaagagaaccctggtccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaactagtgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgca

SEQ ID NO: 68 - DNA, 10035 bp, synthetic construct
  misc_feature 1..10035: note = Synthetic polynucleotide
  misc_feature 1..10035: note = CISC vector DNA
  source 1..10035: mol_type = other DNA
agcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatcgaattcccacggggttggacgcgtaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgctagcaccggtgccgccaccatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagcaaaacttggtgattccttgggccccagaaaatctcacgcttcacaagttgtccgaatcccagctcgagctcaactggaataatagatttcttaatcattgtttggaacacctggttcaatatagaacggattgggaccactcatggaccgagcagtcagttgactaccgccacaaattttcacttcccagcgtagatgggcagaagaggtacacatttagggtcagatccaggtttaatcctctgtgtggttctgctcaacactggtctgagtggagccatccgatccactggggctcaaatacctctaaagaaaatccgttcctctttgcgctcgaagccgttgttatcagcgtcggaagcatgggacttatcatttcccttctctgcgtgtacttctggctggagcggacgatgccgcggattccgacgctcaaaaacctggaggaccttgtaacagaatatcacggtaatttctccgcttggagtggcgtatcaaaggggcttgctgagtcccttcaaccggattactctgagcgcctctgcttggtgtccgagatacctcccaaaggaggtgcacttggggaggggccaggcgcgtccccttgcaatcagcatagtccgtattgggcgcccccctgttataccctcaaaccggaaacgggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagaaaccttttgagaaccttagactgatggcgcccatctctctgcaggtagttcacgttgagacccatagatgcaatataagctgggaaatctcacaagccagccattactttgaacggcatttggaattcgaggcccgaacactttcccccggtcatacgtgggaagaagctcctctcttgacgctgaagcagaagcaggagtggatttgtctggagactttgactcctgatactcagtatgagttccaagttcgggtgaaaccactccaaggcgagttcacgacgtggtctccgtggagtcaaccgttggcgttccgcacgaagcccgctgcccttggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcttgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccccccacccccggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatctggtgctactaatttttctcttttgaagcaagctggagatgttgaagagaaccctggtccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaactagtgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgca

SEQ ID NO: 69 - protein, 4 aa, synthetic construct
  REGION 1..4: note = Synthetic polypeptide
  REGION 1..4: note = misc_feature - Linker spacer polypeptide
PAAL

SEQ ID NO: 70 - DNA, 267 bp, synthetic construct
  misc_feature 1..267: note = Synthetic polynucleotide
  misc_feature 1..267: note = Naked FRB domain nucleic acid sequence
  source 1..267: mol_type = other DNA
gagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgacccaagcgtgggacctgtactatcatgtctttaggcgcattagtaag

SEQ ID NO: 71 - DNA, 353 bp, synthetic construct
  misc_feature 1..353: note = Synthetic polynucleotide
  misc_feature 1..353: note = MND promoter
  source 1..353: mol_type = other DNA
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgctagc

SEQ ID NO: 72 - DNA, 2190 bp, synthetic construct
  misc_feature 1..2190: note = Synthetic polynucleotide
  misc_feature 1..2190: note = FOXP3cDNA_P2A_LNGFR kozak_start codon_FOXP3cDNA_P2A_LNGFR_stop codon
  source 1..2190: mol_type = other DNA
gccaccatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggacctggaagcggagcgactaacttcagcctgctgaagcaggccggagatgtggaggaaaaccctggaccgatgggggcaggtgccaccggacgagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtga

SEQ ID NO: 73 - DNA, 2190 bp, synthetic construct
  misc_feature 1..2190: note = Synthetic polynucleotide
  misc_feature 1..2190: note = LNGFR_P2A_FOXP3cDNA kozak_start codon_LNGFR_P2A_FOXP3cDNA_stop codon
  source 1..2190: mol_type = other DNA
gccaccatgggggcaggtgccaccggacgagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggggaagcggagcgactaacttcagcctgctgaagcaggccggagatgtggaggaaaaccctggaccgatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggaccttga

SEQ ID NO: 74 - DNA, 3261 bp, synthetic construct
  misc_feature 1..3261: note = Synthetic polynucleotide
  misc_feature 1..3261: note = FOXP3cDNA_uDISC nucleotide sequence coding sequence only codon optimized our DISC architecture version 6
  source 1..3261: mol_type = other DNA
atgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggacctggaagcggagcgactaacttcagcctgcttaagcaggccggagatgtggaggaaaaccctggaccgatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggagggtcacctggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagccagcagctctcggcaaagacacgattccgtggcttgggcatctgctcgttgggctgagcggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagcttgaatacagacgcttatctctcactgcaggaactgcaaggatctggtgctactaatttttctcttttgaagcaagctggagatgttgaagagaaccccggtccggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcatgtctttaggcgcattagtaagtga

SEQ ID NO: 75 - DNA, 3805 bp, synthetic construct
  misc_feature 1..3805: note = Synthetic polynucleotide
  misc_feature 1..3805: note = MND_FOXP3cDNA_uDISC_SV40 polyA nucleotide sequence codon optimized
  source 1..3805: mol_type = other DNA
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggatctcgaggccaccatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggacctggaagcggagcgactaacttcagcctgcttaagcaggccggagatgtggaggaaaaccctggaccgatgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggagggtcacctggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagccagcagctctcggcaaagacacgattccgtggcttgggcatctgctcgttgggctgagcggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagcttgaatacagacgcttatctctcactgcaggaactgcaaggatctggtgctactaatttttctcttttgaagcaagctggagatgttgaagagaaccccggtccggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcatgtctttaggcgcattagtaagtgagtcgactgctttatttgtgaaatttgtgatgctattgctttatttgtaaccattataagctgcaataaacaagttaacaacaacaattgcattcattttatgtttcaggttcagggggagatgtgggaggttttttaaagc

SEQ ID NO: 76 - protein, 1086 aa, synthetic construct
  REGION 1..1086: note = Synthetic polypeptide
  REGION 1..1086: note = misc_feature - FOXP3cDNA_uDISC amino acid sequence
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGPGSGATNFSLLKQAGDVEENPGPMPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGGSPGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSLNTDAYLSLQELQGSGATNFSLLKQAGDVEENPGPEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 77 - DNA, 4080 bp, synthetic construct
  misc_feature 1..4080: note = Synthetic polynucleotide
  misc_feature 1..4080: note = FOXP3cDNA_LNGFRe_uDISC nucleotide sequence coding sequence only
  source 1..4080: mol_type = other DNA
atgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggacctggaagcggagcgactaacttcagcctgcttaagcaggccggagatgtggaggaaaaccctggaccgatgcctctgggcctgctgtggctgggcctggccctgctgggcgccctgcacgcccaggccatgggggcaggtgccaccggacgagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggagggtcacctggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagccagcagctctcggcaaagacacgattccgtggcttgggcatctgctcgttgggctgagcggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagcttgaatacagacgcttatctctcactgcaggaactgcaaggatctggtgctactaatttttctcttttgaagcaagctggagatgttgaagagaaccccggtccggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcatgtctttaggcgcattagtaag

SEQ ID NO: 78 - protein, 1360 aa, synthetic construct
  REGION 1..1360: note = Synthetic polypeptide
  REGION 1..1360: note = misc_feature - FOXP3cDNA_LNGFRe_uDISC amino acid sequence
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGPGSGATNFSLLKQAGDVEENPGPMPLGLLWLGLALLGALHAQAMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGGSPGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSLNTDAYLSLQELQGSGATNFSLLKQAGDVEENPGPEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 79 - DNA, 3258 bp, synthetic construct
  misc_feature 1..3258: note = Synthetic polynucleotide
  misc_feature 1..3258: note = uDISC_FOXP3cDNA nucleotide sequence coding sequence only
  source 1..3258: mol_type = other DNA
atgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggagggtcacctggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagccagcagctctcggcaaagacacgattccgtggcttgggcatctgctcgttgggctgagcggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagcttgaatacagacgcttatctctcactgcaggaactgcaaggatctggtgctactaatttttctcttttgaagcaagctggagatgttgaagagaaccccggtccggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcatgtctttaggcgcattagtaagggaagcggagcgactaacttcagcctgcttaagcaggccggagatgtggaggaaaaccctggaccgatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggacct

SEQ ID NO: 80 - protein, 1086 aa, synthetic construct
  REGION 1..1086: note = Synthetic polypeptide
  REGION 1..1086: note = misc_feature - uDISC_FOXP3cDNA amino acid sequence
MPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGGSPGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSLNTDAYLSLQELQGSGATNFSLLKQAGDVEENPGPEMWHEGLEEASRLYFGERNVKGMFEVLEPLHAMMERGPQTLKETSFNQAYGRDLMEAQEWCRKYMKSGNVKDLLQAWDLYYHVFRRISKGSGATNFSLLKQAGDVEENPGPMPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 81 - DNA, 4083 bp, synthetic construct
  misc_feature 1..4083: note = Synthetic polynucleotide
  misc_feature 1..4083: note = LNGFRe_uDISC _FOXP3cDNA nucleotide sequence coding sequence only
  source 1..4083: mol_type = other DNA
atgcctctgggcctgctgtggctgggcctggccctgctgggcgccctgcacgcccaggccatgggggcaggtgccaccggacgagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggagggtcacctggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagccagcagctctcggcaaagacacgattccgtggcttgggcatctgctcgttgggctgagcggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagcttgaatacagacgcttatctctcactgcaggaactgcaaggatctggtgctactaatttttctcttttgaagcaagctggagatgttgaagagaaccccggtccggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcatgtctttaggcgcattagtaagggaagcggagcgactaacttcagcctgcttaagcaggccggagatgtggaggaaaaccctggaccgatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggaccttga

SEQ ID NO: 82 - protein, 1360 aa, synthetic construct
  REGION 1..1360: note = Synthetic polypeptide
  REGION 1..1360: note = misc_feature - LNGFRe_uDISC_FOXP3cDNA amino acid sequence
MPLGLLWLGLALLGALHAQAMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGGSPGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSLNTDAYLSLQELQGSGATNFSLLKQAGDVEENPGPEMWHEGLEEASRLYFGERNVKGMFEVLEPLHAMMERGPQTLKETSFNQAYGRDLMEAQEWCRKYMKSGNVKDLLQAWDLYYHVFRRISKGSGATNFSLLKQAGDVEENPGPMPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 83 - DNA, 2463 bp, synthetic construct
  misc_feature 1..2463: note = Synthetic polynucleotide
  misc_feature 1..2463: note = DISC nucleotide sequence coding sequence only codon optimized
  source 1..2463: mol_type = other DNA
atgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggagggtcacctggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagccagcagctctcggcaaagacacgattccgtggcttgggcatctgctcgttgggctgagcggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcttgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccccccacccccggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatctggtgctactaatttttctcttttgaagcaagctggagatgttgaagagaaccccggtccggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcatgtctttaggcgcattagtaag

SEQ ID NO: 84 - protein, 821 aa, synthetic construct
  REGION 1..821: note = Synthetic polypeptide
  REGION 1..821: note = misc_feature - DISC amino acid sequence
MPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGGSPGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVGSGATNFSLLKQAGDVEENPGPEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 85 - DNA, 1899 bp, synthetic construct
  misc_feature 1..1899: note = Synthetic polynucleotide
  misc_feature 1..1899: note = uDISC nucleotide sequence coding sequence only codon optimized
  source 1..1899: mol_type = other DNA
atgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggagggtcacctggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagccagcagctctcggcaaagacacgattccgtggcttgggcatctgctcgttgggctgagcggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagcttgaatacagacgcttatctctcactgcaggaactgcaaggatctggtgctactaatttttctcttttgaagcaagctggagatgttgaagagaaccccggtccggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcatgtctttaggcgcattagtaag

SEQ ID NO: 86 - protein, 633 aa, synthetic construct
  REGION 1..633: note = Synthetic polypeptide
  REGION 1..633: note = misc_feature - uDISC amino acid sequence
MPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGGSPGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSLNTDAYLSLQELQGSGATNFSLLKQAGDVEENPGPEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 87 - DNA, 1632 bp, synthetic construct
  misc_feature 1..1632: note = Synthetic polynucleotide
  misc_feature 1..1632: note = CISCbeta_DN nucleotide sequence coding sequence only
  source 1..1632: mol_type = other DNA
atggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagccagcagctctcggcaaagacacgattccgtggcttgggcatctgctcgttgggctgagcggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcttgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccccccacccccggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatctggtgctactaatttttctcttttgaagcaagctggagatgttgaagagaaccccggtccggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcatgtctttaggcgcattagtaag

SEQ ID NO: 88 - protein, 544 aa, synthetic construct
  REGION 1..544: note = Synthetic polypeptide
  REGION 1..544: note = misc_feature - CISCbeta_DN amino acid sequence
MALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVGSGATNFSLLKQAGDVEENPGPEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 89 - DNA, 3015 bp, synthetic construct
  misc_feature 1..3015: note = Synthetic polynucleotide
  misc_feature 1..3015: note = CISCgamma_FOXP3cDNA_LNGFR nucleotide sequence coding sequence only codon optimized
  source 1..3015: mol_type = other DNA
atgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggagggtcacctggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagcgactaacttcagcctgcttaagcaggccggagatgtggaggaaaaccctggaccgatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggacctggaagcggagcgactaacttcagcctgctgaagcaggccggagatgtggaggaaaaccctggaccgatgggggcaggtgccaccggacgagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtga

SEQ ID NO: 90 - protein, 1004 aa, synthetic construct
  REGION 1..1004: note = Synthetic polypeptide
  REGION 1..1004: note = misc_feature - CISCgamma_FOXP3cDNA_LNGFR amino acid sequence
MPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGGSPGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPGPMPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGPGSGATNFSLLKQAGDVEENPGPMGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 91 - DNA, 3015 bp, synthetic construct
  misc_feature 1..3015: note = Synthetic polynucleotide
  misc_feature 1..3015: note = CISCgamma_LNGFR_FOXP3cDNA nucleotide sequence coding sequence only codon optimized
  source 1..3015: mol_type = other DNA
atgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggagggtcacctggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagcgactaacttcagcctgcttaagcaggccggagatgtggaggaaaaccctggaccgatgggggcaggtgccaccggacgagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggggaagcggagcgactaacttcagcctgctgaagcaggccggagatgtggaggaaaaccctggaccgatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggaccttga

SEQ ID NO: 92 - DNA, 3015 bp, synthetic construct; identical to SEQ ID NO: 91
  misc_feature 1..3015: note = Synthetic polynucleotide
  misc_feature 1..3015: note = CISCgamma_LNGFR_FOXP3cDNA amino acid sequence
  source 1..3015: mol_type = other DNA

SEQ ID NO: 93 - protein, 251 aa, synthetic construct; identical to SEQ ID NO: 47
  REGION 1..251: note = Synthetic polypeptide
  REGION 1..251: note = misc_feature - IL2Rgamma_CISC amino acid sequence

SEQ ID NO: 94 - protein, 429 aa, synthetic construct; identical to SEQ ID NO: 51
  REGION 1..429: note = Synthetic polypeptide
  REGION 1..429: note = misc_feature - IL2Rbeta_CISC

SEQ ID NO: 95 - protein, 352 aa, synthetic construct; identical to SEQ ID NO: 48
  REGION 1..352: note = Synthetic polypeptide
  REGION 1..352: note = misc_feature - IL2Rgamma_CISC

SEQ ID NO: 96 - protein, 543 aa, synthetic construct; identical to SEQ ID NO: 52
  REGION 1..543: note = Synthetic polypeptide
  REGION 1..543: note = misc_feature - IL2Rbeta_CISC

SEQ ID NO: 97 - protein, 349 aa, synthetic construct; identical to SEQ ID NO: 49
  REGION 1..349: note = Synthetic polypeptide
  REGION 1..349: note = misc_feature - IL2Rgamma_CISC

SEQ ID NO: 98 - protein, 541 aa, synthetic construct; identical to SEQ ID NO: 53
  REGION 1..541: note = Synthetic polypeptide
  REGION 1..541: note = misc_feature - IL2Rbeta_CISC

SEQ ID NO: 99 - protein, 251 aa, synthetic construct; identical to SEQ ID NO: 50
  REGION 1..251: note = Synthetic polypeptide
  REGION 1..251: note = misc_feature - IL2Rgamma_CISC

SEQ ID NO: 100 - protein, 379 aa, synthetic construct; identical to SEQ ID NO: 54
  REGION 1..379: note = Synthetic polypeptide
  REGION 1..379: note = misc_feature - IL2Rbeta_CISC

SEQ ID NO: 101 - protein, 345 aa, synthetic construct; identical to SEQ ID NO: 55
  REGION 1..345: note = Synthetic polypeptide
  REGION 1..345: note = misc_feature - IL7Ralpha_CISC

SEQ ID NO: 102 - protein, 443 aa, synthetic construct; identical to SEQ ID NO: 62
  REGION 1..443: note = Synthetic polypeptide
  REGION 1..443: note = misc_feature - IL2Rbeta_CISC

SEQ ID NO: 103 - protein, 251 aa, synthetic construct; identical to SEQ ID NO: 61
  REGION 1..251: note = Synthetic polypeptide
  REGION 1..251: note = misc_feature - IL2Rgamma_CISC

SEQ ID NO: 104 - protein, 358 aa, synthetic construct; identical to SEQ ID NO: 63
  REGION 1..358: note = Synthetic polypeptide
  REGION 1..358: note = misc_feature - IL2Ralpha_CISC

SEQ ID NO: 105 - protein, 358 aa, synthetic construct; identical to SEQ ID NO: 63
  REGION 1..358: note = Synthetic polypeptide
  REGION 1..358: note = misc_feature - IL7Ralpha_CISC

SEQ ID NO: 106 - protein, 276 aa, synthetic construct; identical to SEQ ID NO: 65
  REGION 1..276: note = Synthetic polypeptide
  REGION 1..276: note = misc_feature - MPL_CISC

SEQ ID NO: 107 - DNA, 10053 bp, synthetic construct; identical to SEQ ID NO: 67
  misc_feature 1..10053: note = Synthetic polynucleotide
  misc_feature 1..10053: note = expression vector
  source 1..10053: mol_type = other DNA

SEQ ID NO: 108 - DNA, 10035 bp, synthetic construct; identical to SEQ ID NO: 68
  misc_feature 1..10035: note = Synthetic polynucleotide
  misc_feature 1..10035: note = expression vector
  source 1..10035: mol_type = other DNA

SEQ ID NO: 109 - DNA, 9405 bp, synthetic construct; identical to SEQ ID NO: 66
  misc_feature 1..9405: note = Synthetic polynucleotide
  misc_feature 1..9405: note = expression vector
  source 1..9405: mol_type = other DNA

SEQ ID NO: 110 - DNA, 1293 bp, synthetic construct
  misc_feature 1..1293: note = Synthetic polynucleotide
  misc_feature 1..1293: note = Codon optimized human FOXP3 cDNA Without stop codon
  source 1..1293: mol_type = other DNA
atgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggacct

SEQ ID NO: 111 - DNA, 1296 bp, synthetic construct
  misc_feature 1..1296: note = Synthetic polynucleotide
  misc_feature 1..1296: note = Codon optimized human FOXP3 cDNA With stop codon
  source 1..1296: mol_type = other DNA
atgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggaccttga

SEQ ID NO: 112 - protein, 90 aa, synthetic construct; identical to SEQ ID NO: 37
  REGION 1..90: note = Synthetic polypeptide
  REGION 1..90: note = misc_feature - naked FRB domain

SEQ ID NO: 113 - protein, 90 aa, synthetic construct; identical to SEQ ID NO: 38
  REGION 1..90: note = Synthetic polypeptide
  REGION 1..90: note = misc_feature - naked FRB domain

SEQ ID NO: 114 - DNA, 1299 bp, synthetic construct
  misc_feature 1..1299: note = Synthetic polynucleotide
  misc_feature 1..1299: note = CISCbeta FRB_IL2Rbeta nucleotide sequence coding sequence only
  source 1..1299: mol_type = other DNA
atggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagccagcagctctcggcaaagacacgattccgtggcttgggcatctgctcgttgggctgagcggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcttgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccccccacccccggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgta

SEQ ID NO: 115 - protein, 433 aa, synthetic construct
  REGION 1..433: note = Synthetic polypeptide
  REGION 1..433: note = misc_feature - CISCbeta FRB_IL2Rbeta amino acid sequence
MALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 116 - DNA, 765 bp, synthetic construct
  misc_feature 1..765: note = Synthetic polynucleotide
  misc_feature 1..765: note = CISCgamma FKBP_IL2Rgamma nucleotide sequence coding sequence only codon optimized
  source 1..765: mol_type = other DNA
atgcctctgggcctgctgtggctgggcctggccctgctgggcgccctgcacgcccaggccggcgtgcaggtggagacaatctccccaggcgacggacgcacattccctaagcggggccagacctgcgtggtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggagggtcacctggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaact

SEQ ID NO: 117 - protein, 255 aa, synthetic construct
  REGION 1..255: note = Synthetic polypeptide
  REGION 1..255: note = misc_feature - CISCgamma FKBP_IL2Rgamma amino acid sequence
MPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGGSPGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 118 - DNA, 2463 bp, synthetic construct; identical to SEQ ID NO: 83
  misc_feature 1..2463: note = Synthetic polynucleotide
  misc_feature 1..2463: note = DISC CISC_FRB nucleotide sequence coding sequence only codon optimized
  source 1..2463: mol_type = other DNA

SEQ ID NO: 119 - protein, 821 aa, synthetic construct; identical to SEQ ID NO: 84
  REGION 1..821: note = Synthetic polypeptide
  REGION 1..821: note = misc_feature - DISC CISC_FRB uDISC amino acid sequence

SEQ ID NO: 120 - DNA, 267 bp, synthetic construct; identical to SEQ ID NO: 70
  misc_feature 1..267: note = Synthetic polynucleotide
  misc_feature 1..267: note = FRB express intracellularly to function as a decoy for rapamycin FRB nucleotide sequence coding sequence only decoy codon optimized WT
  source 1..267: mol_type = other DNA

SEQ ID NO: 121 - protein, 89 aa, synthetic construct
  REGION 1..89: note = Synthetic polypeptide
  REGION 1..89: note = misc_feature - FRB amino acid sequence
EMWHEGLEEASRLYFGERNVKGMFEVLEPLHAMMERGPQTLKETSFNQAYGRDLMEAQEWCRKYMKSGNVKDLTQAWDLYYHVFRRISK

SEQ ID NO: 122 - DNA, 825 bp, synthetic construct
  misc_feature 1..825: note = Synthetic polynucleotide
  misc_feature 1..825: note = LNGFR coding sequence with stop codon
  source 1..825: mol_type = other DNA
atgggggcaggtgccaccggacgagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtga

SEQ ID NO: 123 - DNA, 66 bp, synthetic construct
  misc_feature 1..66: note = Synthetic polynucleotide
  misc_feature 1..66: note = LNGFRe LNGFR epitope coding sequence 2A P2A self cleaving peptide
  source 1..66: mol_type = other DNA
ggaagcggagcgactaacttcagcctgctgaagcaggccggagatgtggaggaaaaccctggaccg

SEQ ID NO: 124 - DNA, 258 bp, synthetic construct
  misc_feature 1..258: note = Synthetic polynucleotide
  misc_feature 1..258: note = 0_25kb human FOXP3 5'HA designed for both TALEN and Cas9
  source 1..258: mol_type = other DNA
tgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctac

SEQ ID NO: 125 - DNA, 296 bp, synthetic construct
  misc_feature 1..296: note = Synthetic polynucleotide
  misc_feature 1..296: note = 0_3kb human FOXP3 5'HA for Cas9_T9
  source 1..296: mol_type = other DNA
catgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatcccc

SEQ ID NO: 126 - DNA, 452 bp, synthetic construct
  misc_feature 1..452: note = Synthetic polynucleotide
  misc_feature 1..452: note = 0_45kb human FOXP3 5'HA for Cas9_T9
  source 1..452: mol_type = other DNA
agcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatcccc

SEQ ID NO: 127 - DNA, 600 bp, synthetic construct
  misc_feature 1..600: note = Synthetic polynucleotide
  misc_feature 1..600: note = 0_6kb human FOXP3 5'HA for Cas9_T9
  source 1..600: mol_type = other DNA
atcacttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatcccc

SEQ ID NO: 128 - DNA, 785 bp, synthetic construct
  misc_feature 1..785: note = Synthetic polynucleotide
  misc_feature 1..785: note = 0_8kb human FOXP3 5'HA for Cas9_T9
  source 1..785: mol_type = other DNA
atctcaggtaatgtcagctcggtccttccagctgctcaagctaaaacccatgtcactttgactctccctcttgcccactacatccaagctgctagcactgctcctgatccagcttcagattaagtctcagaatctacccacttctcgccttctccactgccaccagcccattctgtgccagcatcatcacttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatcccc

SEQ ID NO: 129 - DNA, 275 bp, synthetic construct
  misc_feature 1..275: note = Synthetic polynucleotide
  misc_feature 1..275: note = 0_3kb human FOXP3 5'HA for Cas9_T3
  source 1..275: mol_type = other DNA
gacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatccccaggagcctcgcccagctggagggctgcacccaaagcctcagacctgctgggggcccggggcccagggggaaccttcca

SEQ ID NO: 130 - DNA, 449 bp, synthetic construct
  misc_feature 1..449: note = Synthetic polynucleotide
  misc_feature 1..449: note = 0_45kb human FOXP3 5'HA for Cas9_T3
  source 1..449: mol_type = other DNA
catagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatccccaggagcctcgcccagctggagggctgcacccaaagcctcagacctgctgggggcccggggcccagggggaaccttcca

SEQ ID NO: 131 - DNA, 600 bp, synthetic construct
  misc_feature 1..600: note = Synthetic polynucleotide
  misc_feature 1..600: note = 0_6kb human FOXP3 5'HA for Cas9_T3
  source 1..600: mol_type = other DNA
ctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcccaaccccaggcctggcaagccctcggccccttccttggcccttggcccatccccaggagcctcgcccagctggagggctgcacccaaagcctcagacctgctgggggcccggggcccagggggaaccttcca

SEQ ID NO: 132 - DNA, 245 bp, synthetic construct
  misc_feature 1..245: note = Synthetic polynucleotide
  misc_feature 1..245: note = 0_25kb human FOXP3 3'HA designed for both TALEN and Cas9
  source 1..245: mol_type = other DNA
gtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgt

SEQ ID NO: 133 - DNA, 300 bp, synthetic construct
  misc_feature 1..300: note = Synthetic polynucleotide
  misc_feature 1..300: note = 0_3kb human FOXP3 3'HA for Cas9_T9
  source 1..300: mol_type = other DNA
gcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgtt

SEQ ID NO: 134 - DNA, 450 bp, synthetic construct
  misc_feature 1..450: note = Synthetic polynucleotide
  misc_feature 1..450: note = 0_45kb human FOXP3 3'HA for Cas9_T9
  source 1..450: mol_type = other DNA
gcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagagga

SEQ ID NO: 135 - DNA, 600 bp, synthetic construct
  misc_feature 1..600: note = Synthetic polynucleotide
  misc_feature 1..600: note = 0_6kb human FOXP3 3'HA for Cas9_T9
  source 1..600: mol_type = other DNA
gcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgca

SEQ ID NO: 136 - DNA, 794 bp, synthetic construct
  misc_feature 1..794: note = Synthetic polynucleotide
  misc_feature 1..794: note = 0_8kb human FOXP3 3'HA for Cas9_T9
  source 1..794: mol_type = other DNA
gcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccacactgcccctagtcatggtggcaccctccggggcacggctgggccccttgccccacttacaggcactcctccaggacaggccacatttcatgcaccaggtatggacggtgaat

SEQ ID NO: 137 - DNA, 300 bp, synthetic construct
  misc_feature 1..300: note = Synthetic polynucleotide
  misc_feature 1..300: note = 0_3kb human FOXP3 3'HA for Cas9_T3
  source 1..300: mol_type = other DNA
cgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcagga

SEQ ID NO: 138 - DNA, 451 bp, synthetic construct
  misc_feature 1..451: note = Synthetic polynucleotide
  misc_feature 1..451: note = 0_45kb human FOXP3 3'HA for Cas9_T3
  source 1..451: mol_type = other DNA
cgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactc

SEQ ID NO: 139 - DNA, 600 bp, synthetic construct
  misc_feature 1..600: note = Synthetic polynucleotide
  misc_feature 1..600: note = 0_6kb human FOXP3 3'HA for Cas9_T3
  source 1..600: mol_type = other DNA
cgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgc

SEQ ID NO: 140 - DNA, 250 bp, synthetic construct
  misc_feature 1..250: note = Synthetic polynucleotide
  misc_feature 1..250: note = 0_25kb AAVS1 5'HA for Cas9_P1 and Cas9_N2
  source 1..250: mol_type = other DNA
tagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgt

SEQ ID NO: 141 - DNA, 600 bp, synthetic construct
  misc_feature 1..600: note = Synthetic polynucleotide
  misc_feature 1..600: note = 0_6kb AAVS1 5'HA for Cas9_P1 and Cas9_N2
  source 1..600: mol_type = other DNA
aggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgt

SEQ ID NO: 142 - DNA, 250 bp, synthetic construct
  misc_feature 1..250: note = Synthetic polynucleotide
  misc_feature 1..250: note = 0_25kb AAVS1 3'HA for Cas9_P1 and Cas9_N2
  source 1..250: mol_type = other DNA
ctctggttctgggtacttttatctgtcccctccaccccacagtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggaggga

SEQ ID NO: 143 - DNA, 600 bp, synthetic construct
  misc_feature 1..600: note = Synthetic polynucleotide
  misc_feature 1..600: note = 0_6kb AAVS1 3'HA for Cas9_P1 and Cas9_N2
  source 1..600: mol_type = other DNA
ctctggttctgggtacttttatctgtcccctccaccccacagtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccc

SEQ ID NO: 144 - protein, 274 aa, synthetic construct
  REGION 1..274: note = Synthetic polypeptide
  REGION 1..274: note = misc_feature - Selection marker LNGFR RQR8 EGFRt LNGFRt protein sequence
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 145 - protein, 157 aa, synthetic construct
  REGION 1..157: note = Synthetic polypeptide
  REGION 1..157: note = misc_feature - RQR8 protein sequence
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 146 - protein, 357 aa, synthetic construct
  REGION 1..357: note = Synthetic polypeptide
  REGION 1..357: note = misc_feature - EGFRt with GM_CSFR signal peptide
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 147 - DNA, 347 bp, synthetic construct
  misc_feature 1..347: note = Synthetic polynucleotide
  misc_feature 1..347: note = MND promoter
  source 1..347: mol_type = other DNA
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatc

SEQ ID NO: 148 - DNA, 523 bp, synthetic construct
  misc_feature 1..523: note = Synthetic polynucleotide
  misc_feature 1..523: note = PGK promoter
  source 1..523: mol_type = other DNA
ccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatcc

SEQ ID NO: 149 - DNA, 231 bp, synthetic construct
  misc_feature 1..231: note = Synthetic polynucleotide
  misc_feature 1..231: note = EF1 promoter
  source 1..231: mol_type = other DNA
aggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacaca

SEQ ID NO: 150 - DNA, 135 bp, synthetic construct
  misc_feature 1..135: note = Synthetic polynucleotide
  misc_feature 1..135: note = SV40 polyA
  source 1..135: mol_type = other DNA
tgctttatttgtgaaatttgtgatgctattgctttatttgtaaccattataagctgcaataaacaagttaacaacaacaattgcattcattttatgtttcaggttcagggggagatgtgggaggttttttaaagc

SEQ ID NO: 151 - DNA, 898 bp, synthetic construct
  misc_feature 1..898: note = Synthetic polynucleotide
  misc_feature 1..898: note = 3'UTR of FOXP3
  source 1..898: mol_type = other DNA
cctcaagatcaaggaaaggaggatggacgaacaggggccaaactggtgggaggcagaggtggtgggggcagggatgataggccctggatgtgcccacagggaccaagaagtgaggtttccactgtcttgcctgccagggcccctgttcccccgctggcagccaccccctcccccatcatatcctttgccccaaggctgctcagaggggccccggtcctggccccagcccccacctccgccccagacacaccccccagtcgagccctgcagccaaacagagccttcacaaccagccacacagagcctgcctcagctgctcgcacagattacttcagggctggaaaagtcacacagacacacaaaatgtcacaatcctgtccctcactcaacacaaaccccaaaacacagagagcctgcctcagtacactcaaacaacctcaaagctgcatcatcacacaatcacacacaagcacagccctgacaacccacacaccccaaggcacgcacccacagccagcctcagggcccacaggggcactgtcaacacaggggtgtgcccagaggcctacacagaagcagcgtcagtaccctcaggatctgaggtcccaacacgtgctcgctcacacacacggcctgttagaattcacctgtgtatctcacgcatatgcacacgcacagccccccagtgggtctcttgagtcccgtgcagacacacacagccacacacactgccttgccaaaaataccccgtgtctcccctgccactcacctcactcccattccctgagccctgatccatgcctcagcttagactgcagaggaactactcatttatttgggatccaaggcccccaacccacagtaccgtccccaataaactgcagccgagctccccaca

SEQ ID NO: 152 - DNA, 822 bp, synthetic construct
  misc_feature 1..822: note = Synthetic polynucleotide
  misc_feature 1..822: note = LNGFR coding sequence without stop codon
  source 1..822: mol_type = other DNA
atgggggcaggtgccaccggacgagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagagg

SEQ ID NO: 153 - DNA, 1899 bp, synthetic construct; identical to SEQ ID NO: 85
  misc_feature 1..1899: note = Synthetic polynucleotide
  misc_feature 1..1899: note = uDISC uCISC_FRB nucleotide sequence coding sequence only codon optimized
  source 1..1899: mol_type = other DNA

SEQ ID NO: 154 - protein, 633 aa, synthetic construct; identical to SEQ ID NO: 86
  REGION 1..633: note = Synthetic polypeptide
  REGION 1..633: note = misc_feature - uDISC uCISC_FRB amino acid sequence

SEQ ID NO: 155 - DNA, 19 bp, synthetic construct
  misc_feature 1..19: note = Synthetic polynucleotide
  misc_feature 1..19: note = NHEJ_F
  source 1..19: mol_type = other DNA
cacgtgtgactcctttccc

SEQ ID NO: 156 - DNA, 20 bp, synthetic construct
  misc_feature 1..20: note = Synthetic polynucleotide
  misc_feature 1..20: note = NHEJ_R
  source 1..20: mol_type = other DNA
cccagtgccacagtaaaggt

SEQ ID NO: 157 - DNA, 20 bp, synthetic construct
  misc_feature 1..20: note = Synthetic polynucleotide
  misc_feature 1..20: note = FAM_NHEJ probe
  source 1..20: mol_type = other DNA
agggccgagatcttcgaggc

SEQ ID NO: 158 - DNA, 20 bp, synthetic construct
  misc_feature 1..20: note = Synthetic polynucleotide
  misc_feature 1..20: note = Control_F
  source 1..20: mol_type = other DNA
cgacacttcaccccttttct

SEQ ID NO: 159 - DNA, 20 bp, synthetic construct
  misc_feature 1..20: note = Synthetic polynucleotide
  misc_feature 1..20: note = Control_R
  source 1..20: mol_type = other DNA
ctccccaatgtgcctatgag

SEQ ID NO: 160 - DNA, 20 bp, synthetic construct
  misc_feature 1..20: note = Synthetic polynucleotide
  misc_feature 1..20: note = HEX_Control probe
  source 1..20: mol_type = other DNA
gtggcggtgactgggatggc

SEQ ID NO: 161 - DNA, 7219 bp, synthetic construct
  misc_feature 1..7219: note = Synthetic polynucleotide
  misc_feature 1..7219: note = 3232_pAAV_FOXP3_0_8HA_ATG_FOXP3cDNA_WPRE3_pA_T3 specific
  source 1..7219: mol_type = other DNA
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcatttaatgccagactcttcatgtctatctacacctgcacttttgcacccaatccaactccccgccatgtcccccatctcaggtaatgtcagctcggtccttccagctgctcaagctaaaacccatgtcactttgactctccctcttgcccactacatccaagctgctagcactgctcctgatccagcttcagattaagtctcagaatctacccacttctcgccttctccactgccaccagcccattctgtgccagcatcatcacttgccaggactgttacaatagcctcctcactagccccactcacagcagccagatgaatcttttgagtccatgcctagtcactggggcaaaataggactccgaggagaaagtccgagaccagctccggcaagatgagcaaacacagcctgtgcagggtgcagggagggctagaggcctgaggcttgaaacagctctcaagtggagggggaaacaaccattgccctcatagaggacacatccacaccagggctgtgctagcgtgggcaggcaagccaggtgctggacctctgcacgtggggcatgtgtgggtatgtacatgtacctgtgttcttggtgtgtgtgtgtgtgtgtgtgtgtgtgtgtgtctagagctggggtgcaactatggggcccctcgggacatgtcccagccaatgcctgctttgaccagaggagtgtccacgtggctcaggtggtcgagtatctcataccgccctagcacacgtgtgactcctttcccctattgtctacgcagcctgcccttggacaaggacccgatgcctaatcctcggcctggaaagcctagcgctccttctcttgctctgggaccttctcctggcgcctctccatcttggagagccgctcctaaagccagcgatctgctgggagctagaggacctggcggcacatttcagggcagagatcttagaggcggagcccacgctagctcctccagccttaatcctatgcctcctagccagctccagctgcctacactgcctctggttatggtggctcctagcggagctagactgggccctctgcctcatctgcaagctctgctgcaggacagaccccacttcatgcaccagctgagcaccgtggatgcccacgcaagaacacctgtgctgcaggttcaccctctggaatccccagccatgatcagcctgacacctccaacaacagccaccggcgtgttcagcctgaaagccagacctggactgcctcctggcatcaatgtggccagcctggaatgggtgtccagagaacctgctctgctgtgcacattccccaatccaagcgctcccagaaaggacagcacactgtctgccgtgcctcagagcagctatcccctgcttgctaacggcgtgtgcaagtggcctggatgcgagaaggtgttcgaggaacccgaggacttcctgaagcactgccaggccgatcatctgctggacgagaaaggcagagcccagtgtctgctccagcgcgagatggtgcagtctctggaacagcagctggtcctggaaaaagaaaagctgagcgccatgcaggcccacctggccggaaaaatggccctgacaaaggccagcagcgtggcctcttctgataagggcagctgctgcattgtggccgctggatctcagggacctgtggttcctgcttggagcggacctagagaggcccctgattctctgtttgccgtgcggagacacctgtggggctctcacggcaactctactttccccgagttcctgcacaacatggactacttcaagttccacaacatgcggcctccattcacctacgccacactgatcagatgggccattctggaagcccctgagaagcagagaaccctgaacgagatctaccactggtttacccggatgttcgccttcttccggaatcaccctgccacctggaagaacgccatccggcacaatctgagcctgcacaagtgcttcgtgcgcgtggaatctgagaaaggcgccgtgtggacagtggacgagctggaattcagaaagaagagaagccagcggcctagccggtgcagcaatcctacacctggaccttgaaagcttgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtgggtcgactgctttatttgtgaaatttgtgatgctattgctttatttgtaaccattataagctgcaataaacaagttaacaacaacaattgcattcattttatgtttcaggttcagggggagatgtgggaggttttttaaagcactagtcgagatcttcgaggcggggcccatgcctcctcttcttccttgaaccccatgccaccatcgcagctgcaggtgaggccctgggcccaggatggggcaggcagggtggggtacctggacctacaggtgccgacctttactgtggcactgggcgggaggggggctggctggggcacaggaagtggtttctgggtcccaggcaagtctgtgacttatgcagatgttgcagggccaagaaaatccccacctgccaggcctcagagattggaggctctccccgacctcccaatccctgtctcaggagaggaggaggccgtattgtagtcccatgagcatagctatgtgtccccatccccatgtgacaagagaagaggactggggccaagtaggtgaggtgacagggctgaggccagctctgcaacttattagctgtttgatctttaaaaagttactcgatctccatgagcctcagtttccatacgtgtaaaagggggatgatcatagcatctaccatgtgggcttgcagtgcagagtatttgaattagacacagaacagtgaggatcaggatggcctctcacccacctgcctttctgcccagctgcccacactgcccctagtcatggtggcaccctccggggcacggctgggccccttgccccacttacaggcactcctccaggacaggccacatttcatgcaccaggtatggacggtgaatgggcagggaggagggagcaggtgggagaactgtggggaggggccccgagtcaggctgaaccggatcctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagacccc

(19) United States
(12) Patent Application Publication
(10) Pub. No.: US 2024/0117352 A1
(43) Pub. Date: Apr. 11, 2024

(54) EXPRESSION OF FOXP3 IN EDITED CD34+ CELLS

(71) Applicant: Seattle Children's Hospital (dba Seattle Children's Research Institute), Seattle, WA (US)

(72) Inventors: David J. Rawlings, Seattle, WA (US); Iram F. Khan, Seattle, WA (US); Yuchi Chiang Honaker, Seattle, WA (US); Swati Singh, Seattle, WA (US); Karen Sommer, Seattle, WA (US); Andrew M. Scharenberg, Seattle, WA (US)

(21) Appl. No.: 18/336,276

(22) Filed: Jun. 16, 2023

Related U.S. Application Data
(63) Continuation of application No. 16/981,223, filed on Sep. 15, 2020, now Pat. No. 11,713,459.
(60) Provisional application No. 62/663,545, filed on Apr. 27, 2018.

(51) Int. Cl. C12N 15/113; A61K 35/15; C12N 5/0783; C12N 9/22

(52) U.S. Cl. CPC C12N 15/113; A61K 35/15; C12N 5/0637; C12N 9/22; C12N 2310/20; C12N 2501/60; C12N 2800/80

(57) ABSTRACT

Aspects of the invention described herein concern the incorporation of a FOXP3 cDNA (e.g., full-length human codon-optimized cDNA) into a FOXP3 gene or a non-FOXP3 locus so as to provide constitutive or regulated FOXP3 expression in a primary human CD34⁺ cells or cells derived from edited CD34⁺ cells. In some embodiments, guide RNA sequences that are directed to FOXP3, AAVS1, or other candidate loci are used for CRISPR/Cas9-mediated gene regulation, and gene delivery cassettes for HDR based gene-modification are provided.

### CROSS REFERENCE TO RELATED APPLICATIONS

This application is a continuation of U.S. application Ser. No. 16/981,223 filed Sep. 15, 2020 which is the U.S. National Phase Application of PCT International Application Number PCT/US2019/029082, filed on Apr. 25, 2019, designating the United States of America and published in the English language, which is an International Application of and claims the benefit of priority to U.S. Provisional Application No. 62/663,545, filed on Apr. 27, 2018. The disclosures of the above-referenced applications are hereby expressly incorporated by reference in their entireties.

### REFERENCE TO SEQUENCE LISTING

The present application is being filed along with a Sequence Listing in electronic format. The Sequence Listing is provided as a file entitled SCRI188C1SEQLISTING, created Jun. 16, 2023, which is approximately 425491 bytes in size. The information in the electronic format of the Sequence Listing is incorporated herein by reference in its entirety.

### FIELD

Aspects of the invention described herein concern the incorporation of a FOXP3 coding sequence into a FOXP3 gene or a non-FOXP3 locus in CD34⁺ cells to provide constitutive or regulated FOXP3 expression in the edited CD34⁺ cells or cells derived therefrom, such as T cells.

### BACKGROUND

Lentiviral gene transfer of FOXP3 (also known as forkhead box protein P3, forkhead box P3, AAID, DIETER, IPEX, JM2, PIDX, XPID, or scurfin) has been previously described by Chen, C. et al. (2011). Transplant. Proc. 43(5):2031-2048, Passerini, L. et al. (2013). Sci. Transl. Med, 5(215):215ra174, and Passerini, L. et al. (2017). Front. Immunol. 8:1282; all are hereby expressly incorporated by reference in their entireties. Passerini et al. (2017) had previously reported the development of methods to restore Treg function in T lymphocytes from patients carrying mutations in FOXP3. As described by Passerini et al. (2017), lentiviral mediated gene transfer was used in CD4⁺ T cells and effector T cells which were converted into effector T cells, which exhibited characteristics of Treg-like cells and endowed the cells with potent in vitro and in vivo suppressive activity. Passerini also demonstrated conversion of CD4⁺ T cells into Treg cells after lentiviral mediated FOXP3 gene transfer, in which the cells were shown to be stable in inflammatory conditions Passerini et al. (2013). Chen et al. (2011) also describes the adoptive transfer of engineered T cells, in which the T cells were infected with a lentiviral vector encoding a FOXP3-IRES-GFP fragment. These cells were shown to protect recipients from GvHD in a murine model. The need for new approaches to express and regulate FOXP3 in a primary human lymphocytes is manifest.

Many investigators are interested in treating auto-immune diseases with regulatory T cells, due to the possibility for these cells to induce antigen specific tolerance. There are many forms of regulatory T cells (“T_{regs}”), with current nomenclature dividing T_{regs} into those which are generated in the thymus in the course of T cell development, denoted as thymic regulatory T cells or “tT_{regs}”, and peripherally induced regulatory T cells, denoted as peripheral regulatory T cells or “pT_{regs}.”

A key aspect of regulatory T cell biology is the expression of the transcription factor FOXP3. FOXP3 is thought to be required to specify the regulatory T cell lineage. This concept is based on the observation that humans who lack FOXP3 develop severe autoimmune disease starting in the neonatal period. The use of either tT_{regs} or pT_{regs} for therapy of autoimmune disease may not be optimal because FOXP3 expression is believed to be subject to epigenetic regulation. In tT_{regs}, an upstream region in the FOXP3 gene known as the “thymus specific demethylated region” is demethylated, a state which is thought to result in stable FOXP3 expression. Generally, full demethylation is not observed in pT_{regs}. Under inflammatory conditions, FOXP3 may be silenced epigenetically in pT_{regs}, and possibly tT_{regs} (although some investigators believe that tT_{regs} are completely stable), potentially resulting in conversion of pT_{regs} to pro-inflammatory CD4 T cells. The potential lack of stability of pT_{regs} is a significant concern, as infusion of pT_{regs} that revert to an inflammatory phenotype may result in a worsening of auto-immune symptoms.

### SUMMARY

Described herein is a system comprising: a deoxyribonucleic acid (DNA) endonuclease or nucleic acid encoding the DNA endonuclease; a guide RNA (gRNA) comprising a spacer sequence that is complementary to a sequence within a FOXP3 gene, AAVS1 locus, or a TRA gene in a CD34⁺ cell, or nucleic acid encoding the gRNA; and a donor template comprising a nucleic acid sequence encoding a FOXP3 or a functional derivative thereof. In some embodiments, the gRNA comprises: i) a spacer sequence from any one of SEQ ID NOs: 1-7, 15-20, and 27-29 or a variant thereof having no more than 3 mismatches compared to any one of SEQ ID NOs: 1-7, 15-20, and 27-29; ii) a spacer sequence from any one of SEQ ID NOs: 1-7 or a variant thereof having no more than 3 mismatches compared to any one of SEQ ID NOs: 1-7; or iii) a spacer sequence from any one of SEQ ID NOs: 2, 3, and 5 or a variant thereof having no more than 3 mismatches compared to any one of SEQ ID NOs: 2, 3, and 5. In some embodiments, the FOXP3 or functional derivative thereof is wild-type human FOXP3. In some embodiments, the DNA endonuclease is a Cas9. In some embodiments, the nucleic acid encoding the DNA endonuclease is an mRNA. In some embodiments, the donor template is encoded in an adeno-associated virus (AAV) vector. In some embodiments, the DNA endonuclease or nucleic acid encoding the DNA endonuclease is formulated in a liposome or lipid nanoparticle.

Also described herein is a method of editing a genome in a CD34⁺ cell, the method comprising providing any one of the systems described herein to the cell. In some embodiments, the CD34⁺ cell is not a germ cell.

The present disclosure also describes a genetically modified CD34⁺ cell, and a composition comprising a genetically modified CD34⁺ cell, in which the genome of the cell is edited by any one of the methods described herein. In some embodiments, the genetically modified CD34⁺ cell is not a germ cell.

Further described is a method of treating a disease or condition associated with FOXP3 in a subject, comprising providing any one of the systems described herein to a CD34⁺ cell in the subject. The disease or condition can be an inflammatory disease or an autoimmune disease, such as IPEX syndrome or Graft-versus-Host disease (GVHD). In some embodiments, the genetically modified CD34⁺ cell is not a germ cell.

### BRIEF DESCRIPTION OF THE DRAWINGS

FIG. 1 shows schematics for two different AAV donor template designs configured for integration of a donor cassette into a FOXP3 gene, one for expression of FOXP3 from a heterologous FOXP3 cDNA under the control of an endogenous FOXP3 promoter (top schematic), and the other for expression of GFP under a heterologous MND promoter (bottom schematic). HA: homology arms; MND: MND promoter; pA: SV40 polyadenylation signal.

FIG. 2 shows results for the viability of CD34⁺ cells treated with AAV donor template alone (#3037 or #3008), Cas9/gRNA RNPs (T3 gRNA or T9 gRNA)+AAV donor template (#3037 or #3008), or mock treated at day 1 (D1), day 2 (D2), or day 5 (D5) following treatment.

FIG. 3 shows the percent homologous recombination in CD34⁺ cells that have been edited using Cas9/gRNA RNPs (T3 gRNA or T9 gRNA) and the AAV donor templates shown in FIG. 1.

FIG. 4 is a bar graph showing the comparison of cell viabilities of CD34⁺ cells treated with RNPs containing Cas9 from two different sources (Alt-R S.p. Cas9 Nuclease V3 from IDT or SpyFi Cas9 from Aldevron) and two different gRNAs targeting FOXP3 (T3 or T9).

FIG. 5 is a bar graph showing the comparison of cell viabilities of CD34⁺ cells edited with RNPs containing Cas9 from two different sources (Alt-R S.p. Cas9 Nuclease V3 from IDT or SpyFi Cas9 from Aldevron) along with AAV donor templates and two different gRNAs targeting FOXP3 (T3 or T9).

FIG. 6 shows exemplary results for the percent GFP⁺ among total hCD45⁺ cells recovered from the spleens of NSGW41 mice engrafted with mock cells or cells edited by SpyFi Cas9/gRNA RNPs targeting FOXP3 (T3). Mean±SEM labeled on graph.

FIG. 7 shows exemplary results for the percent GFP⁺ cells among human CD19⁺ cells recovered from the spleens of NSGW41 mice engrafted with mock cells or cells edited by SpyFi Cas9/gRNA RNPs targeting FOXP3 (T3). Mean±SEM labeled on graph.

FIG. 8 shows exemplary results for the percent GFP⁺ cells among human CD33⁺ cells recovered from the spleens of NSGW41 mice engrafted with mock cells or cells edited by SpyFi Cas9/gRNA RNPs targeting FOXP3 (T3). Mean±SEM labeled on graph.

### DETAILED DESCRIPTION

Expression of FOXP3 from a DNA sequence (e.g., codon-optimized DNA sequence, such as for expression in human cells) that is integrated in a FOXP3 gene or a non-FOXP3 locus is described herein. Guide RNAs are used to target a FOXP3 gene (e.g., murine, human and nonhuman primate) or a non-FOXP3 locus for CRISPR/Cas-mediated genome editing. Accordingly, aspects of the invention concern the utilization of novel guide RNAs in combination with Cas proteins to create DNA breaks at a FOXP3 gene or non-FOXP3 loci to facilitate integration of a FOXP3 coding sequence. In some embodiments, the integration is by non-homologous end joining (NHEJ) or homology directed repair (HDR) in association with a donor template containing the FOXP3 coding sequence. Several embodiments described herein can be used in combination with a broad range of selection markers such as LNG FR, RQR8, CISC/DISC/uDISC or others and can be multiplexed with editing of other loci or co-expression of other gene products including cytokines.

As described in greater detail below, Applicant has identified guide RNAs, which in combination with Cas9 protein and novel AAV donor templates containing gene delivery cassettes, generate a high frequency of on-target cleavage and integration of the gene delivery cassette into a FOXP3 gene in primary human CD34⁺ cells. In addition, sustained engraftment of the edited CD34⁺ cells in NSG recipient mice was achieved, along with long-term expression of a GFP reporter construct integrated into a FOXP3 gene. These findings demonstrate that the genome editing systems such as the CRISPR/Cas systems described herein are capable of resulting in efficient editing to effect expression of a human wild-type FOXP3 in human hematopoietic stem cells and sustained engraftment at levels that are predicted to provide a clinical benefit in diseases or disorders having aberrant FOXP3 function, e.g., following autologous adoptive cell therapy in IPEX subjects. Previous studies suggested that IPEX subjects with as little as a 5% donor chimerism exhibit clinical benefit following allogeneic stem cell transplantation. See, Seidel, M. G. et al. (2009). Blood, 113(22):5689-5691.

The use of CRISPR/Cas systems including gRNAs and donor templates configured to insert the cDNA for a FOXP3 gene at an endogenous FOXP3 gene offers a promising therapy for inflammatory diseases, such as the autoimmune disease IPEX syndrome. In the context of treating IPEX syndrome, this disease can be caused by a diversity of mutations spread over the entire gene, and thus inserting the entire FOXP3 cDNA (e.g., human codon optimized) at the start codon may be desired. Utilizing the endogenous FOXP3 promoter upon cell differentiation from the CD34⁺ cell is expected to provide the necessary transcriptional signals required for optimal levels of FOXP3 expression.

### Definitions

As used herein, the terms “nucleic acid” and “nucleic acid molecule” include but are not limited to, for example, polynucleotides or oligonucleotides such as deoxyribonucleic acid (DNA) or ribonucleic acid (RNA), oligonucleotides, fragments generated by the polymerase chain reaction (PCR), and fragments generated by any of ligation, scission, endonuclease action, exonuclease action, and by synthetic generation. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, or azido groups, or sugars can be functionalized as ethers or esters. Moreover, the entire sugar moiety can be replaced with sterically and electronically similar structures, such as aza-sugars or carbocyclic sugar analogs. Examples of modifications in a base moiety include alkylated purines or pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded.

“Coding strand” as used herein includes but is not limited to, for example, the DNA strand which has the same base sequence as the RNA transcript produced (although with thymine replaced by uracil). It is this strand, which contains codons, while the non-coding strand contains anti-codons.

“Regulatory element” as used herein includes but is not limited to, for example, a segment of a nucleic acid molecule, which is capable of increasing or decreasing the expression of specific genes within an organism, e.g., one that has the ability to affect the transcription and/or translation of an operably linked transcribable DNA molecule. Regulatory elements such as promoters (e.g. an MND promoter), leaders, introns, or transcription termination regions are DNA molecules that have gene regulatory activity and play an integral part in the overall expression of genes in living cells. Isolated regulatory elements, such as promoters, that function in plants are therefore useful for modifying plant phenotypes through the methods of genetic engineering. Regulation of gene expression is an essential feature of all living organisms and viruses. Without being limiting, examples of regulatory elements can include, CAAT box, CCAAT box, Pribnow box, TATA box, SECIS element, mRNA polyadenylation signals, A-box, Z-box, C-box, E-box, G-box, hormone responsive elements, such as insulin gene regulatory sequences, DNA binding domains, activation domains, and/or enhancer domains.

In some embodiments, a guide RNA includes an additional segment at either the 5′ or 3′ end that provides for any of the features described above. For example, a suitable third segment can include a 5′ cap (e.g. a 7-methylguanylate cap (m7G)); a 3′ polyadenylated tail (e.g., a 3′ poly(A) tail); a riboswitch sequence (e.g. to allow for regulated stability and/or regulated accessibility by proteins and protein complexes); a stability control sequence; a sequence that forms a dsRNA duplex (e.g., a hairpin)); a sequence that targets the RNA to a subcellular location (e.g. nucleus, mitochondria, or chloroplasts, and the like); a modification or sequence that provides for tracking (e.g. direct conjugation to a fluorescent molecule, conjugation to a moiety that facilitates fluorescent detection, a sequence that allows for fluorescent detection, etc.); a modification or sequence that provides a binding site for proteins (e.g. proteins that act on DNA. including transcriptional activators, transcriptional repressors, DNA methyltransferases, DNA demethylases, histone acetyltransferases, or histone deacetylases, and the like); and combinations thereof.

A guide RNA and a Cas endonuclease (e.g., a Cas9 endonuclease) may form a ribonucleoprotein complex (e.g., bind via non-covalent interactions). The guide RNA provides target specificity to the complex by comprising a nucleotide sequence that is complementary to a sequence of a target DNA. The site-specific modifying enzyme of the complex provides the endonuclease activity. In other words, the site-specific modifying enzyme is guided to a target DNA sequence (e.g. a target sequence in a chromosomal nucleic acid; a target sequence in an extrachromosomal nucleic acid, e.g. an episomal nucleic acid, a minicircle, etc.; a target sequence in a mitochondrial nucleic acid; a target sequence in a chloroplast nucleic acid; or a target sequence in a plasmid; etc.) by virtue of its association with the protein-binding segment of the guide RNA.

“FOXP3” as used herein includes but is not limited to, for example, a protein that is involved in immune system responses. The FOXP3 gene (also known as forkhead box protein P3, forkhead box P3, AAID, DIETER, IPEX, JM2, PIDX, XPID, or scurfin) contains 11 coding exons. FOXP3 is a specific marker of natural T regulatory cells (nT_{regs}, a lineage of T cells) and adaptive/induced T regulatory cells (a/iT_{regs}). Induction or administration of FOXP3 positive T cells has, in animal studies, lead to marked reductions in (autoimmune) disease severity in models of diabetes, multiple sclerosis, asthma, inflammatory bowel disease, thyroiditis or renal disease. However, T cells have been able to show plasticity in studies. Thus, the use of regulatory T cells in therapy can be risky, as the T regulatory cell transferred to the subject may change into T helper 17 (Th17) cells, which are pro-inflammatory rather than regulatory cells. As such, methods are provided herein to avoid the risks that may arise from regulatory cells changing into pro-inflammatory cells. For example, FOXP3 expressed from an iTreg is used as a master regulator of the immune system and is used for tolerance and immune suppression. Treg are believed to play a critical role in multiple autoimmune diseases, such as IPEX syndrome, Type 1 diabetes, systemic lupus erythrematosus, and rheumatoid arthritis. Approaches to augment human Treg number or function are in current trials including low-dose IL-2 and adoptive transfer of autologous expanded Treg. The efficacy of IL-2 therapy is limited due to its pleotropic activity and potential “off target” effects that may increase inflammation. Adoptive Treg therapy is likely limited by in vivo stability and viability of expanded T_{regs} and their lack of relevant antigen specificity.

“Nuclease” as used herein includes but is not limited to, for example, a protein or an enzyme capable of cleaving the phosphodiester bonds between the nucleotide subunits of nucleic acids. The nuclease described herein, is used for “gene editing” which is a type of genetic engineering in which DNA is inserted, deleted or replaced in the genome of a living organism using a nuclease or an engineered nuclease or nucleases. Without being limiting, the nuclease can be of a CRISPR/Cas system (e.g., a CRISPR/Cas9 system), a zinc finger nuclease, or TALEN nuclease. The nuclease can be used to target a locus, e.g., a locus on a nucleic acid sequence.

“Coding exon” as used herein includes but is not limited to, for example, any part of a gene that will encode a part of the final mature RNA produced by that gene after introns have been removed by RNA splicing. The term exon refers to both the DNA sequence within a gene and to the corresponding sequence in RNA transcripts. In RNA splicing, introns are removed and exons are covalently joined to one another as part of generating the mature messenger RNA.

“Cas endonuclease” or “Cas nuclease” as used herein includes but is not limited to, for example, an RNA-guided DNA endonuclease enzyme associated with the CRISPR (Clustered Regularly Interspaced Short Palindromic Repeats) adaptive immunity system. Herein, “Cas endonuclease” refers to both naturally-occurring and recombinant Cas endonucleases.

“Cas9” or “CAS9” (also known as Csn1 and Csx12) as used herein includes but is not limited to, for example, an RNA-guided DNA endonuclease enzyme associated with the CRISPR (Clustered Regularly Interspaced Short Palindromic Repeats) adaptive immunity system. Herein, the “Cas9” refers to both naturally-occurring and recombinant Cas9s.

“Zinc finger nuclease” as used herein includes but is not limited to, for example, an artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes.

“TALEN” or “Transcription activator-like effector nuclease” as used herein includes but is not limited to, for example, restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind practically any desired DNA sequence, so when combined with a nuclease, DNA can be cut at specific locations. The restriction enzymes can be introduced into cells, for use in gene editing or for genome editing in situ, a technique known as genome editing with engineered nucleases. Alongside zinc finger nucleases and CRISPR/Cas, TALEN is a tool in the field of genome editing.

The term “knock-in” includes but is not limited to, for example, a genetic engineering method that involves the one-for-one substitution of DNA sequence information with a wild-type copy in a genetic locus or the insertion of sequence information not found within the locus.

A “promoter” as used herein includes but is not limited to, for example, nucleotide sequence that directs the transcription of a structural gene. In some embodiments, a promoter is located in the 5′ non-coding region of a gene, proximal to the transcriptional start site of a structural gene. Sequence elements within promoters that function in the initiation of transcription are often characterized by consensus nucleotide sequences. It is a region of DNA that initiates transcription of a particular gene. Promoters are located near the transcription start sites of genes, on the same strand and upstream on the DNA (towards the 5′ region of the sense strand). Promoters can be at or about 100, 200, 300, 400, 500, 600, 700, 800, or 1000 base pairs long or within a range defined by any two of the aforementioned lengths. As used herein, a promoter can be constitutively active, repressible or inducible. If a promoter is an inducible promoter, then the rate of transcription increases in response to an inducing agent. In contrast, the rate of transcription is not regulated by an inducing agent if the promoter is a constitutive promoter. Repressible promoters are also known. Without being limiting, examples of promoters can include a constitutive promoter, a heterologous weak promoter (e.g., a promoter that generates less expression than the endogenous promoter and/or a constitutive promoter) or inducible promoters. Examples can include EF1 alpha promoter, a PGK promoter, an MND promoter, KI promoter, Ki-67 gene promoter, or a promoter inducible by a drug such as tamoxifen and/or its metabolites. Commonly used constitutive promoters can include but are not limited to SV40, CMV, UBC, EF1A, PGK, or CAGG for mammalian systems.

“Transcriptional enhancer domain” as used herein includes but is not limited to, for example, a short (50-1500 bp) region of DNA that can be bound by proteins (activators) to increase or promote or enhance the likelihood that transcription of a particular gene will occur or the level of transcription that takes place. These activator proteins are usually referred to as transcription factors. Enhancers are generally cis-acting, located up to 1 Mbp (1,000,000 bp) away from the gene and can be upstream or downstream from the start site, and either in the forward or backward direction. An enhancer may be located upstream or downstream of the gene it regulates. A plurality of enhancer domains may be used In some embodiments, to generate greater transcription e.g., multimerized activation binding domains can be used to further enhance or increase the level of transcription. Furthermore, an enhancer doesn't need to be located near the transcription initiation site to affect transcription, as some have been found located in several hundred thousand base pairs upstream or downstream of the start site. Enhancers do not act on the promoter region itself, but are bound by activator proteins. These activator proteins interact with the mediator complex, which recruits polymerase II and the general transcription factors, which then begin transcribing the genes. Enhancers can also be found within introns. An enhancer's orientation may even be reversed without affecting its function. Additionally, an enhancer may be excised and inserted elsewhere in the chromosome, and still affect gene transcription. In some embodiments, the enhancers are used to silence the inhibition mechanisms that prevent transcription of the FOXP3 gene. An example of an enhancer binding domain is the TCR alpha enhancer. In some embodiments, the enhancer domain is a TCR alpha enhancer. In some embodiments, the enhancer binding domain is placed upstream from a promoter such that it activates the promoter to increase transcription of the protein. In some embodiments, the enhancer binding domain is placed upstream of a promoter to activate the promoter to increase transcription of the FOXP3 gene.

“Transcriptional activator domains” or “Transcriptional activation domain” as used herein include but are not limited to, for example, specific DNA sequences that can be bound by a transcription factor, in which the transcription factor can thereby control the rate of transcription of genetic information from DNA to messenger RNA. Specific transcription factors can include but is not limited to SP1, API, C/EBP, heat shock factor, ATF/CREB, c-Myc, Oct-1 or NF-1. In some embodiments, the activator domains are used to silence the inhibition mechanisms that prevent transcription of the FOXP3 gene.

“Ubiquitous chromatin opening element,” (UCOE) as used herein includes but is not limited to, for example, elements that are characterized by unmethylated CpG islands spanning dual, divergently transcribed promoters of housekeeping genes. The UCOE represent promising tools to avoid silencing and sustain transgene expression in a wide variety of cellular models including cell lines, multipotent hematopoietic stem cells, as well as PSCs and their differentiated progeny.

“Operably linked” as used herein includes but is not limited to, for example, functional linkage between a regulatory sequence and a heterologous nucleic acid sequence resulting in expression of the latter. In some embodiments, the first molecule is joined to a second molecule, wherein the molecules are so arranged that the first molecule affects the function of the second molecule. The two molecules may be part of a single contiguous molecule and may be adjacent. For example, a promoter is operably linked to a transcribable DNA molecule if the promoter modulates transcription of the transcribable DNA molecule of interest in a cell.

The term “concentration” used in the context of a molecule such as peptide fragment refers to an amount of molecule, e.g., the number of moles of the molecule, present in a given volume of solution.

The terms “individual,” “subject” and “host” are used interchangeably herein and refer to any subject for whom diagnosis, treatment, or therapy is desired. In some aspects, the subject is a mammal. In some aspects, the subject is a human being. In some aspects, the subject is a human patient. In some aspects, the subject can have or is suspected of having a disorder or health condition associated with FOXP3. In some aspects, the subject is a human who is diagnosed with a risk of disorder or health condition associated with FOXP3 at the time of diagnosis or later. In some cases, the diagnosis with a risk of disorder or health condition associated with FOXP3 can be determined based on the presence of one or more mutations in an endogenous gene encoding the FOXP3 or nearby genomic sequence that may affect the expression of a FOXP3. For example, in some aspects, the subject can have or is suspected of having an autoimmune disorder and/or has one or more symptoms of an autoimmune disorder. In some aspects, the subject is a human who is diagnosed with a risk of an autoimmune disorder at the time of diagnosis or later. In some cases, the diagnosis with a risk of an autoimmune disorder can be determined based on the presence of one or more mutations in an endogenous FOXP3 gene or genomic sequence near the FOXP3 gene in the genome that may affect the expression of the FOXP3 gene.

The term “treatment,” when used in referring to a disease or condition, means that at least an amelioration of the symptoms associated with the condition afflicting an individual is achieved, where amelioration is used in a broad sense to refer to at least a reduction in the magnitude of a parameter, e.g., a symptom, associated with the condition (e.g., an autoimmune disorder) being treated. As such, treatment also includes situations where the pathological condition, or at least symptoms associated therewith, are completely inhibited, e.g., prevented from happening, or eliminated entirely such that the host no longer suffers from the condition, or at least the symptoms that characterize the condition. Thus, treatment includes: (i) prevention, that is, reducing the risk of development of clinical symptoms, including causing the clinical symptoms not to develop, e.g., preventing disease progression; (ii) inhibition, that is, arresting the development or further development of clinical symptoms, e.g., mitigating or completely inhibiting an active disease.

The terms “effective amount,” “pharmaceutically effective amount,” or “therapeutically effective amount” as used herein mean a sufficient amount of the composition to provide the desired utility when administered to a subject having a particular condition. In the context of ex vivo treatment of an autoimmune disorder, the term “effective amount” refers to the amount of a population of therapeutic cells or their progeny needed to prevent or alleviate at least one or more signs or symptoms of an autoimmune disorder, and relates to a sufficient amount of a composition having the therapeutic cells or their progeny to provide the desired effect, e.g., to treat symptoms of an autoimmune disorder of a subject. The term “therapeutically effective amount” therefore refers to a number of therapeutic cells or a composition having therapeutic cells that is sufficient to promote a particular effect when administered to a subject in need of treatment, such as one who has or is at risk for an autoimmune disorder. An effective amount would also include an amount sufficient to prevent or delay the development of a symptom of the disease, alter the course of a symptom of the disease (for example but not limited to, slow the progression of a symptom of the disease), or reverse a symptom of the disease. In the context of in vivo treatment of an autoimmune disorder in a subject (e.g., a patient) or genome edition in a cell cultured in vitro, an effective amount refers to an amount of components used for genome edition such as gRNA, donor template and/or a site-directed polypeptide (e.g. DNA endonuclease) needed to edit the genome of the cell in the subject or the cell cultured in vitro. It is understood that for any given case, an appropriate “effective amount” can be determined by one of ordinary skill in the art using routine experimentation.

“Autoimmune disorder” as used herein includes but is not limited to, for example, abnormally low activity or over activity of the immune system. In cases of immune system over activity, the body attacks and damages its own tissues (autoimmune diseases). Immune deficiency diseases decrease the body's ability to fight invaders, causing vulnerability to infections. Without being limiting, examples of autoimmune disorders or autoimmune diseases, which can be inhibited, ameliorated or treated by using the compositions and methods described herein can include, for example, systemic lupus, scleroderma, hemolytic anemia, vasculitis, type I diabetes, Graves disease, rheumatoid arthritis, multiple sclerosis, Goodpasture's syndrome, myopathy, severe combined immunodeficiency, DiGeorge syndrome, Hyperimmunoglobulin E syndrome, Common variable immunodeficiency, Chronic granulomatous disease, Wiskott-Aldrich syndrome, Autoimmune lymphoproliferative syndrome, Hyper IgM syndrome, Leukocyte adhesion deficiency, NF-κB Essential Modifier (NEMO) Mutations, Selective immunoglobulin A deficiency, X-linked agammaglobulinemia, X-linked lymphoproliferative disease, IPEX, Immune dysregulation, polyendocrinopathy, enteropathy, immunodysregulation polyendocrinopathy enteropathy X-linked (IPEX) syndrome or Ataxia-telangiectasia. Immune disorders can be analyzed, for example, by examination of the profile of neural-specific autoantibodies or other biomarkers when detected in serum or cerebrospinal fluid in subjects. In some exemplary methods provided herein, the methods are for treatment, amelioration, or inhibition of autoimmune disorders. In some embodiments, the autoimmune disorder is systemic lupus, scleroderma, hemolytic anemia, vasculitis, type I diabetes, Graves disease, rheumatoid arthritis, multiple sclerosis, Goodpasture's syndrome, myopathy, severe combined immunodeficiency, DiGeorge syndrome, Hyperimmunoglobulin E syndrome, Common variable immunodeficiency, Chronic granulomatous disease, Wiskott-Aldrich syndrome, Autoimmune lymphoproliferative syndrome, Hyper IgM syndrome, Leukocyte adhesion deficiency, NF-κB Essential Modifier (NEMO) Mutations, Selective immunoglobulin A deficiency, X-linked agammaglobulinemia, X-linked lymphoproliferative disease, IPEX, Immune dysregulation, polyendocrinopathy, enteropathy, immunodysregulation polyendocrinopathy enteropathy X-linked (IPEX) syndrome or Ataxia-telangiectasia or any combination thereof.

“IPEX syndrome” refers to immunodysregulation polyendocrinopathy enteropathy X-linked syndrome and is a rare disease linked to the dysfunction of the transcription factor FOXP3, widely considered to be the master regulator of the regulatory T cell lineage. Subjects suffering from IPEX syndrome may have symptoms such as autoimmune enteropathy, psoriasiform or eczematous dermatitis, nail dystrophy, autoimmune endocrinopathies, or autoimmune skin conditions such as alopecia universalis or bullous pemphigoid. IPEX syndrome is an autoimmune disease in which the immune system attacks the body's own tissues and organs. The syndrome leads to loss of CD4⁺ CD25⁺ T regulatory cells, and loss of the expression of the transcription factor FOXP3. FOXP3 decrease is believed to be a consequence of unchecked T cell activation, which is secondary to loss of regulatory T cells.

“Organ transplantation” as used herein includes but is not limited to, for example, the moving of an organ from one body to another or from a donor site to another location on the person's own body, to replace the recipient's damaged or absent organ. Organs and/or tissues that are transplanted within the same person's body are called autografts. Transplants that are recently performed between two subjects of the same species are called allografts. Allografts can either be from a living or cadaveric source. In some embodiments described herein, a method of treating, inhibiting, or ameliorating side effects of organ transplantation in a subject, such as organ rejection is provided.

Organs that can be transplanted, for example, are the heart, kidneys, liver, lungs, pancreas, intestine, or thymus. Tissues for transplant can include, for example, bones, tendons (both referred to as musculoskeletal grafts), cornea, skin, heart valves, nerves or veins. Kidneys, liver or the heart are the most commonly transplanted organs. Cornea or musculoskeletal grafts are the most commonly transplanted tissues.

In some embodiments described herein, a method of treating, inhibiting, or ameliorating side effects of organ transplantation in a subject, such as organ rejection is provided. In some embodiments, the subject is also selected to receive anti-rejection medications. In some embodiments, the anti-rejection medications comprise Prednisone, Imuran (azathioprine), Collect (mycophenolate mofetil, or MMF), Myfortic (mycophenolic acid), Rapamune (sirolimus), Neoral (cyclosporine), or Prograf (tacrolimus).

In some embodiments, the subject is selected for inhibition, amelioration, or treatment with the engineered cells set forth in the embodiments herein. In some embodiments, the subject has side effects to anti-inflammatory drugs or anti-rejection drugs. As such, the selected subjects are provided with the exemplary cells or compositions provided herein. Side effects from anti-rejection drugs can include interactions with other medications that can raise or lower tacrolimus levels in the blood, kidney toxicity, high blood pressure, neurotoxicity (tremor, headache, tingling, and/or insomnia), Diabetes mellitis (high blood sugar), diarrhea, nausea, hair loss or high potassium or any combination thereof. As such, the subjects are selected for the methods of treatment, inhibition, or amelioration described herein. Such selection or identification can be made by clinical or diagnostic evaluation.

“Organ rejection” or “transplant rejection” as used herein includes but is not limited to, for example, transplanted tissue rejected by the recipient's immune system, which destroys the transplanted tissue.

“Graft-versus-Host disease” (GVHD or GvHD) as used herein includes but is not limited to, for example, a medical complication following the receipt of transplanted tissue from a genetically different person. GVHD is commonly associated with stem cell or bone marrow transplant but the term also applies to other forms of tissue graft. Immune cells in the donated tissue recognize the recipient as foreign and not “self” In some embodiments herein, the methods provided can be used for preventing or ameliorating the complications that can arise from GVHD.

“Pharmaceutical excipient” as used herein includes but is not limited to, for example, the inert substance that the cells in the composition are provided in.

A “chimeric antigen receptor” (CAR) described herein, also known as chimeric T cell receptor, includes but is not limited to, for example, an artificial T cell receptor or a genetically engineered receptor, which grafts a desired specificity onto an immune effector cell. A CAR may be a synthetically designed receptor comprising a ligand binding domain of an antibody or other protein sequence that binds to a molecule associated with the disease or disorder and is linked via a spacer domain to one or more intracellular signaling domains of a T cell or other receptors, such as a costimulatory domain. In some embodiments, a cell, such as a mammalian cell, is manufactured wherein the cell comprises a nucleic acid encoding a fusion protein and wherein the cell comprises a chimeric antigen receptor. These receptors can be used to graft the specificity of a monoclonal antibody or a binding portion thereof onto a T cell, for example. In some embodiments herein, the genetically engineered cell further comprises a sequence that encodes a chimeric antigen receptor. In some embodiments, the chimeric antigen receptor is specific for a molecule on a tumor cell. A chimeric antigen receptor or an engineered cell expressing a T cell receptor can be used to target a specific tissue in need for FOXP3. Some embodiments herein comprise methods for targeting specific tissues for providing and delivering a FOXP3. In some embodiments, the tissue is a transplanted tissue. In some embodiments, the chimeric antigen receptor is specific for a target molecule on the transplanted tissue.

As described herein, the genetically-engineered cells are engineered to express FOXP3, and as such, they are also described in the embodiments herein as “Treg-phenotype” cells. The cells can be CD34⁺ cells, e.g., CD34⁺ hematopoietic stem cells.

As used herein, “protein sequence” includes but is not limited to, for example, a polypeptide sequence of amino acids that is the primary structure of a protein. As used herein “upstream” refers to positions 5′ of a location on a polynucleotide, and positions toward the N-terminus of a location on a polypeptide. As used herein “downstream” refers to positions 3′ of a location on nucleotide, and positions toward the C-terminus of a location on a polypeptide. Thus, the term “N-terminal” refers to the position of an element or location on a polynucleotide toward the N-terminus of a location on a polypeptide.

As used herein, the term “expression,” or “protein expression” refers to the translation of a transcribed RNA molecule into a protein molecule. Protein expression may be characterized by its temporal, spatial, developmental, or morphological qualities, as well as, by quantitative or qualitative indications. In some embodiments, the protein or proteins are expressed such that the proteins are positioned for dimerization in the presence of a ligand.

The functional equivalent or fragment of the functional equivalent, in the context of a protein, may have one or more conservative amino acid substitutions. The term “conservative amino acid substitution” refers to substitution of an amino acid for another amino acid that has similar properties as the original amino acid. The groups of conservative amino acids are as follows:

| Group | Name of the amino acids |
|---|---|
| Aliphatic | Gly, Ala, Val, Leu, Ile |
| Hydroxyl or Sulfhydryl/ | Ser, Cys, Thr, Met |
| Selenium-containing | |
| Cyclic | Pro |
| Aromatic | Phe, Tyr, Trp |
| Basic | His, Lys, Arg |
| Acidic and their Amide | Asp, Glu, Asn, Gln |

Conservative substitutions may be introduced in any position of a predetermined peptide or fragment thereof. It may however also be desirable to introduce non-conservative substitutions, particularly, but not limited to, a non-conservative substitution in any one or more positions. A non-conservative substitution leading to the formation of a functionally equivalent fragment of the peptide would for example differ substantially in polarity, in electric charge, and/or in steric bulk while maintaining the functionality of the derivative or variant fragment.

“Percentage of sequence identity” is determined by comparing two optimally aligned sequences over a comparison window, wherein the portion of the polynucleotide or polypeptide sequence in the comparison window may have additions or deletions (i.e., gaps) as compared to the reference sequence (which does not have additions or deletions) for optimal alignment of the two sequences. In some cases, the percentage can be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity” in the context of two or more nucleic acid or polypeptide sequences, refer to two or more sequences or subsequences that are the same or have a specified percentage of amino acid residues or nucleotides that are the same (e.g., 60%, 65%, 70%, 75%, 80%, 85%, 90%, 95%, 98%, or 99% identity over a specified region, e.g., the entire polypeptide sequences or individual domains of the polypeptides), when compared and aligned for maximum correspondence over a comparison window or designated region as measured using one of the following sequence comparison algorithms or by manual alignment and visual inspection. Such sequences are then said to be “substantially identical.” This definition also refers to the complement of a test sequence.

The term “complementary” or “substantially complementary,” interchangeably used herein, means that a nucleic acid (e.g., DNA or RNA) has a sequence of nucleotides that enables it to non-covalently bind, i.e., form Watson-Crick base pairs or G/U base pairs, to another nucleic acid in a sequence-specific, antiparallel, manner (i.e., a nucleic acid specifically binds to a complementary nucleic acid). As is known in the art, standard Watson-Crick base-pairing includes: adenine (A) pairing with thymidine (T), adenine (A) pairing with uracil (U), and guanine (G) pairing with cytosine (C).

A DNA sequence that “encodes” a particular RNA is a DNA nucleic acid sequence that can be transcribed into RNA. A DNA polynucleotide may encode an RNA (mRNA) that is translated into protein, or a DNA polynucleotide may encode an RNA that is not translated into protein (e.g., tRNA, rRNA, or a guide RNA; also referred to herein as “non-coding” RNA or “ncRNA”). A “protein coding sequence or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences.

As used herein, “codon” refers to a sequence of three nucleotides that together form a unit of genetic code in a DNA or RNA molecule. As used herein the term “codon degeneracy” refers to the nature in the genetic code permitting variation of the nucleotide sequence without affecting the amino acid sequence of an encoded polypeptide.

The term “codon-optimized” or “codon optimization” refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA. Such optimization includes replacing at least one, or more than one, or a significant number, of codons with one or more codons that are more frequently used in the genes of that organism. Codon usage tables are readily available, for example, at the “Codon Usage Database”. By utilizing the knowledge on codon usage or codon preference in each organism, one of ordinary skill in the art can apply the frequencies to any given polypeptide sequence and produce a nucleic acid fragment of a codon-optimized coding region which encodes the polypeptide, but which uses codons optimal for a given species. Codon-optimized coding regions can be designed by various methods known to those skilled in the art.

The term “recombinant” or “engineered” when used with reference, for example, to a cell, a nucleic acid, a protein, or a vector, indicates that the cell, nucleic acid, protein, or vector has been modified by or is the result of laboratory methods. Thus, for example, recombinant or engineered proteins include proteins produced by laboratory methods. Recombinant or engineered proteins can include amino acid residues not found within the native (non-recombinant or wild-type) form of the protein or can be include amino acid residues that have been modified, e.g., labeled. The term can include any modifications to the peptide, protein, or nucleic acid sequence. Such modifications may include the following: any chemical modifications of the peptide, protein, or nucleic acid sequence, including of one or more amino acids, deoxyribonucleotides, or ribonucleotides; addition, deletion, or substitution of one or more of amino acids in the peptide or protein; or addition, deletion, or substitution of one or more of nucleic acids in the nucleic acid sequence.

The term “genomic DNA” or “genomic sequence” refers to the DNA of a genome of an organism including, but not limited to, the DNA of the genome of a bacterium, fungus, archaeon, plant, or animal.

As used herein, “transgene,” “exogenous gene” or “exogenous sequence,” in the context of nucleic acid, refers to a nucleic acid sequence or gene that was not present in the genome of a cell but artificially introduced into the genome, e.g., via genome-edition.

As used herein, “endogenous gene” or “endogenous sequence,” in the context of nucleic acid, refers to a nucleic acid sequence or gene that is naturally present in the genome of a cell, without being introduced via any artificial means.

“Vector,” “expression vector,” or “construct” is a nucleic acid used to introduce heterologous nucleic acids into a cell that has regulatory elements to provide expression of the heterologous nucleic acids in the cell. Vectors include but are not limited to plasmid, minicircles, yeast, and viral genomes. In some embodiments, the vectors are plasmid, minicircles, yeast, or viral genomes. In some embodiments, the vector is a viral vector. In some embodiments, the viral vector is a lentivirus. In some embodiments, the vector is an adeno-associated viral (AAV) vector. In some embodiments, the vector is for protein expression in a bacterial system such as E. coli. As used herein, the term “expression,” or “protein expression” refers to refers to the translation of a transcribed RNA molecule into a protein molecule. Protein expression may be characterized by its temporal, spatial, developmental, or morphological qualities as well as by quantitative or qualitative indications. In some embodiments, the protein or proteins are expressed such that the proteins are positioned for dimerization in the presence of a ligand. In some embodiments, the vector is a viral vector. In some embodiments, the viral vector is a lentivirus. In some embodiments, the vector is an adeno-associated viral (AAV) vector (such as, without limitation, AAV1, AAV2, AAV3, AAV4, AAV5, AAV6, AAV7, AAV8, AAV9, AAV10, or AAV11).

As used herein, “fusion proteins” or “chimeric proteins” include but are not limited to, for example, proteins created through the joining of two or more genes that originally coded for separate proteins or portions of proteins. The fusion proteins can also be made up of specific protein domains from two or more separate proteins. Translation of this fusion gene can result in a single or multiple polypeptides with functional properties derived from each of the original proteins. Recombinant fusion proteins can be created artificially by recombinant DNA technology for use in biological research or therapeutics. Such methods for creating fusion proteins are known to those skilled in the art. Some fusion proteins combine whole peptides and therefore can contain all domains, especially functional domains, of the original proteins. However, other fusion proteins, especially those that are non-naturally occurring, combine only portions of coding sequences and therefore do not maintain the original functions of the parental genes that formed them. In some embodiments, a fusion protein is provided, wherein the fusion protein comprises an interferon or a PD-1 protein or both.

A “conditional” or “inducible” promoter as used herein includes but is not limited to, for example, a nucleic acid construct that comprises a promoter that provides for gene expression in the presence of an inducer and does not substantially provide for gene expression in the absence of the inducer.

“Constitutive” as used herein refer to the nucleic acid construct that comprises a promoter that is constitutive, and thus provides for expression of a polypeptide that is continuously produced.

In some embodiments, the inducible promoter has a low level of basal activity. In some embodiments, wherein a lentiviral vector is used, the level of basal activity in uninduced cells is 20%, 15%, 10%, 5%, 4%, 3%, 2%, 1% or less (but not zero) or within a range defined by any two of the aforementioned values, as compared to when cells are induced to express the gene. The level of basal activity can be determined by measuring the amount of the expression of the transgene (e.g. marker gene) in the absence of the inducer (e.g. drug) using flow cytometry. In some embodiments described herein a marker protein such as Akt is used for determination of expression.

In some embodiments, the inducible promoter provides for a high level of induced activity, as compared to uninduced or basal activity. In some embodiments, the level of activity in the induced state is 2, 4, 6, 8, 9 or 10 fold or greater than the activity level in the uninduced state or within a range defined by any two of the aforementioned values. In some embodiments, transgene expression under control of the inducible promoter is turned off in the absence of a transactivator in less than 10, 8, 6, 4, 2, or 1 days excluding 0 days or within a range defined by any two of the aforementioned time periods.

In some embodiments, an inducible promoter is designed or modified to provide for a low level of basal activity, a high level of inducibility, and/or a short time for reversibility.

“Woodchuck Hepatitis Virus (WHP) Posttranscriptional Regulatory Element (WPRE) is a DNA sequence that, when transcribed creates a tertiary structure enhancing expression. These elements may be used to increase expression of genes delivered by viral vectors. In the embodiments described herein, the WPRE3 element is used to enhance the expression of the delivered nucleic acid, such as delivered cDNA.

In some embodiments, the immunomodulatory imide drug used in the approaches described herein may comprise: thalidomide (including analogues, derivatives, or pharmaceutically acceptable salts thereof. Thalidomide may include Immunoprin, Thalomid, Talidex, Talizer, Neurosedyn, α-(N-Phthalimido)glutarimide, 2-(2,6-dioxopiperidin-3-yl)-2,3-dihydro-1H-isoindole-1,3-dione); or pomalidomide (including analogues, derivatives, or pharmaceutically acceptable salts thereof. Pomalidomide may include Pomalyst, Imnovid, (RS)-4-Amino-2-(2,6-dioxopiperidin-3-yl)isoindole-1,3-dione); or lenalidomide (including analogues, derivatives, or pharmaceutically acceptable salts thereof. Lenalidomide may include Revlimid, (RS)-3-(4-Amino-1-oxo-1,3-dihydro-2H-isoindol-2-yl)piperidine-2,6-dione); or apremilast (including analogues, derivatives, or pharmaceutically acceptable salts thereof. Apremilast may include Otezla, CC-10004, N-{2-[(1S)-1-(3-Ethoxy-4-methoxyphenyl)-2-(methylsulfonyl) ethyl]-1,3-dioxo-2,3-dihydro-1H-isoindol-4-yl}acetamide); or any combinations thereof.

As used herein, the term “extracellular binding domain” refers to a domain of a complex that is outside of the cell, and which is configured to bind to a specific atom or molecule. In some embodiments, the extracellular binding domain of a CISC is a FKBP domain or a portion thereof. In some embodiments, the extracellular binding domain is an FRB domain or a portion thereof. In some embodiments, the extracellular binding domain is configured to bind a ligand or agent, thereby stimulating dimerization of two CISC components. In some embodiments, the extracellular binding domain is configured to bind to a cytokine receptor modulator.

The CISC (chemically induced signaling complex) is a multicomponent synthetic protein complex configured for co-expression in a host cell as two chimeric proteins as described in International Patent Application No. PCT/US2017/065746, the disclosure of which is expressly incorporated by reference herein in its entirety. Each chimeric protein component of the CISC has one half of a rapamycin binding complex as an extracellular domain, fused to one half of an intracellular signaling complex. Delivery of nucleic acids encoding the CISC to host cells permits intracellular signaling in the cells that can be controlled by the presence of rapamycin or a rapamycin-related chemical compound.

Rapamycin-driven CISC dimerization can trigger intracellular signaling, the presence of rapamycin can also inhibit the growth and the viability of host cells, thereby limiting their utility for use in therapeutic, as well as, research endeavors. Consequently, new compositions and methods are needed, which permit the use of rapamycin-mediated CISC intracellular signaling but which remediate the negative effects that rapamycin or rapamycin-related compounds have on the growth and viability of host cells.

“Dimeric chemical-induced signaling complex,” “dimeric CISC,” or “dimer” as used herein refers to two components of a CISC, which may or may not be fusion protein complexes that join together. “Dimerization” refers to the process of the joining together of two separate entities into a single entity, for example in response to binding of the entities to a ligand (for example, rapamycin). In some embodiments, a ligand or agent stimulates dimerization. In some embodiments, dimerization refers to homodimerization, or the joining of two identical entities, such as two identical CISC components. In some embodiments, dimerization refers to heterodimerization, of the joining of two different entities, such as two different and distinct CISC components. In some embodiments, the dimerization of the CISC components results in a cellular signaling pathway. In some embodiments, the dimerization of the CISC components allows for the selective expansion of a cell or a population of cells. Additional CISC systems can include a CISC gibberellin CISC dimerization system, or a SLF-TMP CISC dimerization system. Other chemically inducible dimerization (CID) systems and component parts may be used.

As used herein, “chemical-induced signaling complex” or “CISC” refers to an engineered complex that initiates a signal into the interior of a cell as a direct outcome of ligand-induced dimerization. A CISC may be a homodimer (dimerization of two identical components) or a heterodimer (dimerization of two distinct components). Thus, as used herein the term “homodimer” refers to a dimer of two protein components described herein with identical amino acid sequences. The term “heterodimer” refers to a dimer of two protein components described herein with non-identical amino acid sequences.

The CISC may be a synthetic complex as described herein in greater detail. “Synthetic” as used herein refers to a complex, protein, dimer, or composition, as described herein, which is not natural, or that is not found in nature. In some embodiments, an IL2R-CISC refers to a signaling complex that involves interleukin-2 receptor components. In some embodiments, an IL2/15-CISC refers to a signaling complex that involves receptor signaling subunits that are shared by interleukin-2 and interleukin-15. In some embodiments, an IL7-CISC refers to a signaling complex that involves an interleukin-7 receptor components. A CISC may thus be termed according to the component parts that make up the components of a given CISC. One of skill in the art will recognize that the component parts of the chemical-induced signaling complex may be composed of a natural or a synthetic component useful for incorporation into a CISC. Thus, the examples provided herein are not intended to be limiting.

“FKBP” as used herein, is a FK506 binding protein domain. FKBP refers to a family of proteins that have prolyl isomerase activity and are related to the cyclophilins in function, though not in amino acid sequence. FKBPs have been identified in many eukaryotes from yeast to humans and function as protein folding chaperones for proteins containing proline residues. Along with cyclophilin, FKBPs belong to the immunophilin family. The term FKBP comprises, for example, FKBP12 as well as, proteins encoded by the genes AIP; AIPL1; FKBP1A; FKBP1B; FKBP2; FKBP3; FKBP5; FKBP6; FKBP7; FKBP8; FKBP9; FKBP9L; FKBP10; FKBP11; FKBP14; FKBP15; FKBP52; or LOC541473; comprising homologs thereof and functional protein fragments thereof.

“FRB” as used herein, as a FKBP rapamycin binding domain. FRB domains are polypeptide regions (protein “domains”) that are configured to form a tripartite complex with an FKBP protein and rapamycin or a rapalog thereof. FRB domains are present in a number of naturally occurring proteins, comprising mTOR proteins (also referred to in the literature as FRAP, RAPT 1, or RAFT) from human and other species; yeast proteins comprising Tor1 or Tor2; or a Candida FRAP homolog. Both FKBP and FRB are major constituents in the mammalian target of rapamycin (mTOR) signaling.

A “naked FKBP rapamycin binding domain polypeptide” or a “naked FRB domain polypeptide” (which can also be referred to as an “FKBP rapamycin binding domain polypeptide” or an “FRB domain polypeptide”) refers to a polypeptide comprising only the amino acids of an FRB domain or a protein wherein at or about 90%, 91%, 92%, 93%, 94%, 95%, 96%, 97%, 98%, 99%, or 100% of the amino acids of the protein are amino acids of an FRB domain. The FRB domain can be expressed as a 12 kDa soluble protein (Chen, J. et al. (1995). Proc. Natl. Acad. Sci. U.S.A., 92(11):4947-4951). The FRB domain forms a four helix bundle, a common structural motif in globular proteins. Its overall dimensions are 30 Å by 45 Å by 30 Å, and all four helices) have short underhand connections similar to the cytochrome b562 fold (Choi, J. et al. (1996). Science, 273(5272):239-242). In some embodiments, the naked FRB domain comprises the amino acids of

SEQ ID NO: 37:
(MEMWHEGLEEASRLYFGERNVKGMFEVLEPLHAMMERGPQTLKETSFNQ
AYGRDLMEAQEWCRKYMKSGNVKDLTQAWDLYYHVFRRISK; SEQ ID
NO: 37),
or
SEQ ID NO: 38:
(MEMWHEGLEEASRLYFGERNVKGMFEVLEPLHAMMERGPQTLKETSFNQ
AYGRDLMEAQEWCRKYMKSGNVKDLLQAWDLYYHVFRRISK; SEQ ID
NO: 38).

As used herein, the term “activate” refers to an increase in at least one biological activity of a protein of interest. Similarly, the term “activation” refers to a state of a protein of interest being in a state of increased activity. The term “activatable” refers to the ability of a protein of interest to become activated in the presence of a signal, an agent, a ligand, a compound, or a stimulus. In some embodiments, a dimer, as described herein, is activated in the presence of a signal, an agent, a ligand, a compound, or a stimulus, and becomes a signaling competent dimer. As used herein, the term “signaling competent” refers to the ability or configuration of the dimer so as to be capable of initiating or sustaining a downstream signaling pathway.

As used herein, the term “signaling domain” refers to a domain of the fusion protein or CISC component that is involved in a signaling cascade inside the cell, such as a mammalian cell. A signaling domain refers to a signaling moiety that provides to cells, such as T cells, a signal which, in addition to the primary signal provided by for instance the CD3 zeta chain of the TCR/CD3 complex, mediates a cellular response, such as a T cell response, comprising, but not limited to, activation, proliferation, differentiation, or cytokine secretion or any combination thereof. In some embodiments, the signaling domain is N-terminal to the transmembrane domain, the hinge domain, and the extracellular domain. In some embodiments, the signaling domain is a synthetic or a natural domain. In some embodiments, the signaling domain is a concatenated cytoplasmic signaling domain. In some embodiments, the signaling domain is a cytokine signaling domain. In some embodiments, the signaling domain is an antigen signaling domain. In some embodiments, the signaling domain is an interleukin-2 receptor subunit gamma (IL2Rγ or IL2Rg) domain. In some embodiments, the signaling domain is an interleukin-2 receptor subunit beta (IL2Rβ or IL2Rb) domain or a truncated IL2Rβ domain (such as the truncated IL2Rβ domain comprising the amino acid sequence of SEQ ID NO:5). In some embodiments, binding of an agent or ligand to the extracellular binding domain causes a signal transduction through the signaling domain by the activation of a signaling pathway, as a result of dimerization of the CISC components. As used herein, the term “signal transduction” refers to the activation of a signaling pathway by a ligand or an agent binding to the extracellular domain. Activation of a signal is a result of the binding of the extracellular domain to the ligand or agent, resulting in CISC dimerization.

As used herein, the term “IL2Rb” or “IL2Rβ” refers to an interleukin-2 receptor subunit beta. Similarly, the term “IL2Rg” or IL2Rγ” refers to an interleukin-2 receptor subunit gamma, and the term “IL2Ra” or “IL2Rα” refers to an interleukin-2 receptor subunit alpha. The IL-2 receptor has three forms, or chains, alpha, beta, and gamma, which are also subunits for receptors for other cytokines. IL2Rβ and IL2Rγ are members of the type I cytokine receptor family. “IL2R” as used herein refers to interleukin-2 receptor, which is involved in T cell-mediated immune responses. IL2R is involved in receptor-mediated endocytosis and transduction of mitogenic signals from interleukin 2. Similarly, the term

“IL-2/15R” refers to a receptor signaling subunit that is shared by IL-2 and IL-15, and may include a subunit alpha (IL2/15Ra or IL2/15Ra), beta (IL2/15Rb or IL2/15RO, or gamma (IL2/15Rg or IL2/15Ry).

In some embodiments, a chemical-induced signaling complex is a heterodimerization activated signaling complex comprising two components. In some embodiments, the first component comprises an extracellular binding domain that is one part of a heterodimerization pair, an optional hinge domain, a transmembrane domain, and one or more concatenated cytoplasmic signaling domains. In some embodiments, the second component comprises an extracellular binding domain that is the other part of a heterodimizeration pair, an optional hinge domain, a transmembrane domain, and one or more concatenated cytoplasmic signaling domains. Thus, in some embodiments, there are two distinct modification events. In some embodiments, the two CISC components are expressed in a cell, such as a mammalian cell. In some embodiments, the cell, such as a mammalian cell, or a population of cells, such as a population of mammalian cells, is contacted with a ligand or agent that causes heterodimerization, thereby initiating a signal. In some embodiments, a homodimerization pair dimerize, whereby a single CISC component is expressed in a cell, such as a mammalian cell, and the CISC components homodimerize to initiate a signal.

As used herein, the term “selective expansion” refers to an ability of a desired cell, such as a mammalian cell, or a desired population of cells, such as a population of mammalian cells, to expand. In some embodiments, selective expansion refers to the generation or expansion of a pure population of cells, such as mammalian cells, that have undergone two genetic modification events. One component of a dimerization CISC is part of one modification and the other component is the other modification. Thus, one component of the heterodimerizing CISC is associated with each genetic modification. Exposure of the cells to a ligand allows for selective expansion of only the cells, such as mammalian cells, having both desired modifications. Thus, in some embodiments, the only cells, such as mammalian cells, that will be able to respond to contact with a ligand are those that express both components of the heterodimerization CISC.

As used herein, the term “cytokine receptor modulator” refers to an agent, which modulates the phosphorylation of a downstream target of a cytokine receptor, the activation of a signal transduction pathway associated with a cytokine receptor, and/or the expression of a particular protein such as a cytokine. Such an agent may directly or indirectly modulate the phosphorylation of a downstream target of a cytokine receptor, the activation of a signal transduction pathway associated with a cytokine receptor, and/or the expression of a particular protein such as a cytokine. Thus, examples of cytokine receptor modulators include, but are not limited to, cytokines, fragments of cytokines, fusion proteins or antibodies or binding portions thereof that immunospecifically bind to a cytokine receptor or a fragment thereof. Further, examples of cytokine receptor modulators include, but are not limited to, peptides, polypeptides (e.g., soluble cytokine receptors), fusion proteins or antibodies or binding portions thereof that immunospecifically bind to a cytokine or a fragment thereof.

As used herein, the term “hinge domain” refers to a domain that links the extracellular binding domain to the transmembrane domain, and may confer flexibility to the extracellular binding domain. In some embodiments, the hinge domain positions the extracellular domain close to the plasma membrane to minimize the potential for recognition by antibodies or binding fragments thereof. In some embodiments, the extracellular binding domain is located N-terminal to the hinge domain. In some embodiments, the hinge domain may be natural or synthetic.

As used herein, the term “transmembrane domain” or “TM domain” refers to a domain that is stable in a membrane, such as in a cell membrane. The terms “transmembrane span,” “integral protein,” and “integral domain” are also used herein. In some embodiments, the hinge domain and the extracellular domain is located N-terminal to the transmembrane domain. In some embodiments, the transmembrane domain is a natural or a synthetic domain. In some embodiments, the transmembrane domain is an IL-2 transmembrane domain.

As used herein, “host cell” comprises any cell type, such as a mammalian cell, that is susceptible to transformation, transfection, or transduction, with a nucleic acid construct or vector. In some embodiments, the host cell, such as a mammalian cell, is a T cell or a T regulatory cell (abbreviated herein as “Treg” or “T_{reg}”). In some embodiments, the host cell, such as a mammalian cell, is a hematopoietic stem cell. In some embodiments, the host cell is a CD34⁺ cell, e.g., a CD34⁺ hematopoietic stem cell. As used herein, the term “population of cells” refers to a group of cells, such as mammalian cells, comprising more than one cell. In some embodiments, a cell, such as a mammalian cell, is manufactured, wherein the cell comprises the protein sequence as described herein or an expression vector that encodes the protein sequence as described herein.

As used herein, the term “transformed” or “transfected” refers to a cell, such as a mammalian cell, tissue, organ, or organism into which a foreign polynucleotide molecule, such as a construct, has been introduced. The introduced polynucleotide molecule may be integrated into the genomic DNA of the recipient cell, such as a mammalian cell, tissue, organ, or organism such that the introduced polynucleotide molecule is inherited by subsequent progeny. A “transgenic” or “transfected” cell, such as a mammalian cell, or organism also comprises progeny of the cell or organism and progeny produced from a breeding program employing such a transgenic organism as a parent in a cross and exhibiting an altered phenotype resulting from the presence of a foreign polynucleotide molecule. The term “transgenic” refers to a bacteria, fungi, or plant containing one or more heterologous polynucleic acid molecules. “Transduction” refers to virus-mediated gene transfer into cells, such as mammalian cells.

As used herein, a “mammal” comprises, without limitation, mice, rats, rabbits, guinea pigs, dogs, cats, sheep, goats, cows, horses, primates, such as monkeys, chimpanzees, or apes, and, in particular, humans. In some embodiments, the subject is human.

A “marker sequence,” as described herein, encodes a protein that is used for selecting or tracking a protein or cell, such as a mammalian cell, that has a protein of interest. In the embodiments described herein, the fusion protein provided can comprise a marker sequence that can be selected in experiments, such as flow cytometry.

“Epitope” as used herein, refers to a part of an antigen or molecule that is recognized by the immune system comprising antibodies, T cells, or B-cells. Epitopes usually have at least 7 amino acids and can be a linear or a conformational epitope. In some embodiments, a cell, such as a mammalian cell, expressing a fusion protein is provided, wherein the cell further comprises a chimeric antigen receptor. In some embodiments, the chimeric antigen receptor comprises a scFv that can recognize an epitope on a cancer cell. “Isolating,” or “purifying” when used to describe the various polypeptides or nucleic acids disclosed herein, refers to a polypeptide or nucleic acid that has been identified and separated and/or recovered from a component of its natural environment. In some embodiments, the isolated polypeptide or nucleic acid is free of association with all components with which it is naturally associated. Contaminant components of its natural environment are materials that would generally interfere with diagnostic or therapeutic uses for the polypeptide or nucleic acid, and can include enzymes, hormones, or other proteinaceous or non-proteinaceous solutes. In some embodiments, a method is provided wherein the method comprises delivering the nucleic acid of any one of the embodiments described herein or the expression vector of any one of the embodiments described herein to a bacterial cell, mammalian cell or insect cell, growing the cell up in a culture, inducing expression of the fusion protein and purifying the fusion protein for treatment.

“Percent (%) amino acid sequence identity” with respect to the CISC sequences identified herein is defined as the percentage of amino acid residues in a candidate sequence that are identical with the amino acid residues in the reference sequence for each of the extracellular binding domain, hinge domain, transmembrane domain, and/or the signaling domain, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, comprising any algorithms needed to achieve maximal alignment over the full-length of the sequences being compared. For example, % amino acid sequence identity values generated using the WU-BLAST-2 computer program (Altschul, S. F. et al. (1996). Methods Enzymol., 266:460-480) uses several search parameters, most of which are set to the default values. Those that are not set to default values (e.g., the adjustable parameters) are set with the following values: overlap span=1, overlap fraction=0.125, word threshold (T)=11 and scoring matrix=BLOSUM62. In some embodiments of the CISC, the CISC comprises an extracellular binding domain, a hinge domain, a transmembrane domain, and a signaling domain, wherein each domain comprises a natural, synthetic, or a mutated or truncated form of the native domain (such as a truncated interleukin 2 receptor beta signaling domain). In some embodiments, a mutated or truncated form of any given domain comprises an amino acid sequence with 100%, 95%, 90%, 85% sequence identity, or a percent sequence identity that is within a range defined by any two of the aforementioned percentages to a sequence set forth in a sequence provided herein.

“T cells” or “T lymphocytes” as used herein can be from any mammalian, e.g., primate, species, comprising monkeys, dogs, and humans. In some embodiments, the T cells are allogeneic (from the same species but different donor) as the recipient subject; In some embodiments the T cells are autologous (the donor and the recipient are the same); In some embodiments, the T cells are syngeneic (the donor and the recipients are different but are identical twins).

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “comprising at least.” When used in the context of a process, the term “comprising” means that the process comprises at least the recited steps, but may include additional steps. When used in the context of a compound, composition or device, the term “comprising” means that the compound, composition or device comprises at least the recited features or components, but may also include additional features or components.

### Genome Editing Systems

Provided herein are systems for genome editing in a cell, e.g., a CD34⁺ cell, to modulate the expression, function, or activity of a FOXP3, such as by targeted integration of a nucleic acid encoding a FOXP3 or a functional derivative thereof into the genome of the cell. The disclosures also provide, inter alia, systems for providing a therapy to a subject having or suspected of having a disorder or health condition associated with FOXP3, employing ex vivo and/or in vivo genome editing. In some embodiments, the subject has or is suspected of having an autoimmune disease (e.g., IPEX syndrome) or a disorder that results from organ transplant (e.g., Graft-versus-Host Disease (GVHD)).

Some embodiments relate to a system comprising (a) a DNA endonuclease or nucleic acid encoding the DNA endonuclease; (b) a gRNA (e.g., an sgRNA) or nucleic acid encoding the gRNA, wherein the gRNA is capable of targeting the DNA endonuclease to a FOXP3 gene or a non-FOXP3 locus (e.g., AAVS1 (i.e., adeno-associated virus integration site in the genome of a cell)), and (c) a donor template comprising a FOXP3 coding sequence. In some embodiments, the DNA endonuclease is selected from the group consisting of a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 endonuclease, or a functional derivative thereof. In some embodiments, the DNA endonuclease is a Cas endonuclease, such as a Cas9 endonuclease (e.g., a Cas9 endonuclease from Streptococcus pyogenes). In some embodiments, the gRNA comprises a spacer sequence complementary to a target sequence in a FOXP3 gene. In some embodiments, the gRNA comprises a spacer sequence complementary to a target sequence in exon 1 of a FOXP3 gene. In some embodiments, the gRNA comprises a spacer sequence from any one of SEQ ID NOs: 1-7 and 27-29 or a variant thereof having no more than 3 mismatches compared to any one of SEQ ID NOs: 1-7 and 27-29. In some embodiments, the gRNA comprises a spacer sequence from any one of SEQ ID NOs: 1-7 or a variant thereof having no more than 3 mismatches compared to any one of SEQ ID NOs: 1-7. In some embodiments, the gRNA comprises a spacer sequence from any one of SEQ ID NOs: 2, 3, and 5, or a variant thereof having no more than 3 mismatches compared to any one of SEQ ID NOs: 2, 3, and 5. In some embodiments, the gRNA comprises a spacer sequence complementary to a target sequence in a non-FOXP3 locus (e.g., AAVS1). In some embodiments, the gRNA comprises a spacer sequence from any one of SEQ ID NOs: 15-20 or a variant thereof having no more than 3 mismatches compared to any one of SEQ ID NOs: 15-20. In some embodiments, the FOXP3 coding sequence encodes FOXP3 or a functional derivative thereof. In some embodiments, the FOXP3 coding sequence is a FOXP3 cDNA. An exemplary FOXP3 cDNA sequence can be found in the AAV donor template having the nucleotide sequence of SEQ ID NO: 34. In some embodiments, the nucleic acid sequence encoding a FOXP3 or a functional derivative thereof has at least or at least about 70% sequence identity, e.g., at least or at least about 75%, 80%, 85%, 90%, 92%, 93%, 94%, 95%, 96%, 97%, 98%, 99% or greater sequence identity, to a sequence according to SEQ ID NO: 110 or 111. In some embodiments, the system comprises the DNA endonuclease. In some embodiments, the system comprises nucleic acid encoding the DNA endonuclease. In some embodiments, the system comprises the gRNA. In some embodiments, the gRNA is an sgRNA. In some embodiments, the system comprises nucleic acid encoding the gRNA. In some embodiments, the system further comprises one or more additional gRNAs or nucleic acid encoding the one or more additional gRNAs.

In some embodiments, according to any of the systems described herein, the gRNA comprises a spacer sequence from any one of SEQ ID NOs: 1-7, 15-20, and 27-29, or a variant thereof having no more than 3 mismatches compared to any one of SEQ ID NOs: 1-7, 15-20, and 27-29. In some embodiments, the gRNA comprises a spacer sequence from any one of SEQ ID NOs: 1-7 or a variant thereof having no more than 3 mismatches compared to any one of SEQ ID NOs: 1-7. In some embodiments, the gRNA comprises a spacer sequence from any one of SEQ ID NOs: 2, 3, and 5 or a variant thereof having no more than 3 mismatches compared to any one of SEQ ID NOs: 2, 3, and 5. In some embodiments, the gRNA comprises a spacer sequence from SEQ ID NO: 2 or a variant thereof having no more than 3 mismatches compared to SEQ ID NO: 2. In some embodiments, the gRNA comprises a spacer sequence from SEQ ID NO: 3 or a variant thereof having no more than 3 mismatches compared to SEQ ID NO: 3. In some embodiments, the gRNA comprises a spacer sequence from SEQ ID NO: 5 or a variant thereof having no more than 3 mismatches compared to SEQ ID NO: 5.

In some embodiments, according to any of the systems described herein, the Cas DNA endonuclease is a Cas9 endonuclease. In some embodiments, the Cas9 endonuclease is from Streptococcus pyogenes (spCas9). In some embodiments, the Cas9 is from Staphylococcus lugdunensis (SluCas9).

In some embodiments, according to any of the systems described herein, the system comprises a nucleic acid encoding the DNA endonuclease. In some embodiments, the nucleic acid encoding the DNA endonuclease is codon-optimized for expression in a host cell. In some embodiments, the nucleic acid encoding the DNA endonuclease is codon-optimized for expression in a human cell. In some embodiments, the nucleic acid encoding the DNA endonuclease is DNA, such as a DNA plasmid. In some embodiments, the nucleic acid encoding the DNA endonuclease is RNA, such as mRNA.

In some embodiments, according to any of the systems described herein, the nucleic acid sequence encoding a FOXP3 or a functional derivative thereof is codon-optimized for expression in a host cell. In some embodiments, the nucleic acid sequence encoding the FOXP3 or a functional derivative thereof is codon-optimized for expression in a human cell.

In some embodiments, according to any of the systems described herein, the donor template comprises a donor cassette comprising the nucleic acid sequence encoding a FOXP3 or a functional derivative thereof, and a promoter configured to express the FOXP3 or functional derivative thereof. Exemplary promoters include the MND promoter, PGK promoter, and EF1 promoter. In some embodiments, the promoter has a sequence of any one of SEQ ID NOS: 147-149, or a variant having at least 85%, 90%, 91%, 92%, 93%, 94%, 95%, 96%, 97%, 98%, or 99% identity to any one of SEQ ID NOS: 147-149. In some embodiments, the donor template is encoded in an Adeno Associated Virus (AAV) vector. In some embodiments, the AAV vector is an AAV6 vector.

In some embodiments, according to any of the systems described herein, the donor template comprises a donor cassette comprising the nucleic acid sequence encoding a FOXP3 or a functional derivative thereof, and lacks an exogenous promoter configured to express the FOXP3 or functional derivative thereof. In some embodiments, the cell is a CD34⁺ cell, and expression of the FOXP3 or functional derivative thereof relies on an endogenous promoter in the cell. In some embodiments, the donor template is encoded in an Adeno Associated Virus (AAV) vector. In some embodiments, the AAV vector is an AAV6 vector.

In some embodiments, according to any of the systems described herein, the donor template comprises a donor cassette comprising the nucleic acid sequence encoding a FOXP3 or a functional derivative thereof, and the donor template is configured such that the donor cassette is capable of being integrated into a genomic locus targeted by a gRNA in the system by homology directed repair (HDR). In some embodiments, the donor cassette is flanked on both sides by homology arms corresponding to sequences in the targeted genomic locus. In some embodiments, the homology arms are at least or at least about 0.2 kb (such as at least or at least about any of 0.3 kb, 0.4 kb, 0.5 kb, 0.6 kb, 0.7 kb, 0.8 kb, 0.9 kb, 1 kb, or greater) in length. In some embodiments, the homology arms are at least or at least about 0.6 kb in length. Exemplary homology arms include homology arms from donor templates having the sequence of SEQ ID NO: 34 or 161. In some embodiments, the donor template is encoded in an Adeno Associated Virus (AAV) vector. In some embodiments, the AAV vector is an AAV6 vector.

In some embodiments, according to any of the systems described herein, the donor template comprises a donor cassette comprising the nucleic acid sequence encoding a FOXP3 or a functional derivative thereof, and the donor template is configured such that the donor cassette is capable of being integrated into a genomic locus targeted by a gRNA in the system by non-homologous end joining (NHEJ). In some embodiments, the donor cassette is flanked on one or both sides by a gRNA target site. In some embodiments, the donor cassette is flanked on both sides by a gRNA target site. In some embodiments, the gRNA target site is a target site for a gRNA in the system. In some embodiments, the gRNA target site of the donor template is the reverse complement of a cell genome gRNA target site for a gRNA in the system. In some embodiments, the donor template is encoded in an Adeno Associated Virus (AAV) vector. In some embodiments, the AAV vector is an AAV6 vector.

In some embodiments, according to any of the systems described herein, the donor template comprises a donor cassette comprising the nucleic acid sequence encoding a FOXP3 or a functional derivative thereof, and the donor template further comprises a regulatory element enhancing stable expression. Exemplary regulatory elements enhancing stable expression include WPRE and UCOE. In some embodiments, the WPRE is a full-length WPRE. In some embodiments, the WPRE is a truncated WPRE. Exemplary WPREs include WPREs from a donor template having the sequence of any one of SEQ ID NOs: 33, 34, and 161. In some embodiments, the donor template is encoded in an Adeno Associated Virus (AAV) vector. In some embodiments, the AAV vector is an AAV6 vector.

In some embodiments, according to any of the systems described herein, the donor template comprises a donor cassette comprising the nucleic acid sequence encoding a FOXP3 or a functional derivative thereof, and the donor template further comprises a nucleic acid encoding a selectable marker. In some embodiments, the selectable marker is a surface marker that allows for selection of cells expressing the selectable marker. In some embodiments, the selectable marker is a low-affinity nerve growth factor receptor (LNGFR) polypeptide, a green fluorescent protein (GFP), or a functional derivative thereof. In some embodiments, the LNGFR polypeptide or a functional derivative thereof comprises an amino acid sequence of SEQ ID NO: 144 or a variant thereof having at least 85%, 90%, 91%, 92%, 93%, 94%, 95%, 96%, 97%, 98%, or 99% identity to the amino acid sequence of SEQ ID NO: 144. In some embodiments, the nucleic acid encoding the GFP or functional derivative thereof has a nucleic acid sequence of the GFP encoding region of any one of SEQ ID NOS: 33, 35, and 36. In some embodiments, the donor template is encoded in an Adeno Associated Virus (AAV) vector. In some embodiments, the AAV vector is an AAV6 vector.

In some embodiments, according to any of the systems described herein, the donor template comprises a donor cassette comprising the nucleic acid sequence encoding a FOXP3 or a functional derivative thereof, and the donor template further comprises a nucleic acid encoding a 2A self-cleaving peptide between adjacent system component-encoding nucleic acids. In some embodiments, the donor template comprise nucleic acid encoding a 2A self-cleaving peptide between each of the adjacent system component-encoding nucleic acids. In some embodiments, each of the 2A self-cleaving peptides is, independently, a T2A self-cleaving peptide or a P2A self-cleaving peptide. For example, in some embodiments, the donor template comprises, in order from 5′ to 3′, a nucleic acid encoding expression of a FOXP3 or functional variant thereof, nucleic acid encoding a 2A self-cleaving peptide, and a nucleic acid encoding a selectable marker. In some embodiments, the donor template comprises a nucleic acid of any one of SEQ ID NOS: 72 and 73, or a variant of a nucleic acid having at least 85%, 90%, 91%, 92%, 93%, 94%, 95%, 96%, 97%, 98%, or 99% identity to any one of SEQ ID NOS: 72 and 73. In some embodiments, the donor template is encoded in an Adeno Associated Virus (AAV) vector. In some embodiments, the AAV vector is an AAV6 vector.

Exemplary donor templates include donor templates having any one of the sequences of SEQ ID NOS:33-36 and 161. In some embodiments, the donor template comprises the sequence of SEQ ID NO: 34 or 161. In some embodiments, the donor template is encoded in an Adeno Associated Virus (AAV) vector. In some embodiments, the AAV vector is an AAV6 vector.

In some embodiments, according to any of the systems described herein, the DNA endonuclease or nucleic acid encoding the DNA endonuclease is formulated in a liposome or lipid nanoparticle. In some embodiments, the liposome or lipid nanoparticle also comprises the gRNA. In some embodiments, the liposome or lipid nanoparticle is a lipid nanoparticle. In some embodiments, the system comprises a lipid nanoparticle comprising nucleic acid encoding the DNA endonuclease and the gRNA. In some embodiments, the nucleic acid encoding the DNA endonuclease is an mRNA encoding the DNA endonuclease.

In some embodiments, according to any of the systems described herein, the DNA endonuclease is complexed with the gRNA, forming a ribonucleoprotein (RNP) complex.

### Nucleic Acids

Genome-Targeting Nucleic Acid or Guide RNA

The present disclosure provides a genome-targeting nucleic acid that can direct the activities of an associated polypeptide (e.g., a site-directed polypeptide or DNA endonuclease) to a specific target sequence within a target nucleic acid. In some embodiments, the genome-targeting nucleic acid is an RNA. A genome-targeting RNA is referred to as a “guide RNA” or “gRNA” herein. A guide RNA has at least a spacer sequence that can hybridize to a target nucleic acid sequence of interest and a CRISPR repeat sequence. In Type II systems, the gRNA also has a second RNA referred to as a tracrRNA sequence. In the Type II guide RNA (gRNA), the CRISPR repeat sequence and tracrRNA sequence hybridize to each other to form a duplex. In the Type V guide RNA (gRNA), the crRNA forms a duplex. In both systems, the duplex binds a site-directed polypeptide such that the guide RNA and site-direct polypeptide form a complex. The genome-targeting nucleic acid provides target specificity to the complex by virtue of its association with the site-directed polypeptide. The genome-targeting nucleic acid thus directs the activity of the site-directed polypeptide.

In some embodiments, the genome-targeting nucleic acid is a double-molecule guide RNA. In some embodiments, the genome-targeting nucleic acid is a single-molecule guide RNA. A double-molecule guide RNA has two strands of RNA. The first strand has in the 5′ to 3′ direction, an optional spacer extension sequence, a spacer sequence and a minimum CRISPR repeat sequence. The second strand has a minimum tracrRNA sequence (complementary to the minimum CRISPR repeat sequence), a 3′ tracrRNA sequence and an optional tracrRNA extension sequence. A single-molecule guide RNA (sgRNA) in a Type II system has, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may have elements that contribute additional functionality (e.g., stability) to the guide RNA. The single-molecule guide linker links the minimum CRISPR repeat and the minimum tracrRNA sequence to form a hairpin structure. The optional tracrRNA extension has one or more hairpins. A single-molecule guide RNA (sgRNA) in a Type V system has, in the 5′ to 3′ direction, a minimum CRISPR repeat sequence and a spacer sequence.

By way of illustration, guide RNAs used in the CRISPR/Cas/Cpf1 system, or other smaller RNAs can be readily synthesized by chemical means as illustrated below and described in the art. While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs, such as those encoding a Cas endonuclease (e.g., a Cas9 or Cpf1 endonuclease), are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

In some embodiments, provided herein is a guide RNA (gRNA) comprising a spacer sequence that is complementary to a genomic sequence within or near a FOXP3 gene in a cell. In some embodiments, the gRNA comprises a spacer sequence from any one of SEQ ID NOs: 1-7 and 27-29 or a variant thereof having no more than 3 mismatches compared to any one of SEQ ID NOs: 1-7 and 27-29. In some embodiments, the gRNA comprises a spacer sequence from any one of SEQ ID NOs: 1-7 or a variant thereof having no more than 3 mismatches compared to any one of SEQ ID NOs: 1-7. In some embodiments, the gRNA comprises a spacer sequence from any one of SEQ ID NOs: 2, 3, and 5 or a variant thereof having no more than 3 mismatches compared to any one of SEQ ID NOs: 2, 3, and 5.

In some embodiments, provided herein is a guide RNA (gRNA) comprising a spacer sequence that is complementary to a genomic sequence within or near an AAVS1 locus in a cell. In some embodiments, the gRNA comprises a spacer sequence from any one of SEQ ID NOs: 15-20 or a variant thereof having no more than 3 mismatches compared to any one of SEQ ID NOs: 15-20.

Guide RNA made by in vitro transcription may contain mixtures of full length and partial guide RNA molecules. Chemically synthesized guide RNA molecules are generally composed of >75% full length guide molecules and in addition may contain chemically modified bases, such as those that make the guide RNA more resistant to cleavage by nucleases in the cell.

Spacer Extension Sequence

In some embodiments of genome-targeting nucleic acids, a spacer extension sequence can modify activity, provide stability or provide a location for modifications of a genome-targeting nucleic acid. A spacer extension sequence can modify on- or off-target activity or specificity. In some embodiments, a spacer extension sequence is provided. A spacer extension sequence can have a length of more than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 1000, 2000, 3000, 4000, 5000, 6000, or 7000 or more nucleotides. A spacer extension sequence can have a length of or about 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 1000, 2000, 3000, 4000, 5000, 6000, or 7000 or more nucleotides. A spacer extension sequence can have a length of less than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 1000, 2000, 3000, 4000, 5000, 6000, 7000, or more nucleotides. In some embodiments, a spacer extension sequence is less than 10 nucleotides in length. In some embodiments, a spacer extension sequence is between 10-30 nucleotides in length. In some embodiments, a spacer extension sequence is between 30-70 nucleotides in length.

In some embodiments, the spacer extension sequence has another moiety (e.g., a stability control sequence, an endoribonuclease binding sequence, or a ribozyme). In some embodiments, the moiety decreases or increases the stability of a nucleic acid targeting nucleic acid. In some embodiments, the moiety is a transcriptional terminator segment (i.e., a transcription termination sequence). In some embodiments, the moiety functions in a eukaryotic cell. In some embodiments, the moiety functions in a prokaryotic cell. In some embodiments, the moiety functions in both eukaryotic and prokaryotic cells. Non-limiting examples of suitable moieties include: a 5′ cap (e.g., a 7-methylguanylate cap (m7 G)), a riboswitch sequence (e.g., to allow for regulated stability and/or regulated accessibility by proteins and protein complexes), a sequence that forms a dsRNA duplex (i.e., a hairpin), a sequence that targets the RNA to a subcellular location (e.g., nucleus, mitochondria, or chloroplasts, and the like), a modification or sequence that provides for tracking (e.g., direct conjugation to a fluorescent molecule, conjugation to a moiety that facilitates fluorescent detection, or a sequence that allows for fluorescent detection, etc.), or a modification or sequence that provides a binding site for proteins (e.g., proteins that act on DNA, including transcriptional activators, transcriptional repressors, DNA methyltransferases, DNA demethylases, histone acetyltransferases, or histone deacetylases, and the like).

Spacer Sequence

The spacer sequence hybridizes to a sequence in a target nucleic acid of interest. The spacer of a genome-targeting nucleic acid interacts with a target nucleic acid in a sequence-specific manner via hybridization (i.e., base pairing). The nucleotide sequence of the spacer thus varies depending on the sequence of the target nucleic acid of interest.

In a CRISPR/Cas system herein, the spacer sequence is designed to hybridize to a target nucleic acid that is located 5′ of a PAM of a Cas endonuclease used in the system. The spacer can perfectly match the target sequence or can have mismatches. Each Cas endonuclease has a particular PAM sequence that it recognizes in a target DNA. For example, Cas9 from S. pyogenes recognizes in a target nucleic acid a PAM that has the sequence 5′-NRG-3′, where R has either A or G, where N is any nucleotide and N is immediately 3′ of the target nucleic acid sequence targeted by the spacer sequence.

In some embodiments, the target nucleic acid sequence has 20 nucleotides. In some embodiments, the target nucleic acid has less than 20 nucleotides but not zero. In some embodiments, the target nucleic acid has more than 20 nucleotides. In some embodiments, the target nucleic acid has at least: 5, 10, 15, 16, 17, 18, 19, 20, 21, 22, 23, 24, 25, 30, or more nucleotides. In some embodiments, the target nucleic acid has at most: 5, 10, 15, 16, 17, 18, 19, 20, 21, 22, 23, 24, 25, 30, or more nucleotides. In some embodiments, the target nucleic acid sequence has 20 bases immediately 5′ of the first nucleotide of the PAM. In some embodiments, the PAM sequence used in the compositions and methods of the present disclosure as a sequence recognized by S. pyogenes Cas9 is NGG.

In some embodiments, the spacer sequence that hybridizes to the target nucleic acid has a length of at least or at least about 6 nucleotides (nt). The spacer sequence can be at least or at least about 6 nt, at or about 10 nt, at or about 15 nt, at or about 18 nt, at or about 19 nt, at or about 20 nt, at or about 25 nt, at or about 30 nt, at or about 35 nt or at or about 40 nt, from or from about 6 nt to or to about 80 nt, from or from about 6 nt to or to about 50 nt, from or from about 6 nt to or to about 45 nt, from or from about 6 nt to or to about 40 nt, from or from about 6 nt to or to about 35 nt, from or from about 6 nt to or to about 30 nt, from or from about 6 nt to or to about 25 nt, from or from about 6 nt to or to about 20 nt, from or from about 6 nt to or to about 19 nt, from or from about 10 nt to or to about 50 nt, from or from about 10 nt to or to about 45 nt, from or from about 10 nt to or to about 40 nt, from or from about 10 nt to or to about 35 nt, from or from about 10 nt to or to about 30 nt, from or from about 10 nt to or to about 25 nt, from or from about 10 nt to or to about 20 nt, from or from about 10 nt to or to about 19 nt, from or from about 19 nt to or to about 25 nt, from or from about 19 nt to or to about 30 nt, from or from about 19 nt to or to about 35 nt, from or from about 19 nt to or to about 40 nt, from or from about 19 nt to or to about 45 nt, from or from about 19 nt to or to about 50 nt, from or from about 19 nt to or to about 60 nt, from or from about 20 nt to or to about 25 nt, from or from about 20 nt to or to about 30 nt, from or from about 20 nt to or to about 35 nt, from or from about 20 nt to or to about 40 nt, from or from about 20 nt to or to about 45 nt, from or from about 20 nt to or to about 50 nt, or from or from about 20 nt to or to about 60 nt. In some embodiments, the spacer sequence has 20 nucleotides. In some embodiments, the spacer has 19 nucleotides. In some embodiments, the spacer has 18 nucleotides. In some embodiments, the spacer has 17 nucleotides. In some embodiments, the spacer has 16 nucleotides. In some embodiments, the spacer has 15 nucleotides.

In some embodiments, the percent complementarity between the spacer sequence and the target nucleic acid is at least or at least about 30%, at least or at least about 40%, at least or at least about 50%, at least or at least about 60%, at least or at least about 65%, at least or at least about 70%, at least or at least about 75%, at least or at least about 80%, at least or at least about 85%, at least or at least about 90%, at least or at least about 95%, at least or at least about 97%, at least or at least about 98%, at least or at least about 99%, or 100%. In some embodiments, the percent complementarity between the spacer sequence and the target nucleic acid is at most or at most about 30%, at most or at most about 40%, at most or at most about 50%, at most or at most about 60%, at most or at most about 65%, at most or at most about 70%, at most or at most about 75%, at most or at most about 80%, at most or at most about 85%, at most or at most about 90%, at most or at most about 95%, at most or at most about 97%, at most or at most about 98%, at most or at most about 99%, or 100%. In some embodiments, the percent complementarity between the spacer sequence and the target nucleic acid is 100% over the six contiguous 5′-most nucleotides of the target sequence of the complementary strand of the target nucleic acid. In some embodiments, the percent complementarity between the spacer sequence and the target nucleic acid is at least 60% over or over about 20 contiguous nucleotides. In some embodiments, the length of the spacer sequence and the target nucleic acid can differ by 1 to 6 nucleotides, which can be thought of as a bulge or bulges.

In some embodiments, the spacer sequence is designed or chosen using a computer program. The computer program can use variables, such as predicted melting temperature, secondary structure formation, predicted annealing temperature, sequence identity, genomic context, chromatin accessibility, % GC, frequency of genomic occurrence (e.g., of sequences that are identical or are similar but vary in one or more spots as a result of mismatch, insertion, or deletion), methylation status, presence of SNPs, and the like.

Minimum CRISPR Repeat Sequence

In some embodiments, a minimum CRISPR repeat sequence is a sequence with at least or at least about 30%, at or about 40%, at or about 50%, at or about 60%, at or about 65%, at or about 70%, at or about 75%, at or about 80%, at or about 85%, at or about 90%, at or about 95%, or 100% sequence identity to a reference CRISPR repeat sequence (e.g., crRNA from S. pyogenes).

In some embodiments, a minimum CRISPR repeat sequence has nucleotides that can hybridize to a minimum tracrRNA sequence in a cell. The minimum CRISPR repeat sequence and a minimum tracrRNA sequence form a duplex, i.e., a base-paired double-stranded structure. Together, the minimum CRISPR repeat sequence and the minimum tracrRNA sequence bind to the site-directed polypeptide. At least a part of the minimum CRISPR repeat sequence hybridizes to the minimum tracrRNA sequence. In some embodiments, at least a part of the minimum CRISPR repeat sequence has at least or at least about 30%, at or about 40%, at or about 50%, at or about 60%, at or about 65%, at or about 70%, at or about 75%, at or about 80%, at or about 85%, at or about 90%, at or about 95%, or 100% complementarity to the minimum tracrRNA sequence. In some embodiments, at least a part of the minimum CRISPR repeat sequence has at most or at most about 30%, at or about 40%, at or about 50%, at or about 60%, at or about 65%, at or about 70%, at or about 75%, at or about 80%, at or about 85%, at or about 90%, at or about 95%, or 100% complementarity to the minimum tracrRNA sequence.

The minimum CRISPR repeat sequence can have a length from or from about 7 nucleotides to or to about 100 nucleotides. For example, the length of the minimum CRISPR repeat sequence is from or from about 7 nucleotides (nt) to or to about 50 nt, from or from about 7 nt to or to about 40 nt, from or from about 7 nt to or to about 30 nt, from or from about 7 nt to or to about 25 nt, from or from about 7 nt to or to about 20 nt, from or from about 7 nt to or to about 15 nt, from or from about 8 nt to or to about 40 nt, from or from about 8 nt to or to about 30 nt, from or from about 8 nt to or to about 25 nt, from or from about 8 nt to or to about 20 nt, from or from about 8 nt to or to about 15 nt, from or from about 15 nt to or to about 100 nt, from or from about 15 nt to or to about 80 nt, from or from about 15 nt to or to about 50 nt, from or from about 15 nt to or to about 40 nt, from or from about 15 nt to or to about 30 nt, or from or from about 15 nt to or to about 25 nt. In some embodiments, the minimum CRISPR repeat sequence is approximately 9 nucleotides in length. In some embodiments, the minimum CRISPR repeat sequence is approximately 12 nucleotides in length.

In some embodiments, the minimum CRISPR repeat sequence is at least or at least about 60% identical to a reference minimum CRISPR repeat sequence (e.g., wild-type crRNA from S. pyogenes) over a stretch of at least 6, 7, or 8 contiguous nucleotides. For example, the minimum CRISPR repeat sequence is at least or at least about 65% identical, at least or at least about 70% identical, at least or at least about 75% identical, at least or at least about 80% identical, at least or at least about 85% identical, at least or at least about 90% identical, at least or at least about 95% identical, at least or at least about 98% identical, at least or at least about 99% identical or 100% identical to a reference minimum CRISPR repeat sequence over a stretch of at least 6, 7, or 8 contiguous nucleotides.

Minimum tracrRNA Sequence

In some embodiments, a minimum tracrRNA sequence is a sequence with at least or at least about 30%, at or about 40%, at or about 50%, at or about 60%, at or about 65%, at or about 70%, at or about 75%, at or about 80%, at or about 85%, at or about 90%, at or about 95%, or 100% sequence identity to a reference tracrRNA sequence (e.g., wild type tracrRNA from S. pyogenes).

In some embodiments, a minimum tracrRNA sequence has nucleotides that hybridize to a minimum CRISPR repeat sequence in a cell. A minimum tracrRNA sequence and a minimum CRISPR repeat sequence form a duplex, i.e., a base-paired double-stranded structure. Together, the minimum tracrRNA sequence and the minimum CRISPR repeat bind to a site-directed polypeptide. At least a part of the minimum tracrRNA sequence can hybridize to the minimum CRISPR repeat sequence. In some embodiments, the minimum tracrRNA sequence is at least or at least about 30%, at or about 40%, at or about 50%, at or about 60%, at or about 65%, at or about 70%, at or about 75%, at or about 80%, at or about 85%, at or about 90%, at or about 95%, or 100% complementarity to the minimum CRISPR repeat sequence.

The minimum tracrRNA sequence can have a length from or from about 7 nucleotides to or to about 100 nucleotides. For example, the minimum tracrRNA sequence can be from or from about 7 nucleotides (nt) to or to about 50 nt, from or from about 7 nt to or to about 40 nt, from or from about 7 nt to or to about 30 nt, from or from about 7 nt to or to about 25 nt, from or from about 7 nt to or to about 20 nt, from or from about 7 nt to or to about 15 nt, from or from about 8 nt to or to about 40 nt, from or from about 8 nt to or to about 30 nt, from or from about 8 nt to or to about 25 nt, from or from about 8 nt to or to about 20 nt, from or from about 8 nt to or to about 15 nt, from or from about 15 nt to or to about 100 nt, from or from about 15 nt to or to about 80 nt, from or from about 15 nt to or to about 50 nt, from or from about 15 nt to or to about 40 nt, from or from about 15 nt to or to about 30 nt or from or from about 15 nt to or to about 25 nt long. In some embodiments, the minimum tracrRNA sequence is approximately 9 nucleotides in length. In some embodiments, the minimum tracrRNA sequence is approximately 12 nucleotides. In some embodiments, the minimum tracrRNA consists of tracrRNA nt 23-48 described in Jinek, M. et al. (2012). Science, 337(6096):816-821.

In some embodiments, the minimum tracrRNA sequence is at least or at least about 60% identical to a reference minimum tracrRNA (e.g., wild type, tracrRNA from S. pyogenes) sequence over a stretch of at least 6, 7, or 8 contiguous nucleotides. For example, the minimum tracrRNA sequence is at least or at least about 65% identical, at or about 70% identical, at or about 75% identical, at or about 80% identical, at or about 85% identical, at or about 90% identical, at or about 95% identical, at or about 98% identical, at or about 99% identical or 100% identical to a reference minimum tracrRNA sequence over a stretch of at least 6, 7, or 8 contiguous nucleotides.

In some embodiments, the duplex between the minimum CRISPR RNA and the minimum tracrRNA has a double helix. In some embodiments, the duplex between the minimum CRISPR RNA and the minimum tracrRNA has at least or at least about 1, 2, 3, 4, 5, 6, 7, 8, 9, or 10 or more nucleotides. In some embodiments, the duplex between the minimum CRISPR RNA and the minimum tracrRNA has at most or at most about 1, 2, 3, 4, 5, 6, 7, 8, 9, or 10 or more nucleotides.

In some embodiments, the duplex has a mismatch (i.e., the two strands of the duplex are not 100% complementary). In some embodiments, the duplex has at least or at least about 1, 2, 3, 4, or 5 or mismatches. In some embodiments, the duplex has at most or at most about 1, 2, 3, 4, or 5 or mismatches. In some embodiments, the duplex has no more than 2 mismatches.

Bulges

In some embodiments, there is a “bulge” in the duplex between the minimum CRISPR RNA and the minimum tracrRNA. The bulge is an unpaired region of nucleotides within the duplex. In some embodiments, the bulge contributes to the binding of the duplex to the site-directed polypeptide. A bulge has, on one side of the duplex, an unpaired 5′-XXXY-3′ where X is any purine and Y has a nucleotide that can form a wobble pair with a nucleotide on the opposite strand, and an unpaired nucleotide region on the other side of the duplex. The number of unpaired nucleotides on the two sides of the duplex can be different.

In one example, the bulge has an unpaired purine (e.g., adenine) on the minimum CRISPR repeat strand of the bulge. In some embodiments, a bulge has an unpaired 5′-AAGY-3′ of the minimum tracrRNA sequence strand of the bulge, where Y has a nucleotide that can form a wobble pairing with a nucleotide on the minimum CRISPR repeat strand.

In some embodiments, a bulge on the minimum CRISPR repeat side of the duplex has at least 1, 2, 3, 4, or 5 or more unpaired nucleotides. In some embodiments, a bulge on the minimum CRISPR repeat side of the duplex has at most 1, 2, 3, 4, or 5 or more unpaired nucleotides. In some embodiments, a bulge on the minimum CRISPR repeat side of the duplex has 1 unpaired nucleotide.

In some embodiments, a bulge on the minimum tracrRNA sequence side of the duplex has at least 1, 2, 3, 4, 5, 6, 7, 8, 9, or 10 or more unpaired nucleotides. In some embodiments, a bulge on the minimum tracrRNA sequence side of the duplex has at most 1, 2, 3, 4, 5, 6, 7, 8, 9, or 10 or more unpaired nucleotides. In some embodiments, a bulge on a second side of the duplex (e.g., the minimum tracrRNA sequence side of the duplex) has 4 unpaired nucleotides.

In some embodiments, a bulge has at least one wobble pairing. In some embodiments, a bulge has at most one wobble pairing. In some embodiments, a bulge has at least one purine nucleotide. In some embodiments, a bulge has at least 3 purine nucleotides. In some embodiments, a bulge sequence has at least 5 purine nucleotides. In some embodiments, a bulge sequence has at least one guanine nucleotide. In some embodiments, a bulge sequence has at least one adenine nucleotide.

Hairpins

In various embodiments, one or more hairpins are located 3′ to the minimum tracrRNA in the 3′ tracrRNA sequence.

In some embodiments, the hairpin starts at least or at least about 1, 2, 3, 4, 5, 6, 7, 8, 9, 10, 15, or 20 or more nucleotides 3′ from the last paired nucleotide in the minimum CRISPR repeat and minimum tracrRNA sequence duplex. In some embodiments, the hairpin can start at most or at most about 1, 2, 3, 4, 5, 6, 7, 8, 9, or 10 or more nucleotides 3′ of the last paired nucleotide in the minimum CRISPR repeat and minimum tracrRNA sequence duplex.

In some embodiments, a hairpin has at least or at least about 1, 2, 3, 4, 5, 6, 7, 8, 9, 10, 15, or 20 or more consecutive nucleotides. In some embodiments, a hairpin has at most or at most about 1, 2, 3, 4, 5, 6, 7, 8, 9, 10, 15, or more consecutive nucleotides.

In some embodiments, a hairpin has a CC di-nucleotide (i.e., two consecutive cytosine nucleotides).

In some embodiments, a hairpin has duplexed nucleotides (e.g., nucleotides in a hairpin, hybridized together). For example, a hairpin has a CC di-nucleotide that is hybridized to a GG di-nucleotide in a hairpin duplex of the 3′ tracrRNA sequence.

One or more of the hairpins can interact with guide RNA-interacting regions of a site-directed polypeptide.

In some embodiments there are two or more hairpins, and in some embodiments there are three or more hairpins.

3′ tracrRNA Sequence

In some embodiments, a 3′ tracrRNA sequence has a sequence with at least or at least about 30%, at or about 40%, at or about 50%, at or about 60%, at or about 65%, at or about 70%, at or about 75%, at or about 80%, at or about 85%, at or about 90%, at or about 95%, or 100% sequence identity to a reference tracrRNA sequence (e.g., a tracrRNA from S. pyogenes).

In some embodiments, the 3′ tracrRNA sequence has a length from or from about 6 nucleotides to or to about 100 nucleotides. For example, the 3′ tracrRNA sequence can have a length from or from about 6 nucleotides (nt) to or to about 50 nt, from or from about 6 nt to or to about 40 nt, from or from about 6 nt to or to about 30 nt, from or from about 6 nt to or to about 25 nt, from or from about 6 nt to or to about 20 nt, from or from about 6 nt to or to about 15 nt, from or from about 8 nt to or to about 40 nt, from or from about 8 nt to or to about 30 nt, from or from about 8 nt to or to about 25 nt, from or from about 8 nt to or to about 20 nt, from or from about 8 nt to or to about 15 nt, from or from about 15 nt to or to about 100 nt, from or from about 15 nt to or to about 80 nt, from or from about 15 nt to or to about 50 nt, from or from about 15 nt to or to about 40 nt, from or from about 15 nt to or to about 30 nt, or from or from about 15 nt to or to about 25 nt. In some embodiments, the 3′ tracrRNA sequence has a length of approximately 14 nucleotides.

In some embodiments, the 3′ tracrRNA sequence is at least or at least about 60% identical to a reference 3′ tracrRNA sequence (e.g., wild type 3′ tracrRNA sequence from S. pyogenes) over a stretch of at least 6, 7, or 8 contiguous nucleotides. For example, the 3′ tracrRNA sequence is at least or at least about 60% identical, at or about 65% identical, at or about 70% identical, at or about 75% identical, at or about 80% identical, at or about 85% identical, at or about 90% identical, at or about 95% identical, at or about 98% identical, at or about 99% identical, or 100% identical, to a reference 3′ tracrRNA sequence (e.g., wild type 3′ tracrRNA sequence from S. pyogenes) over a stretch of at least 6, 7, or 8 contiguous nucleotides.

In some embodiments, a 3′ tracrRNA sequence has more than one duplexed region (e.g., hairpin, hybridized region). In some embodiments, a 3′ tracrRNA sequence has two duplexed regions.

In some embodiments, the 3′ tracrRNA sequence has a stem loop structure. In some embodiments, a stem loop structure in the 3′ tracrRNA has at least 1, 2, 3, 4, 5, 6, 7, 8, 9, 10, 15, or 20 or more nucleotides. In some embodiments, the stem loop structure in the 3′ tracrRNA has at most 1, 2, 3, 4, 5, 6, 7, 8, 9, or 10 or more nucleotides. In some embodiments, the stem loop structure has a functional moiety. For example, the stem loop structure can have an aptamer, a ribozyme, a protein-interacting hairpin, a CRISPR array, an intron, or an exon. In some embodiments, the stem loop structure has at least or at least about 1, 2, 3, 4, or 5 or more functional moieties. In some embodiments, the stem loop structure has at most or at most about 1, 2, 3, 4, or 5 or more functional moieties.

In some embodiments, the hairpin in the 3′ tracrRNA sequence has a P-domain. In some embodiments, the P-domain has a double-stranded region in the hairpin.

tracrRNA Extension Sequence

In some embodiments, a tracrRNA extension sequence can be provided whether the tracrRNA is in the context of single-molecule guides or double-molecule guides. In some embodiments, a tracrRNA extension sequence has a length from or from about 1 nucleotide to or to about 400 nucleotides. In some embodiments, a tracrRNA extension sequence has a length of more than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, or 400 nucleotides. In some embodiments, a tracrRNA extension sequence has a length from or from about 20 to or to about 5000 or more nucleotides. In some embodiments, a tracrRNA extension sequence has a length of more than 1000 nucleotides. In some embodiments, a tracrRNA extension sequence has a length of less than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, or more nucleotides but not zero. In some embodiments, a tracrRNA extension sequence can have a length of less than 1000 nucleotides but not zero. In some embodiments, a tracrRNA extension sequence has less than 10 nucleotides in length but not zero. In some embodiments, a tracrRNA extension sequence is 10-30 nucleotides in length. In some embodiments, tracrRNA extension sequence is 30-70 nucleotides in length.

In some embodiments, the tracrRNA extension sequence has a functional moiety (e.g., a stability control sequence, ribozyme, or endoribonuclease binding sequence). In some embodiments, the functional moiety has a transcriptional terminator segment (e.g., a transcription termination sequence). In some embodiments, the functional moiety has a total length from or from about 10 nucleotides (nt) to or to about 100 nucleotides, from or from about 10 nt to or to about 20 nt, from or from about 20 nt to or to about 30 nt, from or from about 30 nt to or to about 40 nt, from or from about 40 nt to or to about 50 nt, from or from about 50 nt to or to about 60 nt, from or from about 60 nt to or to about 70 nt, from or from about 70 nt to or to about 80 nt, from or from about 80 nt to or to about 90 nt, or from or from about 90 nt to or to about 100 nt, from or from about 15 nt to or to about 80 nt, from or from about 15 nt to or to about 50 nt, from or from about 15 nt to or to about 40 nt, from or from about 15 nt to or to about 30 nt, or from or from about 15 nt to or to about 25 nt. In some embodiments, the functional moiety functions in a eukaryotic cell. In some embodiments, the functional moiety functions in a prokaryotic cell. In some embodiments, the functional moiety functions in both eukaryotic and prokaryotic cells.

Non-limiting examples of suitable tracrRNA extension functional moieties include a 3′ poly-adenylated tail, a riboswitch sequence (e.g., to allow for regulated stability and/or regulated accessibility by proteins and protein complexes), a sequence that forms a dsRNA duplex (i.e., a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), or a modification or sequence that provides a binding site for proteins (e.g., proteins that act on DNA, including transcriptional activators, transcriptional repressors, DNA methyltransferases, DNA demethylases, histone acetyltransferases, or histone deacetylases, and the like). In some embodiments, a tracrRNA extension sequence has a primer binding site or a molecular index (e.g., barcode sequence). In some embodiments, the tracrRNA extension sequence has one or more affinity tags.

Single-Molecule Guide Linker Sequence

In some embodiments, the linker sequence of a single-molecule guide nucleic acid has a length from or from about 3 nucleotides to or to about 100 nucleotides. In Jinek, M. et al. (2012). Science, 337(6096):816-821, for example, a simple 4 nucleotide “tetraloop” (-GAAA-) was used. An illustrative linker has a length from or from about 3 nucleotides (nt) to or to about 90 nt, from or from about 3 nt to or to about 80 nt, from or from about 3 nt to or to about 70 nt, from or from about 3 nt to or to about 60 nt, from or from about 3 nt to or to about 50 nt, from or from about 3 nt to or to about 40 nt, from or from about 3 nt to or to about 30 nt, from or from about 3 nt to or to about 20 nt, from or from about 3 nt to or to about 10 nt. For example, the linker can have a length from or from about 3 nt to or to about 5 nt, from or from about 5 nt to or to about 10 nt, from or from about 10 nt to or to about 15 nt, from or from about 15 nt to or to about 20 nt, from or from about 20 nt to or to about 25 nt, from or from about 25 nt to or to about 30 nt, from or from about 30 nt to or to about 35 nt, from or from about 35 nt to or to about 40 nt, from or from about 40 nt to or to about 50 nt, from or from about 50 nt to or to about 60 nt, from or from about 60 nt to or to about 70 nt, from or from about 70 nt to or to about 80 nt, from or from about 80 nt to or to about 90 nt, or from or from about 90 nt to or to about 100 nt. In some embodiments, the linker of a single-molecule guide nucleic acid is between 4 and 40 nucleotides. In some embodiments, a linker is at least or at least about 100, 500, 1000, 1500, 2000, 2500, 3000, 3500, 4000, 4500, 5000, 5500, 6000, 6500, or 7000 or more nucleotides. In some embodiments, a linker is at most or at most about 100, 500, 1000, 1500, 2000, 2500, 3000, 3500, 4000, 4500, 5000, 5500, 6000, 6500, or 7000 or more nucleotides.

Linkers can have any of a variety of sequences, although in some embodiments, the linker will not have sequences that have extensive regions of homology with other portions of the guide RNA, which might cause intramolecular binding that could interfere with other functional regions of the guide. In Jinek, M. et al. (2012). Science, 337(6096):816-821, a simple 4 nucleotide sequence -GAAA- was used, but numerous other sequences, including longer sequences can likewise be used.

In some embodiments, the linker sequence has a functional moiety. For example, the linker sequence can have one or more features, including an aptamer, a ribozyme, a protein-interacting hairpin, a protein binding site, a CRISPR array, an intron, or an exon. In some embodiments, the linker sequence has at least or at least about 1, 2, 3, 4, or 5 or more functional moieties. In some embodiments, the linker sequence has at most or at most about 1, 2, 3, 4, or 5 or more functional moieties.

In some embodiments, a genomic location targeted by gRNAs in accordance with the preset disclosure can be at, within, or near the FOXP3 gene in a genome, e.g., a human genome. Exemplary guide RNAs targeting such locations include the spacer sequences of SEQ ID NOs: 1-7, 15-20, and 27-29. For example, a gRNA including a spacer sequence from SEQ ID NO: 1 can have a spacer sequence including i) the sequence of SEQ ID NO: 1, ii) the sequence from position 2 to position 20 of SEQ ID NO: 1, iii) the sequence from position 3 to position 20 of SEQ ID NO: 1, iv) the sequence from position 4 to position 20 of SEQ ID NO: 1, and so forth. As is understood by the person of ordinary skill in the art, each guide RNA is designed to include a spacer sequence complementary to its genomic target sequence. For example, each of the spacer sequences of SEQ ID NOs: 1-7, 15-20, and 27-29 can be put into a single RNA chimera or a crRNA (along with a corresponding tracrRNA). See Jinek, M. et al. (2012). Science, 337(6096):816-821, and Deltcheva, E. et al. (2011). Nature, 471:602-607.

### Donor DNA or Donor Template

Site-directed polypeptides, such as a DNA endonuclease, can introduce double-strand breaks or single-strand breaks in nucleic acids, e.g., genomic DNA. The double-strand break can stimulate a cell's endogenous DNA-repair pathways (e.g., homology-dependent repair (HDR) or non-homologous end joining or alternative non-homologous end joining (A-NHEJ) or microhomology-mediated end joining (MMEJ). NHEJ can repair cleaved target nucleic acid without the need for a homologous template. This can sometimes result in small deletions or insertions (indels) in the target nucleic acid at the site of cleavage and can lead to disruption or alteration of gene expression. HDR, which is also known as homologous recombination (HR) can occur when a homologous repair template, or donor, is available.

The homologous donor template has sequences that are homologous to sequences flanking the target nucleic acid cleavage site. The sister chromatid is generally used by the cell as the repair template. However, for the purposes of genome editing, the repair template is often supplied as an exogenous nucleic acid, such as a plasmid, duplex oligonucleotide, single-strand oligonucleotide, double-stranded oligonucleotide, or viral nucleic acid. With exogenous donor templates, it is common to introduce an additional nucleic acid sequence (such as a transgene) or modification (such as a single or multiple base change or a deletion) between the flanking regions of homology so that the additional or altered nucleic acid sequence also becomes incorporated into the target locus. MMEJ results in a genetic outcome that is similar to NHEJ in that small deletions and insertions can occur at the cleavage site. MMEJ makes use of homologous sequences of a few base pairs flanking the cleavage site to drive a favored end-joining DNA repair outcome. In some instances, it can be possible to predict likely repair outcomes based on analysis of potential microhomologies in the nuclease target regions.

Thus, in some cases, homologous recombination is used to insert an exogenous polynucleotide sequence into the target nucleic acid cleavage site. An exogenous polynucleotide sequence is termed a donor polynucleotide (or donor or donor sequence or polynucleotide donor template) herein. In some embodiments, the donor polynucleotide, a portion of the donor polynucleotide, a copy of the donor polynucleotide, or a portion of a copy of the donor polynucleotide is inserted into the target nucleic acid cleavage site. In some embodiments, the donor polynucleotide is an exogenous polynucleotide sequence, i.e., a sequence that does not naturally occur at the target nucleic acid cleavage site.

When an exogenous DNA molecule is supplied in sufficient concentration inside the nucleus of a cell in which the double-strand break occurs, the exogenous DNA can be inserted at the double-strand break during the NHEJ repair process and thus become a permanent addition to the genome. These exogenous DNA molecules are referred to as donor templates in some embodiments. If the donor template contains a coding sequence for a gene of interest such as a FOXP3 gene optionally together with relevant regulatory sequences such as promoters, enhancers, polyA sequences and/or splice acceptor sequences (also referred to herein as a “donor cassette”), the gene of interest can be expressed from the integrated copy in the genome resulting in permanent expression for the life of the cell. Moreover, the integrated copy of the donor DNA template can be transmitted to the daughter cells when the cell divides.

In the presence of sufficient concentrations of a donor DNA template that contains flanking DNA sequences with homology to the DNA sequence either side of the double-strand break (referred to as homology arms), the donor DNA template can be integrated via the HDR pathway. The homology arms act as substrates for homologous recombination between the donor template and the sequences either side of the double-strand break. This can result in an error-free insertion of the donor template in which the sequences either side of the double-strand break are not altered from that in the unmodified genome.

Supplied donors for editing by HDR vary markedly but generally contain the intended sequence with small or large flanking homology arms to allow annealing to the genomic DNA. The homology regions flanking the introduced genetic changes can be 30 bp or smaller, or as large as a multi-kilobase cassette that can contain promoters, cDNAs, etc. Both single-stranded and double-stranded oligonucleotide donors can be used. These oligonucleotides range in size from less than 100 nt to over many kb, though longer ssDNA can also be generated and used. Double-stranded donors are often used, including PCR amplicons, plasmids, and mini-circles. In general, it has been found that an AAV vector is a very effective means of delivery of a donor template, though the packaging limits for individual donors is <5 kb. Active transcription of the donor increased HDR three-fold, indicating the inclusion of promoter can increase conversion. Conversely, CpG methylation of the donor can decrease gene expression and HDR.

In some embodiments, the donor DNA can be supplied with the nuclease or independently by a variety of different methods, for example by transfection, nanoparticle, micro-injection, or viral transduction. A range of tethering options can be used to increase the availability of the donors for HDR in some embodiments. Examples include attaching the donor to the nuclease, attaching to DNA binding proteins that bind nearby, or attaching to proteins that are involved in DNA end binding or repair.

In addition to genome editing by NHEJ or HDR, site-specific gene insertions can be conducted that use both the NHEJ pathway and HR. A combination approach can be applicable in certain settings, possibly including intron/exon borders. NHEJ can prove effective for ligation in the intron, while the error-free HDR can be better suited in the coding region.

In some embodiments, an exogenous sequence that is intended to be inserted into a genome is a nucleotide sequence encoding a FOXP3 or a functional derivative thereof. The functional derivative of a FOXP3 can include a derivative of the FOXP3 that has a substantial activity of a wild-type FOXP3, such as the wild-type human FOXP3, e.g., at least or at least about 30%, at or about 40%, at or about 50%, at or about 60%, at or about 70%, at or about 80%, at or about 90%, at or about 95% or at or about 100% of the activity that the wild-type FOXP3 exhibits. In some embodiments, the functional derivative of a FOXP3 can have at least or at least about 30%, at or about 40%, at or about 50%, at or about 60%, at or about 70%, at or about 80%, at or about 85%, at or about 90%, at or about 95%, at or about 96%, at or about 97%, at or about 98% or at or about 99% amino acid sequence identity to the FOXP3, e.g., the wild-type FOXP3. In some embodiments, one having ordinary skill in the art can use a number of methods known in the field to test the functionality or activity of a compound, e.g., a peptide or protein. The functional derivative of the FOXP3 can also include any fragment of the wild-type FOXP3 or fragment of a modified FOXP3 that has conservative modification on one or more of amino acid residues in the full length, wild-type FOXP3. Thus, in some embodiments, a nucleic acid sequence encoding a functional derivative of a FOXP3 can have at least or at least about 30%, at or about 40%, at or about 50%, at or about 60%, at or about 70%, at or about 80%, at or about 85%, at or about 90%, at or about 95%, at or about 96%, at or about 97%, at or about 98% or at or about 99% nucleic acid sequence identity to a nucleic acid sequence encoding the FOXP3, e.g., the wild-type FOXP3. In some embodiments, the FOXP3 is human wild-type FOXP3.

In some embodiments where the insertion of a nucleic acid encoding a FOXP3 or a functional derivative thereof is concerned, a cDNA of the FOXP3gene or a functional derivative thereof can be inserted into a genome of a subject having a defective FOXP3 gene or its regulatory sequences. In such a case, a donor DNA or donor template can be an expression cassette or vector construct having a sequence encoding the FOXP3 or a functional derivative thereof, e.g., a cDNA sequence.

In some embodiments, according to any of the donor templates described herein comprising a donor cassette, the donor cassette is flanked on one or both sides by a gRNA target site. For example, such a donor template may comprise a donor cassette with a gRNA target site 5′ of the donor cassette and/or a gRNA target site 3′ of the donor cassette. In some embodiments, the donor template comprises a donor cassette with a gRNA target site 5′ of the donor cassette. In some embodiments, the donor template comprises a donor cassette with a gRNA target site 3′ of the donor cassette. In some embodiments, the donor template comprises a donor cassette with a gRNA target site 5′ of the donor cassette and a gRNA target site 3′ of the donor cassette. In some embodiments, the donor template comprises a donor cassette with a gRNA target site 5′ of the donor cassette and a gRNA target site 3′ of the donor cassette, and the two gRNA target sites comprise the same sequence. In some embodiments, the donor template comprises at least one gRNA target site, and the at least one gRNA target site in the donor template comprises the same sequence as a gRNA target site in a target locus into which the donor cassette of the donor template is to be integrated. In some embodiments, the donor template comprises at least one gRNA target site, and the at least one gRNA target site in the donor template comprises the reverse complement of a gRNA target site in a target locus into which the donor cassette of the donor template is to be integrated. In some embodiments, the donor template comprises a donor cassette with a gRNA target site 5′ of the donor cassette and a gRNA target site 3′ of the donor cassette, and the two gRNA target sites in the donor template comprises the same sequence as a gRNA target site in a target locus into which the donor cassette of the donor template is to be integrated. In some embodiments, the donor template comprises a donor cassette with a gRNA target site 5′ of the donor cassette and a gRNA target site 3′ of the donor cassette, and the two gRNA target sites in the donor template comprises the reverse complement of a gRNA target site in a target locus into which the donor cassette of the donor template is to be integrated.

In some embodiments, provided herein is a donor template comprising a nucleotide sequence encoding a FOXP3 or a functional derivative thereof for targeted integration into a FOXP3 gene, wherein the donor template comprises, from 5′ to 3′, i) a first gRNA target site; ii) a splice acceptor; iii) the nucleotide sequence encoding a FOXP3 or a functional derivative thereof, and iv) a polyadenylation signal. In some embodiments, the donor template further comprises a second gRNA target site downstream of the iv) polyadenylation signal. In some embodiments, the first gRNA target site and the second gRNA target site are the same. In some embodiments, the donor template further comprises a polynucleotide spacer between the i) first gRNA target site and the ii) splice acceptor. In some embodiments, the polynucleotide spacer is 18 nucleotides in length. In some embodiments, the donor template is flanked on one side by a first AAV ITR and/or flanked on the other side by a second AAV ITR. In some embodiments, the first AAV ITR is an AAV2 ITR and/or the second AAV ITR is an AAV2 ITR. In some embodiments, the FOXP3 is human wild-type FOXP3.

Nucleic Acid Encoding a Site-Directed Polypeptide or DNA Endonuclease

In some embodiments, the methods of genome edition and compositions therefore can use a nucleic acid sequence (or oligonucleotide) encoding a site-directed polypeptide or DNA endonuclease. The nucleic acid sequence encoding the site-directed polypeptide can be DNA or RNA. If the nucleic acid sequence encoding the site-directed polypeptide is RNA, it can be covalently linked to a gRNA sequence or exist as a separate sequence. In some embodiments, a peptide sequence of the site-directed polypeptide or DNA endonuclease can be used instead of the nucleic acid sequence thereof.

Vectors

In another aspect, the present disclosure provides a nucleic acid having a nucleotide sequence encoding a genome-targeting nucleic acid of the disclosure, a site-directed polypeptide of the disclosure, and/or any nucleic acid or proteinaceous molecule necessary to carry out the embodiments of the methods of the disclosure. In some embodiments, such a nucleic acid is a vector (e.g., a recombinant expression vector).

Expression vectors contemplated include, but are not limited to, viral vectors based on vaccinia virus, poliovirus, adenovirus, adeno-associated virus, SV40, herpes simplex virus, human immunodeficiency virus, retrovirus (e.g., Murine Leukemia Virus, spleen necrosis virus, or vectors derived from retroviruses such as Rous Sarcoma Virus, Harvey Sarcoma Virus, avian leukosis virus, a lentivirus, human immunodeficiency virus, myeloproliferative sarcoma virus, or mammary tumor virus) or other recombinant vectors. Other vectors contemplated for eukaryotic target cells include, but are not limited to, the vectors pXTI, pSG5, pSVK3, pBPV, pMSG, or pSVLSV40 (Pharmacia). Additional vectors contemplated for eukaryotic target cells include, but are not limited to, the vectors pCTx-1, pCTx-2, or pCTx-3. Other vectors can be used so long as they are compatible with the host cell.

In some embodiments, a vector has one or more transcription and/or translation control elements. Depending on the host/vector system utilized, any of a number of suitable transcription and translation control elements, including constitutive and inducible promoters, transcription enhancer elements, transcription terminators, etc. can be used in the expression vector. In some embodiments, the vector is a self-inactivating vector that either inactivates the viral sequences or the components of the CRISPR machinery or other elements.

Non-limiting examples of suitable eukaryotic promoters (i.e., promoters functional in a eukaryotic cell) include those from cytomegalovirus (CMV) immediate early, herpes simplex virus (HSV) thymidine kinase, early or late SV40, long terminal repeats (LTRs) from retrovirus, human elongation factor-1 promoter (EF1), a hybrid construct having the cytomegalovirus (CMV) enhancer fused to the chicken beta-actin promoter (CAG), murine stem cell virus promoter (MSCV), phosphoglycerate kinase-1 locus promoter (PGK), or mouse metallothionein-I.

For expressing small RNAs, including guide RNAs used in connection with Cas endonuclease, various promoters such as RNA polymerase III promoters, including for example U6 or H1, can be useful. Descriptions of and parameters for enhancing the use of such promoters are known in art, and additional information and approaches are regularly being described; see, e.g., Ma, H. et al. (2014). Molecular Therapy—Nucleic Acids 3, ei61, doi:10.1038/mtna.2014.12.

The expression vector can also contain a ribosome binding site for translation initiation and a transcription terminator. The expression vector can also include appropriate sequences for amplifying expression. The expression vector can also include nucleotide sequences encoding non-native tags (e.g., histidine tag, hemagglutinin tag, or green fluorescent protein, etc.) that are fused to the site-directed polypeptide, thus resulting in a fusion protein. In some embodiments, a promoter is an inducible promoter (e.g., a heat shock promoter, tetracycline-regulated promoter, steroid-regulated promoter, metal-regulated promoter, or estrogen receptor-regulated promoter, etc.). In some embodiments, a promoter is a constitutive promoter (e.g., CMV promoter, or UBC promoter). In some embodiments, the promoter is a spatially restricted or temporally restricted promoter (e.g., a tissue specific promoter, or a cell type specific promoter, etc.). In some embodiments, a vector does not have a promoter for at least one gene to be expressed in a host cell if the gene is going to be expressed, after it is inserted into a genome, under an endogenous promoter present in the genome.

### Site-Directed Polypeptide or DNA Endonuclease

Modifications of a target DNA due to NHEJ and/or HDR can lead to, for example, mutations, deletions, alterations, integrations, gene correction, gene replacement, gene tagging, transgene insertion, nucleotide deletion, gene disruption, translocations, and/or gene mutation. The process of integrating non-native nucleic acid into genomic DNA is an example of genome editing.

A site-directed polypeptide is a nuclease used in genome editing to cleave DNA. The site-directed polypeptide can be administered to a cell or a subject as either: one or more polypeptides, or one or more mRNAs encoding the polypeptide.

In the context of a CRISPR/Cas or CRISPR/Cpf1 system, the site-directed polypeptide can bind to a guide RNA that, in turn, specifies the site in the target DNA to which the polypeptide is directed. In embodiments of CRISPR/Cas or CRISPR/Cpf1 systems herein, the site-directed polypeptide is an endonuclease, such as a DNA endonuclease.

In some embodiments, a site-directed polypeptide has a plurality of nucleic acid-cleaving (e.g., nuclease) domains. Two or more nucleic acid-cleaving domains can be linked together via a linker. In some embodiments, the linker has a flexible linker. Linkers can have 1, 2, 3, 4, 5, 6, 7, 8, 9, 10, 11, 12, 13, 14, 15, 16, 17, 18, 19, 20, 21, 22, 23, 24, 25, 30, 35, 40, or more amino acids in length.

Naturally-occurring wild-type Cas9 enzymes have two nuclease domains, an HNH nuclease domain and a RuvC domain. Cas9 enzymes contemplated herein have an HNH or HNH-like nuclease domain, and/or a RuvC or RuvC-like nuclease domain.

HNH or HNH-like domains have a McrA-like fold. HNH or HNH-like domains has two antiparallel β-strands and an α-helix. HNH or HNH-like domains has a metal binding site (e.g., a divalent cation binding site). HNH or HNH-like domains can cleave one strand of a target nucleic acid (e.g., the complementary strand of the crRNA targeted strand).

RuvC or RuvC-like domains have an RNaseH or RNaseH-like fold. RuvC/RNaseH domains are involved in a diverse set of nucleic acid-based functions including acting on both RNA and DNA. The RNaseH domain has 5 β-strands surrounded by a plurality of α-helices. RuvC/RNaseH or RuvC/RNaseH-like domains have a metal binding site (e.g., a divalent cation binding site). RuvC/RNaseH or RuvC/RNaseH-like domains can cleave one strand of a target nucleic acid (e.g., the non-complementary strand of a double-stranded target DNA).

In some embodiments, the site-directed polypeptide has an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to a wild-type exemplary site-directed polypeptide [e.g., Cas9 from S. pyogenes, US2014/0068797 Sequence ID No. 8 or Sapranauskas, R. et al. (2011). Nucleic Acids Res, 39(21): 9275-9282], and various other site-directed polypeptides).

In some embodiments, the site-directed polypeptide has an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to the nuclease domain of a wild-type exemplary site-directed polypeptide (e.g., Cas9 from S. pyogenes, supra).

In some embodiments, a site-directed polypeptide has at least 70, 75, 80, 85, 90, 95, 97, 99, or 100% identity to a wild-type site-directed polypeptide (e.g., Cas9 from S. pyogenes, supra) over 10 contiguous amino acids. In some embodiments, a site-directed polypeptide has at most: 70, 75, 80, 85, 90, 95, 97, 99, or 100% identity to a wild-type site-directed polypeptide (e.g., Cas9 from S. pyogenes, supra) over 10 contiguous amino acids. In some embodiments, a site-directed polypeptide has at least: 70, 75, 80, 85, 90, 95, 97, 99, or 100% identity to a wild-type site-directed polypeptide (e.g., Cas9 from S. pyogenes, supra) over 10 contiguous amino acids in an HNH nuclease domain of the site-directed polypeptide. In some embodiments, a site-directed polypeptide has at most: 70, 75, 80, 85, 90, 95, 97, 99, or 100% identity to a wild-type site-directed polypeptide (e.g., Cas9 from S. pyogenes, supra) over 10 contiguous amino acids in an HNH nuclease domain of the site-directed polypeptide. In some embodiments, a site-directed polypeptide has at least: 70, 75, 80, 85, 90, 95, 97, 99, or 100% identity to a wild-type site-directed polypeptide (e.g., Cas9 from S. pyogenes, supra) over 10 contiguous amino acids in a RuvC nuclease domain of the site-directed polypeptide. In some embodiments, a site-directed polypeptide has at most: 70, 75, 80, 85, 90, 95, 97, 99, or 100% identity to a wild-type site-directed polypeptide (e.g., Cas9 from S. pyogenes, supra) over 10 contiguous amino acids in a RuvC nuclease domain of the site-directed polypeptide.

In some embodiments, the site-directed polypeptide has a modified form of a wild-type exemplary site-directed polypeptide. The modified form of the wild-type exemplary site-directed polypeptide has a mutation that reduces the nucleic acid-cleaving activity of the site-directed polypeptide. In some embodiments, the modified form of the wild-type exemplary site-directed polypeptide has less than 90%, less than 80%, less than 70%, less than 60%, less than 50%, less than 40%, less than 30%, less than 20%, less than 10%, less than 5%, or less than 1% of the nucleic acid-cleaving activity of the wild-type exemplary site-directed polypeptide (e.g., Cas9 from S. pyogenes, supra) but not zero. The modified form of the site-directed polypeptide can also have no substantial nucleic acid-cleaving activity. When a site-directed polypeptide is a modified form that has no substantial nucleic acid-cleaving activity, it is referred to herein as “enzymatically inactive.”

In some embodiments, the modified form of the site-directed polypeptide has a mutation such that it can induce a single-strand break (SSB) on a target nucleic acid (e.g., by cutting only one of the sugar-phosphate backbones of a double-strand target nucleic acid). In some embodiments, the mutation results in less than 90%, less than 80%, less than 70%, less than 60%, less than 50%, less than 40%, less than 30%, less than 20%, less than 10%, less than 5%, or less than 1% of the nucleic acid-cleaving activity in one or more of the plurality of nucleic acid-cleaving domains of the wild-type site directed polypeptide (e.g., Cas9 from S. pyogenes, supra) but not zero. In some embodiments, the mutation results in one or more of the plurality of nucleic acid-cleaving domains retaining the ability to cleave the complementary strand of the target nucleic acid, but reducing its ability to cleave the non-complementary strand of the target nucleic acid. In some embodiments, the mutation results in one or more of the plurality of nucleic acid-cleaving domains retaining the ability to cleave the non-complementary strand of the target nucleic acid, but reducing its ability to cleave the complementary strand of the target nucleic acid. For example, residues in the wild-type exemplary S. pyogenes Cas9 polypeptide, such as Asp10, His840, Asn854, and Asn856, are mutated to inactivate one or more of the plurality of nucleic acid-cleaving domains (e.g., nuclease domains). In some embodiments, the residues to be mutated correspond to residues Asp10, His840, Asn854, and Asn856 in the wild-type exemplary S. pyogenes Cas9 polypeptide (e.g., as determined by sequence and/or structural alignment). Non-limiting examples of mutations include D10A, H840A, N854A, or N856A. One skilled in the art will recognize that mutations other than alanine substitutions are suitable.

In some embodiments, a D10A mutation is combined with one or more of H840A, N854A, or N856A mutations to produce a site-directed polypeptide substantially lacking DNA cleavage activity. In some embodiments, a H840A mutation is combined with one or more of D10A, N854A, or N856A mutations to produce a site-directed polypeptide substantially lacking DNA cleavage activity. In some embodiments, a N854A mutation is combined with one or more of H840A, D10A, or N856A mutations to produce a site-directed polypeptide substantially lacking DNA cleavage activity. In some embodiments, a N856A mutation is combined with one or more of H840A, N854A, or D10A mutations to produce a site-directed polypeptide substantially lacking DNA cleavage activity. Site-directed polypeptides that have one substantially inactive nuclease domain are referred to as “nickases”.

In some embodiments, variants of RNA-guided endonucleases, for example Cas9, can be used to increase the specificity of CRISPR-mediated genome editing. Wild type Cas endonucleases are generally guided by a single guide RNA designed to hybridize with a specified ˜20 nucleotide sequence in the target sequence (such as an endogenous genomic locus). However, several mismatches can be tolerated between the guide RNA and the target locus, effectively reducing the length of required homology in the target site to, for example, as little as 13 nt of homology, and thereby resulting in elevated potential for binding and double-strand nucleic acid cleavage by a CRISPR/Cas complex elsewhere in the target genome—also known as off-target cleavage. Because nickase variants of Cas endonucelases each only cut one strand, to create a double-strand break it is necessary for a pair of nickases to bind in close proximity and on opposite strands of the target nucleic acid, thereby creating a pair of nicks, which is the equivalent of a double-strand break. This requires that two separate guide RNAs—one for each nickase—must bind in close proximity and on opposite strands of the target nucleic acid. This requirement essentially doubles the minimum length of homology needed for the double-strand break to occur, thereby reducing the likelihood that a double-strand cleavage event will occur elsewhere in the genome, where the two guide RNA sites—if they exist—are unlikely to be sufficiently close to each other to enable the double-strand break to form. As described in the art, nickases can also be used to promote HDR versus NHEJ. HDR can be used to introduce selected changes into target sites in the genome through the use of specific donor sequences that effectively mediate the desired changes. Descriptions of various CRISPR/Cas systems for use in gene editing can be found, e.g., in International Patent Application no. WO2013/176772, and in Sander, J. D. et al. (2014). Nature Biotechnology, 32(4):347-355, and references cited therein.

In some embodiments, the site-directed polypeptide (e.g., variant, mutated, enzymatically inactive or conditionally enzymatically inactive site-directed polypeptide) targets nucleic acid. In some embodiments, the site-directed polypeptide (e.g., variant, mutated, enzymatically inactive or conditionally enzymatically inactive endoribonuclease) targets DNA. In some embodiments, the site-directed polypeptide (e.g., variant, mutated, enzymatically inactive or conditionally enzymatically inactive endoribonuclease) targets RNA.

In some embodiments, the site-directed polypeptide has one or more non-native sequences (e.g., the site-directed polypeptide is a fusion protein).

In some embodiments, the site-directed polypeptide has an amino acid sequence having at least 15% amino acid identity to a Cas endonuclease from a bacterium (e.g., S. pyogenes), a nucleic acid binding domain, and two nucleic acid cleaving domains (e.g., an HNH domain and a RuvC domain).

In some embodiments, the site-directed polypeptide has an amino acid sequence having at least 15% amino acid identity to a Cas endonuclease from a bacterium (e.g., S. pyogenes), and two nucleic acid cleaving domains (e.g., an HNH domain and a RuvC domain).

In some embodiments, the site-directed polypeptide has an amino acid sequence having at least 15% amino acid identity to a Cas endonuclease from a bacterium (e.g., S. pyogenes), and two nucleic acid cleaving domains, wherein one or both of the nucleic acid cleaving domains have at least 50% amino acid identity to a nuclease domain from a Cas endonuclease from a bacterium (e.g., S. pyogenes).

In some embodiments, the site-directed polypeptide has an amino acid sequence having at least 15% amino acid identity to a Cas endonuclease from a bacterium (e.g., S. pyogenes), two nucleic acid cleaving domains (e.g., an HNH domain and a RuvC domain), and non-native sequence (for example, a nuclear localization signal) or a linker linking the site-directed polypeptide to a non-native sequence.

In some embodiments, the site-directed polypeptide has an amino acid sequence having at least 15% amino acid identity to a Cas endonuclease from a bacterium (e.g., S. pyogenes), two nucleic acid cleaving domains (e.g., an HNH domain and a RuvC domain), wherein the site-directed polypeptide has a mutation in one or both of the nucleic acid cleaving domains that reduces the cleaving activity of the nuclease domains by at least 50%.

In some embodiments, the site-directed polypeptide has an amino acid sequence having at least 15% amino acid identity to a Cas endonuclease from a bacterium (e.g., S. pyogenes), and two nucleic acid cleaving domains (e.g., an HNH domain and a RuvC domain), wherein one of the nuclease domains has mutation of aspartic acid 10, and/or wherein one of the nuclease domains has mutation of histidine 840, and wherein the mutation reduces the cleaving activity of the nuclease domain(s) by at least 50%.

In some embodiments, the one or more site-directed polypeptides, e.g., DNA endonucleases, include two nickases that together effect one double-strand break at a specific locus in the genome, or four nickases that together effect two double-strand breaks at specific loci in the genome. Alternatively, one site-directed polypeptide, e.g., DNA endonuclease, affects one double-strand break at a specific locus in the genome.

In some embodiments, a polynucleotide encoding a site-directed polypeptide can be used to edit genome. In some of such embodiments, the polynucleotide encoding a site-directed polypeptide is codon-optimized according to methods known in the art for expression in the cell containing the target DNA of interest. For example, if the intended target nucleic acid is in a human cell, a human codon-optimized polynucleotide encoding a Cas endonuclease (e.g., a Cas9) is contemplated for use for producing the Cas endonuclease polypeptide.

The following provides some examples of site-directed polypeptides that can be used in various embodiments of the disclosures.

CRISPR Endonuclease System

A CRISPR (Clustered Regularly Interspaced Short Palindromic Repeats) genomic locus can be found in the genomes of many prokaryotes (e.g., bacteria and archaea). In prokaryotes, the CRISPR locus encodes products that function as a type of immune system to help defend the prokaryotes against foreign invaders, such as virus and phage. There are three stages of CRISPR locus function: integration of new sequences into the CRISPR locus, expression of CRISPR RNA (crRNA), and silencing of foreign invader nucleic acid. Five types of CRISPR systems (e.g., Type I, Type II, Type III, Type U, and Type V) have been identified.

A CRISPR locus includes a number of short repeating sequences referred to as “repeats.” When expressed, the repeats can form secondary hairpin structures (e.g., hairpins) and/or unstructured single-stranded sequences. The repeats usually occur in clusters and frequently diverge between species. The repeats are regularly interspaced with unique intervening sequences referred to as “spacers,” resulting in a repeat-spacer-repeat locus architecture. The spacers are identical to or have high homology with known foreign invader sequences. A spacer-repeat unit encodes a crisprRNA (crRNA), which is processed into a mature form of the spacer-repeat unit. A crRNA has a “seed” or spacer sequence that is involved in targeting a target nucleic acid (in the naturally occurring form in prokaryotes, the spacer sequence targets the foreign invader nucleic acid). A spacer sequence is located at the 5′ or 3′ end of the crRNA.

A CRISPR locus also has polynucleotide sequences encoding CRISPR Associated (Cas) genes. Cas genes encode endonucleases involved in the biogenesis and the interference stages of crRNA function in prokaryotes. Some Cas genes have homologous secondary and/or tertiary structures.

Type II CRISPR Systems

crRNA biogenesis in a Type II CRISPR system in nature requires a trans-activating CRISPR RNA (tracrRNA). The tracrRNA is modified by endogenous RNaseIII, and then hybridizes to a crRNA repeat in the pre-crRNA array. Endogenous RNaseIII is recruited to cleave the pre-crRNA. Cleaved crRNAs are subjected to exoribonuclease trimming to produce the mature crRNA form (e.g., 5′ trimming). The tracrRNA remains hybridized to the crRNA, and the tracrRNA and the crRNA associate with a site-directed polypeptide (e.g., a Cas endonuclease, such as a Cas9). The crRNA of the crRNA-tracrRNA-Cas complex guides the complex to a target nucleic acid to which the crRNA can hybridize. Hybridization of the crRNA to the target nucleic acid activates the Cas endonuclease for targeted nucleic acid cleavage. The target nucleic acid in a Type II CRISPR system is referred to as a protospacer adjacent motif (PAM). In nature, the PAM is essential to facilitate binding of a site-directed polypeptide (e.g., Cas9) to the target nucleic acid. Type II systems (also referred to as Nmeni or CASS4) are further subdivided into Type II-A (CASS4) and II-B (CASS4a). Jinek, M. et al. (2012). Science, 337(6096):816-821 showed that the CRISPR/Cas9 system is useful for RNA-programmable genome editing, and International Patent Application no. WO 2013/176772 provides numerous examples and applications of the CRISPR/Cas endonuclease system for site-specific gene editing.

Type V CRISPR Systems

Type V CRISPR systems have several important differences from Type II systems. For example, Cpf1 is a single RNA-guided endonuclease that, in contrast to Type II systems, lacks tracrRNA. In fact, Cpf1-associated CRISPR arrays are processed into mature crRNAs without the requirement of an additional trans-activating tracrRNA. The Type V CRISPR array is processed into short mature crRNAs of 42-44 nucleotides in length, with each mature crRNA beginning with 19 nucleotides of direct repeat followed by 23-25 nucleotides of spacer sequence. In contrast, mature crRNAs in Type II systems start with 20-24 nucleotides of spacer sequence followed by about 22 nucleotides of direct repeat. Also, Cpf1 utilizes a T-rich protospacer-adjacent motif such that Cpf1-crRNA complexes efficiently cleave target DNA preceded by a short T-rich PAM, which is in contrast to the G-rich PAM following the target DNA for Type II systems. Thus, Type V systems cleave at a point that is distant from the PAM, while Type II systems cleave at a point that is adjacent to the PAM. In addition, in contrast to Type II systems, Cpf1 cleaves DNA via a staggered DNA double-stranded break with a 4 or 5 nucleotide 5′ overhang. Type II systems cleave via a blunt double-stranded break. Similar to Type II systems, Cpf1 contains a predicted RuvC-like endonuclease domain, but lacks a second HNH endonuclease domain, which is in contrast to Type II systems.

Cas Genes/Polypeptides and Protospacer Adjacent Motifs

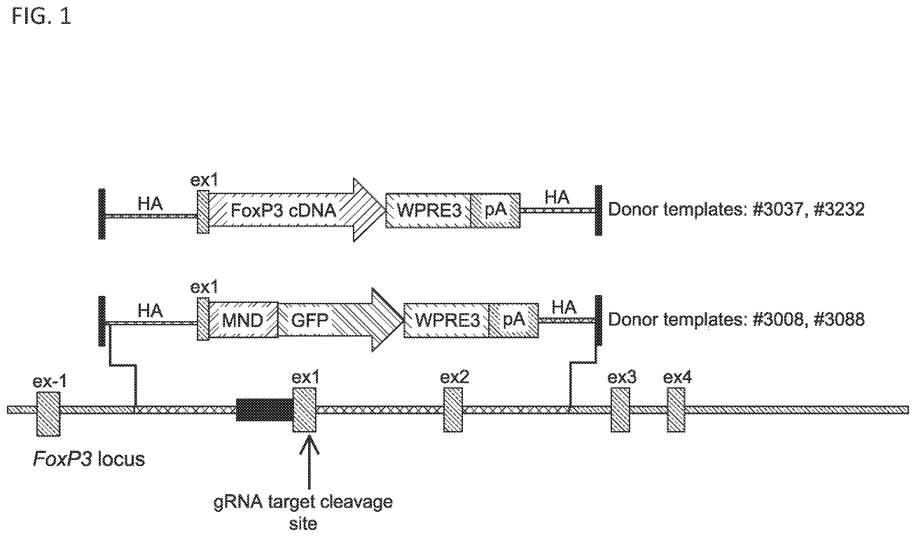

Exemplary CRISPR/Cas polypeptides include the Cas9 polypeptides in Fig. 1 of Fonfara, I. et al. (2014). Nucleic Acids Res., 42(4):2577-2590. The CRISPR/Cas gene naming system has undergone extensive rewriting since the Cas genes were discovered. Fig. 5 of Fonfara, supra, provides PAM sequences for the Cas9 polypeptides from various species.

Complexes of a Genome-Targeting Nucleic Acid and a Site-Directed Polypeptide

A genome-targeting nucleic acid interacts with a site-directed polypeptide (e.g., a nucleic acid-guided nuclease such as Cas9), thereby forming a complex. The genome-targeting nucleic acid (e.g., gRNA) guides the site-directed polypeptide to a target nucleic acid.

As stated previously, in some embodiments the site-directed polypeptide and genome-targeting nucleic acid can each be administered separately to a cell or a subject. On the other hand, in some other embodiments the site-directed polypeptide can be pre-complexed with one or more guide RNAs, or one or more crRNA together with a tracrRNA. The pre-complexed material can then be administered to a cell or a subject. Such pre-complexed material is known as a ribonucleoprotein particle (RNP).

### Methods of Editing Genome

One approach to express a FOXP3 protein or functional derivative thereof in an organism in need thereof is to use genome editing to target the integration of a nucleic acid comprising a coding sequence encoding the FOXP3 protein into an endogenous FOXP3 gene or a non-FOXP3 gene that is sufficiently expressed in a relevant cell type (e.g., T cell) in such a way that expression of the integrated coding sequence is driven by the endogenous promoter of the endogenous FOXP3 gene or non-FOXP3 gene. In some embodiments, where a non-FOXP3 gene is targeted, it is desirable that the expression of the non-FOXP3 gene be specific to the targeted cell type, e.g., CD34⁺ cells such as CD34⁺ hematopoietic stem cells, or cells derived therefrom (e.g., T cells) to avoid expression in non-relevant cell types.

In some embodiments, a knock-in strategy involves knocking-in a sequence encoding a FOXP3 or a functional derivative thereof, such as a wild-type FOXP3 gene (e.g., a wild-type human FOXP3 gene), a FOXP3 cDNA, or a FOXP3 minigene (having natural or synthetic enhancer and promoter, one or more exons, and natural or synthetic introns, and natural or synthetic 3′UTR and polyadenylation signal) into a genomic sequence. In some embodiments, the genomic sequence where the FOXP3-encoding sequence is inserted is at, within, or near the FOXP3 gene. In some embodiments, the genomic sequence where the FOXP3-encoding sequence is inserted is at, within, or near exon 1 of the FOXP3 gene.

In some embodiments, provided herein are methods to knock-in a sequence encoding a FOXP3 or a functional derivative thereof into a genome. In one aspect, the present disclosure provides insertion of a nucleic acid comprising a sequence encoding a FOXP3 or a functional derivative thereof into a genome of a cell. In some embodiments, the FOXP3-encoding sequence encodes a wild-type FOXP3. The functional derivative of FOXP3 can include a derivative of FOXP3 that has a substantial activity of a wild-type FOXP3, such as the wild-type human FOXP3, e.g., at least or at least about 30%, at or about 40%, at or about 50%, at or about 60%, at or about 70%, at or about 80%, at or about 90%, at or about 95% or at or about 100% of the activity that the wild-type FOXP3 exhibits. In some embodiments, the functional derivative of FOXP3 has at least or at least about 30%, at or about 40%, at or about 50%, at or about 60%, at or about 70%, at or about 80%, at or about 85%, at or about 90%, at or about 95%, at or about 96%, at or about 97%, at or about 98% or at or about 99% amino acid sequence identity to a FOXP3, e.g., a wild-type FOXP3. In some embodiments, the FOXP3 is encoded by a nucleotide sequence that lacks introns (e.g., FOXP3 cDNA). One having ordinary skill in the art can use methods known in the art to test the functionality or activity of a FOXP3 derivative. The functional derivative of a FOXP3 can also include any fragment of a wild-type FOXP3 that has conservative modifications on one or more amino acid residues in a full length, wild-type FOXP3. Thus, in some embodiments, a nucleic acid sequence encoding a functional derivative of a FOXP3 can have at least or at least about 30%, at or about 40%, at or about 50%, at or about 60%, at or about 70%, at or about 80%, at or about 85%, at or about 90%, at or about 95%, at or about 96%, at or about 97%, at or about 98% or at or about 99% nucleic acid sequence identity to a nucleic acid sequence encoding the FOXP3, e.g., a wild-type FOXP3. In some embodiments, the FOXP3 or a functional variant thereof is a human wild-type FOXP3.

In some embodiments, the genome editing methods utilize a DNA endonuclease such as a CRISPR/Cas endonuclease to genetically introduce (knock-in) a sequence encoding a FOXP3 or a functional derivative thereof. In some embodiments, the DNA endonuclease is a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, a homolog thereof, a recombinant of the naturally occurring molecule, a codon-optimized, or modified version thereof, or a combination of any of the foregoing. In some embodiments, the DNA endonuclease is a Cas9. In some embodiments, the Cas9 is from Streptococcus pyogenes (spCas9). In some embodiments, the Cas9 is from Staphylococcus lugdunensis (SluCas9).

In some embodiments, the cell subject to the genome-edition has one or more mutation(s) in the genome which results in a decrease of the expression of an endogenous FOXP3 gene as compared to the expression in a normal cell that does not have such mutation(s). The normal cell can be a healthy or control cell that is originated (or isolated) from a different subject who does not have FOXP3 gene defects. In some embodiments, the cell subject to the genome-edition can be originated (or isolated) from a subject who is in need of treatment of a FOXP3 gene related condition or disorder, e.g. a subject suffering from an autoimmune disorder (e.g., IPEX syndrome). Therefore, in some embodiments the expression of an endogenous FOXP3 gene in such cell is at or about 10%, at or about 20%, at or about 30%, at or about 40%, at or about 50%, at or about 60%, at or about 70%, at or about 80%, at or about 90% or at or about 100% decreased as compared to the expression of an endogenous FOXP3 gene in the normal cell.

In some embodiments, provided herein is a method of editing a genome in a CD34⁺ cell, the method comprising providing the following to the CD34⁺ cell: (a) a Cas DNA endonuclease (e.g., a Cas9 endonuclease) or nucleic acid encoding the Cas DNA endonuclease; (b) a gRNA (e.g., an sgRNA) or nucleic acid encoding the gRNA, wherein the gRNA is capable of targeting the Cas DNA endonuclease to a FOXP3 gene or a non-FOXP3 locus (e.g., AAVS1) in the genome of a cell, and (c) a donor template comprising a FOXP3 coding sequence. In some embodiments, the Cas DNA endonuclease is a Cas9 endonuclease (e.g., a Cas9 endonuclease from Streptococcus pyogenes). In some embodiments, the gRNA comprises a spacer sequence complementary to a target sequence in a FOXP3 gene. In some embodiments, the gRNA comprises a spacer sequence complementary to a target sequence in exon 1 of a FOXP3 gene. In some embodiments, the gRNA comprises a spacer sequence from any one of SEQ ID NOs: 1-7 and 27-29 or a variant thereof having no more than 3 mismatches compared to any one of SEQ ID NOs: 1-7 and 27-29. In some embodiments, the gRNA comprises a spacer sequence from any one of SEQ ID NOs: 1-7 or a variant thereof having no more than 3 mismatches compared to any one of SEQ ID NOs: 1-7. In some embodiments, the gRNA comprises a spacer sequence from any one of SEQ ID NOs: 2, 3, and 5, or a variant thereof having no more than 3 mismatches compared to any one of SEQ ID NOs: 2, 3, and 5. In some embodiments, the gRNA comprises a spacer sequence complementary to a target sequence in a non-FOXP3 locus (e.g., AAVS1). In some embodiments, the gRNA comprises a spacer sequence from any one of SEQ ID NOs: 15-20 or a variant thereof having no more than 3 mismatches compared to any one of SEQ ID NOs: 15-20. In some embodiments, the FOXP3 coding sequence encodes FOXP3 or a functional derivative thereof. In some embodiments, the FOXP3 coding sequence is a FOXP3 cDNA. An exemplary FOXP3 cDNA sequence can be found in the AAV donor template having the nucleotide sequence of SEQ ID NO: 34. In some embodiments, the method comprises providing to the CD34⁺ cell the Cas DNA endonuclease. In some embodiments, the method comprises providing to the CD34⁺ cell nucleic acid encoding the Cas DNA endonuclease. In some embodiments, the method comprises providing to the CD34⁺ cell the gRNA. In some embodiments, the gRNA is an sgRNA. In some embodiments, the method comprises providing to the CD34⁺ cell nucleic acid encoding the gRNA. In some embodiments, the method further comprises providing to the CD34⁺ cell one or more additional gRNAs or nucleic acid encoding the one or more additional gRNAs.

In some embodiments, according to any of the methods of editing a genome in a cell described herein, the DNA endonuclease is a Cas9. In some embodiments, the Cas9 is from Streptococcus pyogenes (spCas9). In some embodiments, the Cas9 is from Staphylococcus lugdunensis (SluCas9).

In some embodiments, according to any of the methods of editing a genome in a cell described herein, the nucleic acid sequence encoding a FOXP3 or a functional derivative thereof is codon-optimized for expression in the cell. In some embodiments, the cell is a human cell.

In some embodiments, according to any of the methods of editing a genome in a cell described herein, the method employs a nucleic acid encoding the DNA endonuclease. In some embodiments, the nucleic acid encoding the DNA endonuclease is codon-optimized for expression in the cell. In some embodiments, the cell is a human cell, e.g., a human CD34⁺ cell. In some embodiments, the nucleic acid encoding the DNA endonuclease is DNA, such as a DNA plasmid. In some embodiments, the nucleic acid encoding the DNA endonuclease is RNA, such as mRNA.

In some embodiments, according to any of the methods of editing a genome in a cell described herein, the donor template comprises a donor cassette comprising the nucleic acid sequence encoding a FOXP3 or a functional derivative thereof, and the donor template is configured such that the donor cassette is capable of being integrated into the genomic locus targeted by the gRNA of (b) by homology directed repair (HDR). In some embodiments, the donor cassette is flanked on both sides by homology arms corresponding to sequences in the targeted genomic locus. In some embodiments, the homology arms are at least or at least about 0.2 kb (such as at least or at least about any of 0.3 kb, 0.4 kb, 0.5 kb, 0.6 kb, 0.7 kb, 0.8 kb, 0.9 kb, or 1 kb, or greater) in length. In some embodiments, the homology arms are at least or at least about 0.8 kb in length. Exemplary homology arms include homology arms from donor templates having the sequence of SEQ ID NO: 34 or 161. Exemplary donor templates include donor templates having the sequence of SEQ ID NO: 34 or 161. In some embodiments, the donor template is encoded in an Adeno Associated Virus (AAV) vector. In some embodiments, the AAV vector is an AAV6 vector.

In some embodiments, according to any of the methods of editing a genome in a cell described herein, the donor template comprises a donor cassette comprising the nucleic acid sequence encoding a FOXP3 or a functional derivative thereof, and the donor template is configured such that the donor cassette is capable of being integrated into the genomic locus targeted by the gRNA of (b) by non-homologous end joining (NHEJ). In some embodiments, the donor cassette is flanked on one or both sides by a gRNA target site. In some embodiments, the donor cassette is flanked on both sides by a gRNA target site. In some embodiments, the gRNA target site is a target site for a gRNA in the system. In some embodiments, the gRNA target site of the donor template is the reverse complement of a cell genome gRNA target site for a gRNA in the system. In some embodiments, the donor template is encoded in an Adeno Associated Virus (AAV) vector. In some embodiments, the AAV vector is an AAV6 vector.

In some embodiments, according to any of the methods of editing a genome in a cell described herein, the DNA endonuclease or nucleic acid encoding the DNA endonuclease is formulated in a liposome or lipid nanoparticle. In some embodiments, the liposome or lipid nanoparticle also comprises the gRNA. In some embodiments, the liposome or lipid nanoparticle is a lipid nanoparticle. In some embodiments, the method employs a lipid nanoparticle comprising nucleic acid encoding the DNA endonuclease and the gRNA. In some embodiments, the nucleic acid encoding the DNA endonuclease is an mRNA encoding the DNA endonuclease.

In some embodiments, according to any of the methods of editing a genome in a cell described herein, the DNA endonuclease is pre-complexed with the gRNA, forming a ribonucleoprotein (RNP) complex. In some embodiments, the RNP complex is provided to the cell by electroporation. In some embodiments, the donor template is an AAV donor template encoded in an AAV vector (e.g., an AAV6 vector). In some embodiments, the AAV donor template is provided to the cell at or around the same time that the RNP complex is provided to the cell. For example, in some embodiments, the cell is electroporated with the RNP complex and transduced with the AAV donor template on the same day. In some embodiments, the cell is electroporated with the RNP complex and transduced with the AAV donor template, wherein the electroporation and transduction are carried out no greater than or no greater than about 12 hours (such as no greater than or no greater than about any of 11 hours, 10 hours, 9 hours, 8 hours, 7 hours, 6 hours, 5 hours, 4 hours, 3 hours, 2 hours, or 1 hour, or less) apart. In some embodiments, the cell is electroporated with the RNP complex, plated, and transduced with the AAV donor template. In some embodiments, the cell is pre-stimulated in the presence of cytokines (e.g., TPO, SCF, FLT3L, or IL6 or any combination thereof) and/or small molecules (e.g., UM171 or StemRegenin (SR1)) capable of promoting HSC proliferation or self-renewal prior to providing the RNP and AAV donor template to the cell. In some embodiments, the pre-stimulation is carried out for at least or at least about 12 hours (such as at least or at least about any of 16 hours, 20 hours, 24 hours, 36 hours, or 48 hours, or more). In some embodiments, the pre-stimulation is carried out for at least or at least about 48 hours. In some embodiments, the pre-stimulation is carried out in a cell composition comprising the cell, and the concentration of cells in the cell composition and/or the culture media are such that at least or at least about 10% (e.g., at least or at least about 20%, 30%, 40%, or 50%) of the cells in the cell composition remain quiescent at the end of the pre-stimulation. In some embodiments, from or from about 10% to or to about 60% (e.g., from or from about 10% to or to about 50%, from or from about 10% to or to about 40%, or from or from about 10% to or to about 30%) of the cells in the cell composition remain quiescent at the end of the pre-stimulation. In some embodiments, the concentration of cells in the cell composition is no greater than or no greater than about 5×10⁵ (such as no greater than or no greater than about any of 4×10⁵, 3×10⁵, 2.5×10⁵, 2×10⁵, 1×10⁵, 0.5×10⁵, or fewer) cells/ml. In some embodiments, the concentration of cells in the cell composition is no greater than or no greater than about 2.5×10⁵ cells/ml.

In some embodiments, according to any of the methods of editing a genome in a cell described herein, the frequency of targeted integration of the donor template into a FOXP3 gene in the cell genome is from or from about 0.1% to or to about 99%. In some embodiments, the frequency of targeted integration is from or from about 2% to or to about 70% (such as from or from about 2% to or to about 65%, from or from about 2% to or to about 55%, from or from about 3% to or to about 70%, from or from about 5% to or to about 70%, from or from about 5% to or to about 60%, from or from about 5% to or to about 50%, from or from about 10% to or to about 60%, or from or from about 10% to or to about 50%). In some embodiments, the cell is a cell in a subject, such as a human subject.

### Target Sequence Selection

In some embodiments, shifts in the location of the 5′ boundary or the 3′ boundary or both relative to particular reference loci are used to facilitate or enhance particular applications of gene editing, which depend in part on the endonuclease system selected for the editing, as further described and illustrated herein.

In a first, non-limiting aspect of such target sequence selection, many endonuclease systems have rules or criteria that guide the initial selection of potential target sites for cleavage, such as the requirement of a PAM sequence motif in a particular position adjacent to the DNA cleavage sites in the case of CRISPR Type II or Type V endonucleases.

In another, non-limiting aspect of target sequence selection or optimization, the frequency of “off-target” activity for a particular combination of target sequence and gene editing endonuclease (e.g., the frequency of DSBs occurring at sites other than the selected target sequence) is assessed relative to the frequency of on-target activity. In some cases, cells that have been correctly edited at the desired locus can have a selective advantage relative to other cells. Illustrative, but non-limiting, examples of a selective advantage include the acquisition of attributes such as enhanced rates of replication, persistence, resistance to certain conditions, enhanced rates of successful engraftment or persistence in vivo following introduction into a subject, and other attributes associated with the maintenance or increased numbers or viability of such cells. In other cases, cells that have been correctly edited at the desired locus can be positively selected for by one or more screening methods used to identify, sort, or otherwise select for cells that have been correctly edited. Both selective advantage and directed selection methods can take advantage of the phenotype associated with the correction. In some embodiments, cells can be edited two or more times to create a second modification that creates a new phenotype that is used to select or purify the intended population of cells. Such a second modification could be created by adding a second gRNA for a selectable or screenable marker. In some cases, cells can be correctly edited at the desired locus using a DNA fragment that contains the cDNA and also a selectable marker.

In embodiments, whether any selective advantage is applicable or any directed selection is to be applied in a particular case, target sequence selection is also guided by consideration of off-target frequencies to enhance the effectiveness of the application and/or reduce the potential for undesired alterations at sites other than the desired target. As described further and illustrated herein and in the art, the occurrence of off-target activity is influenced by a number of factors including similarities and dissimilarities between the target site and various off-target sites, as well as the particular endonuclease used. Bioinformatics tools are available that assist in the prediction of off-target activity, and frequently such tools can also be used to identify the most likely sites of off-target activity, which can then be assessed in experimental settings to evaluate relative frequencies of off-target to on-target activity, thereby allowing the selection of sequences that have higher relative on-target activities. Illustrative examples of such techniques are provided herein, and others are known in the art.

Another aspect of target sequence selection relates to homologous recombination events. Sequences sharing regions of homology can serve as focal points for homologous recombination events that result in deletion of intervening sequences. Such recombination events occur during the normal course of replication of chromosomes and other DNA sequences, and also at other times when DNA sequences are being synthesized, such as in the case of repairs of double-strand breaks (DSBs), which occur on a regular basis during the normal cell replication cycle but can also be enhanced by the occurrence of various events (such as UV light and other inducers of DNA breakage) or the presence of certain agents (such as various chemical inducers). Many such inducers cause DSBs to occur indiscriminately in the genome, and DSBs are regularly being induced and repaired in normal cells. During repair, the original sequence can be reconstructed with complete fidelity, however, in some cases, small insertions or deletions (referred to as “indels”) are introduced at the DSB site.

DSBs can also be specifically induced at particular locations, as in the case of the endonucleases systems described herein, which can be used to cause directed or preferential gene modification events at selected chromosomal locations. The tendency for homologous sequences to be subject to recombination in the context of DNA repair (as well as replication) can be taken advantage of in a number of circumstances, and is the basis for one application of gene editing systems, such as CRISPR, in which homology directed repair is used to insert a sequence of interest, provided through use of a “donor” polynucleotide, into a desired chromosomal location.

Regions of homology between particular sequences, which can be small regions of “microhomology” that can have as few as ten base pairs or less, can also be used to bring about desired deletions. For example, a single DSB is introduced at a site that exhibits microhomology with a nearby sequence. During the normal course of repair of such DSB, a result that occurs with high frequency is the deletion of the intervening sequence as a result of recombination being facilitated by the DSB and concomitant cellular repair process.

In some circumstances, however, selecting target sequences within regions of homology can also give rise to much larger deletions, including gene fusions (when the deletions are in coding regions), which can or cannot be desired given the particular circumstances.

The examples provided herein further illustrate the selection of various target regions for the creation of DSBs designed to insert a FOXP3-encoding gene, as well as the selection of specific target sequences within such regions that are designed to minimize off-target events relative to on-target events. In some embodiments, the target locus is selected from a FOXP3 gene, an AAVS1 locus, and a TRA gene.

### Nucleic Acid Modifications

In some embodiments, polynucleotides introduced into cells have one or more modifications that can be used individually or in combination, for example, to enhance activity, stability, or specificity, alter delivery, reduce innate immune responses in host cells, or for other enhancements, as further described herein and known in the art.

In certain embodiments, modified polynucleotides are used in a CRISPR/Cas system (e.g., a CRISPR/Cas9 system), in which case the guide RNAs (either single-molecule guides or double-molecule guides) and/or a DNA or an RNA encoding a Cas endonuclease introduced into a cell can be modified, as described and illustrated below. Such modified polynucleotides can be used in the CRISPR/Cas system to edit any one or more genomic loci.

Using a CRISPR/Cas system for purposes of non-limiting illustrations of such uses, modifications of guide RNAs can be used to enhance the formation or stability of a CRISPR/Cas genome editing complex having guide RNAs, which can be single-molecule guides or double-molecule, and a Cas endonuclease. Modifications of guide RNAs can also or alternatively be used to enhance the initiation, stability, or kinetics of interactions between the genome editing complex with the target sequence in the genome, which can be used, for example, to enhance on-target activity. Modifications of guide RNAs can also or alternatively be used to enhance specificity, e.g., the relative rates of genome editing at the on-target site as compared to effects at other (off-target) sites.

Modifications can also or alternatively be used to increase the stability of a guide RNA, e.g., by increasing its resistance to degradation by ribonucleases (RNases) present in a cell, thereby causing its half-life in the cell to be increased. Modifications enhancing guide RNA half-life can be particularly useful in embodiments in which a Cas endonuclease is introduced into the cell to be edited via an RNA that needs to be translated to generate endonuclease, because increasing the half-life of guide RNAs introduced at the same time as the RNA encoding the endonuclease can be used to increase the time that the guide RNAs and the encoded Cas or Cpf1 endonuclease co-exist in the cell.

Modifications can also or alternatively be used to decrease the likelihood or degree to which RNAs introduced into cells elicit innate immune responses. Such responses, which have been well characterized in the context of RNA interference (RNAi), including small-interfering RNAs (siRNAs), as described below and in the art, tend to be associated with reduced half-life of the RNA and/or the elicitation of cytokines or other factors associated with immune responses.

One or more types of modifications can also be made to RNAs encoding an endonuclease that are introduced into a cell, including, without limitation, modifications that enhance the stability of the RNA (such as by increasing its degradation by RNAses present in the cell), modifications that enhance translation of the resulting product (e.g., the endonuclease), and/or modifications that decrease the likelihood or degree to which the RNAs introduced into cells elicit innate immune responses.

Combinations of modifications, such as the foregoing and others, can likewise be used. In the case of CRISPR/Cas, for example, one or more types of modifications can be made to guide RNAs (including those exemplified above), and/or one or more types of modifications can be made to RNAs encoding Cas endonuclease (including those exemplified above).

### Delivery

In some embodiments, any nucleic acid molecules used in the methods provided herein, e.g., a nucleic acid encoding a genome-targeting nucleic acid of the disclosure or a site-directed polypeptide, are packaged into or on the surface of delivery vehicles for delivery to cells. Delivery vehicles contemplated include, but are not limited to, nanospheres, liposomes, quantum dots, nanoparticles, polyethylene glycol particles, hydrogels, or micelles. As described in the art, a variety of targeting moieties can be used to enhance the preferential interaction of such vehicles with desired cell types or locations.

Introduction of the complexes, polypeptides, or nucleic acids of the disclosure into cells can occur by viral or bacteriophage infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, or nanoparticle-mediated nucleic acid delivery, and the like.

In embodiments, guide RNA polynucleotides (RNA or DNA) and/or endonuclease polynucleotide(s) (RNA or DNA) can be delivered by viral or non-viral delivery vehicles known in the art. Alternatively, endonuclease polypeptide(s) can be delivered by viral or non-viral delivery vehicles known in the art, such as electroporation or lipid nanoparticles. In some embodiments, the DNA endonuclease can be delivered as one or more polypeptides, either alone or pre-complexed with one or more guide RNAs, or one or more crRNA together with a tracrRNA.

In embodiments, polynucleotides can be delivered by non-viral delivery vehicles including, but not limited to, nanoparticles, liposomes, ribonucleoproteins, positively charged peptides, small molecule RNA-conjugates, aptamer-RNA chimeras, or RNA-fusion protein complexes. Some exemplary non-viral delivery vehicles are described in Peer, D. et al. (2011). Gene Therapy, 18: 1127-1133 (which focuses on non-viral delivery vehicles for siRNA that are also useful for delivery of other polynucleotides).

In embodiments, polynucleotides, such as guide RNA, sgRNA, or mRNA encoding an endonuclease, can be delivered to a cell or a subject by a lipid nanoparticle (LNP).

While several non-viral delivery methods for nucleic acids have been tested both in animal models and in humans the most well developed system is lipid nanoparticles. Lipid nanoparticles (LNP) are generally composed of an ionizable cationic lipid and 3 or more additional components, generally cholesterol, DOPE, and a polyethylene glycol (PEG) containing lipid, see, e.g. Example 2. The cationic lipid can bind to the positively charged nucleic acid forming a dense complex that protects the nucleic from degradation. During passage through a micro fluidics system the components self-assemble to form particles in the size range of 50 to 150 nM in which the nucleic acid is encapsulated in the core complexed with the cationic lipid and surrounded by a lipid bilayer like structure. After injection into the circulation of a subject these particles can bind to apolipoprotein E (apoE). ApoE is a ligand for the LDL receptor and mediates uptake into the hepatocytes of the liver via receptor mediated endocytosis. LNP of this type have been shown to efficiently deliver mRNA and siRNA to the hepatocytes of the liver of rodents, primates, or humans. After endocytosis, the LNP are present in endosomes. The encapsulated nucleic acid undergoes a process of endosomal escape mediate by the ionizable nature of the cationic lipid. This delivers the nucleic acid into the cytoplasm where mRNA can be translated into the encoded protein. After endosomal escape a Cas mRNA (e.g., a Cas9 mRNA) is translated into Cas protein and can form a complex with the gRNA. In some embodiments, inclusion of a nuclear localization signal into the Cas protein sequence promotes translocation of the Cas protein/gRNA complex to the nucleus. Alternatively, the small gRNA crosses the nuclear pore complex and form complexes with Cas protein in the nucleus. Once in the nucleus the gRNA/Cas complex scan the genome for homologous target sites and generate double-strand breaks preferentially at the desired target site in the genome. The half-life of RNA molecules in vivo is generally short, on the order of hours to days. Similarly, the half-life of proteins tends to be short, on the order of hours to days. Thus, in some embodiments, delivery of the gRNA and Cas mRNA using an LNP can result in only transient expression and activity of the gRNA/Cas complex. This can provide the benefit of reducing the frequency of off-target cleavage and, thus minimize the risk of genotoxicity in some embodiments. LNP are generally less immunogenic than viral particles. While many humans have preexisting immunity to AAV there is no pre-existing immunity to LNP. In additional and adaptive immune response against LNP is unlikely to occur which enables repeat dosing of LNP.

Several different ionizable cationic lipids have been developed for use in LNP. These include C12-200 (Love, K. T. et al. (2010). Proc. Natl. Acad. Sci. U.S.A., 107(5):1864-1869), MC3, LN16, MD1 among others. In one type of LNP a GalNac moiety is attached to the outside of the LNP and acts as a ligand for uptake into the liver via the asialyloglycoprotein receptor. Any of these cationic lipids are used to formulate LNP for delivery of gRNA and Cas mRNA to the liver.

In some embodiments, an LNP refers to any particle having a diameter of less than 1000 nm, 500 nm, 250 nm, 200 nm, 150 nm, 100 nm, 75 nm, 50 nm, or 25 nm. Alternatively, a nanoparticle can range in size from 1-1000 nm, 1-500 nm, 1-250 nm, 25-200 nm, 25-100 nm, 35-75 nm, or 25-60 nm.

LNPs can be made from cationic, anionic, or neutral lipids. Neutral lipids, such as the fusogenic phospholipid DOPE or the membrane component cholesterol, can be included in LNPs as ‘helper lipids’ to enhance transfection activity and nanoparticle stability. Limitations of cationic lipids include low efficacy owing to poor stability and rapid clearance, as well as, the generation of inflammatory or anti-inflammatory responses. LNPs can also have hydrophobic lipids, hydrophilic lipids, or both hydrophobic and hydrophilic lipids.

Any lipid or combination of lipids that are known in the art can be used to produce an LNP. Examples of lipids used to produce LNPs are: DOTMA, DOSPA, DOTAP, DMRIE, DC-cholesterol, DOTAP-cholesterol, GAP-DMORIE-DPyPE, or GL67A-DOPE-DMPE-polyethylene glycol (PEG). Examples of cationic lipids are: 98N12-5, C12-200, DLin-KC2-DMA (KC2), DLin-MC3-DMA (MC3), XTC, MD1, or 7C1. Examples of neutral lipids are: DPSC, DPPC, POPC, DOPE, or SM. Examples of PEG-modified lipids are: PEG-DMG, PEG-CerC14, or PEG-CerC20.

In embodiments, the lipids can be combined in any number of molar ratios to produce an LNP. In addition, the polynucleotide(s) can be combined with lipid(s) in a wide range of molar ratios to produce an LNP.

In embodiments, the site-directed polypeptide and genome-targeting nucleic acid can each be administered separately to a cell or a subject. On the other hand, the site-directed polypeptide can be pre-complexed with one or more guide RNAs, or one or more crRNA together with a tracrRNA. The pre-complexed material can then be administered to a cell or a subject. Such pre-complexed material is known as a ribonucleoprotein particle (RNP).

RNA can form specific interactions with RNA or DNA. While this property is exploited in many biological processes, it also comes with the risk of promiscuous interactions in a nucleic acid-rich cellular environment. One solution to this problem is the formation of ribonucleoprotein particles (RNPs), in which the RNA is pre-complexed with an endonuclease. Another benefit of the RNP is protection of the RNA from degradation.

In some embodiments, the endonuclease in the RNP can be modified or unmodified. Likewise, the gRNA, crRNA, tracrRNA, or sgRNA can be modified or unmodified. Numerous modifications are known in the art and can be used.

The endonuclease and sgRNA can be generally combined in a 1:1 molar ratio. Alternatively, the endonuclease, crRNA, and tracrRNA can be generally combined in a 1:1:1 molar ratio. However, a wide range of molar ratios can be used to produce an RNP.

In some embodiments, a recombinant adeno-associated virus (AAV) vector can be used for delivery. Techniques to produce rAAV particles, in which an AAV genome to be packaged that includes the polynucleotide to be delivered, rep, and cap genes, and helper virus functions are provided to a cell are known in the art. Production of rAAV requires that the following components are present within a single cell (denoted herein as a packaging cell): a rAAV genome, AAV rep and cap genes separate from (e.g., not in) the rAAV genome, and helper virus functions. The AAV rep and cap genes can be from any AAV serotype for which recombinant virus can be derived, and can be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, or AAV rh.74. Production of pseudotyped rAAV is disclosed in, for example, International Patent Application no. WO 01/83692. Table 1 lists AAV serotype and Genbank Accession No. of some selected AAVs.

TABLE 1

| AAV | Genbank |
|---|---|
| Serotype | Accession No. |
| AAV-1 | NC_002077.1 |
| AAV-2 | NC_001401.2 |
| AAV-3 | NC_001729.1 |
| AAV-3B | AF028705.1 |
| AAV-4 | NC_001829.1 |
| AAV-5 | NC_006152.1 |
| AAV-6 | AF028704.1 |
| AAV-7 | NC_006260.1 |
| AAV-8 | NC_006261.1 |
| AAV-9 | AX753250.1 |
| AAV-10 | AY631965.1 |
| AAV-11 | AY631966.1 |
| AAV-12 | DQ813647.1 |
| AAV-13 | EU285562.1 |

In some embodiments, a method of generating a packaging cell involves creating a cell line that stably expresses all of the necessary components for AAV particle production. For example, a plasmid (or multiple plasmids) having a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski, R. J. et al. (1982). Proc. Natl. Acad. Sci. U.S.A., 79(6):2077-2081), addition of synthetic linkers containing restriction endonuclease cleavage sites (Laughlin, C. A. et al. (1983). Gene, 23(1):65-73) or by direct, blunt-end ligation (Senapathy, P. et al. (1984). J. Biol. Chem., 259:4661-4666). The packaging cell line is then infected with a helper virus, such as adenovirus. The benefits of this method are that the cells are selectable and are suitable for large-scale production of rAAV. Other examples of suitable methods employ adenovirus or baculovirus, rather than plasmids, to introduce rAAV genomes and/or rep and cap genes into packaging cells.

General principles of rAAV production are reviewed in, for example, Carter, B. J. (1992). Curr. Opin. Biotechnol., 3(5):533-539; and Muzyczka, M. (1992). Curr. Top. Microbiol. Immunol., 158:97-129). Various approaches are described in Tratschin, J. D. et al. (1984). Mol. Cell. Biol., 4(10):2072-2081; Hermonat, P. L. et al. (1984). Proc. Natl. Acad. Sci. U.S.A., 81(20):6466-6470; Tratschin, J. D. et al. (1985). Mol. Cell. Biol., 5(11):3251-3260; McLaughlin, S. K. et al. (1988). J. Virol., 62(6):1963-1973; and Lebkowski, J. S. et al. (1988). Mol. Cell. Biol., 8(10):3988-3996. Samulski, R. J. et al. (1989), J. Virol., 63(9):3822-3828; U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin, P. et al. (1995). Vaccine, 13(13):1244-1250; Paul, R. W. et al. (1993). Hum. Gene Ther., 4(5):609-615; Clark, K. R. et al. (1996). Gene Ther. 3(12):1124-1132; U.S. Pat. Nos. 5,786,211; 5,871,982; and 6,258,595.

AAV vector serotypes can be matched to target cell types. For example, the following exemplary cell types can be transduced by the indicated AAV serotypes among others. For instance, the serotypes of AAV vectors suitable to hematopoietic stem cell include, but not limited to, AAV2 and AAV6. In some embodiments, the AAV vector serotype is AAV6.

In some embodiments, the AAV vector comprises a nucleic acid sequence having at least or at least about 90% sequence identity (e.g., at least 92%, 93%, 94%, 95%, 96%, 97%, 98%, 99%, 99.2%, 99.5%, 99.6%, 99.7%, 99.8%, 99.9%, or greater) to any one of SEQ ID NOs: 33-36 and 161. In some embodiments, the AAV vector comprises a nucleic acid sequence having at least or at least about 90% sequence identity (e.g., at least 92%, 93%, 94%, 95%, 96%, 97%, 98%, 99%, 99.2%, 99.5%, 99.6%, 99.7%, 99.8%, 99.9%, or greater) to SEQ ID NO: 33. In some embodiments, the AAV vector comprises a nucleic acid sequence having at least or at least about 90% sequence identity (e.g., at least 92%, 93%, 94%, 95%, 96%, 97%, 98%, 99%, 99.2%, 99.5%, 99.6%, 99.7%, 99.8%, 99.9%, or greater) to SEQ ID NO: 34. In some embodiments, the AAV vector comprises a nucleic acid sequence having at least or at least about 90% sequence identity (e.g., at least 92%, 93%, 94%, 95%, 96%, 97%, 98%, 99%, 99.2%, 99.5%, 99.6%, 99.7%, 99.8%, 99.9%, or greater) to SEQ ID NO: 35. In some embodiments, the AAV vector comprises a nucleic acid sequence having at least or at least about 90% sequence identity (e.g., at least 92%, 93%, 94%, 95%, 96%, 97%, 98%, 99%, 99.2%, 99.5%, 99.6%, 99.7%, 99.8%, 99.9%, or greater) to SEQ ID NO: 36. In some embodiments, the AAV vector comprises a nucleic acid sequence having at least or at least about 90% sequence identity (e.g., at least 92%, 93%, 94%, 95%, 96%, 97%, 98%, 99%, 99.2%, 99.5%, 99.6%, 99.7%, 99.8%, 99.9%, or greater) to SEQ ID NO: 161.

In addition to adeno-associated viral vectors, other viral vectors can be used. Such viral vectors include, but are not limited to, lentivirus, alphavirus, enterovirus, pestivirus, baculovirus, herpesvirus, Epstein Barr virus, papovavirus, poxvirus, vaccinia virus, or herpes simplex virus.

In some embodiments, Cas mRNA (e.g., Cas9 mRNA), sgRNA targeting one or two loci in FOXP3 genes, and donor DNA are each separately formulated into lipid nanoparticles, or are all co-formulated into one lipid nanoparticle, or co-formulated into two or more lipid nanoparticles.

In some embodiments, Cas mRNA (e.g., Cas9 mRNA) is formulated in a lipid nanoparticle, while sgRNA and donor DNA are delivered in an AAV vector. In some embodiments, Cas mRNA and sgRNA are co-formulated in a lipid nanoparticle, while donor DNA is delivered in an AAV vector.

Options are available to deliver a Cas endonuclease (e.g., a Cas9 endonuclease) as a DNA plasmid, as mRNA or as a protein. The guide RNA can be expressed from the same DNA, or can be delivered as an RNA. The RNA can be chemically modified to alter or improve its half-life and/or decrease the likelihood or degree of immune response. The endonuclease protein can be complexed with the gRNA prior to delivery. Viral vectors allow efficient delivery; split versions of Cas endonucleases and smaller orthologs of Cas endonucleases can be packaged in AAV, as can donors for HDR. A range of non-viral delivery methods also exist that can deliver each of these components, or non-viral and viral methods can be employed in tandem. For example, nanoparticles can be used to deliver the protein and guide RNA, while AAV can be used to deliver a donor DNA.

In some embodiments that are related to deliver genome-editing components for therapeutic treatments, at least two components are delivered into the nucleus of a cell to be transformed, e.g., CD34⁺ cells; a sequence-specific nuclease and a DNA donor template. In some embodiments, the AAV is selected from the serotypes AAV2 or AAV6. In some embodiments, the AAV packaged DNA donor template is administered to a subject, e.g., a patient, first by peripheral IV injection followed by the sequence-specific nuclease. The advantage of delivering an AAV packaged donor DNA template first is that the delivered donor DNA template will be stably maintained in the nucleus of the transduced CD34⁺ cells which allows for the subsequent administration of the sequence-specific nuclease, which will create a double-strand break in the genome with subsequent integration of the DNA donor by HDR or NHEJ. It is desirable in some embodiments that the sequence-specific nuclease remain active in the target cell only for the time required to promote targeted integration of the transgene at sufficient levels for the desired therapeutic effect. If the sequence-specific nuclease remains active in the cell for an extended duration this will result in an increased frequency of double-strand breaks at off-target sites. Specifically, the frequency of off-target cleavage is a function of the off-target cutting efficiency multiplied by the time over which the nuclease is active. Delivery of a sequence-specific nuclease in the form of a mRNA results in a short duration of nuclease activity in the range of hours to a few days because the mRNA and the translated protein are short lived in the cell. Thus, delivery of the sequence-specific nuclease into cells that already contain the donor template is expected to result in the highest possible ratio of targeted integration relative to off-target integration.

In some embodiments, the sequence-specific nuclease is a Cas endonuclease (e.g., a Cas9 endonuclease) used in a CRISPR/Cas system which is composed of a sgRNA directed to a FOXP3 gene together with the Cas endonuclease. In some embodiments, the Cas endonuclease is delivered as a mRNA encoding the Cas protein operably fused to one or more nuclear localization signals (NLS). In some embodiments, the sgRNA and the Cas mRNA are delivered to a CD34⁺ cell, e.g., a CD34⁺ hematopoietic stem cell, by packaging into a lipid nanoparticle.

In some embodiments, to promote nuclear localization of a donor template, DNA sequence that can promote nuclear localization of plasmids, e.g., a 366 bp region of the simian virus 40 (SV40) origin of replication and early promoter, can be added to the donor template. Other DNA sequences that bind to cellular proteins can also be used to improve nuclear entry of DNA.

### Genetically Modified Cells and Cell Populations

In one aspect, the disclosures herewith provide a method of editing a genome in a cell, thereby creating a genetically modified cell. In some aspects, a population of genetically modified cells are provided. The genetically modified cell therefore refers to a cell that has at least one genetic modification introduced by genome editing (e.g., using a CRISPR/Cas system). In some embodiments, the genetically modified cell is a genetically modified hematopoietic stem cell, e.g. a CD34⁺ cell such as a CD34⁺ hematopoietic stem cell. A genetically modified cell having an integrated FOXP3 coding sequence is contemplated herein. In some embodiments, the genetically cell is not a germ cell.

In the embodiments described herein, the cells for therapeutic application are engineered to have stable FOXP3 expression through the use of a gene editing nuclease to modify the regulatory elements of the FOXP3 gene to provide for stable FOXP3 expression. In the exemplary data provided, a promoter is placed upstream of the FOXP3 coding exons (examples of constitutive promoters include EF1 alpha promoter, the PGK promoter, or the MND promoter, among many others) to drive FOXP3 expression, but a variety of approaches are envisioned to modify the regulatory elements so as to allow for stable FOXP3 expression. By several approaches used to modify the endogenous regulatory elements, the claimed therapeutic cell exhibits constitutive expression of the native FOXP3 gene, such that it is no longer susceptible to regulation that could result in FOXP3 gene silencing and reversion to a non-suppressive cell phenotype. Accordingly, in the exemplary methods described herein, the problem of loss of FOXP3 expression due to epigenetic influences on the native regulatory sequences and promoter has been solved.

The proposed method of enforcing FOXP3 expression in a bulk population of CD34⁺ cells is contemplated. In subjects with auto-immune disease or who are rejecting an organ graft, the endogenous TCR repertoire in the inflammatory T cell population includes TCR's that have the correct binding specificity to recognize the inflamed tissue or the foreign tissue in the organ. These T cells are thought to mediate the auto-inflammatory reaction or organ rejection. By converting a portion of the bulk T cell population to a regulatory phenotype, the TCR specificities present in the pro-inflammatory population will be represented in the therapeutic cell population. This is an improvement over therapies based on thymic regulatory T cells, which is thought to have a distinct and non-overlapping TCR repertoire from inflammatory T cells. In addition, presumably in subjects with auto-immune disease or organ rejection, the existing tT_{reg} population has failed to produce the tolerance necessary to avoid inflammation. The methods described herein can be used for therapy of auto-immune disease and for induction of tolerance to transplanted organs.

A significant disadvantage is the need to use gene editing tools that can efficiently carry out the recombination at the FOXP3 gene. As such, the methods provided show that the use of TALEN nuclease can carry this reaction out efficiently, but in principle, any nuclease platform would serve equally well.

The regulatory T cell therapies can be used for tolerance applications in transplantation and in auto-immunity. Currently, Treg infusions are expanded ex vivo. Phase I studies have shown marginal if any efficacy in T1D, and in some cases there have been benefits in post-transplant GVHD. For next generation engineered regulatory T cells, in some embodiments, these can be chimeric antigen receptor (CAR) directed natural T_{regs}. Effector T cells can also be converted to T_{regs} by FOXP3 expression.

However, there may also be differences between engineered versus natural T_{regs} for methods of treatment. Natural Treg therapy has been considered safe, however too few natural T_{regs} causes autoimmunity. Treg play a critical role in multiple autoimmune diseases (IPEX, T1D, SLE, RA, and EAE, etc). Approaches to augment human Treg number or function are in current trials including low-dose IL-2 and adoptive transfer of autologous expanded Treg. The efficacy of IL-2 therapy is limited due to its pleotropic activity and potential “off target” effects that may increase inflammation. Adoptive Treg therapy is likely limited by in vivo stability and viability of expanded T_{regs} and their lack of relevant antigen specificity.

There are also potential flaws with the use of natural T_{regs}. For example, autoimmune subjects can be genetically predisposed to Treg instability. For example, it is plausible for a CAR bearing nTreg to convert to a CAR T effector cell. nTreg also retain the potential for epigenetic regulation of FOXP3, which may lead to the down regulation of the desired FOXP3 induction. Also, natural T_{regs} might not include the correct TCR (T cell receptor) specificities. The Treg function may also be linked to a selectable marker in which the expanded native Treg cell population may always have contaminating inflammatory cells. Thus, the methods provided herein are an improvement over using the transfer of natural T_{regs} by using engineered cells as there is potential for linking CAR expression to regulatory T cell function to avoid potential engraftment of CAR T_{regs} that have the potential to convert to pro inflammatory CAR T cells.

In some embodiments, the genome of a cell can be edited by inserting a nucleic acid sequence encoding a FOXP3 or a functional derivative thereof into a genomic sequence of the cell. In some embodiments, the cell subject to the genome-edition has one or more mutation(s) in the genome which results in reduction of the expression of endogenous FOXP3 gene as compared to the expression in a normal that does not have such mutation(s). The normal cell can be a healthy or control cell that is originated (or isolated) from a different subject who does not have FOXP3 gene defects. In some embodiments, the cell subject to the genome-edition can be originated (or isolated) from a subject who is in need of treatment of FOXP3 gene related condition or disorder. Therefore, in some embodiments the expression of endogenous FOXP3 gene in such cell is at or about 10%, at or about 20%, at or about 30%, at or about 40%, at or about 50%, at or about 60%, at or about 70%, at or about 80%, at or about 90% or at or about 100% increased as compared to the expression of endogenous FOXP3 gene expression in the normal cell.

Upon successful insertion of the transgene, e.g., a nucleic acid encoding a FOXP3 or a functional derivative thereof, the expression of the introduced nucleic acid encoding a FOXP3 or a functional derivative thereof in the cell can be at least or at least about 10%, at or about 20%, at or about 30%, at or about 40%, at or about 50%, at or about 60%, at or about 70%, at or about 80%, at or about 90%, at or about 100%, at or about 200%, at or about 300%, at or about 400%, at or about 500%, at or about 600%, at or about 700%, at or about 800%, at or about 900%, at or about 1,000%, at or about 2,000%, at or about 3,000%, at or about 5,000%, at or about 10,000% or more as compared to the expression of an endogenous FOXP3 gene of the cell. In some embodiments, the activity of introduced FOXP3-encoding sequence products, including functional derivatives of the FOXP3, in the genome-edited cell can be at least or at least about 10%, at or about 20%, at or about 30%, at or about 40%, at or about 50%, at or about 60%, at or about 70%, at or about 80%, at or about 90%, at or about 100%, at or about 200%, at or about 300%, at or about 400%, at or about 500%, at or about 600%, at or about 700%, at or about 800%, at or about 900%, at or about 1,000%, at or about 2,000%, at or about 3,000%, at or about 5,000%, at or about 10,000% or more as compared to the activity of an endogenous FOXP3 gene of the cell. In some embodiments, the expression of the introduced FOXP3-encoding sequence in the cell is at least or at least about 2 fold, at or about 3 fold, at or about 4 fold, at or about 5 fold, at or about 6 fold, at or about 7 fold, at or about 8 fold, at or about 9 fold, at or about 10 fold, at or about 15 fold, at or about 20 fold, at or about 30 fold, at or about 50 fold, at or about 100 fold, at or about 1000 fold or more of the expression of endogenous FOXP3 gene of the cell. Also, in some embodiments, the activity of introduced FOXP3-encoding sequence products, including functional derivatives of the FOXP3, in the genome-edited cell can be comparable to or more than the activity of endogenous FOXP3 gene products in a normal, healthy cell.

In one embodiment CD34⁺ cells are genetically modified ex vivo and then re-introduced into the subject where they will give rise to genetically modified T cells that express the inserted FOXP3 gene.

### Methods of Making

In some embodiments, a method of making a genetically engineered cell is provided, the method comprising providing a CD34⁺ cell, wherein the CD34⁺ cell comprises a first nucleic acid comprising at least one locus, providing a Cas endonuclease (e.g., a Cas9 endonuclease) or a second nucleic acid encoding a Cas endonuclease, introducing the Cas endonuclease or the second nucleic acid into the CD34⁺ cell, introducing a third nucleic acid encoding at least one gRNA or a set of nucleic acids encoding at least one gRNA, wherein the at least one gRNA is configured to hybridize to the at least one locus; and introducing a fourth nucleic acid into the CD34⁺ cell, wherein the fourth nucleic acid comprises a gene delivery cassette.

In some embodiments, according to a method of making a genetically engineered cell provided herein, the method further comprises activating the CD34⁺ cell, wherein the activating is performed before the introducing of the second nucleic acid into the CD34⁺ cell. In some embodiments, the activating is performed by contacting the CD34⁺ cell with a cytokine selected from the group consisting of thrombopoietin (TPO), stem cell factor (SCF), FLT3L, and IL-6. The cytokine may be on a bead.

In some embodiments, according to a method of making a genetically engineered cell provided herein, the at least one locus is a FOXP3 gene, AAVS1 locus, or a TRA gene.

In some embodiments, the second nucleic acid, the third nucleic acid, the set of nucleic acids and/or the fourth nucleic acid is provided in one or more vectors. In some embodiments, the one or more vectors is a viral vector. In some embodiments, the viral vector is an Adeno-associated virus (AAV) vector. In some embodiments, the AAV vector is a self-complementary vector. In some embodiments, the AAV vector is a single stranded vector. In some embodiments, the AAV vector is a combination of a self-complementary vector and a single stranded vector.

In some embodiments, the second nucleic acid encoding the Cas endonuclease is an mRNA. In some embodiments, the at least one gRNA comprises a spacer sequence comprising a sequence as set forth in SEQ ID NO: 2, 3 or 5. In some embodiments, the second nucleic acid, the third nucleic acid, the set of nucleic acids and/or the fourth nucleic acid are codon optimized for expression in a eukaryotic cell, such as a human cell. In some embodiments, the fourth nucleic acid comprises a sequence encoding a human codon optimized FOXP3 cDNA sequence. In some embodiments, the fourth nucleic acid further comprises a promoter. In some embodiments, the promoter is an MND promoter, a PGK promoter, or an E2F promoter. In some embodiments, the fourth nucleic acid further comprises a sequence encoding a low affinity nerve growth factor receptor coding sequence (LNGFR), μCISC, CISCγ, FRB or LNGFRe (LNGFR epitope coding sequence). In some embodiments, the fourth nucleic acid further comprises a sequence encoding a low affinity nerve growth factor receptor coding sequence (LNGFR) or LNGFRe (LNGFR epitope coding sequence).

In some embodiments, the method further comprises introducing a fifth nucleic acid into the CD34⁺ cell, wherein the fifth nucleic acid comprises a second gene delivery cassette. In some embodiments, the fifth nucleic acid is comprised in a vector. In some embodiments, the vector is an AAV vector. In some embodiments, the fifth nucleic acid comprises a sequence encoding CISC, FRB, a marker protein, μCISC, and/or βCISC. In some embodiments, the fifth nucleic acid comprises a sequence encoding a marker protein. In some embodiments, the fourth and or the fifth sequence further comprises a sequence encoding a P2A self-cleaving peptide. In some embodiments, the fourth and or the fifth sequence further comprises a sequence encoding a polyA sequence. In some embodiments, the polyA sequence comprises a SV40polyA or 3′UTR of FOXP3. In some embodiments, the fourth sequence comprises a sequence set forth in any one of SEQ ID NO: 37-42.

In some embodiments, a fourth sequence and a fifth sequence are introduced into the CD34⁺ cell, wherein the fourth and fifth sequence comprise a sequence that encodes an expression cassette configured to express: FOXP3cDNA-LNGFR and DISC, FOXP3cDNA-LNGFR and μDISC, LNGFR-FOXP3cDNA and DISC, LNGFR-FOXP3cDNA and μDISC, CISCβ-DN and CISCγ-FOXP3cDNA-LNGFR, or CISCβ-DN and CISCγ-LNGFR-FOXP3cDNA, respectively.

In some embodiments, the fourth nucleic acid comprises at least one homology arm with a locus specific sequence, wherein the homology arm length is configured for efficient packaging into an AAV vector.

In some embodiments, the at least one homology arm comprises a length of 0.25, 0.3, 0.45, 0.6 or 0.8 kb or any length in between a range defined by any two aforementioned values.

In some embodiments, the marker is LNGF, RQR8 or EGFRt.

In some embodiments, the method further comprises introducing into the CD34⁺ cell a sixth nucleic acid encoding a protein or cytokine for co-expression with FOXP3.

In some embodiments, the method further comprises selecting the CD34⁺ cells by enrichment of the marker.

In some embodiments, the CD34⁺ cell is contacted with a medium comprising hTPO, hFlt3, hSCF or hIL6.

In some embodiments, a CD34⁺ cell for expression of FOXP3 is provided, wherein the cell is manufactured by the method of any one of the embodiments herein. In some embodiments, FOXP3 is expressed constitutively or the expression is regulated.

In some embodiments, a CD34⁺ cell for expression of FOXP3 is provided, the CD34⁺ cell comprising a nucleic acid encoding a gene encoding FOXP3. In some embodiments, the gene encoding FOXP3 is introduced in a FOXP3 gene or a non-FOXP3 locus. In some embodiments, the non-FOXP3 locus is an AAVS1 locus or a TRA gene.

In some embodiments, the CD34⁺ cell expresses CISCβ: FRB-IL2Rβ, DISC, CISC-FRB, μDISC, μCISC-FRB, FRB, LNGFR or LNGFRe. In some embodiments, the CD34⁺ cell comprises a Treg phenotype.

In some embodiments, a composition comprising the CD34⁺ cell of any one of the embodiments is provided.

In some embodiments, a method for treating, ameliorating, and/or inhibiting a disease and/or a condition in a subject is provided, the method comprising: providing to a subject having a disease and/or a condition the CD34⁺ cell or the composition of any one of the embodiments herein. In some embodiments, the disease is an autoimmune disease. In some embodiments, the disease is immunodysregulation polyendocrinopathy enteropathy X-linked (IPEX) syndrome. In some embodiments, the condition is Graft-versus-Host Disease (GVHD).

In the embodiments herein, a method of making a genetically engineered cell is provided, the method comprising: providing a CD34⁺ cell, wherein the CD34⁺ cell comprises a first nucleic acid comprising at least one locus; providing a Cas endonuclease (e.g., a Cas9 endonuclease) or a second nucleic acid encoding a Cas endonuclease; introducing the Cas endonuclease or the second nucleic acid into the CD34⁺ cell; introducing a third nucleic acid encoding at least one CRISPR spacer sequence or a set of nucleic acids encoding at least one CRISPR spacer sequence, wherein the at least one CRISPR spacer sequence is configured to hybridize to the at least one locus; and introducing a fourth nucleic acid into the CD34⁺ cell, wherein the fourth nucleic acid comprises a gene delivery cassette. In some embodiments, the fourth nucleic acid further comprises a promoter. In some embodiments, the promoter is a MND promoter, a PGK promoter or an E2F promoter. In some embodiments, the promoter is a MND promoter. As described in the embodiments herein, the MND promoter is provided in the vector #3008 (pAAV_FoxP3.0.6 kb.MND.GFP.WPRE3.pA) (SEQ ID NO: 33).

In some embodiments, the cells differentiate into T cells, and the T cells express FOXP3. In some embodiments, the endogenous FOXP3 promoter drives expression of the introduced FOXP3 cDNA.

A weak promoter produces less mRNA expression than a stronger promoter, if both are driving expression of the same coding sequences. This can be compared by analyzing, for example, an agarose gel. An example of promoters subject to regulation by proximal chromatin is the EF1 alpha short promoter, which is highly active in some loci, but nearly inactive in other loci (Eyquem, J. et al. (2013). Biotechnol. Bioeng., 110(8):2225-2235).

### Therapeutic Approach

One aspect provided herein is a gene therapy approach for providing therapy to a subject having or suspected of having a disorder or health condition associated with a FOXP3 protein by editing the genome of the subject. For example, in some embodiments, the disorder or health condition is an autoimmune disease (e.g., IPEX syndrome) or a disorder that results from organ transplant (e.g., GVHD). In some embodiments, the gene therapy approach integrates a nucleic acid comprising a sequence encoding a functional FOXP3 gene into the genome of a relevant cell type in subjects and this can provide a permanent cure for the disorder or health condition. In some embodiments, a cell type subject to the gene therapy approach in which to integrate the FOXP3-encoding sequence is the CD34⁺ cell, e.g., CD34⁺ hematopoietic stem cell, because these cells can efficiently differentiate into T cells in the subject.

In another aspect, provided herein are cellular, ex vivo and in vivo methods for using genome engineering tools to create permanent changes to a cell genome by knocking-in a coding sequence encoding a FOXP3 or a functional derivative thereof into a gene locus in the cell genome and restoring FOXP3 activity. Such methods use endonucleases, such as CRISPR-associated (CRISPR/Cas9, Cpf1, and the like) nucleases, to permanently delete, insert, edit, correct, or replace any sequences from the cell genome or insert an exogenous sequence, e.g., a FOXP3-encoding sequence, in a genomic locus in the cell. In this way, the examples set forth in the present disclosure restore the activity of FOXP3 with a single therapeutic step (rather than requiring the delivery of alternative therapies for the lifetime of the subject).

In some embodiments, an ex vivo cell-based therapy is performed using a CD34⁺ cell that is isolated from a subject, e.g., a CD34⁺ cell derived from cord blood. Next, the chromosomal DNA of these cells is edited using the systems, compositions, and methods described herein. Finally, the edited cells are implanted into the subject.

One benefit of an ex vivo cell therapy approach is the ability to conduct a comprehensive analysis of the therapeutic prior to administration. All nuclease-based therapeutics have some level of off-target effects. Performing gene correction ex vivo allows one to fully characterize the corrected cell population prior to implantation. Aspects of the disclosure include sequencing the entire genome of the corrected cells to ensure that the off-target cuts, if any, are in genomic locations associated with minimal risk to the subject. Furthermore, populations of specific cells, including clonal populations, can be isolated prior to implantation.

Another embodiment of such methods is an in vivo based therapy. In this method, the chromosomal DNA of the cells in the subject is corrected using the systems, compositions, and methods described herein. In some embodiments, the cells are CD34⁺ cells.

A benefit of in vivo gene therapy is the ease of therapeutic production and administration. The same therapeutic approach and therapy can be used to treat more than one subject, for example a number of subjects who share the same or similar genotype or allele. In contrast, ex vivo cell therapy generally uses a subject's own cells, which are isolated, manipulated, and returned to the same subject.

In some embodiments, the subject who is in need of the therapy in accordance with the disclosure herein is a subject having symptoms of a disease or condition associated with a FOXP3. For example, in some embodiments, the subject has symptoms of an autoimmune disease (e.g., IPEX syndrome) or a disorder that results from organ transplant (e.g., GVHD). In some embodiments, the subject can be a human suspected of having the disease or condition. Alternatively, the subject can be a human diagnosed with a risk of the disease or condition. In some embodiments, the subject who is in need of the therapy can have one or more genetic defects (e.g., deletion, insertion, and/or mutation) in the endogenous FOXP3 gene or its regulatory sequences such that the activity including the expression level or functionality of the FOXP3 is substantially reduced compared to a normal, healthy subject.

In some embodiments, provided herein is a method of treating or inhibiting a disease or condition associated with a FOXP3 (e.g., an autoimmune disease) in a subject, the method comprising providing the following to a cell in the subject: (a) a guide RNA (gRNA) targeting the FOXP3 gene in the cell genome; (b) a DNA endonuclease or nucleic acid encoding said DNA endonuclease; and (c) a donor template comprising a nucleic acid sequence encoding a FOXP3 or a functional derivative thereof. In some embodiments, the gRNA targets a FOXP3 gene, AAVS1 locus or a TRA gene. In some embodiments, the gRNA comprises a spacer sequence from any one of SEQ ID NOs: 1-7, 15-20, and 27-29.

In some embodiments, provided herein is a method of treating or inhibiting a disease or condition associated with FOXP3 (e.g., an autoimmune disease) in a subject, the method comprising providing the following to a cell in the subject: (a) a gRNA comprising a spacer sequence that is complementary to a genomic sequence within or near an endogenous FOXP3 gene in the cell; (b) a DNA endonuclease or nucleic acid encoding said DNA endonuclease; and (c) a donor template comprising a nucleic acid sequence encoding the FOXP3 or a functional derivative thereof. In some embodiments, the gRNA comprises a spacer sequence from any one of SEQ ID NOs: 1-7 and 27-29 or a variant thereof having no more than 3 mismatches compared to any one of SEQ ID NOs: 1-7 and 27-29. In some embodiments, the gRNA comprises a spacer sequence from any one of SEQ ID NOs: 1-7 or a variant thereof having no more than 3 mismatches compared to any one of SEQ ID NOs: 1-7. In some embodiments, the gRNA comprises a spacer sequence from any one of SEQ ID NOs: 2, 3, and 5 or a variant thereof having no more than 3 mismatches compared to any one of SEQ ID NOs: 2, 3, and 5. In some embodiments, the gRNA comprises a spacer sequence from SEQ ID NO: 2 or a variant thereof having no more than 3 mismatches compared to SEQ ID NO: 2. In some embodiments, the gRNA comprises a spacer sequence from SEQ ID NO: 5 or a variant thereof having no more than 3 mismatches compared to SEQ ID NO: 5. In some embodiments, the cell is a human cell, e.g., a human stem cell, for example a human CD34⁺ hematopoietic stem cell. In some embodiments, the subject is a patient having or suspected of having an autoimmune disease, e.g., IPEX syndrome or Graft-versus-Host disease. In some embodiments, the subject is diagnosed with a risk of an autoimmune disease, e.g., IPEX syndrome or Graft-versus-Host disease.

In some embodiments, provided herein is a method of treating or inhibiting a disease or condition associated with FOXP3 (e.g., an autoimmune disease) in a subject, the method comprising providing to the subject a genetically modified cell prepared by any of the methods of editing a genome in a cell described herein. In some embodiments, the nucleic acid sequence encoding a FOXP3 or a functional derivative thereof is expressed under the control of the endogenous FOXP3 promoter. In some embodiments, the nucleic acid sequence encoding a FOXP3 or a functional derivative thereof is codon-optimized for expression in the cell. In some embodiments, the cell is a CD34⁺ cell. In some embodiments, the genetically modified cell is autologous to the subject. In some embodiments, the method further comprises obtaining a biological sample from the subject, wherein the biological sample comprises an input cell, and wherein the genetically modified cell is prepared from the input cell. In some embodiments, the input cell is a CD34⁺ cell.

Some embodiments include a medicament for use in treating or inhibiting a disease or condition associated with FOXP3 (e.g., an autoimmune disease) in a subject. More embodiments concern a genetically modified CD34⁺ cell in which the genome of the cell is edited by one of the methods described herein for use in inhibiting or treating a disease or condition associated with FOXP3, such as an inflammatory disease or an autoimmune disease. Additional embodiments concern use of a genetically modified CD34⁺ cell in which the genome of the cell is edited by any one of the methods herein as a medicament.

Implanting Cells into a Subject

In some embodiments, the ex vivo methods of the disclosure involve implanting the genome-edited cells into a subject who is in need of such method. This implanting step can be accomplished using any method of implantation known in the art. For example, the genetically modified cells can be injected directly in the subject's blood or otherwise administered to the subject.

In some embodiments, the methods disclosed herein include administering, which can be interchangeably used with “introducing” and “transplanting,” genetically modified, therapeutic cells into a subject, by a method or route that results in at least partial localization of the introduced cells at a desired site such that a desired effect(s) is produced. The therapeutic cells or their differentiated progeny can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the life time of the subject, e.g., long-term engraftment.

When provided prophylactically, the therapeutic cells described herein can be administered to a subject in advance of any symptom of a disease or condition associated with a FOXP3 (e.g., an autoimmune disease, such as IPEX syndrome). Accordingly, in some embodiments the prophylactic administration of a genetically modified stem cell population serves to prevent the occurrence of symptoms of the disease or condition.

When provided therapeutically in some embodiments, genetically modified stem cells are provided at (or after) the onset of a symptom or indication of a disease or condition associated with a FOXP3 (e.g., an autoimmune disease, such as IPEX syndrome), e.g., upon the onset of disease or condition.

For use in the various embodiments described herein, an effective amount of therapeutic cells, e.g., genome-edited stem cells, can be at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The therapeutic cells can be derived from one or more donors or can be obtained from an autologous source. In some embodiments described herein, the therapeutic cells are expanded in culture prior to administration to a subject in need thereof.

In some embodiments, modest and incremental increases in the levels of functional FOXP3 expressed in cells of subjects having a disease or condition associated with the FOXP3 (e.g., IPEX syndrome) can be beneficial for ameliorating one or more symptoms of the disease or condition, for increasing long-term survival, and/or for reducing side effects associated with other treatments. Upon administration of such cells to human subjects, the presence of therapeutic cells that are producing increased levels of functional FOXP3 is beneficial. In some embodiments, effective treatment of a subject gives rise to at least or at least about 1%, 3%, 5%, or 7% functional FOXP3 relative to total FOXP3 in the treated subject. In some embodiments, functional FOXP3 is at least or at least about 10% of total FOXP3. In some embodiments, functional FOXP3 is at least, about, or at most 20%, 30%, 40%, 50%, 60%, 70%, 80%, 90%, or 100% of total FOXP3. Similarly, the introduction of even relatively limited subpopulations of cells having significantly elevated levels of functional FOXP3 can be beneficial in various subjects because in some situations normalized cells will have a selective advantage relative to diseased cells. However, even modest levels of therapeutic cells with elevated levels of functional FOXP3 can be beneficial for ameliorating one or more aspects of the disease or condition in subjects. In some embodiments, at or about 10%, at or about 20%, at or about 30%, at or about 40%, at or about 50%, at or about 60%, at or about 70%, at or about 80%, at or about 90% or more of the therapeutic in subjects to whom such cells are administered are producing increased levels of functional FOXP3.

In embodiments, the delivery of a therapeutic cell composition (e.g., a composition comprising a plurality of cells according to any of the cells described herein) into a subject by a method or route results in at least partial localization of the cell composition at a desired site. A cell composition can be administered by any appropriate route that results in effective treatment in the subject, e.g., administration results in delivery to a desired location in the subject where at least a portion of the composition delivered, e.g., at least 1×10⁴ cells, is delivered to the desired site for a period of time. Modes of administration include injection, infusion, instillation, or ingestion. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, or intrasternal injection or infusion. In some embodiments, the route is intravenous. For the delivery of cells, administration by injection or infusion can be made.

In one embodiment, the cells are administered systemically, in other words a population of therapeutic cells are administered other than directly into a target site, tissue, or organ, such that it enters, instead, the subject's circulatory system and, thus, is subject to metabolism and other like processes.

The efficacy of a therapy having a composition for the treatment or inhibition of a disease or condition associated with a FOXP3 (e.g., IPEX syndrome) can be determined by the skilled clinician. However, a therapy is considered effective if any one or all of the signs or symptoms of, as but one example, levels of functional FOXP3 are altered in a beneficial manner (e.g., increased by at least 10%), or other clinically accepted symptoms or markers of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Therapy includes any treatment or inhibition of a disease in an individual or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

### Compositions

In one aspect, the present disclosure provides compositions for carrying out the methods disclosed herein. A composition can include one or more of the following: a genome-targeting nucleic acid (e.g., a gRNA); a site-directed polypeptide (e.g., a DNA endonuclease) or a nucleotide sequence encoding the site-directed polypeptide; and a polynucleotide to be inserted (e.g., a donor template) to effect the desired genetic modification of the methods disclosed herein.

In some embodiments, a composition has a nucleotide sequence encoding a genome-targeting nucleic acid (e.g., a gRNA).

In some embodiments, a composition has a site-directed polypeptide (e.g. DNA endonuclease). In some embodiments, a composition has a nucleotide sequence encoding the site-directed polypeptide.

In some embodiments, a composition has a polynucleotide (e.g., a donor template) to be inserted into a genome.

In some embodiments, a composition has (i) a nucleotide sequence encoding a genome-targeting nucleic acid (e.g., a gRNA) and (ii) a site-directed polypeptide (e.g., a DNA endonuclease) or a nucleotide sequence encoding the site-directed polypeptide.

In some embodiments, a composition has (i) a nucleotide sequence encoding a genome-targeting nucleic acid (e.g., a gRNA) and (ii) a polynucleotide (e.g., a donor template) to be inserted into a genome.

In some embodiments, a composition has (i) a site-directed polypeptide (e.g., a DNA endonuclease) or a nucleotide sequence encoding the site-directed polypeptide and (ii) a polynucleotide (e.g., a donor template) to be inserted into a genome.

In some embodiments, a composition has (i) a nucleotide sequence encoding a genome-targeting nucleic acid (e.g., a gRNA), (ii) a site-directed polypeptide (e.g., a DNA endonuclease) or a nucleotide sequence encoding the site-directed polypeptide and (iii) a polynucleotide (e.g., a donor template) to be inserted into a genome.

In some embodiments of any of the above compositions, the composition has a single-molecule guide genome-targeting nucleic acid. In some embodiments of any of the above compositions, the composition has a double-molecule genome-targeting nucleic acid. In some embodiments of any of the above compositions, the composition has two or more double-molecule guides or single-molecule guides. In some embodiments, the composition has a vector that encodes the nucleic acid targeting nucleic acid. In some embodiments, the genome-targeting nucleic acid is configured to be used with a DNA endonuclease, in particular, a Cas endonuclease (e.g., a Cas9 endonuclease).

In some embodiments, a composition can include one or more gRNAs that can be used for genome-edition, in particular, insertion of a sequence encoding a FOXP3 or a derivative thereof into a genome of a cell. The one or more gRNAs can target a genomic site at, within, or near the endogenous FOXP3 gene. Therefore, in some embodiments, the one or more gRNAs can have a spacer sequence complementary to a genomic sequence at, within, or near a FOXP3 gene.

In some embodiments, a gRNA for a composition comprises a spacer sequence selected from any one of SEQ ID NOs: 1-7, 15-20, or 27-29, and variants thereof having at least or at least about 50%, at or about 55%, at or about 60%, at or about 65%, at or about 70%, at or about 75%, at or about 80%, at or about 85%, at or about 90% or at or about 95% identity or homology to any one of SEQ ID NOs: 1-7, 15-20, or 27-29. In some embodiments, the variants of gRNA for the kit comprise a spacer sequence having at least or at least about 85%, 90%, 91%, 92%, 93%, 94%, 95%, 96%, 97%, 98%, or 99% sequence identity to any one of SEQ ID NOs: 1-7, 15-20, or 27-29.

In some embodiments, a gRNA for a composition has a spacer sequence that is complementary to a target site in the genome. In some embodiments, the spacer sequence is 15 bases to 20 bases in length. In some embodiments, a complementarity between the spacer sequence to the genomic sequence is at least 80%, at least 85%, at least 90%, at least 95%, at least 96%, at least 97%, at least 98%, at least 99% or at least 100%.

In some embodiments, a composition can have a DNA endonuclease or a nucleic acid encoding the DNA endonuclease and/or a donor template having a nucleic acid sequence encoding a FOXP3 or a functional derivative thereof. In some embodiments, the DNA endonuclease is a Cas endonuclease (e.g., a Cas9 endonuclease). In some embodiments, the nucleic acid encoding the DNA endonuclease is DNA or RNA.

In some embodiments, one or more of any nucleic acids for the kit can be encoded in an Adeno Associated Virus (AAV) vector. Therefore, in some embodiments, a gRNA can be encoded in an AAV vector. In some embodiments, a nucleic acid encoding a DNA endonuclease can be encoded in an AAV vector. In some embodiments, a donor template can be encoded in an AAV vector. In some embodiments, two or more nucleic acids can be encoded in a single AAV vector. Thus, in some embodiments, a gRNA sequence and a DNA endonuclease-encoding nucleic acid can be encoded in a single AAV vector.

In some embodiments, a composition can have a liposome or a lipid nanoparticle. Therefore, in some embodiments, any compounds (e.g., a DNA endonuclease or a nucleic acid encoding thereof, gRNA, and donor template) of the composition can be formulated in a liposome or lipid nanoparticle. In some embodiments, one or more such compounds are associated with a liposome or lipid nanoparticle via a covalent bond or non-covalent bond. In some embodiments, any of the compounds can be separately or together contained in a liposome or lipid nanoparticle. Therefore, in some embodiments, each of a DNA endonuclease or a nucleic acid encoding thereof, gRNA, and donor template is separately formulated in a liposome or lipid nanoparticle. In some embodiments, a DNA endonuclease is formulated in a liposome or lipid nanoparticle with gRNA. In some embodiments, a DNA endonuclease or a nucleic acid encoding thereof, gRNA, and donor template are formulated in a liposome or lipid nanoparticle together.

In some embodiments, a composition described above further has one or more additional reagents, where such additional reagents are selected from a buffer, a buffer for introducing a polypeptide or polynucleotide into a cell, a wash buffer, a control reagent, a control vector, a control RNA polynucleotide, a reagent for in vitro production of the polypeptide from DNA, adaptors for sequencing and the like. A buffer can be a stabilization buffer, a reconstituting buffer, a diluting buffer, or the like. In some embodiments, a composition can also include one or more components that can be used to facilitate or enhance the on-target binding or the cleavage of DNA by the endonuclease, or improve the specificity of targeting.

In some embodiments, any components of a composition are formulated with pharmaceutically acceptable excipients such as carriers, solvents, stabilizers, adjuvants, diluents, etc., depending upon the particular mode of administration and dosage form. In embodiments, guide RNA compositions are generally formulated to achieve a physiologically compatible pH, and range from a pH of or about 3 to a pH of or about 11, of or about pH 3 to or to about pH 7, depending on the formulation and route of administration. In some embodiments, the pH is adjusted to a range from or from about pH 5.0 to or to about pH 8. In some embodiments, the composition has a therapeutically effective amount of at least one compound as described herein, together with one or more pharmaceutically acceptable excipients. Optionally, the composition can have a combination of the compounds described herein, or can include a second active ingredient useful in the treatment or prevention of bacterial growth (for example and without limitation, anti-bacterial or anti-microbial agents), or can include a combination of reagents of the disclosure. In some embodiments, gRNAs are formulated with other one or more nucleic acids, e.g., nucleic acid encoding a DNA endonuclease and/or a donor template. Alternatively, a nucleic acid encoding a DNA endonuclease and a donor template, separately or in combination with other nucleic acids, are formulated with the method described above for gRNA formulation.

Suitable excipients can include, for example, carrier molecules that include large, slowly metabolized macromolecules such as proteins, polysaccharides, polylactic acids, polyglycolic acids, polymeric amino acids, amino acid copolymers, or inactive virus particles. Other exemplary excipients include antioxidants (for example and without limitation, ascorbic acid), chelating agents (for example and without limitation, EDTA), carbohydrates (for example and without limitation, dextrin, hydroxyalkylcellulose, or hydroxyalkylmethylcellulose), stearic acid, liquids (for example and without limitation, oils, water, saline, glycerol, or ethanol), wetting or emulsifying agents, or pH buffering substances, and the like.

In some embodiments, any compounds (e.g., a DNA endonuclease or a nucleic acid encoding thereof, gRNA, and donor template) of a composition can be delivered into a cell via transfection, such as chemical transfection (e.g., lipofection) or electroporation. In some embodiments, a DNA endonuclease can be pre-complexed with a gRNA, forming a ribonucleoprotein (RNP) complex, prior to the provision to the cell. In some embodiments, the RNP complex is delivered into the cell via transfection. In such embodiments, the donor template is delivered into the cell via transfection.

In some embodiments, a composition refers to a therapeutic composition having therapeutic cells that are used in an ex vivo treatment method.

In embodiments, therapeutic compositions contain a physiologically tolerable carrier together with the cell composition, and optionally at least one additional bioactive agent as described herein, dissolved or dispersed therein as an active ingredient. In some embodiments, the therapeutic composition is not substantially immunogenic when administered to a mammal or human subject for therapeutic purposes, unless so desired.

In general, the genetically modified, therapeutic cells described herein are administered as a suspension with a pharmaceutically acceptable carrier. One of skill in the art will recognize that a pharmaceutically acceptable carrier to be used in a cell composition will not include buffers, compounds, cryopreservation agents, preservatives, or other agents in amounts that substantially interfere with the viability of the cells to be delivered to the subject. A formulation having cells can include e.g., osmotic buffers that permit cell membrane integrity to be maintained, and optionally, nutrients to maintain cell viability or enhance engraftment upon administration. Such formulations and suspensions are known to those of skill in the art and/or can be adapted for use with the progenitor cells, as described herein, using routine experimentation.

In some embodiments, a cell composition can also be emulsified or presented as a liposome composition, provided that the emulsification procedure does not adversely affect cell viability. The cells and any other active ingredient can be mixed with one or more excipients that are pharmaceutically acceptable and compatible with the active ingredient, and in amounts suitable for use in the therapeutic methods described herein.

Additional agents included in a cell composition can include pharmaceutically acceptable salts of the components therein. Pharmaceutically acceptable salts include the acid addition salts (formed with the free amino groups of the polypeptide) that are formed with inorganic acids, such as, for example, hydrochloric or phosphoric acids, or such organic acids as acetic, tartaric, or mandelic, and the like. Salts formed with the free carboxyl groups can also be derived from inorganic bases, such as, for example, sodium, potassium, ammonium, calcium, or ferric hydroxides, or such organic bases as isopropylamine, trimethylamine, 2-ethylamino ethanol, histidine, or procaine, and the like.

Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium or potassium chlorides, dextrose, or polyethylene glycol and other solutes. Liquid compositions can also contain liquid phases in addition to and to the exclusion of water. Exemplary of such additional liquid phases are glycerin, vegetable oils such as cottonseed oil, or water-oil emulsions. The amount of an active compound used in the cell compositions that is effective in the treatment of a particular disorder or condition will depend on the nature of the disorder or condition, and can be determined by known clinical techniques.

### Kits

Some embodiments provide a kit that contains any of the above-described compositions, e.g., a composition for genome edition or a cell composition (e.g., a therapeutic cell composition), and one or more additional components.

In some embodiments, a kit can have one or more additional therapeutic agents that can be administered simultaneously or in sequence with the composition for a desired purpose, e.g., genome edition or cell therapy.

In some embodiments, a kit can further include instructions for using the components of the kit to practice the methods. The instructions for practicing the methods are generally recorded on a suitable recording medium. For example, the instructions can be printed on a substrate, such as paper or plastic, etc. The instructions can be present in the kits as a package insert, in the labeling of the container of the kit or components thereof (i.e., associated with the packaging or subpackaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

### FURTHER EMBODIMENTS

In some embodiments, a method of making a genetically engineered cell is provided, the method comprising: providing a CD34⁺ cell, wherein the CD34⁺ cell comprises a first nucleic acid comprising at least one locus; providing a CAS9 protein or a second nucleic acid encoding a CAS9 protein; introducing the CAS9 protein or the second nucleic acid into the CD34⁺ cell; introducing a third nucleic acid encoding at least one CRISPR spacer sequence or a set of nucleic acids encoding at least one CRISPR spacer sequence, wherein the at least one CRISPR spacer sequence is configured to hybridize to the at least one locus; and introducing a fourth nucleic acid into the CD34⁺ cell, wherein the fourth nucleic acid comprises a gene delivery cassette.

In some embodiments, the method further comprises activating the CD34⁺ cell, wherein the activating is performed before the introducing of the second nucleic acid into the CD34⁺ cell. In some embodiments, activating is performed by contacting the CD34⁺ cell with a cytokine selected from the group consisting of thrombopoietin (TPO), stem cell factor (SCF), FLT3L, and IL-6. In some embodiments, the at least one locus is a FOXP3 gene, AAVS1 locus or a TRA gene. In some embodiments, the second nucleic acid, the third nucleic acid, the set of nucleic acids and/or the fourth nucleic acid is provided in one or more vectors. In some embodiments, the one or more vectors is a viral vector. In some embodiments, the viral vector is an Adeno-associated virus (AAV) vector.

In some embodiments, the AAV vector is a self-complementary vector. In some embodiments, the AAV vector is a single stranded vector. In some embodiments, the AAV vector is a combination of a self-complementary vector and a single stranded vector. In some embodiments, the second nucleic acid encoding the CAS9 protein is an mRNA. In some embodiments, the at least one spacer sequence comprises a sequence as set forth in SEQ ID NO: 2, 3 or 5. In some embodiments, the second nucleic acid, the third nucleic acid, the set of nucleic acids and/or the fourth nucleic acid are codon optimized for expression in a eukaryotic cell, such as a human. In some embodiments, the fourth nucleic acid comprises a sequence encoding a human codon optimized FOXP3 cDNA sequence. In some embodiments, the fourth nucleic acid further comprises a promoter. In some embodiments, the promoter is a MND promoter, a PGK promoter or an E2F promoter. In some embodiments, the fourth nucleic acid further comprises a sequence encoding a low affinity nerve growth factor receptor coding sequence (LNGFR), μCISC, CISCγ, FRB and/or LNGFRe (LNGFR epitope coding sequence). In some embodiments, the fourth nucleic acid further comprises a sequence encoding a low affinity nerve growth factor receptor coding sequence (LNGFR) and/or LNGFRe (LNGFR epitope coding sequence).

In some embodiments, the method further comprises introducing a fifth nucleic acid into the CD34⁺ cell, wherein the fifth nucleic acid comprises a second gene delivery cassette. In some embodiments, the fifth nucleic acid is comprised in a vector. In some embodiments, the vector is an AAV vector. In some embodiments, the fifth nucleic acid comprises a sequence encoding CISC, FRB, a marker protein, μCISC, and/or βCISC. In some embodiments, the fifth nucleic acid comprises a sequence encoding a marker protein. In some embodiments, the fourth and or the fifth nucleic acid further comprises a sequence encoding a P2A self-cleaving peptide. In some embodiments, the fourth and or the fifth nucleic acid further comprises a sequence encoding a polyA sequence. In some embodiments, the polyA sequence comprises a SV40polyA or 3′UTR of FOXP3. In some embodiments, the fourth nucleic acid comprises a WPRE3 element. In some embodiments, the fourth and/or fifth nucleic acid are introduced into the CD34⁺ cell, wherein the fourth and/or fifth nucleic acid comprises a sequence that encodes an expression cassette for expression of FOXP3cDNA-LNGFR and DISC, FOXP3cDNA LNGFR and μDISC, LNGFR-FOXP3cDNA and DISC, LNGFR-FOXP3cDNA and DISC, CISCβ-DN and CISCγ-FOXP3cDNA-LNGFR, or CISCβ-DN and CISCγ-LNGFR-FOXP3cDNA, respectively. In some embodiments, the fourth and/or fifth nucleic acid are introduced into the CD34⁺ cell, wherein the fourth and/or fifth nucleic acid comprises a sequence that encodes an expression cassette. In some embodiments, the fourth nucleic acid comprises at least one homology arm with a locus specific sequence, wherein the homology arm length is configured for efficient packaging into an AAV vector. In some embodiments, the at least one homology arm comprises a length of 0.25, 0.3, 0.45, 0.6, 0.8 kb or 1 kb or any length in between a range defined by any two aforementioned values. In some embodiments, the marker is LNGF, RQR8 or EGFRt. In some embodiments, the method further comprises introducing into the CD34⁺ cell a sixth nucleic acid encoding a protein or cytokine for co-expression with FOXP3. In some embodiments, the protein or cytokine is a T cell receptor, chimeric antigen receptor, or IL10. In some embodiments, the fourth nucleic acid comprises a sequence set forth in SEQ ID NO: 34 or 36. In some embodiments, the method further comprises selecting the CD34⁺ cells by enrichment of the marker. In some embodiments, the CD34⁺ cell is contacted with a medium comprising hTPO, hFlt3, hSCF and/or hIL6.

In some embodiments, a CD34⁺ cell for expression of FOXP3 is provided, wherein the cell is manufactured by the method of any one of the embodiments described herein. In some embodiments, FOXP3 is expressed constitutively or the expression is regulated. The method comprises: providing a CD34⁺ cell, wherein the CD34⁺ cell comprises a first nucleic acid comprising at least one locus; providing a CAS9 protein or a second nucleic acid encoding a CAS9 protein; introducing the CAS9 protein or the second nucleic acid into the CD34⁺ cell; introducing a third nucleic acid encoding at least one CRISPR spacer sequence or a set of nucleic acids encoding at least one CRISPR spacer sequence, wherein the at least one CRISPR spacer sequence is configured to hybridize to the at least one locus; and introducing a fourth nucleic acid into the CD34⁺ cell, wherein the fourth nucleic acid comprises a gene delivery cassette. In some embodiments, the method further comprises activating the CD34⁺ cell, wherein the activating is performed before the introducing of the second nucleic acid into the CD34⁺ cell. In some embodiments, the activating is performed by contacting the CD34⁺ cell with a cytokine selected from the group consisting of thrombopoietin (TPO), stem cell factor (SCF), FLT3L, and IL-6. In some embodiments, the at least one locus is a FOXP3 gene, AAVS1 locus or a TRA gene. In some embodiments, the second nucleic acid, the third nucleic acid, the set of nucleic acids and/or the fourth nucleic acid is provided in one or more vectors. In some embodiments, the one or more vectors is a viral vector. In some embodiments, the viral vector is an Adeno-associated virus (AAV) vector. In some embodiments, the AAV vector is a self-complementary vector. In some embodiments, the AAV vector is a single stranded vector. In some embodiments, the AAV vector is a combination of a self-complementary vector and a single stranded vector. In some embodiments, the second nucleic acid encoding the CAS9 protein is an mRNA. In some embodiments, the at least one spacer sequence comprises a sequence as set forth in SEQ ID NO: 2, 3 or 5. In some embodiments, the second nucleic acid, the third nucleic acid, the set of nucleic acids and/or the fourth nucleic acid are codon optimized for expression in a eukaryotic cell, such as a human. In some embodiments, the fourth nucleic acid comprises a sequence encoding a human codon optimized FOXP3 cDNA sequence. In some embodiments, the fourth nucleic acid further comprises a promoter. In some embodiments, the promoter is a MND promoter, a PGK promoter or an E2F promoter. In some embodiments, the fourth nucleic acid further comprises a sequence encoding a low affinity nerve growth factor receptor coding sequence (LNGFR), μCISC, CISCγ, FRB and/or LNGFRe (LNGFR epitope coding sequence). In some embodiments, the fourth nucleic acid further comprises a sequence encoding a low affinity nerve growth factor receptor coding sequence (LNGFR) and/or LNGFRe (LNGFR epitope coding sequence). In some embodiments, the method further comprises introducing a fifth nucleic acid into the CD34⁺ cell, wherein the fifth nucleic acid comprises a second gene delivery cassette. In some embodiments, the fifth nucleic acid is comprised in a vector. In some embodiments, the vector is an AAV vector. In some embodiments, the fifth nucleic acid comprises a sequence encoding CISC, FRB, a marker protein, μCISC, and/or βCISC. In some embodiments, the fifth nucleic acid comprises a sequence encoding a marker protein. In some embodiments, the fourth and or the fifth nucleic acid further comprises a sequence encoding a P2A self-cleaving peptide. In some embodiments, the fourth and/or the fifth nucleic acid further comprises a sequence encoding a polyA sequence. In some embodiments, the polyA sequence comprises a SV40polyA or 3′UTR of FOXP3. In some embodiments, the fourth nucleic acid comprises a WPRE3 element. In some embodiments, the fourth and/or fifth nucleic acid are introduced into the CD34⁺ cell, wherein the fourth and/or fifth nucleic acid comprises a sequence that encodes an expression cassette for expression of FOXP3cDNA LNGFR and DISC, FOXP3cDNA-LNGFR and DISC, LNGFR-FOXP3cDNA and DISC, LNGFR-FOXP3cDNA and DISC, CISCβ-DN and CISCγ-FOXP3cDNA-LNGFR, or CISCβ-DN and CISCγ-LNGFR-FOXP3cDNA, respectively. In some embodiments, the fourth and/or fifth nucleic acid are introduced into the CD34⁺ cell, wherein the fourth and/or fifth nucleic acid comprises a sequence that encodes an expression cassette. In some embodiments, the fourth nucleic acid comprises at least one homology arm with a locus specific sequence, wherein the homology arm length is configured for efficient packaging into an AAV vector. In some embodiments, the at least one homology arm comprises a length of 0.25, 0.3, 0.45, 0.6, 0.8 kb or 1 kb or any length in between a range defined by any two aforementioned values. In some embodiments, the marker is LNGF, RQR8 or EGFRt. In some embodiments, the method further comprises introducing into the CD34⁺ cell a sixth nucleic acid encoding a protein or cytokine for co-expression with FOXP3. In some embodiments, the protein or cytokine is a T cell receptor, chimeric antigen receptor, or IL10. In some embodiments, the fourth nucleic acid comprises a sequence set forth in SEQ ID NO: 34 or 36. In some embodiments, the method further comprises selecting the CD34⁺ cells by enrichment of the marker. In some embodiments, the CD34⁺ cell is contacted with a medium comprising hTPO, hFlt3, hSCF and/or hIL6.

In some embodiments, a CD34⁺ cell for expression of FOXP3 is provided, the CD34⁺ cell comprising: a nucleic acid encoding a gene encoding FOXP3. In some embodiments, the gene encoding FOXP3 is introduced in a FOXP3 gene or a non-FOXP3 locus. In some embodiments, the non-FOXP3 locus is an AAVS1 locus or a TRA gene. In some embodiments, the CD34⁺ cell expresses CISCβ: FRB-IL2Rβ, DISC, CISC-FRB, μDISC, μCISC-FRB, FRB, LNGFR and/or LNGFRe. In some embodiments, the CD34⁺ cell leads to generation progenitors that differentiate within a thymus to generate T cells with a Treg phenotype.

In some embodiments, a composition comprising the CD34⁺ cell of any one of the embodiments herein is provided.

In some embodiments, a method for treating, ameliorating, and/or inhibiting a disease and/or a condition in a subject is provided, the method comprising: providing to a subject having a disease and/or a condition the CD34⁺ cell or the composition of any one of the embodiments described herein. In some embodiments, the disease is an autoimmune disease. In some embodiments, the disease is IPEX syndrome. In some embodiments, the condition is Graft-versus-Host Disease (GVHD).

### EXEMPLARY EMBODIMENTS

Embodiment 1. A method of making a genetically engineered cell, the method comprising: providing a CD34⁺ cell, wherein the CD34⁺ cell comprises a first nucleic acid comprising at least one targeted locus; providing a CAS9 protein or a second nucleic acid encoding a CAS9 protein; introducing the CAS9 protein or the second nucleic acid into the CD34⁺ cell; introducing a third nucleic acid encoding at least one CRISPR spacer sequence or a set of nucleic acids encoding at least one CRISPR spacer sequence, wherein the at least one CRISPR spacer sequence is configured to hybridize to the at least one targeted locus; and introducing a fourth nucleic acid into the CD34⁺ cell, wherein the fourth nucleic acid comprises a gene delivery cassette.

Embodiment 2. The method of Embodiment 1, wherein the method further comprises activating the CD34⁺ cell, wherein the activating is performed before the introducing of the second nucleic acid into the CD34⁺ cell.

Embodiment 3. The method of Embodiment 2, wherein the activating is performed by contacting the CD34⁺ cell with CD3 and/or CD28.

Embodiment 4. The method of any one of Embodiments 1-3, wherein the at least one targeted locus is a FOXP3 gene, AAVS1 locus or a TRA gene.

Embodiment 5. The method of any one of Embodiments 1-4, wherein the second nucleic acid, the third nucleic acid, the set of nucleic acids and/or the fourth nucleic acid is provided in one or more vectors.

Embodiment 6. The method of Embodiment 5, wherein the one or more vectors is a viral vector.

Embodiment 7. The method of Embodiment 6, wherein the viral vector is an Adeno-associated virus (AAV) vector.

Embodiment 8. The method of Embodiment 7 wherein the AAV vector is a self-complementary vector.

Embodiment 9. The method of Embodiment 7 or 8 wherein the AAV vector is a single stranded vector.

Embodiment 10. The method of any one of Embodiments 7-9, wherein the AAV vector is a combination of a self-complementary vector and a single stranded vector.

Embodiment 11. The method of any one of Embodiments 1-4, wherein the second nucleic acid encoding the CAS9 protein is an mRNA.

Embodiment 12. The method of any one of Embodiments 1-11, wherein the at least one spacer sequence comprises a sequence as set forth in SEQ ID NO: 2, 3 or 5.

Embodiment 13. The method of any one of Embodiments 1-12, wherein the second nucleic acid, the third nucleic acid, the set of nucleic acids and/or the fourth nucleic acid are codon optimized for expression in a eukaryotic cell, such as a human.

Embodiment 14. The method of any one of Embodiments 1-13, wherein the fourth nucleic acid comprises a sequence encoding a human codon optimized FOXP3 cDNA sequence.

Embodiment 15. The method of Embodiment 13, wherein the fourth nucleic acid further comprises a promoter.

Embodiment 16. The method of Embodiment 15, wherein the promoter is a MND promoter, a PGK promoter or an E2F promoter.

Embodiment 17. The method of any one of Embodiments 14-16, wherein the fourth nucleic acid further comprises a sequence encoding a low affinity nerve growth factor receptor coding sequence (LNGFR) and/or LNGFRe (LNGFR epitope coding sequence).

Embodiment 18. The method of any one of Embodiments 1-17, wherein the method further comprises introducing a fifth nucleic into the CD34⁺ cell, wherein the fifth nucleic acid comprises a second gene delivery cassette.

Embodiment 19. The method of Embodiment 18, wherein the fifth nucleic acid is comprised in a vector.

Embodiment 20. The method of Embodiment 18, wherein the vector is an AAV vector.

Embodiment 21. The method of any one of Embodiments 18-20, wherein the fifth nucleic acid comprises a sequence encoding a marker protein.

Embodiment 22. The method of any one of Embodiments 1-21, wherein the fourth and or the fifth nucleic acid further comprises a sequence encoding a P2A self-cleaving peptide.

Embodiment 23. The method of any one of Embodiments 1-22, wherein the fourth and or the fifth nucleic acid further comprises a sequence encoding a polyA sequence.

Embodiment 24. The method of Embodiment 23, wherein the polyA sequence comprises a SV40polyA or 3′UTR of FOXP3.

Embodiment 25. The method of any one of Embodiments 1-24, wherein the fourth nucleic acid comprises a WPRE3 element.

Embodiment 26. The method of any one of Embodiments 1-25, wherein the fourth and/or fifth nucleic acid are introduced into the CD34⁺ cell, wherein the fourth and/or fifth nucleic acid comprises a sequence that encodes an expression cassette.

Embodiment 27. The method of any one of Embodiments 1-26, wherein the fourth nucleic acid comprises at least one homology arm with a locus specific sequence, wherein the homology arm length is configured for efficient packaging into an AAV vector.

Embodiment 28. The method of Embodiment 27, wherein the at least one homology arm comprises a length of 0.25, 0.3, 0.45, 0.6, 0.8 kb or 1 kb or any length in between a range defined by any two aforementioned values.

Embodiment 29. The method of any one of Embodiments 21-28, wherein the marker is LNGF, RQR8 or EGFRt.

Embodiment 30. The method of any one of Embodiments 1-29, wherein the method further comprises introducing into the CD34⁺ cell a sixth nucleic acid encoding a protein or cytokine for co-expression with FOXP3.

Embodiment 31. The method of Embodiment 30, wherein the protein or cytokine is a T cell receptor, chimeric antigen receptor, or IL10.

Embodiment 32. The method of any one of Embodiments 1-31, wherein the fourth nucleic acid comprises a sequence set forth in SEQ ID NO: 34 or 36.

Embodiment 33. The method of any one of Embodiments 1-32, wherein the method further comprises selecting the CD34⁺ cells by enrichment of the marker.

Embodiment 34. The method of any one of Embodiments 1-33, wherein the CD34⁺ cell is contacted with a medium comprising hTPO, hFlt3, hSCF and/or hIL6.

Embodiment 35. A CD34⁺ cell for expression of FOXP3, manufactured by the method of any one of claims 1-34.

Embodiment 36. The CD34⁺ cell of Embodiment 35, wherein FOXP3 is expressed constitutively or the expression is regulated.

Embodiment 37. A CD34⁺ cell for expression of FOXP3, the CD34⁺ cell comprising a nucleic acid encoding a gene encoding FOXP3.

Embodiment 38. The CD34⁺ cell of Embodiment 37, wherein the gene encoding FOXP3 is introduced in a FOXP3 gene or a non-FOXP3 locus.

Embodiment 39. The CD34⁺ cell of Embodiment 38, wherein the non-FOXP3 locus is a AAVS1 locus or a TRA gene.

Embodiment 40. The CD34⁺ cell of any one of Embodiments 35-39, wherein the CD34⁺ cell leads to generation progenitors that differentiate within a thymus to generate T cells with a Treg phenotype.

Embodiment 41. A composition comprising the CD34⁺ cell of any one of claims 34-40.

Embodiment 42. A method for treating, ameliorating, and/or inhibiting a disease and/or a condition in a subject, the method comprising: providing to a subject having a disease and/or a condition the CD34⁺ cell of any one of Embodiments 33-39 or the composition of Embodiment 41.

Embodiment 43. The method of Embodiment 42, wherein the disease is an autoimmune disease.

Embodiment 44. The method of Embodiment 42, wherein the disease is X-linked (IPEX) syndrome.

Embodiment 45. The method of Embodiment 42, wherein the condition is Graft-versus-Host Disease (GVHD) or results from organ transplant.

Some embodiments include a medicament for use in treating or inhibiting a disorder related to a FOXP3 mutation.

In some of the foregoing embodiments, the cell is not a germ cell.

### EXAMPLES

### Example 1: Editing of CD34⁺ Cells for FOXP3 Expression

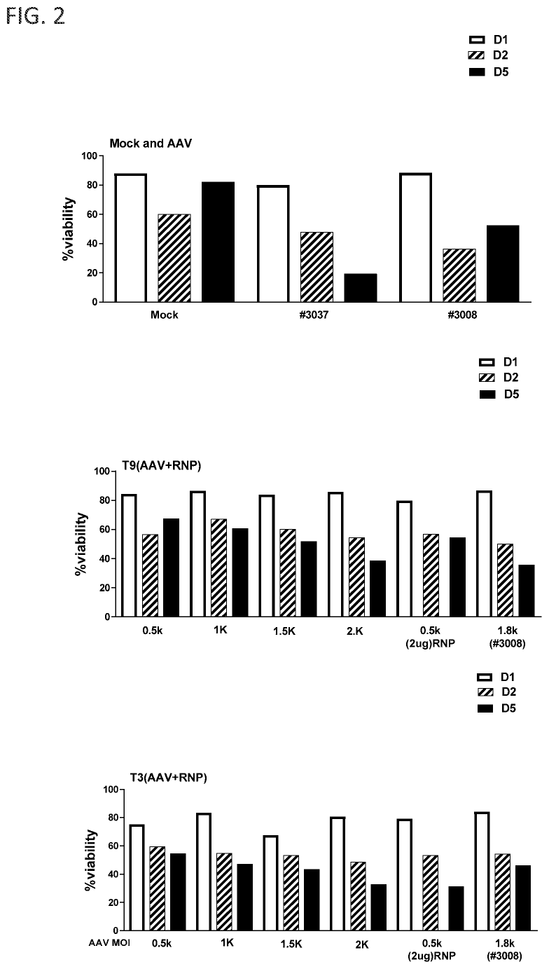

This example demonstrates the successful editing of CD34⁺ cells by targeted integrations of an AAV donor into a FOXP3 gene mediated by Cas9 and gRNAs targeting the FOXP3 gene. CD34⁺ cells were edited at the FOXP3 gene according to the protocol outlined as follows. Cryopreserved CD34⁺ cells enriched from PBMC mobilized adult donors were thawed and plated at 1×10⁶ cells/ml in serum-free stem cell growth media [CellGenix GMP SCGM medium (CellGenix Inc.) with thrombopoietin, stem cell factor, FLT3 ligand, and IL-6 (PeproTech) all at 100 ng/ml]. The CD34⁺ cells were prestimulated in the serum-free stem cell growth media for 48 hours at 37° C., then electroporated with RNPs containing gRNA complexed with Alt-R S.p. Cas9 Nuclease V3 from IDT (“IDT Cas9”) (Integrated DNA Technologies, Inc., Coralville, IA USA) protein (gRNA/Cas9) at a 1.2:1 molar ratio of gRNA to Cas9 using the Neon® Transfection System (ThermoFisher Scientific). gRNAs with spacer sequence T3 (SEQ ID NO: 2) or T9 (SEQ ID NO: 5) were used in this Example. Following electroporation, the cells were dispensed into a 48-well plate containing 400 μL of media per well and AAV donor templates #3008 (SEQ ID NO: 33) or #3037 (SEQ ID NO: 34) were added at MOIs ranging from 0.5k to 1.8k. AAV donor template #3037 contained a FOXP3 cDNA sequence for expression of FOXP3, and AAV donor template #3008 contained a GFP coding sequence under the control of an MND promoter, allowing for estimation of editing rates based on GFP expression (FIG. 1). Twenty-four hours after RNP electroporation and AAV transduction, the media was removed and replaced with fresh stem cell growth media. Analyses of cell viability (FIG. 2) and percent GFP⁺ cells were performed at days 1, 2 and 5 post-editing. Comparable cell viability was observed across the groups.

To assess editing rates with AAV donor template #3037, “in-out” droplet digital PCR (ddPCR) was performed with the forward primer binding within the codon-optimized FOXP3 cDNA and the reverse primer binding the FOXP3 gene outside the region of homology. A control amplicon of similar size was generated for the ActB gene to serve as a control. All reactions were performed in duplicate. The PCR reactions were partitioned into droplets using a QX200 Droplet Generator (Bio-Rad). Amplification was performed using ddPCR Supermix for probes without UTP (Bio-Rad), 900 nM of primers, 250 nM of probe, 50 ng of genomic DNA, and 1% DMSO. Droplets were analyzed on the QX200 Droplet Digital PCR System (Bio-Rad) using QuantaSoft software (Bio-Rad).

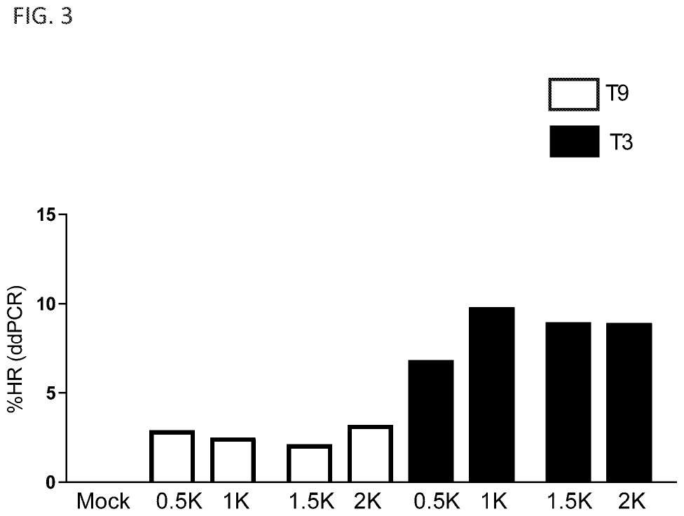

As shown in FIG. 3, the cells edited using the Cas9/gRNA-T3 RNP (containing a spacer having the sequence of SEQ ID NO: 2) had improved HDR as compared to cells edited using the Cas9/gRNA-T9 RNP (containing a spacer having the sequence of SEQ ID NO: 5) upon transduction with AAV donor template #3037 (SEQ ID NO: 34). Additionally, editing using the Cas9/gRNA-T3 gRNA RNP with AAV donor template #3008 led to higher expression of GFP.

| | % live | % GFP⁺ |
|---|---|---|
| Treatment Conditions | cells | cells |
| Mock | 82.1 | 0 |
| AAV | 19.2 | 1.9 |
| Cas9/gRNA-T9 RNP + | 35.6 | 2.8 |
| AAV donor template #3037 | | |
| Cas9/gRNA-T3 RNP + | 46.1 | 10.8 |
| AAV donor template #3008 | | |

An alternative AAV donor template configuration with longer homology arms was tested. AAV donor template #3088 (SEQ ID NO: 35), containing a GFP coding sequence, had 0.8 kb arms, with the 3′ homology arm modified to position the T3 gRNA cleavage site at the 5′ end of the 3′ homology arm. Both AAV donor templates #3008 and #3088 were non-cleavable by the gRNA. The table below shows the percent viability of untreated CD34⁺ cells and CD34⁺ cells treated with Cas9/gRNA RNP containing the T3 gRNA plus either AAV donor template #3008 or #3088. Cells treated with AAV donor template #3088 had a slightly higher percent viability.

| Treatment | % cell viability |
|---|---|
| Conditions | at Day 1 |
| Mock | 84.3 |
| AAV #3088 only | 68.7 |
| T3 RNP + AAV donor | 71.6 |
| template #3008 | |
| T3 RNP + AAV donor | 72.7 |
| template #3088 | |

The editing rate in CD34⁺ cells treated with AAV donor template #3088 alone, Cas9/T3 gRNA RNPs+AAV donor template (#3008 or #3088), or mock treated was determined by FACS analysis for GFP⁺ cells. As shown below, the amount of HR was about 3 fold higher in cells edited using AAV donor template #3088 as compared to AAV donor template #3008. These results suggest that AAV donor templates with 0.8 kb homology arm lengths result in higher editing efficiencies as compared to AAV donor templates with 0.6 kb homology arms. The below tables summarize the results.

| | % HR at |
|---|---|
| Treatment conditions | Day 4 |
| Mock | 0 |
| AAV donor template #3088 only | 5.6 |
| T3 RNP + AAV donor template #3008 | 11.9 |
| T3 RNP + AAV donor template #3088 | 48.1 |

| Conditions | % live cells | % GFP⁺ cells |
|---|---|---|
| Mock | 73.3 | 0 |
| AAV donor template #3088 | 15.7 | 5.6 |
| Cas9/gRNA-T3 RNP + | 42.1 | 48.1 |
| AAV donor template #3088 | | |
| Cas9/gRNA-T3 RNP + | 36.0 | 11.9 |
| AAV donor template #3008 | | |

### Example 2: Embodiments with Other Spacer Sequences

Additional spacer sequences for targeting the FOXP3 gene are also contemplated for use and are shown in Table 1. gRNAs containing the spacer sequences of SEQ ID NOs: 1, 4, 6 or 7 are made and tested for editing efficiency in CD34⁺ cells, for example, according to the studies described in Example 1.

gRNAs containing spacer sequences targeting the AAVS1 locus as shown in Table 2 (SEQ ID NOS: 15-20) are also made. These gRNAs may be used with the editing protocols as described in Example 1. gRNAs containing spacer sequences targeting the murine FOXP3 gene (SEQ ID NO: 27-29) and the human TRA gene are also made and tested using the editing protocols as described in Example 1.

Donor templates are also contemplated, which have the following expression cassettes: FOXP3cDNA-LNGFR, LNGFR-FOXP3cDNA, FOXP3cDNA-μDISC, FOXP3cDNA-LNGFRe-μDISC, μDISC-FOXP3cDNA, LNGFRe-μDISC-FOXP3cDNA, DISC, μDISC, CISCβ-DN, CISCγ-FOXP3cDNA-LNGFR and/or CISCγ-LNGFR-FOXP3cDNA.

### Example 3: Delivery of Cas9 RNPS with Different Guide-RNAs

Results

gRNAs with T3 and T9 spacer sequences delivered in RNPs upon complexing with two different Cas9 nucleases were evaluated for cell viabilities, allelic disruption rates and homology directed repair rates, when co-delivered with AAV donor template #3008 as described below.

The Cas9/gRNA RNP comprising the T3 spacer sequence outperformed the Cas9/gRNA RNP comprising the T9 spacer sequence in inducing higher allelic disruption and higher HDR. However, the Cas9/gRNA RNP having the T3 spacer sequence was also found to cut at off-target site SLC2A6, as summarized below.

A modified Cas9 protein, SpyFi Cas9 from Aldevron (Fargo, ND, USA), has been reported to exhibit reduced off-target cleavage. No off-target cutting was observed at the SLC2A6 site by the RNP comprising SpyFi Cas9/gRNA targeting FOXP3 with T3 spacer sequence, as measured by ICE. No cleavage at the off-target site was observed with the SpyFi Cas9/gRNA RNP containing either the T3 or T9 spacer sequence.

Methods

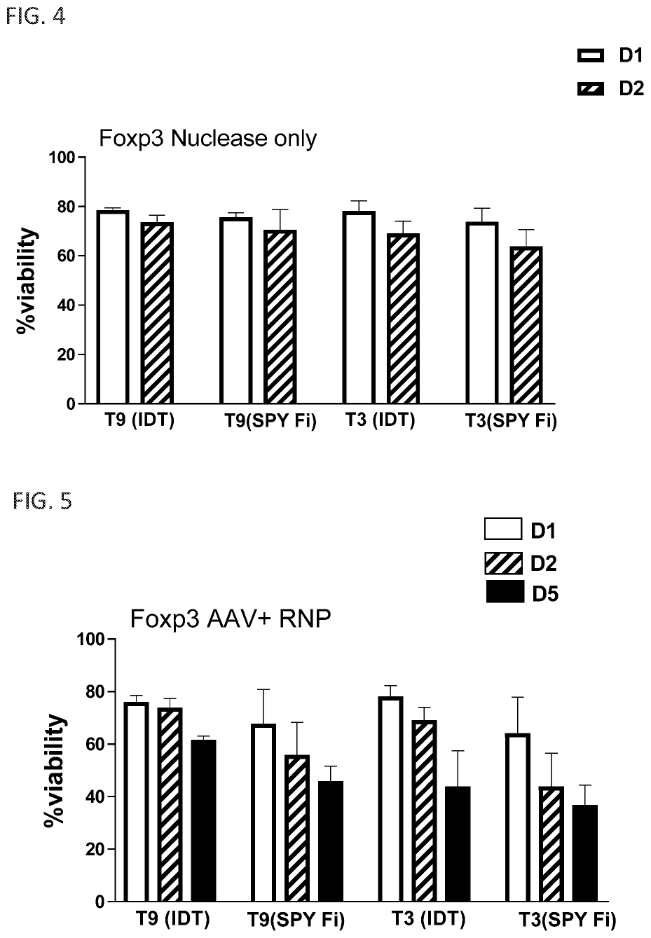

First, to compare the cell viabilities when CD34⁺ cells were treated with Cas9 from two different sources (Alt-R S.p. Cas9 Nuclease V3 from IDT or SpyFi Cas9 from Aldevron), adult human Mobilized CD34⁺ cells were cultured in SCGM media supplemented with TPO, SCF, FLT3L and IL6 (100 ng/ml) for 48 hours, followed by electroporation using Neon® transfection system (Model MPK5000, ThermoFisher Scientific) with 1 μg of Cas9/gRNA RNP comprising T3 or T9 spacer sequence (1:1.2 Cas9:spacer ratio). As shown in FIG. 4, cell viability was assessed by forward and side scatter on days 1 and 2 post editing.

Then, cell viabilities were compared when CD34⁺ cells were edited with RNPs containing Cas9 from two different sources (Alt-R S.p. Cas9 Nuclease V3 from IDT or SpyFi Cas9 from Aldevron) along with AAV donor templates, as shown in FIG. 5. Adult human mobilized CD34⁺ cells were cultured in SCGM media supplemented with TPO, SCF, FLT3L and IL6 (100 ng/ml) for 48 hours, followed by electroporation using Neon with 1 μg of RNP comprising T3 or T9 spacer sequence (1:1.2 Cas9:spacer ratio) and AAV transduction. Cell viability was assessed by forward and side scatter on days 1, 2 and 5.

For the comparison of editing rates at the FOXP3 gene, CD34⁺ cells were edited using RNPs comprising Cas9 from two different sources (Alt-R S.p. Cas9 Nuclease V3 from IDT or SpyFi Cas9 from Aldevron) along with AAV donor templates.

The general in vitro study protocol started from thawed CD34⁺ cells, which were cultured for 2 days before treatment on Day 0 with RNP and AAV donor template immediately thereafter. AAV washout was performed on Day 1, with FACS assays performed on Days 2 and 5.

Specifically, adult human mobilized CD34⁺ cells were cultured in SCGM media supplemented with TPO, SCF, FLT3L and IL6 (100 ng/ml) for 48 hours, followed by electroporation using Neon with 1 μg of Cas9/gRNA RNP comprising T3 or T9 spacer sequence (1:1.2 Cas9: gRNA ratio) and transduction with AAV donor template #3008. GFP expression was assessed at day 5 by flow cytometry and shown in the table below.

| | % GFP^{high} |
|---|---|
| | (HR) after |
| | transduction |
| | with AAV donor |
| Treatment conditions | template #3008 |
| IDT Cas9/gRNA-T9 RNP | 9.6 ± 5.6 |
| SpyFi Cas9/gRNA-T9 RNP | 13.2 ± 6.05 |
| IDT Cas9/gRNA-T3 RNP | 14.8 ± 4.6 |
| SpyFi Cas9/gRNA-T3 RNP | 22 ± 4.8 |

Moreover, the comparison of NHEJ rates (Inference of CRISPR edits (ICE) scores) at the FOXP3 gene in CD34⁺ cells edited using Cas9 from two different sources is shown in the table below. Adult human Mobilized CD34⁺ cells were cultured in SCGM media supplemented with TPO, SCF, FLT3L and IL6 (100 ng/ml) for 48 hours, followed by electroporation using Neon with 1 μg of Cas9/gRNA RNP comprising T3 or T9 spacer sequence (1:1.2 Cas9: gRNA ratio). The cells were cultured for 5 days post editing, followed by genomic DNA extraction. The region around the cut site was amplified, sequenced and analyzed by ICE (Inference of CRISPR Edits) analysis (Hsiau, T. et al. Inference of CRISPR Edits from Sanger Trace Data. bioRxiv 251082). Locus A was a locus on the X chromosome other than FOXP3.

| | ICE Scores after treatment | |
|---|---|---|
| RNP | with RNP comprising | |
| Spacer | indicated Cas9 | |
| Sequence | IDT Cas9 | SpyFi Cas9 |
| T9 | 17 ± 9 | 28 ± 11 |
| T3 | 10 ± 2 | 20.5 ± 2.5 |
| Locus A | 18 ± 2 | 29.5 ± 3.5 |

Then, to compare the cleavage efficiency of RNPs comprising T3 or T9 spacer sequences across three different donors, as shown in the table below, adult human mobilized CD34⁺ cells were cultured in SCGM and transfected using Neon electroporation system. The cells were cultured for 5 days post editing, followed by genomic DNA extraction. The region around the nuclease cut site was amplified, sequenced and analyzed by ICE (Inference of CRISPR Edits) analysis.

| | % cleavage (ICE) by | |
|---|---|---|
| | indicated targeting RNP | |
| Donor | T3 | T9 |
| A | 21 ± 1 | 12.5 ± 0.5 |
| B | 37 ± 1 | 22.5 ± 0.5 |
| C | 30.5 ± 0.5 | 17 ± 1 |

The cleavage efficiency of RNPs comprising T3 or T9 spacer sequences was also compared across three different donors using IDT Cas9, as shown in the table below. Adult human Mobilized CD34⁺ cells were cultured in SCGM media supplemented with TPO, SCF, FLT3L and IL6 (100 ng/ml) for 48 hours, followed by electroporation using Neon with 1 ug of Cas9/gRNA RNP comprising T3 or T9 spacer sequence (1:1.2 Cas9:spacer ratio). The cells were cultured for 5 days post editing, followed by genomic DNA extraction. The region around the cut site was amplified, sequenced and analyzed by ICE (Inference of CRISPR Edits) analysis. The region around an off-target cleavage site (SLC2A6) for RNP having T3 spacer sequence was also amplified and subjected to ICE analysis. The off-target site was identified using CCTop—CRISPR/Cas9 target online predictor tool (Stemmer, M. et al. (2017)Plos One, 12(4): e0176619).

| | % cleavage (ICE) by | |
|---|---|---|
| | indicated targeting RNP | |
| | T3 | |
| Donor | On-target | Off-target |
| A | 21 ± 1 | 2 ± 0 |
| B | 37 ± 1 | 3.5 ± 0.5 |
| C | 30.5 ± 0.5 | 2.5 ± 0.5 |

Further, cleavage efficiency at the FOXP3 gene and at an off-target locus was compared for RNPs comprising T3 or T9 spacer sequences when using IDT Cas9 vs SpyFi Cas9, as shown in the table below. Adult human Mobilized CD34⁺ cells were cultured as described and electroporated using Neon with 1 μg of Cas9/gRNA RNP comprising T3 or T9 spacer sequence (1:1.2 Cas9:spacer ratio). Either Alt-R S.p. Cas9 Nuclease V3 from IDT or SpyFi Cas9 from Aldevron were used. The cells were cultured for 5 days post editing, followed by genomic DNA extraction. The region around the cut site was amplified, sequenced and analyzed by ICE (Inference of CRISPR Edits) analysis. The region around the top off-target cleavage site (SLC2A6) for the IDT Cas9/gRNA-T3 RNP was also amplified and subjected to ICE analysis. The other RNPs did not show off-target cleavage at SLC2A6. The off-target site was identified using CCTop—CRISPR/Cas9 target online predictor tool.

| | % cleavage |
|---|---|
| RNP at indicated site | (ICE) |
| IDT Cas9/gRNA-T3 on-target | 32 |
| IDT Cas9/gRNA-T3 off-target | 2 |
| SpyFi Cas9/gRNA-T3 on-target | 63 |
| IDT Cas9/gRNA-T9 on-target | 35 |
| SpyFi Cas9/gRNA-T9 on-target | 50 |

### Example 4: Modification of CD34⁺ Cell Transfection Protocol

Results

This example describes modified cell transfection protocols for increasing the transfection efficiency of CD34⁺ cells, using the Lonza nucleofector or the Neon electroporation.

Various programs were tested in parallel with the improved protocol using the Neon electroporation device described in Example 3. Comparable cell viability, transfection and HDR rates to Neon using program CM149 on Lonza were achieved, and this program was used subsequently for our in vivo studies. AAV donor template #3088 was used with the SpyFi Cas9/gRNA RNP containing the T3 spacer sequence. AAV donor template #3088 (SEQ ID NO: 33) as DNA donor yielded higher HDR rates under these conditions as compared to AAV donor template #3008.

Next, the previous CD34⁺ culturing protocol (Protocol A) was compared to an alternative protocol (Protocol B). Protocol B required cells to be cultured at a lower density during cytokine stimulation than protocol A and used a different culture media. A higher proportion of quiescent cells was achieved in cells cultured using Protocol B compared to Protocol A, suggesting that Protocol B cultures might maintain a higher fraction of quiescent long-term repopulating HSCs. However, a higher dose of AAV had to be delivered to the cells cultured using Protocol B to achieve comparable HDR rates.

Methods

First, cell viabilities were compared when nucleofecting human CD34⁺ cells with Lonza 4D-Nucleofector™ system (4 different programs) or electroporating with Neon transfection system (Model MPK5000), as shown in table below.

| Electroporation/ | % cell viability after treatment | | |
|---|---|---|---|
| nucleofection | mock | GFP mRNA | T3 RNP |
| Neon | 91.6 | 88.7 | 72.2 |
| Lonza E0100 | 79 | 78.7 | 77.8 |
| Lonza CM149 | 92.8 | 91.3 | 86.6 |
| Lonza DZ-100 | 79.3 | 76.2 | 74.6 |
| Lonza CA137 | 92 | 89.8 | 85.6 |

Adult human mobilized CD34⁺ cells were cultured in SCGM media supplemented with TPO, SCF, FLT3L and IL6 (100 ng/ml) for 48 hours, followed by electroporation using Neon or nucleofection with Lonza. The cells were either mock transfected or transfected with either 1 μg of each GFP mRNA or 1 μg of RNP comprising SpyFi Cas9 and gRNA containing T3 spacer sequence (1:1.2 Cas9: gRNA ratio). Cell viability was assessed by forward and side scatter. Data from a single CD34⁺ donor is shown in the above table.

The comparison of GFP mRNA expression when nucleofecting human CD34⁺ cells with Lonza or electroporating with Neon is shown in table below.

| Electroporation/ | % GFP⁺ cells after treatment | |
|---|---|---|
| nucleofection | Day 1 | Day 4 |
| Neon | 97.3 | 91.1 |
| Lonza E0100 | 46 | 11.6 |
| Lonza CM149 | 90.1 | 58.6 |
| Lonza DZ-100 | 79.3 | 43 |
| Lonza CA137 | 85.7 | 61 |

Adult human mobilized CD34⁺ cells were cultured in SCGM media supplemented with TPO, SCF, FLT3L and IL6 (100 ng/ml) for 48 hours, followed by electroporation using Neon or nucleofection with Lonza. The cells were transfected with 1 μg of GFP mRNA and GFP expression was assessed on days 1 and 4. Data from a single CD34⁺ donor is shown in the above table.

Then, we compared NHEJ rates nucleofecting with Lonza or electroporating human CD34⁺ cells with Neon, as shown in table below.

| | % NHEJ by ddPCR |
|---|---|
| Electroporation/ | after treatment with |
| nucleofection | Cas9/gRNA-T3 RNP |
| Neon | 86 |
| Lonza E0100 | 21 |
| Lonza CM149 | 74 |
| Lonza DZ-100 | 56 |
| Lonza CA137 | 73 |

The general in vitro protocol described in Example 3 was used. Adult human mobilized CD34⁺ cells were cultured in SCGM media supplemented with TPO, SCF, FLT3L and IL6 (100 ng/ml) for 48 hours, followed by electroporation using Neon or nucleofection with Lonza. The cells were transfected with 1 μg of RNP comprising SpyFi Cas9 and gRNA containing T3 spacer sequence (1:1.2 Cas9:spacer ratio). The cells were harvested on day 5 and NHEJ rates were determined using droplet digital PCR. Primers were designed spanning the cut site with the NHEJ probe binding to the T3 spacer sequence cleavage site. A control amplicon of similar size was generated from another region of the FOXP3 gene. Each sample was analyzed in duplicate. The PCR reactions were partitioned into droplets using a QX200 Droplet Generator (Bio-Rad). Amplification was performed using ddPCR Supermix for Probes without UTP (Bio-Rad), 900 nM of primers, 250 nM of Probe, 50 ng of genomic DNA, and 1% DMSO. Droplets were analyzed using the QX200 Droplet Digital PCR System (Bio-Rad) and analyzed using QuantaSoft software (Bio-Rad). Data from a single CD34⁺ donor is shown on the bar graph. The NHEJ rates were calculated using the formula:

$$NHEJ rate = ( ( \frac{Signal from NHEJ probe}{Signal from control probe} ) mock sample - ( \frac{Signal from NHEJ probe}{Signal from control probe} ) T 3 RNP treated sample ) \times 100$$

For the comparison of cell viability when using various nucleofection programs on Lonza versus electroporation by Neon, adult mobilized human CD34⁺ cells were cultured in SCGM media followed by mock electroporation using Neon or nucleofection by Lonza. Cell viability was assessed using forward and side scatter on days 1, 2 and 5. Data from a single CD34⁺ donor is shown in the table below.

| Electroporation/ | % cell viability after mock treatment | | |
|---|---|---|---|
| nucleofection | Day 1 | Day 2 | Day 5 |
| Neon | 84.8 | 86.4 | 82.2 |
| Lonza E0100 | 68 | 68.2 | 77.7 |
| Lonza CM149 | 67.7 | 79.2 | 78.4 |
| Lonza DZ-100 | 64.5 | 67.3 | 80 |
| Lonza CA137 | 76 | 76.7 | 81.8 |

Then, a comparison of cell viability was performed using various nucleofection programs on Lonza versus electroporation by Neon when introducing RNP and AAV. The general in vitro protocol from Example 3 was used. Adult mobilized human CD34⁺ cells were cultured in SCGM media followed by RNP comprising SpyFi Cas9 and gRNA containing T3 spacer sequence (1:1.2 Cas9:spacer ratio) (1 μg) electroporation using Neon or nucleofection by Lonza, followed by transduction with AAV donor template #3088, shown schematically in FIG. 1. Cell viability was assessed using forward and side scatter on days 1, 2 and 5. Data from a single CD34⁺ donor is shown on the bar graph in the table below.

| | % cell viability after treatment | | |
|---|---|---|---|
| | with Cas9/gRNA-T3 RNP + | | |
| Electroporation/ | AAV donor template #3088 | | |
| nucleofection | Day 1 | Day 2 | Day 5 |
| Neon | 69.8 | 54.4 | 55.7 |
| Lonza E0100 | 66.9 | 48 | 49.4 |
| Lonza CM149 | 59.2 | 61.5 | 51.8 |
| Lonza DZ-100 | 57.6 | 43.2 | 37.9 |
| Lonza CA137 | 68.8 | 62.1 | 64.1 |

We then compared the percent GFP expression (HDR) when using various nucleofection programs on Lonza versus electroporation by Neon. AAV donor template #3088 was used for this experiment, which was designed for T3 spacer sequence by placing the T3 spacer sequence cleavage site at the beginning of the 3′ homology arm. The general in vitro protocol in Example 3 was followed. Adult mobilized human CD34⁺ cells were cultured in SCGM media followed by RNP (1 μg) electroporation using Neon or nucleofection by Lonza. This was followed by transduction with AAV donor template (panel A). HDR rates were determined by GFP expression on day 5. Data from a single CD34⁺ donor is shown in the table below. Program CM149 (Lonza) was chosen for future experiments since it yielded the highest editing rates (GFP^{high}) in cells without a significant drop in cell viability.

| | % GFP^{high} after treatment with control or |
|---|---|
| Electroporation/ | Cas9/gRNA-T3 RNP + AAV donor template |
| nucleofection | #3088 |
| Mock | 0 |
| AAV #3088 only | 0.63 |
| Neon | 36.8 |
| Lonza E0100 | 14.4 |
| Lonza CM149 | 43.4 |
| Lonza DZ-100 | 40.4 |
| Lonza CA137 | 26.1 |

The details of the two different in vitro cell genome editing protocols—Protocol A and B—are shown in the table below.

| Conditions | Protocol A | Protocol B |
|---|---|---|
| Media | SCGM | SFEMII |
| Human cytokines | TPO, FLT3L, SCF, IL6 (100 ng/ml) | |
| Small molecule | UM171 and SR1 | |
| Pre-stimulation: cell | 1.00E+06 | 2.50E+05 |
| concentration/ml | | |
| Pre-stimulation time | 48 hours | |
| RNP | 1 ug (1.2:1 molar ratio) | |
| cell concentration | 1 million/ | 1 million/ |
| during EP | 20 μl rxn with | 20 μl rxn with |
| | Neon or Lonza | Lonza |
| AAV MOI | 50 | 50, 100, 200 |
| Cell concentration | 1 million/0.8 ml | 1 million/1 ml |
| for transduction | | |
| 16 hours after transduction | Add media | cells move to |
| | (virus diluted) | 0.25 million cell/ml |
| | | concentration |

For protocol A, mobilized human CD34⁺ cells were cultured in SCGM media supplemented with TPO, SCF, FLT3L and IL6 (100 ng/ml) plus 35 nm UM171 and 1 uM SR1 for 48 hours at a concentration of 1×10⁶ cells/ml, followed by nucleofection of 1 μg of RNP comprising SpyFi Cas9 and gRNA containing T3 spacer sequence (1:1.2 Cas9:spacer ratio) using Lonza. The cells were subsequently transduced with AAV donor template at the MOI of 50. After 16 hours post transduction, the AAV was diluted out by adding more media. For protocol B, CD34⁺ cells were cultured in SFEMII media containing the same supplements as above. The cell density during pre-stimulation was 2.50×10⁵/ml. Following the 48-hour pre-stimulation, the cells were nucleofected with Lonza and plated at a density of 1×10⁶ cells/ml prior to transduction with AAV at MOIs of 50, 100 and 200. After 16 hours post transduction, the cells were re-plated at the density of 2.5×10⁵ cells/ml. Cell viabilities at days 1, 2 and 5 were assessed by forward and side scatter. The data for the comparison of cell viability when using either protocol A or B, from a single CD34⁺ donor, is shown in the table below.

| | % cell viability after transduction with indicated conditions | | | | |
|---|---|---|---|---|---|
| | | AAV | AAV #3088 | AAV #3088 | AAV #3088 |
| | | #3088 | MOI 50 + | MOI 100 + | MOI 200 + |
| Protocol | mock | only | RNP | RNP | RNP |
| A Day 1 | 59.3 | 54.3 | 54.7 | N/A | N/A |
| A Day 2 | 86.1 | 79.2 | 76 | N/A | N/A |
| A Day 5 | 76.6 | 72 | 53.8 | N/A | N/A |
| B Day 1 | 84.3 | 82.6 | 79.7 | 80.4 | 78.6 |
| B Day 2 | 85.5 | 82.1 | 68.1 | 48.9 | 72.3 |
| B Day 5 | 79.1 | 77.2 | 61.9 | 62 | 61.2 |

Then, transduction by AAV donor templates was assessed in CD34⁺ cells cultured by either protocol A or B using flow cytometry. Percent GFP expression at days 1 and 2 are shown in the table below. Data from a single CD34⁺ donor is shown.

| | % GFP⁺ after transduction with indicated AAV donor vector | | | | |
|---|---|---|---|---|---|
| | | AAV #3088 | AAV MOI | AAV MOI | AAV MOI |
| Protocol | mock | only | 50 + RNP | 100 + RNP | 200 + RNP |
| A Day 1 | 3.07 | 16.1 | 47.7 | N/A | N/A |
| A Day 2 | 0.56 | 10.3 | 34.7 | N/A | N/A |
| B Day 1 | 0.31 | 5.02 | 24 | 26.9 | 28.6 |
| B Day 2 | 0.028 | 7.22 | 25.4 | 36.6 | 37.4 |

HDR rates were then determined by stable GFP expression at day 5 in CD34⁺ cells cultured using protocol A or B. GFP expression levels were assessed using flow cytometry. Data from a single CD34⁺ donor is shown in the table below.

| | % GFP⁺ after transduction with indicated AAV donor vector | | | | |
|---|---|---|---|---|---|
| | | AAV #3088 | AAV MOI | AAV MOI | AAV MOI |
| Protocol | mock | only | 50 + RNP | 100 + RNP | 200 + RNP |
| A Day 5 | 0 | 0.7 | 47.7 | N/A | N/A |
| B Day 5 | 0 | 0 | 31.9 | 38.1 | 48.9 |

For the comparison of cell cycle status in cells cultured for 48 hours using protocol A or B, as shown in the table below, adult mobilized CD34were cultured using protocol A or B as previously described and their cell cycle status determined using the Muse™ cell cycle assay kit (Merck KGaA, Darmstadt, Germany), 48 hours post culturing. The bar graph depicts the percent cells in G0/G1, S or G2/M phases with either culturing protocol. DNA content index plots are shown below the respective bar graphs. This suggested that there was a higher proportion of quiescent cells in CD34⁺ cultures with SFEMII media using protocol B than SCGM (protocol A).

| Cell | Day 0 before electroporation % cells with indicated media | |
|---|---|---|
| phase | SFEM II (protocol B) | SCGM (protocol A) |
| G0/G1 | 32 | 15 |
| S | 13 | 7 |
| G2/M | 27 | 38 |

### Example 5: Modification of Editing of CD34⁺ Cells from Healthy Subject

The FOXP3 gene was edited with AAV6 donors designed to introduce a FOXP3 cDNA to enable expression using a WPRE element upon targeted integration. CD34⁺ cells from a single healthy human subject were edited.

FIG. 1 shows a schematic representation of AAV donor template #3232 comprising FOXP3 cDNA vector expressing codon optimized cDNA, WPRE3 element and SV40 polyadenylation site. The table below shows HDR rates when CD34⁺ cells from a healthy human subject were edited using SpyFi Cas9/T3-gRNA (1:1.2) RNPs and the FOXP3 cDNA vector at different MOI using protocol B as previously described. No RNP or no AAV as control did not exhibit measured % HDR.

| Treatment with RNP and AAV donor | % HDR |
|---|---|
| template #3232 at indicated MOI (K) | (by ddPCR) |
| 0.1 | 11.41 |
| 0.2 | 21.3 |
| 0.5 | 34.17 |
| 1.1 | 40.13 |
| 2.2 | 47.35 |

The cell viability of the cells treated as described above at different MOI is shown in the table below. These data suggested that this genome editing approach may provide an effective and sustained long-term cure as it may allow locus specific expression of FOXP3 regardless of the downstream mutation.

| Treatment | AAV MOI (K) | % cell viability at Day 1 |
|---|---|---|
| Mock | None | 80 |
| AAV donor template | 1.1 | 77.7 |
| #3232 | | |
| RNP + AAV donor | 0.1 | 75.3 |
| template #3232 | 0.2 | 73.4 |
| | 0.5 | 75.8 |
| | 1.1 | 74.5 |
| | 2.2 | 73.8 |

### Example 6: HDR-Edited CD34⁺ Cells Engraftment in Mice

Results

The long-term engraftment of genome-edited (GFP) CD34 cells cultured according to Example 4 using either protocol A or B and transfected with AAV donor template #3088 and RNP comprising SpyFi Cas9/T3 gRNA (1:1.2 Cas9:gRNA), using either the Neon or Lonza transfection in NSGW41 recipient mice, was assessed. As illustrated in the table below, acceptable HDR was achieved by treatment with the RNP and AAV donor template combination.

| Treatment conditions | % live cells | % GFP^{high} |
|---|---|---|
| mock | 78.6 | 0 |
| AAV #3088 only | 75.0 | 0.7 |
| AAV #3088 MOI 50 + SpyFi | 61.2 | 25.2 |
| Cas9/T3 RNP (1:1.2 | | |
| Cas9:gRNA) | | |

Experimental mice were analyzed 12-16 weeks post-transfer of edited PBSC for engraftment of hCD45⁺ cells in the bone marrow. As summarized in the tables below, average engraftment of human cells was ˜60% in the bone marrow across multiple experiments and ˜5% of those cells maintained long-term GFP.

| | | % hCD45⁺ | % hCD45⁺CD19⁺ |
|---|---|---|---|
| Treatment | | (bone marrow) | (bone marrow) |
| Protocol A | Mock | 65.37 ± 12.46 | 51.73 ± 4.997 |
| | Edited | 61.8 ± 11.67 | 47.38 ± 4.515 |
| Protocol B | Mock | 69.08 ± 10.01 | 47.74 ± 3.949 |
| | edited | 65.94 ± 5.685 | 57.97 ± 3.54 |

| | | % hCD45⁺CD33⁺ | % hCD45⁺GFP⁺ |
|---|---|---|---|
| Treatment | | (bone marrow) | (bone marrow) |
| Protocol A | Mock | 36.47 ± 4.055 | 0.29 ± 0.08 |
| | Edited | 35.76 ± 3.97 | 4.88 ± 2.26 |
| Protocol B | Mock | 39.27 ± 3.155 | 0.16 ± 0.035 |
| | Edited | 30.52 ± 2.93 | 8.2 ± 2.6 |

These data formally demonstrated that FOXP3 gene HDR-edited HSC retained the ability to engraft long-term and retain expression of a donor gene expression cassette. All recipients harbored edited cells in both the myeloid and B cell populations and these lineages were present at ratios equivalent to recipients of mock-edited human CD34⁺ cells. These data were consistent with editing of a multipotent HSC and indicate that the differentiation capacity of HDR-edited stem cells was not compromised by editing the FOXP3 gene. We did not observe any significant difference in terms of total engraftment with either protocol. Overall, the recipient mice harbored edited cells in the B cell populations and this lineage was present at ratios comparable to recipients of mock-edited human CD34⁺ cells, suggesting that the differentiation capacity was not compromised by editing the FOXP3 gene.

| | | % hCD33⁺GFP⁺ | % hCD19⁺GFP⁺ |
|---|---|---|---|
| Treatment | | (bone marrow) | (bone marrow) |
| Protocol A | Mock | 0.024 ± 0.095 | 0.09 ± 0.04 |
| | Edited | 3.76 ± 1.033 | 2.71 ± 1.498 |
| Protocol B | Mock | 0.31 ± 0.09916 | 0.12 ± 0.008 |
| | Edited | 10.55 ± 2.826** | 7.1 ± 2.363* |

*P value = 0.0332 compared to mock in protocol A
**P value = 0.0021 compared to mock in protocol B or mock in protocol A

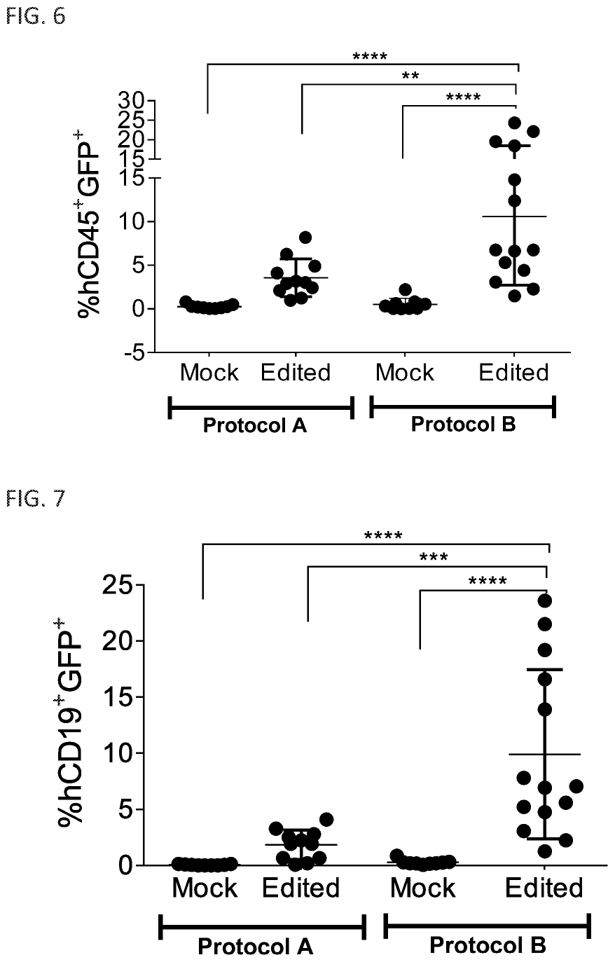

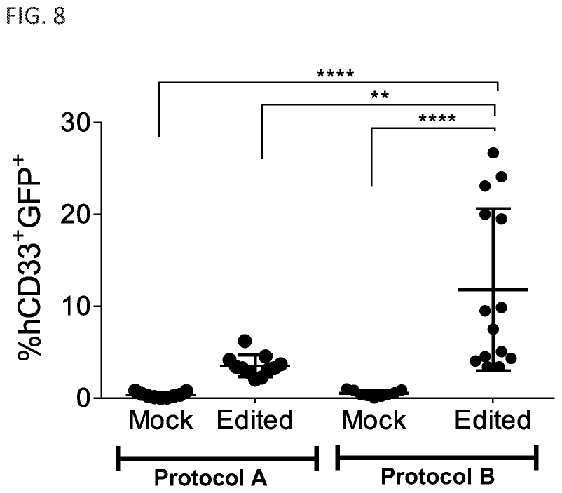

Average engraftment of human hematopoietic cells within the spleen was slightly lower than bone marrow but comparable between recipient animals treated with mock and HDR-edited cells. HDR-edited GFP⁺ cells were present in all cell lineages (B, T, myeloid) and were present in ratios comparable to ratios found in the mock treatment. The successful engraftment of GFP⁺ cells are reflected in the tables below and in FIGS. 6-8.

| | | % hCD45⁺ | % hCD45⁺CD19⁺ |
|---|---|---|---|
| Treatment | | (spleen) | (spleen) |
| Protocol A | Mock | 19.27 ± 4.66 | 73.56 ± 4.18 |
| | Edited | 16.18 ± 3.42 | 70.59 ± 2.84 |
| Protocol B | Mock | 18.58 ± 2.92 | 76.97 ± 3.28 |
| | edited | 11.88 ± 2.25 | 70.96 ± 2.83 |
| Protocol A | Mock | 9.62 ± 1.6 | 0.26 ± 0.08 |
| | Edited | 9.16 ± 1.06 | 3.56 ± 0.65** |
| Protocol B | Mock | 11.16 ± 1.97 | 0.51 ± 0.23 |
| | edited | 12.77 ± 1.49 | 10.58 ± 2.09**** |

**P value = 0.0021 (edited Protocol B vs. edited Protocol A)
****P value < 0.0001 (edited Protocol B vs. mock in either experiment)

| | | % hCD19⁺GFP⁺ | % hCD33⁺GFP⁺ |
|---|---|---|---|
| Treatment | | (spleen) | (spleen) |
| Protocol A | Mock | 0.05 ± 0.01 | 0.33 ± 0.09 |
| | Edited | 1.84 ± 0.39*** | 3.51 ± 0.35** |
| Protocol B | Mock | 0.28 ± 0.07 | 0.51 ± 0.11 |
| | edited | 9.90 ± 2.01**** | 11.79 ± 2.35**** |

**P value = 0.0021 (edited Protocol B vs. edited Protocol A)
***P value = 0.002 (edited Protocol B vs. edited Protocol A)
****P value < 0.0001 (edited Protocol B vs. mock in either experiment)

Additionally, the percent of human CD45⁺ hematopoietic stem cells engrafted within the bone marrow as defined by expression of CD38^{low} CD34⁺ was similar between mock and HDR-edited recipients as shown in the tables below. GFP⁺ cells were present within this population consistent with editing of a stem cell population capable of persisting long-term in vivo.

| | | % | % |
|---|---|---|---|
| | | hCD45⁺CD34⁺CD38^{low} | hCD45⁺CD34⁺CD38^{low}GFP⁺ |
| Treatment | | (bone marrow) | (spleen) |
| Protocol | Mock | 2.69 ± 0.68 | 0.12 ± 0.1 |
| A | Edited | 2.58 ± 0.78 | 4.86 ± 2.27 |
| Protocol | Mock | 5.28 ± 1.13 | 0.37 ± 0.05 |
| B | edited | 5.6 ± 1.1 | 13.89 ± 5.07* |

*P value = 0.0332 compared to mock in protocol A

Methods

For protocol A, mobilized human CD34⁺ cells were cultured in SCGM media supplemented with TPO, SCF, FLT3L and IL6 (each at 100 ng/ml) plus 35 nm UM171 and 1 uM SR1 for 48 hours at a concentration of 1×10⁶ cells/ml, followed by nucleofection of 1 μg of RNP comprising SpyFi Cas9/T3 gRNA (1:1.2 Cas9: gRNA molar ratio) using Neon or Lonza. The cells were subsequently transduced with AAV donor template #3088 at the MOI of 50. Cells (mock or edited, 1.5-2×10⁶ per mouse) cultured with the above protocol were injected into NSGW41 recipient mice that were injected with 12.5 mg/kg busulfan 24 hours prior. The transplanted mice were sacrificed 12-16 weeks later, and bone marrow and spleens were analyzed

For protocol B, CD34⁺ cells were cultured in SFEMII media containing the same supplements and concentrations as above. The cell density during pre-stimulation was 2.50×10⁵/ml. Following the 48 hours pre-stimulation, the cells were nucleofected with Lonza and plated at a density of 1×10⁶ cells/ml prior to transduction with AAV donor template at MOI of 200. Cells (mock or edited, 1.5-2×10⁶ per mouse) cultured with the above protocol were injected into NSGW41 recipient mice that were injected with 12.5 mg/kg busulfan 24 hours prior. The transplanted mice were sacrificed 12-16 weeks later, and bone marrow and spleens were analyzed.

The gating strategy for analyzing cells harvested from the bone marrow of NSGW41 mice 16 weeks following cell transplantation is described below. Bone marrow was harvested from mice transplanted with mock untreated cells. In parallel, bone marrow was harvested from mice transplanted with cells treated with AAV plus RNP. In both cases, the degree of hCD45:mCD45 chimerism was determined, and human CD45-gated CD33⁺ and CD19⁺ staining was performed. GFP expression among hCD45⁺, CD33⁺ and CD19⁺ cells was determined.

These sorted cells were transfected with 1 μg of RNP comprising SpyFi Cas9/T3 gRNA (1:1.2 Cas9: gRNA ratio) and transduced with AAV donor template #3088 (SEQ ID NO: 35) at MOIs ranging from 50-200. The cells were transplanted into NSGW41 mice the following day. Mice were injected with 12.5 mg/kg busulfan one day prior to transplantation of cells. The mice were sacrificed 12-16 weeks post transplantation and analyzed for the presence of human cells. The tables shown above summarize the results of engraftment of the sorted cells. Both mock and RNP-edited cells engrafted at comparable rates in recipient mice.

The gating strategy for analyzing cells harvested from the spleen of NSGW41 mice 16 weeks following cell transplantation is described below. Spleens were harvested from mice transplanted with mock untreated cells. In parallel, spleens were harvested from mice transplanted with cells treated with AAV plus RNP. In each cohort, the degree of hCD45:mCD45 chimerism was determined, and human CD45-gated CD33⁺ and CD19⁺ staining performed. GFP expression among hCD45⁺, CD33⁺ and CD19⁺ cells was determined.

These sorted cells were transfected with 1 μg of RNP comprising SpyFi Cas9/T3 gRNA (1:1.2 Cas9: gRNA ratio) and transduced with AAV donor template #3088 (SEQ ID NO: 35) at MOIs ranging from 50-200k. The cells were transplanted into NSGW41 mice the following day. Mice were injected with 12.5 mg/kg busulfan 1-2 days prior to transplantation of cells. The mice were sacrificed 12-16 weeks post transplantation and analyzed for the presence of human cells. Both mock and RNP-edited cells engrafted at comparable rates in recipient mice.

The gating strategy for analyzing GFP⁺ cells among human CD34⁺ CD38^{low} CD45⁺ cells recovered from the bone marrow of NSGW41 mice engrafted with mock or edited cells, is as follows. The degree of hCD45:mCD45 chimerism was determined, and human CD45-gated CD38^{low}CD34⁺ staining was performed. GFP⁺ cells among the CD38^{low}CD34⁺ population were isolated.

These cells were transfected with 1 μg of RNP comprising SpyFi Cas9/T3 gRNA (1:1.2 Cas9: gRNA ratio) and transduced with AAV donor template #3088 (SEQ ID NO: 35) at MOIs ranging from 50-200. The cells were transplanted into NSGW41 mice the following day. Mice were injected with 12.5 mg/kg busulfan 1-2 days prior to transplantation of cells. The mice were sacrificed 12-16 weeks post transplantation and analyzed for the presence of human cells.

### Sequences

In addition to sequences disclosed elsewhere in the present disclosure, the following sequences are provided as they are mentioned or used in various exemplary embodiments of the disclosures, which are provided for the purpose of illustration.

| SEQ | | |
|---|---|---|
| ID | | |
| NO | Sequence | Description |
| 1 | TTCCAGGGCCGAGATCTTCG | T1 spacer |
| | | targeting |
| | | human |
| | | FOXP3 |
| 2 | CGCCTCGAAGATCTCGGCCC | T3 spacer |
| | | targeting |
| | | human |
| | | FOXP3 |
| 3 | TCGAAGATCTCGGCCCTGGA | T4 spacer |
| | | targeting |
| | | human |
| | | FOXP3 |
| 4 | GGCCCTGGAAGGTTCCCCCT | T7 spacer |
| | | targeting |
| | | human |
| | | FOXP3 |
| 5 | TCCAGCTGGGCGAGGCTCCT | T9 spacer |
| | | targeting |
| | | human |
| | | FOXP3 |
| 6 | TCAGACCTGCTGGGGGCCCG | T18 spacer |
| | | targeting |
| | | human |
| | | FOXP3 |
| 7 | GAGCCCCGCCTCGAAGATCT | R1 spacer |
| | | targeting |
| | | human |
| | | FOXP3 |
| 8 | AGG | PAM |
| | | sequence |
| 9 | TGG | PAM |
| | | sequence |
| 10 | AGG | PAM |
| | | sequence |
| 11 | GGG | PAM |
| | | sequence |
| 12 | GGG | PAM |
| | | sequence |
| 13 | GGG | PAM |
| | | sequence |
| 14 | CGG | PAM |
| | | sequence |
| 15 | ATTCCCAGGGCCGGTTAATG | P1 spacer |
| | | targeting |
| | | human |
| | | AAVS1 |
| 16 | GTCCCCTCCACCCCACAGTG | P3 spacer |
| | | targeting |
| | | human |
| | | AAVS1 |
| 17 | ACCCCACAGTGGGGCCACTA | P4 spacer |
| | | targeting |
| | | human |
| | | AAVS1 |
| 18 | CCTCTAAGGTTTGCTTACGA | N1 spacer |
| | | targeting |
| | | human |
| | | AAVS1 |
| 19 | TATAAGGTGGTCCCAGCTCG | N2 spacer |
| | | targeting |
| | | human |
| | | AAVS1 |
| 20 | CCATCGTAAGCAAACCTTAG | N3 spacer |
| | | targeting |
| | | human |
| | | AAVS1 |
| 21 | TGG | PAM |
| | | sequence |
| 22 | GGG | PAM |
| | | sequence |
| 23 | GGG | PAM |
| | | sequence |
| 24 | TGG | PAM |
| | | sequence |
| 25 | GGG | PAM |
| | | sequence |
| 26 | AGG | PAM |
| | | sequence |
| 27 | GACTCCTGGGGATGGGCCAA | mT20 spacer |
| | | target murine |
| | | FOXP3 |
| 28 | TTGGCCCTTGGCCCATCCCC | mT22 spacer |
| | | target murine |
| | | FOXP3 |
| 29 | CCAGCTTGGCAAGACTCCTG | mT23 spacer |
| | | target murine |
| | | FOXP3 |
| 30 | GGG | PAM |
| | | sequence |
| 31 | AGG | PAM |
| | | sequence |
| 32 | GGG | PAM |
| | | sequence |
| 33 | GTAGAAAAGATCAAAGGATCTTCTTGAGATCCTTTTTTTCTGCGCGTAATCTGCTGCTTGC | #3008 |
| | AAACAAAAAAACCACCGCTACCAGCGGTGGTTTGTTTGCCGGATCAAGAGCTACCAACT | pAAV_FoxP |
| | CTTTTTCCGAAGGTAACTGGCTTCAGCAGAGCGCAGATACCAAATACTGTCCTTCTAGTG | 3.0.6 kb.MN |
| | TAGCCGTAGTTAGGCCACCACTTCAAGAACTCTGTAGCACCGCCTACATACCTCGCTCTG | D.GFP.WPR |
| | CTAATCCTGTTACCAGTGGCTGCTGCCAGTGGCGATAAGTCGTGTCTTACCGGGTTGGAC | E3.pA |
| | TCAAGACGATAGTTACCGGATAAGGCGCAGCGGTCGGGCTGAACGGGGGGTTCGTGCAC | |
| | ACAGCCCAGCTTGGAGCGAACGACCTACACCGAACTGAGATACCTACAGCGTGAGCTAT | |
| | GAGAAAGCGCCACGCTTCCCGAAGGGAGAAAGGCGGACAGGTATCCGGTAAGCGGCAG | |
| | GGTCGGAACAGGAGAGCGCACGAGGGAGCTTCCAGGGGGAAACGCCTGGTATCTTTATA | |
| | GTCCTGTCGGGTTTCGCCACCTCTGACTTGAGCGTCGATTTTTGTGATGCTCGTCAGGGGG | |
| | GCGGAGCCTATGGAAAAACGCCAGCAACGCGGCCTTTTTACGGTTCCTGGCCTTTTGCTG | |
| | GCCTTTTGCTCACATGTTCTTTCCTGCGTTATCCCCTGATTCTGTGGATAACCGTATTACCG | |
| | CCTTTGAGTGAGCTGATACCGCTCGCCGCAGCCGAACGACCGAGCGCAGCGAGTCAGTG | |
| | AGCGAGGAAGCGGAAGAGCGCCCAATACGCAAACCGCCTCTCCCCGCGCGTTGGCCGAT | |
| | TCATTAATGCAGCTGCGCGCTCGCTCGCTCACTGAGGCCGCCCGGGCAAAGCCCGGGCGT | |
| | CGGGCGACCTTTGGTCGCCCGGCCTCAGTGAGCGAGCGAGCGCGCAGAGAGGGAGTGGC | |
| | CAACTCCATCACTAGGGGTTCCTTGTAGTTAATGATTAACCCGCCATGCTACTTATCTACG | |
| | TAGCGGCCGCTCAGAATCTACCCACTTCTCGCCTTCTCCACTGCCACCAGCCCATTCTGTG | |
| | CCAGCATCATCACTTGCCAGGACTGTTACAATAGCCTCCTCACTAGCCCCACTCACAGCA | |
| | GCCAGATGAATCTTTTGAGTCCATGCCTAGTCACTGGGGCAAAATAGGACTCCGAGGAG | |
| | AAAGTCCGAGACCAGCTCCGGCAAGATGAGCAAACACAGCCTGTGCAGGGTGCAGGGA | |
| | GGGCTAGAGGCCTGAGGCTTGAAACAGCTCTCAAGTGGAGGGGGAAACAACCATTGCCC | |
| | TCATAGAGGACACATCCACACCAGGGCTGTGCTAGCGTGGGCAGGCAAGCCAGGTGCTG | |
| | GACCTCTGCACGTGGGGCATGTGTGGGTATGTACATGTACCTGTGTTCTTGGTGTGTGTGT | |
| | GTGTGTGTGTGTGTGTGTGTGTCTAGAGCTGGGGTGCAACTATGGGGCCCCTCGGGACAT | |
| | GTCCCAGCCAATGCCTGCTTTGACCAGAGGAGTGTCCACGTGGCTCAGGTGGTCGAGTAT | |
| | CTCATACCGCCCTAGCACACGTGTGACTCCTTTCCCCTATTGTCTACGCAGCCTGCCCTTG | |
| | GACAAGGACCCGACGCGTAGGAACAGAGAAACAGGAGAATATGGGCCAAACAGGATAT | |
| | CTGTGGTAAGCAGTTCCTGCCCCGGCTCAGGGCCAAGAACAGTTGGAACAGCAGAATAT | |
| | GGGCCAAACAGGATATCTGTGGTAAGCAGTTCCTGCCCCGGCTCAGGGCCAAGAACAGA | |
| | TGGTCCCCAGATGCGGTCCCGCCCTCAGCAGTTTCTAGAGAACCATCAGATGTTTCCAGG | |
| | GTGCCCCAAGGACCTGAAATGACCCTGTGCCTTATTTGAACTAACCAATCAGTTCGCTTC | |
| | TCGCTTCTGTTCGCGCGCTTCTGCTCCCCGAGCTCTATATAAGCAGAGCTCGTTTAGTGAA | |
| | CCGTCAGATCGCCTGGAGACGCCATCCACGCTGTTTTGACTTCCATAGAAGGATCTCGAG | |
| | GCCACCATGGTGAGCAAGGGCGAGGAGCTGTTCACCGGGGTGGTGCCCATCCTGGTCGA | |
| | GCTGGACGGCGACGTAAACGGCCACAAGTTCAGCGTGTCCGGCGAGGGCGAGGGCGATG | |
| | CCACCTACGGCAAGCTGACCCTGAAGTTCATCTGCACCACCGGCAAGCTGCCCGTGCCCT | |
| | GGCCCACCCTCGTGACCACCCTGACCTACGGCGTGCAGTGCTTCAGCCGCTACCCCGACC | |
| | ACATGAAGCAGCACGACTTCTTCAAGTCCGCCATGCCCGAAGGCTACGTCCAGGAGCGC | |
| | ACCATCTTCTTCAAGGACGACGGCAACTACAAGACCCGCGCCGAGGTGAAGTTCGAGGG | |
| | CGACACCCTGGTGAACCGCATCGAGCTGAAGGGCATCGACTTCAAGGAGGACGGCAACA | |
| | TCCTGGGGCACAAGCTGGAGTACAACTACAACAGCCACAACGTCTATATCATGGCCGAC | |
| | AAGCAGAAGAACGGCATCAAGGTGAACTTCAAGATCCGCCACAACATCGAGGACGGCA | |
| | GCGTGCAGCTCGCCGACCACTACCAGCAGAACACCCCCATCGGCGACGGCCCCGTGCTG | |
| | CTGCCCGACAACCACTACCTGAGCACCCAGTCCGCCCTGAGCAAAGACCCCAACGAGAA | |
| | GCGCGATCACATGGTCCTGCTGGAGTTCGTGACCGCCGCCGGGATCACTCTCGGCATGGA | |
| | CGAGCTGTACAAGTAAAAGCTTGATAATCAACCTCTGGATTACAAAATTTGTGAAAGATT | |
| | GACTGGTATTCTTAACTATGTTGCTCCTTTTACGCTATGTGGATACGCTGCTTTAATGCCT | |
| | TTGTATCATGCTATTGCTTCCCGTATGGCTTTCATTTTCTCCTCCTTGTATAAATCCTGGTT | |
| | AGTTCTTGCCACGGCGGAACTCATCGCCGCCTGCCTTGCCCGCTGCTGGACAGGGGCTCG | |
| | GCTGTTGGGCACTGACAATTCCGTGGGTCGACTGCTTTATTTGTGAAATTTGTGATGCTAT | |
| | TGCTTTATTTGTAACCATTATAAGCTGCAATAAACAAGTTAACAACAACAATTGCATTCA | |
| | TTTTATGTTTCAGGTTCAGGGGGAGATGTGGGAGGTTTTTTAAAGCACTAGTGTGAGGCC | |
| | CTGGGCCCAGGATGGGGCAGGCAGGGTGGGGTACCTGGACCTACAGGTGCCGACCTTTA | |
| | CTGTGGCACTGGGCGGGAGGGGGGCTGGCTGGGGCACAGGAAGTGGTTTCTGGGTCCCA | |
| | GGCAAGTCTGTGACTTATGCAGATGTTGCAGGGCCAAGAAAATCCCCACCTGCCAGGCCT | |
| | CAGAGATTGGAGGCTCTCCCCGACCTCCCAATCCCTGTCTCAGGAGAGGAGGAGGCCGT | |
| | ATTGTAGTCCCATGAGCATAGCTATGTGTCCCCATCCCCATGTGACAAGAGAAGAGGACT | |
| | GGGGCCAAGTAGGTGAGGTGACAGGGCTGAGGCCAGCTCTGCAACTTATTAGCTGTTTG | |
| | ATCTTTAAAAAGTTACTCGATCTCCATGAGCCTCAGTTTCCATACGTGTAAAAGGGGGAT | |
| | GATCATAGCATCTACCATGTGGGCTTGCAGTGCAGAGTATTTGAATTAGACACAGAACAG | |
| | TGAGGATCAGGATGGCCTCTCACCCACCTGCCTTTCTGCCCAGCTGCCCACACTGCCCCT | |
| | AGTCATGGTGGCACCCTCCGGGGCACGGCTGGGCCCCTTGCCCCACTTACAGGCACCGCG | |
| | GCGCTACGTAGATAAGTAGCATGGCGGGTTAATCATTAACTACAAGGAACCCCTAGTGAT | |
| | GGAGTTGGCCACTCCCTCTCTGCGCGCTCGCTCGCTCACTGAGGCCGGGCGACCAAAGGT | |
| | CGCCCGACGCCCGGGCTTTGCCCGGGCGGCCTCAGTGAGCGAGCGAGCGCGCCAGCTGG | |
| | CGTAATAGCGAAGAGGCCCGCACCGATCGCCCTTCCCAACAGTTGCGCAGCCTGAATGG | |
| | CGAATGGCGATTCCGTTGCAATGGCTGGCGGTAATATTGTTCTGGATATTACCAGCAAGG | |
| | CCGATAGTTTGAGTTCTTCTACTCAGGCAAGTGATGTTATTACTAATCAAAGAAGTATTG | |
| | CGACAACGGTTAATTTGCGTGATGGACAGACTCTTTTACTCGGTGGCCTCACTGATTATA | |
| | AAAACACTTCTCAGGATTCTGGCGTACCGTTCCTGTCTAAAATCCCTTTAATCGGCCTCCT | |
| | GTTTAGCTCCCGCTCTGATTCTAACGAGGAAAGCACGTTATACGTGCTCGTCAAAGCAAC | |
| | CATAGTACGCGCCCTGTAGCGGCGCATTAAGCGCGGCGGGTGTGGTGGTTACGCGCAGC | |
| | GTGACCGCTACACTTGCCAGCGCCCTAGCGCCCGCTCCTTTCGCTTTCTTCCCTTCCTTTCT | |
| | CGCCACGTTCGCCGGCTTTCCCCGTCAAGCTCTAAATCGGGGGCTCCCTTTAGGGTTCCG | |
| | ATTTAGTGCTTTACGGCACCTCGACCCCAAAAAACTTGATTAGGGTGATGGTTCACGTAG | |
| | TGGGCCATCGCCCTGATAGACGGTTTTTCGCCCTTTGACGTTGGAGTCCACGTTCTTTAAT | |
| | AGTGGACTCTTGTTCCAAACTGGAACAACACTCAACCCTATCTCGGTCTATTCTTTTGATT | |
| | TATAAGGGATTTTGCCGATTTCGGCCTATTGGTTAAAAAATGAGCTGATTTAACAAAAAT | |
| | TTAACGCGAATTTTAACAAAATATTAACGTTTACAATTTAAATATTTGCTTATACAATCTT | |
| | CCTGTTTTTGGGGCTTTTCTGATTATCAACCGGGGTACATATGATTGACATGCTAGTTTTA | |
| | CGATTACCGTTCATCGATTCTCTTGTTTGCTCCAGACTCTCAGGCAATGACCTGATAGCCT | |
| | TTGTAGAGACCTCTCAAAAATAGCTACCCTCTCCGGCATGAATTTATCAGCTAGAACGGT | |
| | TGAATATCATATTGATGGTGATTTGACTGTCTCCGGCCTTTCTCACCCGTTTGAATCTTTA | |
| | CCTACACATTACTCAGGCATTGCATTTAAAATATATGAGGGTTCTAAAAATTTTTATCCTT | |
| | GCGTTGAAATAAAGGCTTCTCCCGCAAAAGTATTACAGGGTCATAATGTTTTTGGTACAA | |
| | CCGATTTAGCTTTATGCTCTGAGGCTTTATTGCTTAATTTTGCTAATTCTTTGCCTTGCCTG | |
| | TATGATTTATTGGATGTTGGAATCGCCTGATGCGGTATTTTCTCCTTACGCATCTGTGCGG | |
| | TATTTCACACCGCATATGGTGCACTCTCAGTACAATCTGCTCTGATGCCGCATAGTTAAG | |
| | CCAGCCCCGACACCCGCCAACACCCGCTGACGCGCCCTGACGGGCTTGTCTGCTCCCGGC | |
| | ATCCGCTTACAGACAAGCTGTGACCGTCTCCGGGAGCTGCATGTGTCAGAGGTTTTCACC | |
| | GTCATCACCGAAACGCGCGAGACGAAAGGGCCTCGTGATACGCCTATTTTTATAGGTTAA | |
| | TGTCATGATAATAATGGTTTCTTAGACGTCAGGTGGCACTTTTCGGGGAAATGTGCGCGG | |
| | AACCCCTATTTGTTTATTTTTCTAAATACATTCAAATATGTATCCGCTCATGAGACAATAA | |
| | CCCTGATAAATGCTTCAATAATATTGAAAAAGGAAGAGTATGAGTATTCAACATTTCCGT | |
| | GTCGCCCTTATTCCCTTTTTTGCGGCATTTTGCCTTCCTGTTTTTGCTCACCCAGAAACGCT | |
| | GGTGAAAGTAAAAGATGCTGAAGATCAGTTGGGTGCACGAGTGGGTTACATCGAACTGG | |
| | ATCTCAACAGCGGTAAGATCCTTGAGAGTTTTCGCCCCGAAGAACGTTTTCCAATGATGA | |
| | GCACTTTTAAAGTTCTGCTATGTGGCGCGGTATTATCCCGTATTGACGCCGGGCAAGAGC | |
| | AACTCGGTCGCCGCATACACTATTCTCAGAATGACTTGGTTGAGTACTCACCAGTCACAG | |
| | AAAAGCATCTTACGGATGGCATGACAGTAAGAGAATTATGCAGTGCTGCCATAACCATG | |
| | AGTGATAACACTGCGGCCAACTTACTTCTGACAACGATCGGAGGACCGAAGGAGCTAAC | |
| | CGCTTTTTTGCACAACATGGGGGATCATGTAACTCGCCTTGATCGTTGGGAACCGGAGCT | |
| | GAATGAAGCCATACCAAACGACGAGCGTGACACCACGATGCCTGTAGCAATGGCAACAA | |
| | CGTTGCGCAAACTATTAACTGGCGAACTACTTACTCTAGCTTCCCGGCAACAATTAATAG | |
| | ACTGGATGGAGGCGGATAAAGTTGCAGGACCACTTCTGCGCTCGGCCCTTCCGGCTGGCT | |
| | GGTTTATTGCTGATAAATCTGGAGCCGGTGAGCGTGGGTCTCGCGGTATCATTGCAGCAC | |
| | TGGGGCCAGATGGTAAGCCCTCCCGTATCGTAGTTATCTACACGACGGGGAGTCAGGCA | |
| | ACTATGGATGAACGAAATAGACAGATCGCTGAGATAGGTGCCTCACTGATTAAGCATTG | |
| | GTAACTGTCAGACCAAGTTTACTCATATATACTTTAGATTGATTTAAAACTTCATTTTTAA | |
| | TTTAAAAGGATCTAGGTGAAGATCCTTTTTGATAATCTCATGACCAAAATCCCTTAACGT | |
| | GAGTTTTCGTTCCACTGAGCGTCAGACCCC | |
| 34 | GTAGAAAAGATCAAAGGATCTTCTTGAGATCCTTTTTTTCTGCGCGTAATCTGCTGCTTGC | #3037 |
| | AAACAAAAAAACCACCGCTACCAGCGGTGGTTTGTTTGCCGGATCAAGAGCTACCAACT | pAAV_FoxP |
| | CTTTTTCCGAAGGTAACTGGCTTCAGCAGAGCGCAGATACCAAATACTGTCCTTCTAGTG | 3.0.6 kb.FoxP |
| | TAGCCGTAGTTAGGCCACCACTTCAAGAACTCTGTAGCACCGCCTACATACCTCGCTCTG | 3cDNA.WPR |
| | CTAATCCTGTTACCAGTGGCTGCTGCCAGTGGCGATAAGTCGTGTCTTACCGGGTTGGAC | E3.pA |
| | TCAAGACGATAGTTACCGGATAAGGCGCAGCGGTCGGGCTGAACGGGGGGTTCGTGCAC | |
| | ACAGCCCAGCTTGGAGCGAACGACCTACACCGAACTGAGATACCTACAGCGTGAGCTAT | |
| | GAGAAAGCGCCACGCTTCCCGAAGGGAGAAAGGCGGACAGGTATCCGGTAAGCGGCAG | |
| | GGTCGGAACAGGAGAGCGCACGAGGGAGCTTCCAGGGGGAAACGCCTGGTATCTTTATA | |
| | GTCCTGTCGGGTTTCGCCACCTCTGACTTGAGCGTCGATTTTTGTGATGCTCGTCAGGGGG | |
| | GCGGAGCCTATGGAAAAACGCCAGCAACGCGGCCTTTTTACGGTTCCTGGCCTTTTGCTG | |
| | GCCTTTTGCTCACATGTTCTTTCCTGCGTTATCCCCTGATTCTGTGGATAACCGTATTACCG | |
| | CCTTTGAGTGAGCTGATACCGCTCGCCGCAGCCGAACGACCGAGCGCAGCGAGTCAGTG | |
| | AGCGAGGAAGCGGAAGAGCGCCCAATACGCAAACCGCCTCTCCCCGCGCGTTGGCCGAT | |
| | TCATTAATGCAGCTGCGCGCTCGCTCGCTCACTGAGGCCGCCCGGGCAAAGCCCGGGCGT | |
| | CGGGCGACCTTTGGTCGCCCGGCCTCAGTGAGCGAGCGAGCGCGCAGAGAGGGAGTGGC | |
| | CAACTCCATCACTAGGGGTTCCTTGTAGTTAATGATTAACCCGCCATGCTACTTATCTACG | |
| | TAGCGGCCGCTCAGAATCTACCCACTTCTCGCCTTCTCCACTGCCACCAGCCCATTCTGTG | |
| | CCAGCATCATCACTTGCCAGGACTGTTACAATAGCCTCCTCACTAGCCCCACTCACAGCA | |
| | GCCAGATGAATCTTTTGAGTCCATGCCTAGTCACTGGGGCAAAATAGGACTCCGAGGAG | |
| | AAAGTCCGAGACCAGCTCCGGCAAGATGAGCAAACACAGCCTGTGCAGGGTGCAGGGA | |
| | GGGCTAGAGGCCTGAGGCTTGAAACAGCTCTCAAGTGGAGGGGGAAACAACCATTGCCC | |
| | TCATAGAGGACACATCCACACCAGGGCTGTGCTAGCGTGGGCAGGCAAGCCAGGTGCTG | |
| | GACCTCTGCACGTGGGGCATGTGTGGGTATGTACATGTACCTGTGTTCTTGGTGTGTGTGT | |
| | GTGTGTGTGTGTGTGTGTGTGTCTAGAGCTGGGGTGCAACTATGGGGCCCCTCGGGACAT | |
| | GTCCCAGCCAATGCCTGCTTTGACCAGAGGAGTGTCCACGTGGCTCAGGTGGTCGAGTAT | |
| | CTCATACCGCCCTAGCACACGTGTGACTCCTTTCCCCTATTGTCTACGCAGCCTGCCCTTG | |
| | GACAAGGACCCGATGCCTAATCCTCGGCCTGGAAAGCCTAGCGCTCCTTCTCTTGCTCTG | |
| | GGACCTTCTCCTGGCGCCTCTCCATCTTGGAGAGCCGCTCCTAAAGCCAGCGATCTGCTG | |
| | GGAGCTAGAGGACCTGGCGGCACATTTCAGGGCAGAGATCTTAGAGGCGGAGCCCACGC | |
| | TAGCTCCTCCAGCCTTAATCCTATGCCTCCTAGCCAGCTCCAGCTGCCTACACTGCCTCTG | |
| | GTTATGGTGGCTCCTAGCGGAGCTAGACTGGGCCCTCTGCCTCATCTGCAAGCTCTGCTG | |
| | CAGGACAGACCCCACTTCATGCACCAGCTGAGCACCGTGGATGCCCACGCAAGAACACC | |
| | TGTGCTGCAGGTTCACCCTCTGGAATCCCCAGCCATGATCAGCCTGACACCTCCAACAAC | |
| | AGCCACCGGCGTGTTCAGCCTGAAAGCCAGACCTGGACTGCCTCCTGGCATCAATGTGGC | |
| | CAGCCTGGAATGGGTGTCCAGAGAACCTGCTCTGCTGTGCACATTCCCCAATCCAAGCGC | |
| | TCCCAGAAAGGACAGCACACTGTCTGCCGTGCCTCAGAGCAGCTATCCCCTGCTTGCTAA | |
| | CGGCGTGTGCAAGTGGCCTGGATGCGAGAAGGTGTTCGAGGAACCCGAGGACTTCCTGA | |
| | AGCACTGCCAGGCCGATCATCTGCTGGACGAGAAAGGCAGAGCCCAGTGTCTGCTCCAG | |
| | CGCGAGATGGTGCAGTCTCTGGAACAGCAGCTGGTCCTGGAAAAAGAAAAGCTGAGCGC | |
| | CATGCAGGCCCACCTGGCCGGAAAAATGGCCCTGACAAAGGCCAGCAGCGTGGCCTCTT | |
| | CTGATAAGGGCAGCTGCTGCATTGTGGCCGCTGGATCTCAGGGACCTGTGGTTCCTGCTT | |
| | GGAGCGGACCTAGAGAGGCCCCTGATTCTCTGTTTGCCGTGCGGAGACACCTGTGGGGCT | |
| | CTCACGGCAACTCTACTTTCCCCGAGTTCCTGCACAACATGGACTACTTCAAGTTCCACA | |
| | ACATGCGGCCTCCATTCACCTACGCCACACTGATCAGATGGGCCATTCTGGAAGCCCCTG | |
| | AGAAGCAGAGAACCCTGAACGAGATCTACCACTGGTTTACCCGGATGTTCGCCTTCTTCC | |
| | GGAATCACCCTGCCACCTGGAAGAACGCCATCCGGCACAATCTGAGCCTGCACAAGTGC | |
| | TTCGTGCGCGTGGAATCTGAGAAAGGCGCCGTGTGGACAGTGGACGAGCTGGAATTCAG | |
| | AAAGAAGAGAAGCCAGCGGCCTAGCCGGTGCAGCAATCCTACACCTGGACCTTGAAAGC | |
| | TTGATAATCAACCTCTGGATTACAAAATTTGTGAAAGATTGACTGGTATTCTTAACTATGT | |
| | TGCTCCTTTTACGCTATGTGGATACGCTGCTTTAATGCCTTTGTATCATGCTATTGCTTCCC | |
| | GTATGGCTTTCATTTTCTCCTCCTTGTATAAATCCTGGTTAGTTCTTGCCACGGCGGAACT | |
| | CATCGCCGCCTGCCTTGCCCGCTGCTGGACAGGGGCTCGGCTGTTGGGCACTGACAATTC | |
| | CGTGGGTCGACTGCTTTATTTGTGAAATTTGTGATGCTATTGCTTTATTTGTAACCATTAT | |
| | AAGCTGCAATAAACAAGTTAACAACAACAATTGCATTCATTTTATGTTTCAGGTTCAGGG | |
| | GGAGATGTGGGAGGTTTTTTAAAGCACTAGTGTGAGGCCCTGGGCCCAGGATGGGGCAG | |
| | GCAGGGTGGGGTACCTGGACCTACAGGTGCCGACCTTTACTGTGGCACTGGGCGGGAGG | |
| | GGGGCTGGCTGGGGCACAGGAAGTGGTTTCTGGGTCCCAGGCAAGTCTGTGACTTATGCA | |
| | GATGTTGCAGGGCCAAGAAAATCCCCACCTGCCAGGCCTCAGAGATTGGAGGCTCTCCC | |
| | CGACCTCCCAATCCCTGTCTCAGGAGAGGAGGAGGCCGTATTGTAGTCCCATGAGCATAG | |
| | CTATGTGTCCCCATCCCCATGTGACAAGAGAAGAGGACTGGGGCCAAGTAGGTGAGGTG | |
| | ACAGGGCTGAGGCCAGCTCTGCAACTTATTAGCTGTTTGATCTTTAAAAAGTTACTCGAT | |
| | CTCCATGAGCCTCAGTTTCCATACGTGTAAAAGGGGGATGATCATAGCATCTACCATGTG | |
| | GGCTTGCAGTGCAGAGTATTTGAATTAGACACAGAACAGTGAGGATCAGGATGGCCTCT | |
| | CACCCACCTGCCTTTCTGCCCAGCTGCCCACACTGCCCCTAGTCATGGTGGCACCCTCCG | |
| | GGGCACGGCTGGGCCCCTTGCCCCACTTACAGGCACCGCGGCGCTACGTAGATAAGTAG | |
| | CATGGCGGGTTAATCATTAACTACAAGGAACCCCTAGTGATGGAGTTGGCCACTCCCTCT | |
| | CTGCGCGCTCGCTCGCTCACTGAGGCCGGGCGACCAAAGGTCGCCCGACGCCCGGGCTTT | |
| | GCCCGGGCGGCCTCAGTGAGCGAGCGAGCGCGCCAGCTGGCGTAATAGCGAAGAGGCCC | |
| | GCACCGATCGCCCTTCCCAACAGTTGCGCAGCCTGAATGGCGAATGGCGATTCCGTTGCA | |
| | ATGGCTGGCGGTAATATTGTTCTGGATATTACCAGCAAGGCCGATAGTTTGAGTTCTTCT | |
| | ACTCAGGCAAGTGATGTTATTACTAATCAAAGAAGTATTGCGACAACGGTTAATTTGCGT | |
| | GATGGACAGACTCTTTTACTCGGTGGCCTCACTGATTATAAAAACACTTCTCAGGATTCT | |
| | GGCGTACCGTTCCTGTCTAAAATCCCTTTAATCGGCCTCCTGTTTAGCTCCCGCTCTGATT | |
| | CTAACGAGGAAAGCACGTTATACGTGCTCGTCAAAGCAACCATAGTACGCGCCCTGTAG | |
| | CGGCGCATTAAGCGCGGCGGGTGTGGTGGTTACGCGCAGCGTGACCGCTACACTTGCCA | |
| | GCGCCCTAGCGCCCGCTCCTTTCGCTTTCTTCCCTTCCTTTCTCGCCACGTTCGCCGGCTTT | |
| | CCCCGTCAAGCTCTAAATCGGGGGCTCCCTTTAGGGTTCCGATTTAGTGCTTTACGGCAC | |
| | CTCGACCCCAAAAAACTTGATTAGGGTGATGGTTCACGTAGTGGGCCATCGCCCTGATAG | |
| | ACGGTTTTTCGCCCTTTGACGTTGGAGTCCACGTTCTTTAATAGTGGACTCTTGTTCCAAA | |
| | CTGGAACAACACTCAACCCTATCTCGGTCTATTCTTTTGATTTATAAGGGATTTTGCCGAT | |
| | TTCGGCCTATTGGTTAAAAAATGAGCTGATTTAACAAAAATTTAACGCGAATTTTAACAA | |
| | AATATTAACGTTTACAATTTAAATATTTGCTTATACAATCTTCCTGTTTTTGGGGCTTTTCT | |
| | GATTATCAACCGGGGTACATATGATTGACATGCTAGTTTTACGATTACCGTTCATCGATTC | |
| | TCTTGTTTGCTCCAGACTCTCAGGCAATGACCTGATAGCCTTTGTAGAGACCTCTCAAAA | |
| | ATAGCTACCCTCTCCGGCATGAATTTATCAGCTAGAACGGTTGAATATCATATTGATGGT | |
| | GATTTGACTGTCTCCGGCCTTTCTCACCCGTTTGAATCTTTACCTACACATTACTCAGGCA | |
| | TTGCATTTAAAATATATGAGGGTTCTAAAAATTTTTATCCTTGCGTTGAAATAAAGGCTTC | |
| | TCCCGCAAAAGTATTACAGGGTCATAATGTTTTTGGTACAACCGATTTAGCTTTATGCTCT | |
| | GAGGCTTTATTGCTTAATTTTGCTAATTCTTTGCCTTGCCTGTATGATTTATTGGATGTTGG | |
| | AATCGCCTGATGCGGTATTTTCTCCTTACGCATCTGTGCGGTATTTCACACCGCATATGGT | |
| | GCACTCTCAGTACAATCTGCTCTGATGCCGCATAGTTAAGCCAGCCCCGACACCCGCCAA | |
| | CACCCGCTGACGCGCCCTGACGGGCTTGTCTGCTCCCGGCATCCGCTTACAGACAAGCTG | |
| | TGACCGTCTCCGGGAGCTGCATGTGTCAGAGGTTTTCACCGTCATCACCGAAACGCGCGA | |
| | GACGAAAGGGCCTCGTGATACGCCTATTTTTATAGGTTAATGTCATGATAATAATGGTTT | |
| | CTTAGACGTCAGGTGGCACTTTTCGGGGAAATGTGCGCGGAACCCCTATTTGTTTATTTTT | |
| | CTAAATACATTCAAATATGTATCCGCTCATGAGACAATAACCCTGATAAATGCTTCAATA | |
| | ATATTGAAAAAGGAAGAGTATGAGTATTCAACATTTCCGTGTCGCCCTTATTCCCTTTTTT | |
| | GCGGCATTTTGCCTTCCTGTTTTTGCTCACCCAGAAACGCTGGTGAAAGTAAAAGATGCT | |
| | GAAGATCAGTTGGGTGCACGAGTGGGTTACATCGAACTGGATCTCAACAGCGGTAAGAT | |
| | CCTTGAGAGTTTTCGCCCCGAAGAACGTTTTCCAATGATGAGCACTTTTAAAGTTCTGCTA | |
| | TGTGGCGCGGTATTATCCCGTATTGACGCCGGGCAAGAGCAACTCGGTCGCCGCATACAC | |
| | TATTCTCAGAATGACTTGGTTGAGTACTCACCAGTCACAGAAAAGCATCTTACGGATGGC | |
| | ATGACAGTAAGAGAATTATGCAGTGCTGCCATAACCATGAGTGATAACACTGCGGCCAA | |
| | CTTACTTCTGACAACGATCGGAGGACCGAAGGAGCTAACCGCTTTTTTGCACAACATGGG | |
| | GGATCATGTAACTCGCCTTGATCGTTGGGAACCGGAGCTGAATGAAGCCATACCAAACG | |
| | ACGAGCGTGACACCACGATGCCTGTAGCAATGGCAACAACGTTGCGCAAACTATTAACT | |
| | GGCGAACTACTTACTCTAGCTTCCCGGCAACAATTAATAGACTGGATGGAGGCGGATAA | |
| | AGTTGCAGGACCACTTCTGCGCTCGGCCCTTCCGGCTGGCTGGTTTATTGCTGATAAATCT | |
| | GGAGCCGGTGAGCGTGGGTCTCGCGGTATCATTGCAGCACTGGGGCCAGATGGTAAGCC | |
| | CTCCCGTATCGTAGTTATCTACACGACGGGGAGTCAGGCAACTATGGATGAACGAAATA | |
| | GACAGATCGCTGAGATAGGTGCCTCACTGATTAAGCATTGGTAACTGTCAGACCAAGTTT | |
| | ACTCATATATACTTTAGATTGATTTAAAACTTCATTTTTAATTTAAAAGGATCTAGGTGAA | |
| | GATCCTTTTTGATAATCTCATGACCAAAATCCCTTAACGTGAGTTTTCGTTCCACTGAGCG | |
| | TCAGACCCC | |
| 35 | GTAGAAAAGATCAAAGGATCTTCTTGAGATCCTTTTTTTCTGCGCGTAATCTGCTGCTTGC | #3088 |
| | AAACAAAAAAACCACCGCTACCAGCGGTGGTTTGTTTGCCGGATCAAGAGCTACCAACT | pAAV_FOX |
| | CTTTTTCCGAAGGTAACTGGCTTCAGCAGAGCGCAGATACCAAATACTGTCCTTCTAGTG | P3.08_MND. |
| | TAGCCGTAGTTAGGCCACCACTTCAAGAACTCTGTAGCACCGCCTACATACCTCGCTCTG | GFP 08_for |
| | CTAATCCTGTTACCAGTGGCTGCTGCCAGTGGCGATAAGTCGTGTCTTACCGGGTTGGAC | T3 |
| | TCAAGACGATAGTTACCGGATAAGGCGCAGCGGTCGGGCTGAACGGGGGGTTCGTGCAC | |
| | ACAGCCCAGCTTGGAGCGAACGACCTACACCGAACTGAGATACCTACAGCGTGAGCTAT | |
| | GAGAAAGCGCCACGCTTCCCGAAGGGAGAAAGGCGGACAGGTATCCGGTAAGCGGCAG | |
| | GGTCGGAACAGGAGAGCGCACGAGGGAGCTTCCAGGGGGAAACGCCTGGTATCTTTATA | |
| | GTCCTGTCGGGTTTCGCCACCTCTGACTTGAGCGTCGATTTTTGTGATGCTCGTCAGGGGG | |
| | GCGGAGCCTATGGAAAAACGCCAGCAACGCGGCCTTTTTACGGTTCCTGGCCTTTTGCTG | |
| | GCCTTTTGCTCACATGTTCTTTCCTGCGTTATCCCCTGATTCTGTGGATAACCGTATTACCG | |
| | CCTTTGAGTGAGCTGATACCGCTCGCCGCAGCCGAACGACCGAGCGCAGCGAGTCAGTG | |
| | AGCGAGGAAGCGGAAGAGCGCCCAATACGCAAACCGCCTCTCCCCGCGCGTTGGCCGAT | |
| | TCATTAATGCAGCTGCGCGCTCGCTCGCTCACTGAGGCCGCCCGGGCAAAGCCCGGGCGT | |
| | CGGGCGACCTTTGGTCGCCCGGCCTCAGTGAGCGAGCGAGCGCGCAGAGAGGGAGTGGC | |
| | CAACTCCATCACTAGGGGTTCCTTGTAGTTAATGATTAACCCGCCATGCTACTTATCTACG | |
| | TAGCGGCCGCCTTGCCCACTACATCCAAGCTGCTAGCACTGCTCCTGATCCAGCTTCAGA | |
| | TTAAGTCTCAGAATCTACCCACTTCTCGCCTTCTCCACTGCCACCAGCCCATTCTGTGCCA | |
| | GCATCATCACTTGCCAGGACTGTTACAATAGCCTCCTCACTAGCCCCACTCACAGCAGCC | |
| | AGATGAATCTTTTGAGTCCATGCCTAGTCACTGGGGCAAAATAGGACTCCGAGGAGAAA | |
| | GTCCGAGACCAGCTCCGGCAAGATGAGCAAACACAGCCTGTGCAGGGTGCAGGGAGGGC | |
| | TAGAGGCCTGAGGCTTGAAACAGCTCTCAAGTGGAGGGGGAAACAACCATTGCCCTCAT | |
| | AGAGGACACATCCACACCAGGGCTGTGCTAGCGTGGGCAGGCAAGCCAGGTGCTGGACC | |
| | TCTGCACGTGGGGCATGTGTGGGTATGTACATGTACCTGTGTTCTTGGTGTGTGTGTGTGT | |
| | GTGTGTGTGTGTGTGTGTCTAGAGCTGGGGTGCAACTATGGGGCCCCTCGGGACATGTCC | |
| | CAGCCAATGCCTGCTTTGACCAGAGGAGTGTCCACGTGGCTCAGGTGGTCGAGTATCTCA | |
| | TACCGCCCTAGCACACGTGTGACTCCTTTCCCCTATTGTCTACGCAGCCTGCCCTTGGACA | |
| | AGGACCCGATGCCCAACCCCAGGCCTGGCAAGCCCTCGGCCCCTTCCTTGGCCCTTGGCC | |
| | CATCCCCAGGAGCCTCGCCCAGCTGGAGGGCTGCACCCAAAGCCTCAGACCTGCTGGGG | |
| | GCCCGGGGCCCAGGGGGAACCTTCCAACGCGTAGGAACAGAGAAACAGGAGAATATGG | |
| | GCCAAACAGGATATCTGTGGTAAGCAGTTCCTGCCCCGGCTCAGGGCCAAGAACAGTTG | |
| | GAACAGCAGAATATGGGCCAAACAGGATATCTGTGGTAAGCAGTTCCTGCCCCGGCTCA | |
| | GGGCCAAGAACAGATGGTCCCCAGATGCGGTCCCGCCCTCAGCAGTTTCTAGAGAACCA | |
| | TCAGATGTTTCCAGGGTGCCCCAAGGACCTGAAATGACCCTGTGCCTTATTTGAACTAAC | |
| | CAATCAGTTCGCTTCTCGCTTCTGTTCGCGCGCTTCTGCTCCCCGAGCTCTATATAAGCAG | |
| | AGCTCGTTTAGTGAACCGTCAGATCGCCTGGAGACGCCATCCACGCTGTTTTGACTTCCA | |
| | TAGAAGGATCTCGAGGCCACCATGGTGAGCAAGGGCGAGGAGCTGTTCACCGGGGTGGT | |
| | GCCCATCCTGGTCGAGCTGGACGGCGACGTAAACGGCCACAAGTTCAGCGTGTCCGGCG | |
| | AGGGCGAGGGCGATGCCACCTACGGCAAGCTGACCCTGAAGTTCATCTGCACCACCGGC | |
| | AAGCTGCCCGTGCCCTGGCCCACCCTCGTGACCACCCTGACCTACGGCGTGCAGTGCTTC | |
| | AGCCGCTACCCCGACCACATGAAGCAGCACGACTTCTTCAAGTCCGCCATGCCCGAAGG | |
| | CTACGTCCAGGAGCGCACCATCTTCTTCAAGGACGACGGCAACTACAAGACCCGCGCCG | |
| | AGGTGAAGTTCGAGGGCGACACCCTGGTGAACCGCATCGAGCTGAAGGGCATCGACTTC | |
| | AAGGAGGACGGCAACATCCTGGGGCACAAGCTGGAGTACAACTACAACAGCCACAACGT | |
| | CTATATCATGGCCGACAAGCAGAAGAACGGCATCAAGGTGAACTTCAAGATCCGCCACA | |
| | ACATCGAGGACGGCAGCGTGCAGCTCGCCGACCACTACCAGCAGAACACCCCCATCGGC | |
| | GACGGCCCCGTGCTGCTGCCCGACAACCACTACCTGAGCACCCAGTCCGCCCTGAGCAA | |
| | AGACCCCAACGAGAAGCGCGATCACATGGTCCTGCTGGAGTTCGTGACCGCCGCCGGGA | |
| | TCACTCTCGGCATGGACGAGCTGTACAAGTAAAAGCTTGATAATCAACCTCTGGATTACA | |
| | AAATTTGTGAAAGATTGACTGGTATTCTTAACTATGTTGCTCCTTTTACGCTATGTGGATA | |
| | CGCTGCTTTAATGCCTTTGTATCATGCTATTGCTTCCCGTATGGCTTTCATTTTCTCCTCCT | |
| | TGTATAAATCCTGGTTAGTTCTTGCCACGGCGGAACTCATCGCCGCCTGCCTTGCCCGCTG | |
| | CTGGACAGGGGCTCGGCTGTTGGGCACTGACAATTCCGTGGGTCGACTGCTTTATTTGTG | |
| | AAATTTGTGATGCTATTGCTTTATTTGTAACCATTATAAGCTGCAATAAACAAGTTAACA | |
| | ACAACAATTGCATTCATTTTATGTTTCAGGTTCAGGGGGAGATGTGGGAGGTTTTTTAAA | |
| | GCACTAGTCGAGATCTTCGAGGCGGGGCCCATGCCTCCTCTTCTTCCTTGAACCCCATGC | |
| | CACCATCGCAGCTGCAGGTGAGGCCCTGGGCCCAGGATGGGGCAGGCAGGGTGGGGTAC | |
| | CTGGACCTACAGGTGCCGACCTTTACTGTGGCACTGGGCGGGAGGGGGGCTGGCTGGGG | |
| | CACAGGAAGTGGTTTCTGGGTCCCAGGCAAGTCTGTGACTTATGCAGATGTTGCAGGGCC | |
| | AAGAAAATCCCCACCTGCCAGGCCTCAGAGATTGGAGGCTCTCCCCGACCTCCCAATCCC | |
| | TGTCTCAGGAGAGGAGGAGGCCGTATTGTAGTCCCATGAGCATAGCTATGTGTCCCCATC | |
| | CCCATGTGACAAGAGAAGAGGACTGGGGCCAAGTAGGTGAGGTGACAGGGCTGAGGCC | |
| | AGCTCTGCAACTTATTAGCTGTTTGATCTTTAAAAAGTTACTCGATCTCCATGAGCCTCAG | |
| | TTTCCATACGTGTAAAAGGGGGATGATCATAGCATCTACCATGTGGGCTTGCAGTGCAGA | |
| | GTATTTGAATTAGACACAGAACAGTGAGGATCAGGATGGCCTCTCACCCACCTGCCTTTC | |
| | TGCCCAGCTGCCCACACTGCCCCTAGTCATGGTGGCACCCTCCGGGGCACGGCTGGGCCC | |
| | CTTGCCCCACTTACAGGCACTCCTCCAGGACAGGCCACATTTCATGCACCAGGTATGGAC | |
| | GGTGAATGGGCAGGGAGGAGGGAGCAGGTGGGAGAACTGTGGGGAGGGGCCCCGAGTC | |
| | AGGCTGAACCGGATCCTACGTAGATAAGTAGCATGGCGGGTTAATCATTAACTACAAGG | |
| | AACCCCTAGTGATGGAGTTGGCCACTCCCTCTCTGCGCGCTCGCTCGCTCACTGAGGCCG | |
| | GGCGACCAAAGGTCGCCCGACGCCCGGGCTTTGCCCGGGCGGCCTCAGTGAGCGAGCGA | |
| | GCGCGCCAGCTGGCGTAATAGCGAAGAGGCCCGCACCGATCGCCCTTCCCAACAGTTGC | |
| | GCAGCCTGAATGGCGAATGGCGATTCCGTTGCAATGGCTGGCGGTAATATTGTTCTGGAT | |
| | ATTACCAGCAAGGCCGATAGTTTGAGTTCTTCTACTCAGGCAAGTGATGTTATTACTAAT | |
| | CAAAGAAGTATTGCGACAACGGTTAATTTGCGTGATGGACAGACTCTTTTACTCGGTGGC | |
| | CTCACTGATTATAAAAACACTTCTCAGGATTCTGGCGTACCGTTCCTGTCTAAAATCCCTT | |
| | TAATCGGCCTCCTGTTTAGCTCCCGCTCTGATTCTAACGAGGAAAGCACGTTATACGTGC | |
| | TCGTCAAAGCAACCATAGTACGCGCCCTGTAGCGGCGCATTAAGCGCGGCGGGTGTGGT | |
| | GGTTACGCGCAGCGTGACCGCTACACTTGCCAGCGCCCTAGCGCCCGCTCCTTTCGCTTT | |
| | CTTCCCTTCCTTTCTCGCCACGTTCGCCGGCTTTCCCCGTCAAGCTCTAAATCGGGGGCTC | |
| | CCTTTAGGGTTCCGATTTAGTGCTTTACGGCACCTCGACCCCAAAAAACTTGATTAGGGT | |
| | GATGGTTCACGTAGTGGGCCATCGCCCTGATAGACGGTTTTTCGCCCTTTGACGTTGGAG | |
| | TCCACGTTCTTTAATAGTGGACTCTTGTTCCAAACTGGAACAACACTCAACCCTATCTCGG | |
| | TCTATTCTTTTGATTTATAAGGGATTTTGCCGATTTCGGCCTATTGGTTAAAAAATGAGCT | |
| | GATTTAACAAAAATTTAACGCGAATTTTAACAAAATATTAACGTTTACAATTTAAATATT | |
| | TGCTTATACAATCTTCCTGTTTTTGGGGCTTTTCTGATTATCAACCGGGGTACATATGATT | |
| | GACATGCTAGTTTTACGATTACCGTTCATCGATTCTCTTGTTTGCTCCAGACTCTCAGGCA | |
| | ATGACCTGATAGCCTTTGTAGAGACCTCTCAAAAATAGCTACCCTCTCCGGCATGAATTT | |
| | ATCAGCTAGAACGGTTGAATATCATATTGATGGTGATTTGACTGTCTCCGGCCTTTCTCAC | |
| | CCGTTTGAATCTTTACCTACACATTACTCAGGCATTGCATTTAAAATATATGAGGGTTCTA | |
| | AAAATTTTTATCCTTGCGTTGAAATAAAGGCTTCTCCCGCAAAAGTATTACAGGGTCATA | |
| | ATGTTTTTGGTACAACCGATTTAGCTTTATGCTCTGAGGCTTTATTGCTTAATTTTGCTAAT | |
| | TCTTTGCCTTGCCTGTATGATTTATTGGATGTTGGAATCGCCTGATGCGGTATTTTCTCCTT | |
| | ACGCATCTGTGCGGTATTTCACACCGCATATGGTGCACTCTCAGTACAATCTGCTCTGAT | |
| | GCCGCATAGTTAAGCCAGCCCCGACACCCGCCAACACCCGCTGACGCGCCCTGACGGGC | |
| | TTGTCTGCTCCCGGCATCCGCTTACAGACAAGCTGTGACCGTCTCCGGGAGCTGCATGTG | |
| | TCAGAGGTTTTCACCGTCATCACCGAAACGCGCGAGACGAAAGGGCCTCGTGATACGCC | |
| | TATTTTTATAGGTTAATGTCATGATAATAATGGTTTCTTAGACGTCAGGTGGCACTTTTCG | |
| | GGGAAATGTGCGCGGAACCCCTATTTGTTTATTTTTCTAAATACATTCAAATATGTATCCG | |
| | CTCATGAGACAATAACCCTGATAAATGCTTCAATAATATTGAAAAAGGAAGAGTATGAG | |
| | TATTCAACATTTCCGTGTCGCCCTTATTCCCTTTTTTGCGGCATTTTGCCTTCCTGTTTTTG | |
| | CTCACCCAGAAACGCTGGTGAAAGTAAAAGATGCTGAAGATCAGTTGGGTGCACGAGTG | |
| | GGTTACATCGAACTGGATCTCAACAGCGGTAAGATCCTTGAGAGTTTTCGCCCCGAAGAA | |
| | CGTTTTCCAATGATGAGCACTTTTAAAGTTCTGCTATGTGGCGCGGTATTATCCCGTATTG | |
| | ACGCCGGGCAAGAGCAACTCGGTCGCCGCATACACTATTCTCAGAATGACTTGGTTGAGT | |
| | ACTCACCAGTCACAGAAAAGCATCTTACGGATGGCATGACAGTAAGAGAATTATGCAGT | |
| | GCTGCCATAACCATGAGTGATAACACTGCGGCCAACTTACTTCTGACAACGATCGGAGG | |
| | ACCGAAGGAGCTAACCGCTTTTTTGCACAACATGGGGGATCATGTAACTCGCCTTGATCG | |
| | TTGGGAACCGGAGCTGAATGAAGCCATACCAAACGACGAGCGTGACACCACGATGCCTG | |
| | TAGCAATGGCAACAACGTTGCGCAAACTATTAACTGGCGAACTACTTACTCTAGCTTCCC | |
| | GGCAACAATTAATAGACTGGATGGAGGCGGATAAAGTTGCAGGACCACTTCTGCGCTCG | |
| | GCCCTTCCGGCTGGCTGGTTTATTGCTGATAAATCTGGAGCCGGTGAGCGTGGGTCTCGC | |
| | GGTATCATTGCAGCACTGGGGCCAGATGGTAAGCCCTCCCGTATCGTAGTTATCTACACG | |
| | ACGGGGAGTCAGGCAACTATGGATGAACGAAATAGACAGATCGCTGAGATAGGTGCCTC | |
| | ACTGATTAAGCATTGGTAACTGTCAGACCAAGTTTACTCATATATACTTTAGATTGATTTA | |
| | AAACTTCATTTTTAATTTAAAAGGATCTAGGTGAAGATCCTTTTTGATAATCTCATGACCA | |
| | AAATCCCTTAACGTGAGTTTTCGTTCCACTGAGCGTCAGACCCC | |
| 36 | GTAGAAAAGATCAAAGGATCTTCTTGAGATCCTTTTTTTCTGCGCGTAATCTGCTGCTTGC | #3089 |
| | AAACAAAAAAACCACCGCTACCAGCGGTGGTTTGTTTGCCGGATCAAGAGCTACCAACT | pAAV_FOX |
| | CTTTTTCCGAAGGTAACTGGCTTCAGCAGAGCGCAGATACCAAATACTGTCCTTCTAGTG | P3.08_MND. |
| | TAGCCGTAGTTAGGCCACCACTTCAAGAACTCTGTAGCACCGCCTACATACCTCGCTCTG | GFP08_for |
| | CTAATCCTGTTACCAGTGGCTGCTGCCAGTGGCGATAAGTCGTGTCTTACCGGGTTGGAC | T9 |
| | TCAAGACGATAGTTACCGGATAAGGCGCAGCGGTCGGGCTGAACGGGGGGTTCGTGCAC | |
| | ACAGCCCAGCTTGGAGCGAACGACCTACACCGAACTGAGATACCTACAGCGTGAGCTAT | |
| | GAGAAAGCGCCACGCTTCCCGAAGGGAGAAAGGCGGACAGGTATCCGGTAAGCGGCAG | |
| | GGTCGGAACAGGAGAGCGCACGAGGGAGCTTCCAGGGGGAAACGCCTGGTATCTTTATA | |
| | GTCCTGTCGGGTTTCGCCACCTCTGACTTGAGCGTCGATTTTTGTGATGCTCGTCAGGGGG | |
| | GCGGAGCCTATGGAAAAACGCCAGCAACGCGGCCTTTTTACGGTTCCTGGCCTTTTGCTG | |
| | GCCTTTTGCTCACATGTTCTTTCCTGCGTTATCCCCTGATTCTGTGGATAACCGTATTACCG | |
| | CCTTTGAGTGAGCTGATACCGCTCGCCGCAGCCGAACGACCGAGCGCAGCGAGTCAGTG | |
| | AGCGAGGAAGCGGAAGAGCGCCCAATACGCAAACCGCCTCTCCCCGCGCGTTGGCCGAT | |
| | TCATTAATGCAGCTGCGCGCTCGCTCGCTCACTGAGGCCGCCCGGGCAAAGCCCGGGCGT | |
| | CGGGCGACCTTTGGTCGCCCGGCCTCAGTGAGCGAGCGAGCGCGCAGAGAGGGAGTGGC | |
| | CAACTCCATCACTAGGGGTTCCTTGTAGTTAATGATTAACCCGCCATGCTACTTATCTACG | |
| | TAGCGGCCGCATCTCAGGTAATGTCAGCTCGGTCCTTCCAGCTGCTCAAGCTAAAACCCA | |
| | TGTCACTTTGACTCTCCCTCTTGCCCACTACATCCAAGCTGCTAGCACTGCTCCTGATCCA | |
| | GCTTCAGATTAAGTCTCAGAATCTACCCACTTCTCGCCTTCTCCACTGCCACCAGCCCATT | |
| | CTGTGCCAGCATCATCACTTGCCAGGACTGTTACAATAGCCTCCTCACTAGCCCCACTCA | |
| | CAGCAGCCAGATGAATCTTTTGAGTCCATGCCTAGTCACTGGGGCAAAATAGGACTCCGA | |
| | GGAGAAAGTCCGAGACCAGCTCCGGCAAGATGAGCAAACACAGCCTGTGCAGGGTGCA | |
| | GGGAGGGCTAGAGGCCTGAGGCTTGAAACAGCTCTCAAGTGGAGGGGGAAACAACCATT | |
| | GCCCTCATAGAGGACACATCCACACCAGGGCTGTGCTAGCGTGGGCAGGCAAGCCAGGT | |
| | GCTGGACCTCTGCACGTGGGGCATGTGTGGGTATGTACATGTACCTGTGTTCTTGGTGTGT | |
| | GTGTGTGTGTGTGTGTGTGTGTGTGTCTAGAGCTGGGGTGCAACTATGGGGCCCCTCGGG | |
| | ACATGTCCCAGCCAATGCCTGCTTTGACCAGAGGAGTGTCCACGTGGCTCAGGTGGTCGA | |
| | GTATCTCATACCGCCCTAGCACACGTGTGACTCCTTTCCCCTATTGTCTACGCAGCCTGCC | |
| | CTTGGACAAGGACCCGATGCCCAACCCCAGGCCTGGCAAGCCCTCGGCCCCTTCCTTGGC | |
| | CCTTGGCCCATCCCCACGCGTAGGAACAGAGAAACAGGAGAATATGGGCCAAACAGGAT | |
| | ATCTGTGGTAAGCAGTTCCTGCCCCGGCTCAGGGCCAAGAACAGTTGGAACAGCAGAAT | |
| | ATGGGCCAAACAGGATATCTGTGGTAAGCAGTTCCTGCCCCGGCTCAGGGCCAAGAACA | |
| | GATGGTCCCCAGATGCGGTCCCGCCCTCAGCAGTTTCTAGAGAACCATCAGATGTTTCCA | |
| | GGGTGCCCCAAGGACCTGAAATGACCCTGTGCCTTATTTGAACTAACCAATCAGTTCGCT | |
| | TCTCGCTTCTGTTCGCGCGCTTCTGCTCCCCGAGCTCTATATAAGCAGAGCTCGTTTAGTG | |
| | AACCGTCAGATCGCCTGGAGACGCCATCCACGCTGTTTTGACTTCCATAGAAGGATCTCG | |
| | AGGCCACCATGGTGAGCAAGGGCGAGGAGCTGTTCACCGGGGTGGTGCCCATCCTGGTC | |
| | GAGCTGGACGGCGACGTAAACGGCCACAAGTTCAGCGTGTCCGGCGAGGGCGAGGGCG | |
| | ATGCCACCTACGGCAAGCTGACCCTGAAGTTCATCTGCACCACCGGCAAGCTGCCCGTGC | |
| | CCTGGCCCACCCTCGTGACCACCCTGACCTACGGCGTGCAGTGCTTCAGCCGCTACCCCG | |
| | ACCACATGAAGCAGCACGACTTCTTCAAGTCCGCCATGCCCGAAGGCTACGTCCAGGAG | |
| | CGCACCATCTTCTTCAAGGACGACGGCAACTACAAGACCCGCGCCGAGGTGAAGTTCGA | |
| | GGGCGACACCCTGGTGAACCGCATCGAGCTGAAGGGCATCGACTTCAAGGAGGACGGCA | |
| | ACATCCTGGGGCACAAGCTGGAGTACAACTACAACAGCCACAACGTCTATATCATGGCC | |
| | GACAAGCAGAAGAACGGCATCAAGGTGAACTTCAAGATCCGCCACAACATCGAGGACG | |
| | GCAGCGTGCAGCTCGCCGACCACTACCAGCAGAACACCCCCATCGGCGACGGCCCCGTG | |
| | CTGCTGCCCGACAACCACTACCTGAGCACCCAGTCCGCCCTGAGCAAAGACCCCAACGA | |
| | GAAGCGCGATCACATGGTCCTGCTGGAGTTCGTGACCGCCGCCGGGATCACTCTCGGCAT | |
| | GGACGAGCTGTACAAGTAAAAGCTTGATAATCAACCTCTGGATTACAAAATTTGTGAAA | |
| | GATTGACTGGTATTCTTAACTATGTTGCTCCTTTTACGCTATGTGGATACGCTGCTTTAAT | |
| | GCCTTTGTATCATGCTATTGCTTCCCGTATGGCTTTCATTTTCTCCTCCTTGTATAAATCCT | |
| | GGTTAGTTCTTGCCACGGCGGAACTCATCGCCGCCTGCCTTGCCCGCTGCTGGACAGGGG | |
| | CTCGGCTGTTGGGCACTGACAATTCCGTGGGTCGACTGCTTTATTTGTGAAATTTGTGATG | |
| | CTATTGCTTTATTTGTAACCATTATAAGCTGCAATAAACAAGTTAACAACAACAATTGCA | |
| | TTCATTTTATGTTTCAGGTTCAGGGGGAGATGTGGGAGGTTTTTTAAAGCACTAGTGCCTC | |
| | GCCCAGCTGGAGGGCTGCACCCAAAGCCTCAGACCTGCTGGGGGCCCGGGGCCCAGGGG | |
| | GAACCTTCCAGGGCCGAGATCTTCGAGGCGGGGCCCATGCCTCCTCTTCTTCCTTGAACC | |
| | CCATGCCACCATCGCAGCTGCAGGTGAGGCCCTGGGCCCAGGATGGGGCAGGCAGGGTG | |
| | GGGTACCTGGACCTACAGGTGCCGACCTTTACTGTGGCACTGGGCGGGAGGGGGGCTGG | |
| | CTGGGGCACAGGAAGTGGTTTCTGGGTCCCAGGCAAGTCTGTGACTTATGCAGATGTTGC | |
| | AGGGCCAAGAAAATCCCCACCTGCCAGGCCTCAGAGATTGGAGGCTCTCCCCGACCTCC | |
| | CAATCCCTGTCTCAGGAGAGGAGGAGGCCGTATTGTAGTCCCATGAGCATAGCTATGTGT | |
| | CCCCATCCCCATGTGACAAGAGAAGAGGACTGGGGCCAAGTAGGTGAGGTGACAGGGCT | |
| | GAGGCCAGCTCTGCAACTTATTAGCTGTTTGATCTTTAAAAAGTTACTCGATCTCCATGAG | |
| | CCTCAGTTTCCATACGTGTAAAAGGGGGATGATCATAGCATCTACCATGTGGGCTTGCAG | |
| | TGCAGAGTATTTGAATTAGACACAGAACAGTGAGGATCAGGATGGCCTCTCACCCACCT | |
| | GCCTTTCTGCCCAGCTGCCCACACTGCCCCTAGTCATGGTGGCACCCTCCGGGGCACGGC | |
| | TGGGCCCCTTGCCCCACTTACAGGCACTCCTCCAGGACAGGCCACATTTCATGCACCAGG | |
| | TATGGACGGTGAATGGATCCTACGTAGATAAGTAGCATGGCGGGTTAATCATTAACTACA | |
| | AGGAACCCCTAGTGATGGAGTTGGCCACTCCCTCTCTGCGCGCTCGCTCGCTCACTGAGG | |
| | CCGGGCGACCAAAGGTCGCCCGACGCCCGGGCTTTGCCCGGGCGGCCTCAGTGAGCGAG | |
| | CGAGCGCGCCAGCTGGCGTAATAGCGAAGAGGCCCGCACCGATCGCCCTTCCCAACAGT | |
| | TGCGCAGCCTGAATGGCGAATGGCGATTCCGTTGCAATGGCTGGCGGTAATATTGTTCTG | |
| | GATATTACCAGCAAGGCCGATAGTTTGAGTTCTTCTACTCAGGCAAGTGATGTTATTACT | |
| | AATCAAAGAAGTATTGCGACAACGGTTAATTTGCGTGATGGACAGACTCTTTTACTCGGT | |
| | GGCCTCACTGATTATAAAAACACTTCTCAGGATTCTGGCGTACCGTTCCTGTCTAAAATC | |
| | CCTTTAATCGGCCTCCTGTTTAGCTCCCGCTCTGATTCTAACGAGGAAAGCACGTTATACG | |
| | TGCTCGTCAAAGCAACCATAGTACGCGCCCTGTAGCGGCGCATTAAGCGCGGCGGGTGT | |
| | GGTGGTTACGCGCAGCGTGACCGCTACACTTGCCAGCGCCCTAGCGCCCGCTCCTTTCGC | |
| | TTTCTTCCCTTCCTTTCTCGCCACGTTCGCCGGCTTTCCCCGTCAAGCTCTAAATCGGGGG | |
| | CTCCCTTTAGGGTTCCGATTTAGTGCTTTACGGCACCTCGACCCCAAAAAACTTGATTAG | |
| | GGTGATGGTTCACGTAGTGGGCCATCGCCCTGATAGACGGTTTTTCGCCCTTTGACGTTG | |
| | GAGTCCACGTTCTTTAATAGTGGACTCTTGTTCCAAACTGGAACAACACTCAACCCTATC | |
| | TCGGTCTATTCTTTTGATTTATAAGGGATTTTGCCGATTTCGGCCTATTGGTTAAAAAATG | |
| | AGCTGATTTAACAAAAATTTAACGCGAATTTTAACAAAATATTAACGTTTACAATTTAAA | |
| | TATTTGCTTATACAATCTTCCTGTTTTTGGGGCTTTTCTGATTATCAACCGGGGTACATAT | |
| | GATTGACATGCTAGTTTTACGATTACCGTTCATCGATTCTCTTGTTTGCTCCAGACTCTCA | |
| | GGCAATGACCTGATAGCCTTTGTAGAGACCTCTCAAAAATAGCTACCCTCTCCGGCATGA | |
| | ATTTATCAGCTAGAACGGTTGAATATCATATTGATGGTGATTTGACTGTCTCCGGCCTTTC | |
| | TCACCCGTTTGAATCTTTACCTACACATTACTCAGGCATTGCATTTAAAATATATGAGGGT | |
| | TCTAAAAATTTTTATCCTTGCGTTGAAATAAAGGCTTCTCCCGCAAAAGTATTACAGGGT | |
| | CATAATGTTTTTGGTACAACCGATTTAGCTTTATGCTCTGAGGCTTTATTGCTTAATTTTGC | |
| | TAATTCTTTGCCTTGCCTGTATGATTTATTGGATGTTGGAATCGCCTGATGCGGTATTTTCT | |
| | CCTTACGCATCTGTGCGGTATTTCACACCGCATATGGTGCACTCTCAGTACAATCTGCTCT | |
| | GATGCCGCATAGTTAAGCCAGCCCCGACACCCGCCAACACCCGCTGACGCGCCCTGACG | |
| | GGCTTGTCTGCTCCCGGCATCCGCTTACAGACAAGCTGTGACCGTCTCCGGGAGCTGCAT | |
| | GTGTCAGAGGTTTTCACCGTCATCACCGAAACGCGCGAGACGAAAGGGCCTCGTGATAC | |
| | GCCTATTTTTATAGGTTAATGTCATGATAATAATGGTTTCTTAGACGTCAGGTGGCACTTT | |
| | TCGGGGAAATGTGCGCGGAACCCCTATTTGTTTATTTTTCTAAATACATTCAAATATGTAT | |
| | CCGCTCATGAGACAATAACCCTGATAAATGCTTCAATAATATTGAAAAAGGAAGAGTAT | |
| | GAGTATTCAACATTTCCGTGTCGCCCTTATTCCCTTTTTTGCGGCATTTTGCCTTCCTGTTT | |
| | TTGCTCACCCAGAAACGCTGGTGAAAGTAAAAGATGCTGAAGATCAGTTGGGTGCACGA | |
| | GTGGGTTACATCGAACTGGATCTCAACAGCGGTAAGATCCTTGAGAGTTTTCGCCCCGAA | |
| | GAACGTTTTCCAATGATGAGCACTTTTAAAGTTCTGCTATGTGGCGCGGTATTATCCCGTA | |
| | TTGACGCCGGGCAAGAGCAACTCGGTCGCCGCATACACTATTCTCAGAATGACTTGGTTG | |
| | AGTACTCACCAGTCACAGAAAAGCATCTTACGGATGGCATGACAGTAAGAGAATTATGC | |
| | AGTGCTGCCATAACCATGAGTGATAACACTGCGGCCAACTTACTTCTGACAACGATCGGA | |
| | GGACCGAAGGAGCTAACCGCTTTTTTGCACAACATGGGGGATCATGTAACTCGCCTTGAT | |
| | CGTTGGGAACCGGAGCTGAATGAAGCCATACCAAACGACGAGCGTGACACCACGATGCC | |
| | TGTAGCAATGGCAACAACGTTGCGCAAACTATTAACTGGCGAACTACTTACTCTAGCTTC | |
| | CCGGCAACAATTAATAGACTGGATGGAGGCGGATAAAGTTGCAGGACCACTTCTGCGCT | |
| | CGGCCCTTCCGGCTGGCTGGTTTATTGCTGATAAATCTGGAGCCGGTGAGCGTGGGTCTC | |
| | GCGGTATCATTGCAGCACTGGGGCCAGATGGTAAGCCCTCCCGTATCGTAGTTATCTACA | |
| | CGACGGGGAGTCAGGCAACTATGGATGAACGAAATAGACAGATCGCTGAGATAGGTGCC | |
| | TCACTGATTAAGCATTGGTAACTGTCAGACCAAGTTTACTCATATATACTTTAGATTGATT | |
| | TAAAACTTCATTTTTAATTTAAAAGGATCTAGGTGAAGATCCTTTTTGATAATCTCATGAC | |
| | CAAAATCCCTTAACGTGAGTTTTCGTTCCACTGAGCGTCAGACCCC | |
| 39 | MEMWHEGLEEASRLYFGERNVKGMFEVLEPLHAMMERGPQTLKETSFNQAYGRDLMEAQE | naked FRB |
| | WCRKYMKSGNVKDLTQAWDLYYHVFRRISK | wild-type |
| | | polypeptide |
| 40 | MEMWHEGLEEASRLYFGERNVKGMFEVLEPLHAMMERGPQTLKETSFNQAYGRDLMEAQE | naked FRB |
| | WCRKYMKSGNVKDLLQAWDLYYHVFRRISK | mutant |
| | | polypeptide |
| 41 | GAACAGAGAAACAGGAGAATATGGGCCAAACAGGATATCTGTGGTAAGCAGTTCCTGCC | DISC vector |
| | CCGGCTCAGGGCCAAGAACAGTTGGAACAGCAGAATATGGGCCAAACAGGATATCTGTG | DNA |
| | GTAAGCAGTTCCTGCCCCGGCTCAGGGCCAAGAACAGATGGTCCCCAGATGCGGTCCCG | |
| | CCCTCAGCAGTTTCTAGAGAACCATCAGATGTTTCCAGGGTGCCCCAAGGACCTGAAATG | |
| | ACCCTGTGCCTTATTTGAACTAACCAATCAGTTCGCTTCTCGCTTCTGTTCGCGCGCTTCT | |
| | GCTCCCCGAGCTCTATATAAGCAGAGCTCGTTTAGTGAACCGTCAGATCGCTAGCACCGG | |
| | TGCCGCCACCATGCCTCTGGGCCTGCTGTGGCTGGGCCTGGCCCTGCTGGGCGCCCTGCA | |
| | CGCCCAGGCCGGCGTGCAGGTGGAGACAATCTCCCCAGGCGACGGACGCACATTCCCTA | |
| | AGCGGGGCCAGACCTGCGTTGTGCACTATACAGGCATGCTGGAGGATGGCAAGAAGTTT | |
| | GACAGCTCCCGGGATAGAAACAAGCCATTCAAGTTTATGCTGGGCAAGCAGGAAGTGAT | |
| | CAGAGGCTGGGAGGAGGGCGTGGCCCAGATGTCTGTGGGCCAGAGGGCCAAGCTGACCA | |
| | TCAGCCCAGACTACGCCTATGGAGCAACAGGCCACCCAGGAATCATCCCACCTCACGCC | |
| | ACCCTGGTGTTCGATGTGGAGCTGCTGAAGCTGGGCGAGGGATCCAACACATCAAAAGA | |
| | GAACCCCTTTCTGTTCGCATTGGAGGCCGTAGTCATATCTGTTGGATCCATGGGACTTATT | |
| | ATCTCCCTGTTGTGTGTGTACTTCTGGCTGGAACGGACTATGCCCAGGATCCCCACGCTC | |
| | AAGAATCTGGAAGATCTCGTCACAGAATACCATGGTAATTTCAGCGCCTGGAGCGGAGT | |
| | CTCTAAGGGTCTGGCCGAATCCCTCCAACCCGATTATTCTGAACGGTTGTGCCTCGTATCC | |
| | GAAATACCACCAAAAGGCGGGGCTCTGGGTGAGGGCCCAGGGGCGAGTCCGTGCAATCA | |
| | ACACAGCCCGTATTGGGCCCCTCCTTGTTATACGTTGAAGCCCGAAACTGGAAGCGGAGC | |
| | TACTAACTTCAGCCTGCTGAAGCAGGCTGGAGACGTGGAGGAGAACCCTGGACCTATGG | |
| | CACTGCCCGTGACCGCCCTGCTGCTGCCTCTGGCCCTGCTGCTGCACGCAGCCCGGCCTA | |
| | TCCTGTGGCACGAGATGTGGCACGAGGGCCTGGAGGAGGCCAGCAGGCTGTATTTTGGC | |
| | GAGCGCAACGTGAAGGGCATGTTCGAGGTGCTGGAGCCTCTGCACGCCATGATGGAGAG | |
| | AGGCCCACAGACCCTGAAGGAGACATCCTTTAACCAGGCCTATGGACGGGACCTGATGG | |
| | AGGCACAGGAGTGGTGCAGAAAGTACATGAAGTCTGGCAATGTGAAGGACCTGCTGCAG | |
| | GCCTGGGATCTGTACTATCACGTGTTTCGGAGAATCTCCAAGGGCAAAGACACGATTCCG | |
| | TGGCTTGGGCATCTGCTCGTTGGGCTGAGTGGTGCGTTTGGTTTCATCATCTTGGTCTATC | |
| | TCTTGATCAATTGCAGAAATACAGGCCCTTGGCTGAAAAAAGTGCTCAAGTGTAATACCC | |
| | CCGACCCAAGCAAGTTCTTCTCCCAGCTTTCTTCAGAGCATGGAGGCGATGTGCAGAAAT | |
| | GGCTCTCTTCACCTTTTCCCTCCTCAAGCTTCTCCCCGGGAGGGCTGGCGCCCGAGATTTC | |
| | ACCTCTTGAGGTACTTGAACGAGACAAGGTTACCCAACTTCTCCTTCAACAGGATAAGGT | |
| | ACCCGAACCTGCGAGCCTTAGCTCCAACCACTCTCTTACGAGCTGCTTCACCAATCAGGG | |
| | ATACTTCTTTTTCCACCTTCCCGATGCGCTGGAAATCGAAGCTTGTCAAGTTTACTTTACC | |
| | TATGATCCATATAGCGAGGAAGATCCCGACGAAGGAGTCGCCGGTGCGCCCACGGGTTC | |
| | CTCACCCCAACCTCTCCAGCCTCTCTCAGGAGAAGATGATGCTTATTGCACTTTTCCCAGT | |
| | AGAGACGATCTCCTCCTCTTTTCTCCATCTCTTTTGGGGGGACCTTCCCCCCCTTCTACGG | |
| | CACCTGGCGGGTCTGGTGCTGGCGAGGAGCGGATGCCGCCGTCCCTCCAGGAGCGAGTA | |
| | CCACGAGATTGGGATCCCCAGCCACTTGGACCCCCCACCCCCGGCGTACCTGACCTTGTC | |
| | GATTTTCAACCTCCCCCTGAATTGGTGCTGCGAGAGGCTGGGGAGGAAGTTCCGGACGCT | |
| | GGGCCGAGGGAGGGCGTGTCCTTTCCATGGAGTAGGCCTCCAGGTCAAGGCGAGTTTAG | |
| | GGCTCTCAACGCGCGGCTGCCGTTGAATACAGACGCTTATCTCTCACTGCAGGAACTGCA | |
| | AGGTCAGGACCCAACACATCTTGTAGGATCTGGTGCTACTAATTTTTCTCTTTTGAAGCAA | |
| | GCTGGAGATGTTGAAGAGAACCCCGGTCCGGAGATGTGGCATGAGGGTCTGGAAGAAGC | |
| | GTCTCGACTGTACTTTGGTGAGCGCAATGTGAAGGGCATGTTTGAAGTCCTCGAACCCCT | |
| | TCATGCCATGATGGAACGCGGACCCCAGACCTTGAAGGAGACAAGTTTTAACCAAGCTT | |
| | ACGGAAGAGACCTGATGGAAGCCCAGGAATGGTGCAGGAAATACATGAAAAGCGGGAA | |
| | TGTGAAGGACTTGCTCCAAGCGTGGGACCTGTACTATCATGTCTTTAGGCGCATTAGTAA | |
| | GGGCAGCGGCGCCACCAACTTCAGCCTGCTGAAGCAGGCCGGCGACGTGGAGGAGAACC | |
| | CCGGCCCCGTGAGCAAGGGCGAGGAGGATAACATGGCCATCATCAAGGAGTTCATGCGC | |
| | TTCAAGGTGCACATGGAGGGCTCCGTGAACGGCCACGAGTTCGAGATCGAGGGCGAGGG | |
| | CGAGGGCCGCCCCTACGAGGGCACCCAGACCGCCAAGCTGAAGGTGACCAAGGGTGGCC | |
| | CCCTGCCCTTCGCCTGGGACATCCTGTCCCCTCAGTTCATGTACGGCTCCAAGGCCTACGT | |
| | GAAGCACCCCGCCGACATCCCCGACTACTTGAAGCTGTCCTTCCCCGAGGGCTTCAAGTG | |
| | GGAGCGCGTGATGAACTTCGAGGACGGCGGCGTGGTGACCGTGACCCAGGACTCCTCTC | |
| | TGCAGGACGGCGAGTTCATCTACAAGGTGAAGCTGCGCGGCACCAACTTCCCCTCCGAC | |
| | GGCCCCGTAATGCAGAAGAAGACCATGGGCTGGGAGGCCTCCTCCGAGCGGATGTACCC | |
| | CGAGGACGGCGCCCTGAAGGGCGAGATCAAGCAGAGGCTGAAGCTGAAGGACGGCGGC | |
| | CACTACGACGCTGAGGTCAAGACCACCTACAAGGCCAAGAAGCCCGTGCAGCTGCCCGG | |
| | CGCCTACAACGTCAACATCAAGTTGGACATCACCTCCCACAACGAGGACTACACCATCGT | |
| | GGAACAGTACGAACGCGCCGAGGGCCGCCACTCCACCGGCGGCATGGACGAGCTGTACA | |
| | AGTGAACTAGTGTCGACAATCAACCTCTGGATTACAAAATTTGTGAAAGATTGACTGGTA | |
| | TTCTTAACTATGTTGCTCCTTTTACGCTATGTGGATACGCTGCTTTAATGCCTTTGTATCAT | |
| | GCTATTGCTTCCCGTATGGCTTTCATTTTCTCCTCCTTGTATAAATCCTGGTTGCTGTCTCT | |
| | TTATGAGGAGTTGTGGCCCGTTGTCAGGCAACGTGGCGTGGTGTGCACTGTGTTTGCTGA | |
| | CGCAACCCCCACTGGTTGGGGCATTGCCACCACCTGTCAGCTCCTTTCCGGGACTTTCGCT | |
| | TTCCCCCTCCCTATTGCCACGGCGGAACTCATCGCCGCCTGCCTTGCCCGCTGCTGGACA | |
| | GGGGCTCGGCTGTTGGGCACTGACAATTCCGTGGTGTTGTCGGGGAAGCTGACGTCCTTT | |
| | CCATGGCTGCTCGCCTGTGTTGCCACCTGGATTCTGCGCGGGACGTCCTTCTGCTACGTCC | |
| | CTTCGGCCCTCAATCCAGCGGACCTTCCTTCCCGCGGCCTGCTGCCGGCTCTGCGGCCTCT | |
| | TCCGCGTCTTCGCCTTCGCCCTCAGACGAGTCGGATCTCCCTTTGGGCCGCCTCCCCGCCT | |
| | GGA | |
| 42 | CCAGCAGCTCTCGGCAAAGACACGATTCCGTGGCTTGGGCATCTGCTCGTTGGGCTGAGC | μDISC DNA |
| | GGTGCGTTTGGTTTCATCATCTTGGTCTATCTCTTGATCAATTGCAGAAATACAGGCCCTT | (cytoplasmic |
| | GGCTGAAAAAAGTGCTCAAGTGTAATACCCCCGACCCAAGCAAGTTCTTCTCCCAGCTTT | tail only; |
| | CTTCAGAGCATGGAGGCGATGTGCAGAAATGGCTCTCTTCACCTTTTCCCTCCTCAAGCTT | codon |
| | CTCCCCGGGAGGGCTGGCGCCCGAGATTTCACCTCTTGAGGTACTTGAACGAGACAAGGT | diverged) |
| | TACCCAACTTCTCCTTCAACAGGATAAGGTACCCGAACCTGCGAGCCTTAGCTTGAATAC | |
| | AGACGCTTATCTCTCACTGCAGGAACTGCAA | |
| 43 | PAALGKDTIPWLGHLLVGLSGAFGFIILVYLLINCRNTGPWLKKVLKCNTPDPSKFFSQLSSEH | μDISC |
| | GGDVQKWLSSPFPSSSFSPGGLAPEISPLEVLERDKVTQLLLQQDKVPEPASLSLNTDAYLSLQ | polypeptide |
| | ELQ | (cytoplasmic |
| | | tail only) |
| 44 | GVQVETISPGDGRTFPKRGQTCVVHYTGMLEDGKKFDSSRDRNKPFKFMLGKQEVIRGWEE | FKBP CISC |
| | GVAQMSVGQRAKLTISPDYAYGATGHPGIIPPHATLVFDVELLKLGE | domain |
| 45 | MALPVTALLLPLALLLHAARPILWHEMWHEGLEEASRLYFGERNVKGMFEVLEPLHAMMER | Entire μDISC |
| | GPQTLKETSFNQAYGRDLMEAQEWCRKYMKSGNVKDLLQAWDLYYHVFRRISKPAALGKD | polypeptide |
| | TIPWLGHLLVGLSGAFGFIILVYLLINCRNTGPWLKKVLKCNTPDPSKFFSQLSSEHGGDVQK | (FRB- |
| | WLSSPFPSSSFSPGGLAPEISPLEVLERDKVTQLLLQQDKVPEPASLSLNTDAYLSLQELQ | truncated |
| | | IL2Rβ) |
| 46 | GAACAGAGAAACAGGAGAATATGGGCCAAACAGGATATCTGTGGTAAGCAGTTCCTGCC | μDISC |
| | CCGGCTCAGGGCCAAGAACAGTTGGAACAGCAGAATATGGGCCAAACAGGATATCTGTG | vector DNA |
| | GTAAGCAGTTCCTGCCCCGGCTCAGGGCCAAGAACAGATGGTCCCCAGATGCGGTCCCG | |
| | CCCTCAGCAGTTTCTAGAGAACCATCAGATGTTTCCAGGGTGCCCCAAGGACCTGAAATG | |
| | ACCCTGTGCCTTATTTGAACTAACCAATCAGTTCGCTTCTCGCTTCTGTTCGCGCGCTTCT | |
| | GCTCCCCGAGCTCTATATAAGCAGAGCTCGTTTAGTGAACCGTCAGATCGCTAGCACCGG | |
| | TGCCGCCACCATGCCTCTGGGCCTGCTGTGGCTGGGCCTGGCCCTGCTGGGCGCCCTGCA | |
| | CGCCCAGGCCGGCGTGCAGGTGGAGACAATCTCCCCAGGCGACGGACGCACATTCCCTA | |
| | AGCGGGGCCAGACCTGCGTTGTGCACTATACAGGCATGCTGGAGGATGGCAAGAAGTTT | |
| | GACAGCTCCCGGGATAGAAACAAGCCATTCAAGTTTATGCTGGGCAAGCAGGAAGTGAT | |
| | CAGAGGCTGGGAGGAGGGCGTGGCCCAGATGTCTGTGGGCCAGAGGGCCAAGCTGACCA | |
| | TCAGCCCAGACTACGCCTATGGAGCAACAGGCCACCCAGGAATCATCCCACCTCACGCC | |
| | ACCCTGGTGTTCGATGTGGAGCTGCTGAAGCTGGGCGAGGGATCCAACACATCAAAAGA | |
| | GAACCCCTTTCTGTTCGCATTGGAGGCCGTAGTCATATCTGTTGGATCCATGGGACTTATT | |
| | ATCTCCCTGTTGTGTGTGTACTTCTGGCTGGAACGGACTATGCCCAGGATCCCCACGCTC | |
| | AAGAATCTGGAAGATCTCGTCACAGAATACCATGGTAATTTCAGCGCCTGGAGCGGAGT | |
| | CTCTAAGGGTCTGGCCGAATCCCTCCAACCCGATTATTCTGAACGGTTGTGCCTCGTATCC | |
| | GAAATACCACCAAAAGGCGGGGCTCTGGGTGAGGGCCCAGGGGCGAGTCCGTGCAATCA | |
| | ACACAGCCCGTATTGGGCCCCTCCTTGTTATACGTTGAAGCCCGAAACTGGAAGCGGAGC | |
| | TACTAACTTCAGCCTGCTGAAGCAGGCTGGAGACGTGGAGGAGAACCCTGGACCTATGG | |
| | CACTGCCCGTGACCGCCCTGCTGCTGCCTCTGGCCCTGCTGCTGCACGCAGCCCGGCCTA | |
| | TCCTGTGGCACGAGATGTGGCACGAGGGCCTGGAGGAGGCCAGCAGGCTGTATTTTGGC | |
| | GAGCGCAACGTGAAGGGCATGTTCGAGGTGCTGGAGCCTCTGCACGCCATGATGGAGAG | |
| | AGGCCCACAGACCCTGAAGGAGACATCCTTTAACCAGGCCTATGGACGGGACCTGATGG | |
| | AGGCACAGGAGTGGTGCAGAAAGTACATGAAGTCTGGCAATGTGAAGGACCTGCTGCAG | |
| | GCCTGGGATCTGTACTATCACGTGTTTCGGAGAATCTCCAAGGGCAAAGACACGATTCCG | |
| | TGGCTTGGGCATCTGCTCGTTGGGCTGAGTGGTGCGTTTGGTTTCATCATCTTGGTCTATC | |
| | TCTTGATCAATTGCAGAAATACAGGCCCTTGGCTGAAAAAAGTGCTCAAGTGTAATACCC | |
| | CCGACCCAAGCAAGTTCTTCTCCCAGCTTTCTTCAGAGCATGGAGGCGATGTGCAGAAAT | |
| | GGCTCTCTTCACCTTTTCCCTCCTCAAGCTTCTCCCCGGGAGGGCTGGCGCCCGAGATTTC | |
| | ACCTCTTGAGGTACTTGAACGAGACAAGGTTACCCAACTTCTCCTTCAACAGGATAAGGT | |
| | ACCCGAACCTGCGAGCCTTAGCTTGAATACAGACGCTTATCTCTCACTGCAGGAACTGCA | |
| | AGGATCTGGTGCTACTAATTTTTCTCTTTTGAAGCAAGCTGGAGATGTTGAAGAGAACCC | |
| | CGGTCCGGAGATGTGGCATGAGGGTCTGGAAGAAGCGTCTCGACTGTACTTTGGTGAGC | |
| | GCAATGTGAAGGGCATGTTTGAAGTCCTCGAACCCCTTCATGCCATGATGGAACGCGGAC | |
| | CCCAGACCTTGAAGGAGACAAGTTTTAACCAAGCTTACGGAAGAGACCTGATGGAAGCC | |
| | CAGGAATGGTGCAGGAAATACATGAAAAGCGGGAATGTGAAGGACTTGCTCCAAGCGTG | |
| | GGACCTGTACTATCATGTCTTTAGGCGCATTAGTAAGGGCAGCGGCGCCACCAACTTCAG | |
| | CCTGCTGAAGCAGGCCGGCGACGTGGAGGAGAACCCCGGCCCCGTGAGCAAGGGCGAG | |
| | GAGGATAACATGGCCATCATCAAGGAGTTCATGCGCTTCAAGGTGCACATGGAGGGCTC | |
| | CGTGAACGGCCACGAGTTCGAGATCGAGGGCGAGGGCGAGGGCCGCCCCTACGAGGGC | |
| | ACCCAGACCGCCAAGCTGAAGGTGACCAAGGGTGGCCCCCTGCCCTTCGCCTGGGACAT | |
| | CCTGTCCCCTCAGTTCATGTACGGCTCCAAGGCCTACGTGAAGCACCCCGCCGACATCCC | |
| | CGACTACTTGAAGCTGTCCTTCCCCGAGGGCTTCAAGTGGGAGCGCGTGATGAACTTCGA | |
| | GGACGGCGGCGTGGTGACCGTGACCCAGGACTCCTCTCTGCAGGACGGCGAGTTCATCT | |
| | ACAAGGTGAAGCTGCGCGGCACCAACTTCCCCTCCGACGGCCCCGTAATGCAGAAGAAG | |
| | ACCATGGGCTGGGAGGCCTCCTCCGAGCGGATGTACCCCGAGGACGGCGCCCTGAAGGG | |
| | CGAGATCAAGCAGAGGCTGAAGCTGAAGGACGGCGGCCACTACGACGCTGAGGTCAAG | |
| | ACCACCTACAAGGCCAAGAAGCCCGTGCAGCTGCCCGGCGCCTACAACGTCAACATCAA | |
| | GTTGGACATCACCTCCCACAACGAGGACTACACCATCGTGGAACAGTACGAACGCGCCG | |
| | AGGGCCGCCACTCCACCGGCGGCATGGACGAGCTGTACAAGTGAACTAGTGTCGACAAT | |
| | CAACCTCTGGATTACAAAATTTGTGAAAGATTGACTGGTATTCTTAACTATGTTGCTCCTT | |
| | TTACGCTATGTGGATACGCTGCTTTAATGCCTTTGTATCATGCTATTGCTTCCCGTATGGC | |
| | TTTCATTTTCTCCTCCTTGTATAAATCCTGGTTGCTGTCTCTTTATGAGGAGTTGTGGCCCG | |
| | TTGTCAGGCAACGTGGCGTGGTGTGCACTGTGTTTGCTGACGCAACCCCCACTGGTTGGG | |
| | GCATTGCCACCACCTGTCAGCTCCTTTCCGGGACTTTCGCTTTCCCCCTCCCTATTGCCAC | |
| | GGCGGAACTCATCGCCGCCTGCCTTGCCCGCTGCTGGACAGGGGCTCGGCTGTTGGGCAC | |
| | TGACAATTCCGTGGTGTTGTCGGGGAAGCTGACGTCCTTTCCATGGCTGCTCGCCTGTGTT | |
| | GCCACCTGGATTCTGCGCGGGACGTCCTTCTGCTACGTCCCTTCGGCCCTCAATCCAGCG | |
| | GACCTTCCTTCCCGCGGCCTGCTGCCGGCTCTGCGGCCTCTTCCGCGTCTTCGCCTTCGCC | |
| | CTCAGACGAGTCGGATCTCCCTTTGGGCCGCCTCCCCGCCTGGA | |
| 47 | MPLGLLWLGLALLGALHAQAGVQVETISPGDGRTFPKRGQTCVVHYTGMLEDGKKFDSSRD | IL2Rγ-CISC |
| | RNKPFKFMLGKQEVIRGWEEGVAQMSVGQRAKLTISPDYAYGATGHPGIIPPHATLVFDVEL | polypeptide |
| | LKLGEGSNTSKENPFLFALEAVVISVGSMGLIISLLCVYFWLERTMPRIPTLKNLEDLVTEYHG | |
| | NFSAWSGVSKGLAESLQPDYSERLCLVSEIPPKGGALGEGPGASPCNQHSPYWAPPCYTLKPE | |
| | T | |
| 48 | MPLGLLWLGLALLGALHAQAGVQVETISPGDGRTFPKRGQTCVVHYTGMLEDGKKFDSSRD | IL2Rγ-CISC |
| | RNKPFKFMLGKQEVIRGWEEGVAQMSVGQRAKLTISPDYAYGATGHPGIIPPHATLVFDVEL | polypeptide |
| | LKLEGGGSQNLVIPWAPENLTLHKLSESQLELNWNNRFLNHCLEHLVQYRTDWDHSWTEQS | |
| | VDYRHKFSLPSVDGQKRYTFRVRSRFNPLCGSAQHWSEWSHPIHWGSNTSKENPFLFALEAV | |
| | VISVGSMGLIISLLCVYFWLERTMPRIPTLKNLEDLVTEYHGNFSAWSGVSKGLAESLQPDYS | |
| | ERLCLVSEIPPKGGALGEGPGASPCNQHSPYWAPPCYTLKPET | |
| 49 | MPLGLLWLGLALLGALHAQAGVQVETISPGDGRTFPKRGQTCVVHYTGMLEDGKKFDSSRD | IL2Rγ-CISC |
| | RNKPFKFMLGKQEVIRGWEEGVAQMSVGQRAKLTISPDYAYGATGHPGIIPPHATLVFDVEL | polypeptide |
| | LKLEGQNLVIPWAPENLTLHKLSESQLELNWNNRFLNHCLEHLVQYRTDWDHSWTEQSVDY | |
| | RHKFSLPSVDGQKRYTFRVRSRFNPLCGSAQHWSEWSHPIHWGSNTSKENPFLFALEAVVISV | |
| | GSMGLIISLLCVYFWLERTMPRIPTLKNLEDLVTEYHGNFSAWSGVSKGLAESLQPDYSERLC | |
| | LVSEIPPKGGALGEGPGASPCNQHSPYWAPPCYTLKPET | |
| 50 | MPLGLLWLGLALLGALHAQAGVQVETISPGDGRTFPKRGQTCVVHYTGMLEDGKKFDSSRD | IL2Rγ-CISC |
| | RNKPFKFMLGKQEVIRGWEEGVAQMSVGQRAKLTISPDYAYGATGHPGIIPPHATLVFDVEL | polypeptide |
| | LKLEGGSNTSKENPFLFALEAVVISVGSMGLIISLLCVYFWLERTMPRIPTLKNLEDLVTEYHG | |
| | NFSAWSGVSKGLAESLQPDYSERLCLVSEIPPKGGALGEGPGASPCNQHSPYWAPPCYTLKPE | |
| | T | |
| 51 | MALPVTALLLPLALLLHAARPILWHEMWHEGLEEASRLYFGERNVKGMFEVLEPLHAMMER | IL2Rβ-CISC |
| | GPQTLKETSFNQAYGRDLMEAQEWCRKYMKSGNVKDLLQAWDLYYHVFRRISKGKDTIPW | polypeptide |
| | LGHLLVGLSGAFGFIILVYLLINCRNTGPWLKKVLKCNTPDPSKFFSQLSSEHGGDVQKWLSS | |
| | PFPSSSFSPGGLAPEISPLEVLERDKVTQLLLQQDKVPEPASLSSNHSLTSCFTNQGYFFFHLPD | |
| | ALEIEACQVYFTYDPYSEEDPDEGVAGAPTGSSPQPLQPLSGEDDAYCTFPSRDDLLLFSPSLL | |
| | GGPSPPSTAPGGSGAGEERMPPSLQERVPRDWDPQPLGPPTPGVPDLVDFQPPPELVLREAGE | |
| | EVPDAGPREGVSFPWSRPPGQGEFRALNARLPLNTDAYLSLQELQGQDPTHLV | |
| 52 | MALPVTALLLPLALLLHAARPILWHEMWHEGLEEASRLYFGERNVKGMFEVLEPLHAMMER | IL2Rβ-CISC |
| | GPQTLKETSFNQAYGRDLMEAQEWCRKYMKSGNVKDLLQAWDLYYHVFRRISKGGSKPFE | polypeptide |
| | NLRLMAPISLQVVHVETHRCNISWEISQASHYFERHLEFEARTLSPGHTWEEAPLLTLKQKQE | |
| | WICLETLTPDTQYEFQVRVKPLQGEFTTWSPWSQPLAFRTKPAALGKDTIPWLGHLLVGLSG | |
| | AFGFIILVYLLINCRNTGPWLKKVLKCNTPDPSKFFQLSSEHGGDVQKWLSSPFPSSSFSPGGL | |
| | APEISPLEVLERDKVTQLLLQQDKVPEPASLSSNHSLTSCFTNQGYFFFHLPDALEIEACQVYF | |
| | TYDPYSEEDPDEGVAGAPTGSSPQPLQPLSGEDDAYCTFPSRDDLLLFSPSLLGGPSPPSTAPG | |
| | GSGAGEERMPPSLQERVPRDWDPQPLGPPTPGVPDLVDFQPPPELVLREAGEEVPDAGPREG | |
| | VSFPWSRPPGQGEFRALNARLPLNTDAYLSLQELQGQDPTHLV | |
| 53 | MALPVTALLLPLALLLHAARPILWHEMWHEGLEEASRLYFGERNVKGMFEVLEPLHAMMER | IL2Rβ-CISC |
| | GPQTLKETSFNQAYGRDLMEAQEWCRKYMKSGNVKDLLQAWDLYYHVFRRISKKPFENLR | polypeptide |
| | LMAPISLQVVHVETHRCNISWEISQASHYFERHLEFEARTLSPGHTWEEAPLLTLKQKQEWIC | |
| | LETLTPDTQYEFQVRVKPLQGEFTTWSPWSQPLAFRTKPAALGKDTIPWLGHLLVGLSGAFG | |
| | FIILVYLLINCRNTGPWLKKVLKCNTPDPSKFFSQLSSEHGGDVQKWLSSPFPSSSFSPGGLAPE | |
| | ISPLEVLERDKVTQLLLQQDKVPEPASLSSNHSLTSCFTNQGYFFFHLPDALEIEACQVYFTYD | |
| | PYSEEDPDEGVAGAPTGSSPQPLQPLSGEDDAYCTFPSRDDLLLFSPSLLGGPSPPSTAPGGSG | |
| | AGEERMPPSLQERVPRDWDPQPLGPPTPGVPDLVDFQPPPELVLREAGEEVPDAGPREGVSFP | |
| | WSRPPGQGEFRALNARLPLNTDAYLSLQELQGQDPTHLV | |
| 54 | MALPVTALLLPLALLLHAARPILWHEMWHEGLEEASRLYFGERNVKGMFEVLEPLHAMMER | IL7Rα-CISC |
| | GPQTLKETSWLGHLLVGLSGAFGFIILVYLLINCRNTGPWLKKVLKCNTPDPSKFFSQLSSEH | polypeptide |
| | GGDVQKWLSSPFPSSSFSPGGLAPEISPLEVLERDKVTQLLLQQDKVPEPASLSSNHSLTSCFT | |
| | NQGYFFFHLPDALEIEACQVYFTYDPYSEEDPDEGVAGAPTGSSPQPLQPLSGEDDAYCTFPS | |
| | RDDLLLFSPSLLGGPSPPSTAPGGSGAGEERMPPSLQERVPRDWDPQPLGPPTPGVPDLVDFQ | |
| | PPPELVLREAGEEVPDAGPREGVSFPWSRPPGQGEFRALNARLPLNTDAYLSLQELQGQDPTH | |
| | LV | |
| 55 | MALPVTALLLPLALLLHAARPILWHEMWHEGLEEASRLYFGERNVKGMFEVLEPLHAMMER | IL7Rα-CISC |
| | GPQTLKETSFNQAYGRDLMEAQEWCRKYMKSGNVKDLLQAWDLYYHVFRRISKGEINNSS | polypeptide |
| | GEMDPILLTISILSFFSVALLVILACVLWKKRIKPIVWPSLPDHKKTLEHLCKKPRKNLNVSFNP | |
| | ESFLDCQIHRVDDIQARDEVEGFLQDTFPQQLEESEKQRLGGDVQSPNCPSEDVVITPESFGRD | |
| | SSLTCLAGNVSACDAPILSSSRSLDCRESGKNGPHVYQDLLLSLGTTNSTLPPPFSLQSGILTLN | |
| | PVAQGQPILTSLGSNQEEAYVTMSSFYQNQ | |
| 56 | GGGS | Linker |
| | | polypeptide |
| 57 | GGGSGGG | Linker |
| | | polypeptide |
| 58 | GGG | Linker |
| | | polypeptide |
| 59 | GGS | Linker |
| | | polypeptide |
| 60 | GGSP | Linker |
| | | polypeptide |
| 61 | MPLGLLWLGLALLGALHAQAGVQVETISPGDGRTFPKRGQTCVVHYTGMLEDGKKVDSSR | IL2Rγ-CISC |
| | DRNKPFKFMLGKQEVIRGWEEGVAQMSVGQRAKLTISPDYAYGATGHPGIIPPHATLVFDVE | polypeptide |
| | LLKLEGGSNTSKENPFLFALEAVVISVGSMGLIISLLCVYFWLERTMPRIPTLKNLEDLVTEYH | |
| | GNFSAWSGVSKGLAESLQPDYSERLCLVSEIPPKGGALGEGPGASPCNQHSPYWAPPCYTLK | |
| | PET | |
| 62 | MPLGLLWLGLALLGALHAQAGVQVETISPGDGRTFPKRGQTCVVHYTGMLEDGKKVDSSR | IL2Rβ-CISC |
| | DRNKPFKFMLGKQEVIRGWEEGVAQMSVGQRAKLTISPDYAYGATGHPGIIPPHATLVFDVE | polypeptide |
| | LLKLEGGKDTIPWLGHLLVGLSGAFGFIILVYLLINCRNTGPWLKKVLKCNTPDPSKFFSQLSS | |
| | EHGGDVQKWLSSPFPSSSFSPGGLAPEISPLEVLERDKVTQLLLQQDKVPEPASLSSNHSLTSC | |
| | FTNQGYFFFHLPDALEIEACQVYFTYDPYSEEDPDEGVAGAPTGSSPQPLQPLSGEDDAYCTF | |
| | PSRDDLLLFSPSLLGGPSPPSTAPGGSGAGEERMPPSLQERVPRDWDPQPLGPPTPGVPDLVDF | |
| | QPPPELVLREAGEEVPDAGPREGVSFPWSRPPGQGEFRALNARLPLNTDAYLSLQELQGQDPT | |
| | HLV | |
| 63 | MPLGLLWLGLALLGALHAQAGVQVETISPGDGRTFPKRGQTCVVHYTGMLEDGKKVDSSR | IL2Rα-CISC |
| | DRNKPFKFMLGKQEVIRGWEEGVAQMSVGQRAKLTISPDYAYGATGHPGIIPPHATLVFDVE | polypeptide |
| | LLKLEGEINNSSGEMDPILLTISILSFFSVALLVILACVLWKKRIKPIVWPSLPDHKKTLEHLCK | |
| | KPRKNLNVSFNPESFLDCQIHRVDDIQARDEVEGFLQDTFPQQLEESEKQRLGGDVQSPNCPS | |
| | EDVVITPESFGRDSSLTCLAGNVSACDAPILSSSRSLDCRESGKNGPHVYQDLLLSLGTTNSTL | |
| | PPPFSLQSGILTLNPVAQGQPILTSLGSNQEEAYVTMSSFYQNQ | |
| 64 | MPLGLLWLGLALLGALHAQAGVQVETISPGDGRTFPKRGQTCVVHYTGMLEDGKKVDSSR | IL7Rα-CISC |
| | DRNKPFKFMLGKQEVIRGWEEGVAQMSVGQRAKLTISPDYAYGATGHPGIIPPHATLVFDVE | polypeptide |
| | LLKLEGEINNSSGEMDPILLTISILSFFSVALLVILACVLWKKRIKPIVWPSLPDHKKTLEHLCK | |
| | KPRKNLNVSFNPESFLDCQIHRVDDIQARDEVEGFLQDTFPQQLEESEKQRLGGDVQSPNCPS | |
| | EDVVITPESFGRDSSLTCLAGNVSACDAPILSSSRSLDCRESGKNGPHVYQDLLLSLGTTNSTL | |
| | PPPFSLQSGILTLNPVAQGQPILTSLGSNQEEAYVTMSSFYQNQ | |
| 65 | MPLGLLWLGLALLGALHAQAGVQVETISPGDGRTFPKRGQTCVVHYTGMLEDGKKVDSSR | MPL-CISC |
| | DRNKPFKFMLGKQEVIRGWEEGVAQMSVGQRAKLTISPDYAYGATGHPGIIPPHATLVFDVE | polypeptide |
| | LLKLGEETAWISLVTALHLVLGLSAVLGLLLLRWQFPAHYRRLRHALWPSLPDLHRVLGQYL | |
| | RDTAALSPPKATVSDTCEEVEPSLLEILPKSSERTPLPLCSSQAQMDYRRLQPSCLGTMPLSVC | |
| | PPMAESGSCCTTHIANHSYLPLSYWQQP | |
| 66 | AGCTTAATGTAGTCTTATGCAATACTCTTGTAGTCTTGCAACATGGTAACGATGAGTTAG | CISC vector |
| | CAACATGCCTTACAAGGAGAGAAAAAGCACCGTGCATGCCGATTGGTGGAAGTAAGGTG | DNA |
| | GTACGATCGTGCCTTATTAGGAAGGCAACAGACGGGTCTGACATGGATTGGACGAACCA | |
| | CTGAATTGCCGCATTGCAGAGATATTGTATTTAAGTGCCTAGCTCGATACAATAAACGGG | |
| | TCTCTCTGGTTAGACCAGATCTGAGCCTGGGAGCTCTCTGGCTAACTAGGGAACCCACTG | |
| | CTTAAGCCTCAATAAAGCTTGCCTTGAGTGCTTCAAGTAGTGTGTGCCCGTCTGTTGTGTG | |
| | ACTCTGGTAACTAGAGATCCCTCAGACCCTTTTAGTCAGTGTGGAAAATCTCTAGCAGTG | |
| | GCGCCCGAACAGGGACTTGAAAGCGAAAGGGAAACCAGAGGAGCTCTCTCGACGCAGG | |
| | ACTCGGCTTGCTGAAGCGCGCACGGCAAGAGGCGAGGGGCGGCGACTGGTGAGTACGCC | |
| | AAAAATTTTGACTAGCGGAGGCTAGAAGGAGAGAGATGGGTGCGAGAGCGTCAGTATTA | |
| | AGCGGGGGAGAATTAGATCGCGATGGGAAAAAATTCGGTTAAGGCCAGGGGGAAAGAA | |
| | AAAATATAAATTAAAACATATAGTATGGGCAAGCAGGGAGCTAGAACGATTCGCAGTTA | |
| | ATCCTGGCCTGTTAGAAACATCAGAAGGCTGTAGACAAATACTGGGACAGCTACAACCA | |
| | TCCCTTCAGACAGGATCAGAAGAACTTAGATCATTATATAATACAGTAGCAACCCTCTAT | |
| | TGTGTGCATCAAAGGATAGAGATAAAAGACACCAAGGAAGCTTTAGACAAGATAGAGGA | |
| | AGAGCAAAACAAAAGTAAGACCACCGCACAGCAAGCGGCCGCTGATCTTCAGACCTGGA | |
| | GGAGGAGATATGAGGGACAATTGGAGAAGTGAATTATATAAATATAAAGTAGTAAAAAT | |
| | TGAACCATTAGGAGTAGCACCCACCAAGGCAAAGAGAAGAGTGGTGCAGAGAGAAAAA | |
| | AGAGCAGTGGGAATAGGAGCTTTGTTCCTTGGGTTCTTGGGAGCAGCAGGAAGCACTAT | |
| | GGGCGCAGCCTCAATGACGCTGACGGTACAGGCCAGACAATTATTGTCTGGTATAGTGC | |
| | AGCAGCAGAACAATTTGCTGAGGGCTATTGAGGCGCAACAGCATCTGTTGCAACTCACA | |
| | GTCTGGGGCATCAAGCAGCTCCAGGCAAGAATCCTGGCTGTGGAAAGATACCTAAAGGA | |
| | TCAACAGCTCCTGGGGATTTGGGGTTGCTCTGGAAAACTCATTTGCACCACTGCTGTGCC | |
| | TTGGAATGCTAGTTGGAGTAATAAATCTCTGGAACAGATTTGGAATCACACGACCTGGAT | |
| | GGAGTGGGACAGAGAAATTAACAATTACACAAGCTTAATACACTCCTTAATTGAAGAAT | |
| | CGCAAAACCAGCAAGAAAAGAATGAACAAGAATTATTGGAATTAGATAAATGGGCAAGT | |
| | TTGTGGAATTGGTTTAACATAACAAATTGGCTGTGGTATATAAAATTATTCATAATGATA | |
| | GTAGGAGGCTTGGTAGGTTTAAGAATAGTTTTTGCTGTACTTTCTATAGTGAATAGAGTT | |
| | AGGCAGGGATATTCACCATTATCGTTTCAGACCCACCTCCCAACCCCGAGGGGACCCGAC | |
| | AGGCCCGAAGGAATAGAAGAAGAAGGTGGAGAGAGAGACAGAGACAGATCCATTCGAT | |
| | TAGTGAACGGATCTCGACGGTATCGGTTAACTTTTAAAAGAAAAGGGGGGATTGGGGGG | |
| | TACAGTGCAGGGGAAAGAATAGTAGACATAATAGCAACAGACATACAAACTAAAGAATT | |
| | ACAAAAACAAATTACAAAAATTCAAAATTTTATCGATCACGAGACTAGCCTCGAGAAGC | |
| | TTGATATCGAATTCCCACGGGGTTGGACGCGTAGGAACAGAGAAACAGGAGAATATGGG | |
| | CCAAACAGGATATCTGTGGTAAGCAGTTCCTGCCCCGGCTCAGGGCCAAGAACAGTTGG | |
| | AACAGCAGAATATGGGCCAAACAGGATATCTGTGGTAAGCAGTTCCTGCCCCGGCTCAG | |
| | GGCCAAGAACAGATGGTCCCCAGATGCGGTCCCGCCCTCAGCAGTTTCTAGAGAACCAT | |
| | CAGATGTTTCCAGGGTGCCCCAAGGACCTGAAATGACCCTGTGCCTTATTTGAACTAACC | |
| | AATCAGTTCGCTTCTCGCTTCTGTTCGCGCGCTTCTGCTCCCCGAGCTCTATATAAGCAGA | |
| | GCTCGTTTAGTGAACCGTCAGATCGCTAGCACCGGTGCCGCCACCATGCCTCTGGGCCTG | |
| | CTGTGGCTGGGCCTGGCCCTGCTGGGCGCCCTGCACGCCCAGGCCGGCGTGCAGGTGGA | |
| | GACAATCTCCCCAGGCGACGGACGCACATTCCCTAAGCGGGGCCAGACCTGCGTGGTGC | |
| | ACTATACAGGCATGCTGGAGGATGGCAAGAAGTTTGACAGCTCCCGGGATAGAAACAAG | |
| | CCATTCAAGTTTATGCTGGGCAAGCAGGAAGTGATCAGAGGCTGGGAGGAGGGCGTGGC | |
| | CCAGATGTCTGTGGGCCAGAGGGCCAAGCTGACCATCAGCCCAGACTACGCCTATGGAG | |
| | CAACAGGCCACCCAGGAATCATCCCACCTCACGCCACCCTGGTGTTCGATGTGGAGCTGC | |
| | TGAAGCTGGGCGAGGGATCCAACACATCAAAAGAGAACCCCTTTCTGTTCGCATTGGAG | |
| | GCCGTAGTCATATCTGTTGGATCCATGGGACTTATTATCTCCCTGTTGTGTGTGTACTTCT | |
| | GGCTGGAACGGACTATGCCCAGGATCCCCACGCTCAAGAATCTGGAAGATCTCGTCACA | |
| | GAATACCATGGTAATTTCAGCGCCTGGAGCGGAGTCTCTAAGGGTCTGGCCGAATCCCTC | |
| | CAACCCGATTATTCTGAACGGTTGTGCCTCGTATCCGAAATACCACCAAAAGGCGGGGCT | |
| | CTGGGTGAGGGCCCAGGGGCGAGTCCGTGCAATCAACACAGCCCGTATTGGGCCCCTCC | |
| | TTGTTATACGTTGAAGCCCGAAACTGGAAGCGGAGCTACTAACTTCAGCCTGCTGAAGCA | |
| | GGCTGGAGACGTGGAGGAGAACCCTGGACCTATGGCACTGCCCGTGACCGCCCTGCTGC | |
| | TGCCTCTGGCCCTGCTGCTGCACGCAGCCCGGCCTATCCTGTGGCACGAGATGTGGCACG | |
| | AGGGCCTGGAGGAGGCCAGCAGGCTGTATTTTGGCGAGCGCAACGTGAAGGGCATGTTC | |
| | GAGGTGCTGGAGCCTCTGCACGCCATGATGGAGAGAGGCCCACAGACCCTGAAGGAGAC | |
| | ATCCTTTAACCAGGCCTATGGACGGGACCTGATGGAGGCACAGGAGTGGTGCAGAAAGT | |
| | ACATGAAGTCTGGCAATGTGAAGGACCTGCTGCAGGCCTGGGATCTGTACTATCACGTGT | |
| | TTCGGAGAATCTCCAAGGGCAAAGACACGATTCCGTGGCTTGGGCATCTGCTCGTTGGGC | |
| | TGAGTGGTGCGTTTGGTTTCATCATCTTGGTCTATCTCTTGATCAATTGCAGAAATACAGG | |
| | CCCTTGGCTGAAAAAAGTGCTCAAGTGTAATACCCCCGACCCAAGCAAGTTCTTCTCCCA | |
| | GCTTTCTTCAGAGCATGGAGGCGATGTGCAGAAATGGCTCTCTTCACCTTTTCCCTCCTCA | |
| | AGCTTCTCCCCGGGAGGGCTGGCGCCCGAGATTTCACCTCTTGAGGTACTTGAACGAGAC | |
| | AAGGTTACCCAACTTCTCCTTCAACAGGATAAGGTACCCGAACCTGCGAGCCTTAGCTCC | |
| | AACCACTCTCTTACGAGCTGCTTCACCAATCAGGGATACTTCTTTTTCCACCTTCCCGATG | |
| | CGCTGGAAATCGAAGCTTGTCAAGTTTACTTTACCTATGATCCATATAGCGAGGAAGATC | |
| | CCGACGAAGGAGTCGCCGGTGCGCCCACGGGTTCCTCACCCCAACCTCTCCAGCCTCTCT | |
| | CAGGAGAAGATGATGCTTATTGCACTTTTCCCAGTAGAGACGATCTCCTCCTCTTTTCTCC | |
| | ATCTCTTTTGGGGGGACCTTCCCCCCCTTCTACGGCACCTGGCGGGTCTGGTGCTGGCGA | |
| | GGAGCGGATGCCGCCGTCCCTCCAGGAGCGAGTACCACGAGATTGGGATCCCCAGCCAC | |
| | TTGGACCCCCCACCCCCGGCGTACCTGACCTTGTCGATTTTCAACCTCCCCCTGAATTGGT | |
| | GCTGCGAGAGGCTGGGGAGGAAGTTCCGGACGCTGGGCCGAGGGAGGGCGTGTCCTTTC | |
| | CATGGAGTAGGCCTCCAGGTCAAGGCGAGTTTAGGGCTCTCAACGCGCGGCTGCCGTTG | |
| | AATACAGACGCTTATCTCTCACTGCAGGAACTGCAAGGTCAGGACCCAACACATCTTGTA | |
| | GGATCTGGTGCTACTAATTTTTCTCTTTTGAAGCAAGCTGGAGATGTTGAAGAGAACCCT | |
| | GGTCCAGTGAGCAAGGGCGAGGAGCTGTTCACCGGGGTGGTGCCCATCCTGGTCGAGCT | |
| | GGACGGCGACGTAAACGGCCACAAGTTCAGCGTGTCCGGCGAGGGCGAGGGCGATGCCA | |
| | CCTACGGCAAGCTGACCCTGAAGTTCATCTGCACCACCGGCAAGCTGCCCGTGCCCTGGC | |
| | CCACCCTCGTGACCACCCTGACCTACGGCGTGCAGTGCTTCAGCCGCTACCCCGACCACA | |
| | TGAAGCAGCACGACTTCTTCAAGTCCGCCATGCCCGAAGGCTACGTCCAGGAGCGCACC | |
| | ATCTTCTTCAAGGACGACGGCAACTACAAGACCCGCGCCGAGGTGAAGTTCGAGGGCGA | |
| | CACCCTGGTGAACCGCATCGAGCTGAAGGGCATCGACTTCAAGGAGGACGGCAACATCC | |
| | TGGGGCACAAGCTGGAGTACAACTACAACAGCCACAACGTCTATATCATGGCCGACAAG | |
| | CAGAAGAACGGCATCAAGGTGAACTTCAAGATCCGCCACAACATCGAGGACGGCAGCGT | |
| | GCAGCTCGCCGACCACTACCAGCAGAACACCCCCATCGGCGACGGCCCCGTGCTGCTGC | |
| | CCGACAACCACTACCTGAGCACCCAGTCCGCCCTGAGCAAAGACCCCAACGAGAAGCGC | |
| | GATCACATGGTCCTGCTGGAGTTCGTGACCGCCGCCGGGATCACTCTCGGCATGGACGAG | |
| | CTGTACAAGTAAACTAGTGTCGACAATCAACCTCTGGATTACAAAATTTGTGAAAGATTG | |
| | ACTGGTATTCTTAACTATGTTGCTCCTTTTACGCTATGTGGATACGCTGCTTTAATGCCTTT | |
| | GTATCATGCTATTGCTTCCCGTATGGCTTTCATTTTCTCCTCCTTGTATAAATCCTGGTTGC | |
| | TGTCTCTTTATGAGGAGTTGTGGCCCGTTGTCAGGCAACGTGGCGTGGTGTGCACTGTGTT | |
| | TGCTGACGCAACCCCCACTGGTTGGGGCATTGCCACCACCTGTCAGCTCCTTTCCGGGAC | |
| | TTTCGCTTTCCCCCTCCCTATTGCCACGGCGGAACTCATCGCCGCCTGCCTTGCCCGCTGC | |
| | TGGACAGGGGCTCGGCTGTTGGGCACTGACAATTCCGTGGTGTTGTCGGGGAAGCTGACG | |
| | TCCTTTCCATGGCTGCTCGCCTGTGTTGCCACCTGGATTCTGCGCGGGACGTCCTTCTGCT | |
| | ACGTCCCTTCGGCCCTCAATCCAGCGGACCTTCCTTCCCGCGGCCTGCTGCCGGCTCTGCG | |
| | GCCTCTTCCGCGTCTTCGCCTTCGCCCTCAGACGAGTCGGATCTCCCTTTGGGCCGCCTCC | |
| | CCGCCTGGAATTCGAGCTCGGTACCTTTAAGACCAATGACTTACAAGGCAGCTGTAGATC | |
| | TTAGCCACTTTTTAAAAGAAAAGGGGGGACTGGAAGGGCTAATTCACTCCCAACGAAGA | |
| | CAAGATCTGCTTTTTGCTTGTACTGGGTCTCTCTGGTTAGACCAGATCTGAGCCTGGGAGC | |
| | TCTCTGGCTAACTAGGGAACCCACTGCTTAAGCCTCAATAAAGCTTGCCTTGAGTGCTTC | |
| | AAGTAGTGTGTGCCCGTCTGTTGTGTGACTCTGGTAACTAGAGATCCCTCAGACCCTTTTA | |
| | GTCAGTGTGGAAAATCTCTAGCAGTAGTAGTTCATGTCATCTTATTATTCAGTATTTATAA | |
| | CTTGCAAAGAAATGAATATCAGAGAGTGAGAGGAACTTGTTTATTGCAGCTTATAATGGT | |
| | TACAAATAAAGCAATAGCATCACAAATTTCACAAATAAAGCATTTTTTTCACTGCATTCT | |
| | AGTTGTGGTTTGTCCAAACTCATCAATGTATCTTATCATGTCTGGCTCTAGCTATCCCGCC | |
| | CCTAACTCCGCCCAGTTCCGCCCATTCTCCGCCCCATGGCTGACTAATTTTTTTTATTTAT | |
| | GCAGAGGCCGAGGCCGCCTCGGCCTCTGAGCTATTCCAGAAGTAGTGAGGAGGCTTTTTT | |
| | GGAGGCCTAGGCTTTTGCGTCGAGACGTACCCAATTCGCCCTATAGTGAGTCGTATTACG | |
| | CGCGCTCACTGGCCGTCGTTTTACAACGTCGTGACTGGGAAAACCCTGGCGTTACCCAAC | |
| | TTAATCGCCTTGCAGCACATCCCCCTTTCGCCAGCTGGCGTAATAGCGAAGAGGCCCGCA | |
| | CCGATCGCCCTTCCCAACAGTTGCGCAGCCTGAATGGCGAATGGCGCGACGCGCCCTGTA | |
| | GCGGCGCATTAAGCGCGGCGGGTGTGGTGGTTACGCGCAGCGTGACCGCTACACTTGCC | |
| | AGCGCCCTAGCGCCCGCTCCTTTCGCTTTCTTCCCTTCCTTTCTCGCCACGTTCGCCGGCTT | |
| | TCCCCGTCAAGCTCTAAATCGGGGGCTCCCTTTAGGGTTCCGATTTAGTGCTTTACGGCAC | |
| | CTCGACCCCAAAAAACTTGATTAGGGTGATGGTTCACGTAGTGGGCCATCGCCCTGATAG | |
| | ACGGTTTTTCGCCCTTTGACGTTGGAGTCCACGTTCTTTAATAGTGGACTCTTGTTCCAAA | |
| | CTGGAACAACACTCAACCCTATCTCGGTCTATTCTTTTGATTTATAAGGGATTTTGCCGAT | |
| | TTCGGCCTATTGGTTAAAAAATGAGCTGATTTAACAAAAATTTAACGCGAATTTTAACAA | |
| | AATATTAACGTTTACAATTTCCCAGGTGGCACTTTTCGGGGAAATGTGCGCGGAACCCCT | |
| | ATTTGTTTATTTTTCTAAATACATTCAAATATGTATCCGCTCATGAGACAATAACCCTGAT | |
| | AAATGCTTCAATAATATTGAAAAAGGAAGAGTATGAGTATTCAACATTTCCGTGTCGCCC | |
| | TTATTCCCTTTTTTGCGGCATTTTGCCTTCCTGTTTTTGCTCACCCAGAAACGCTGGTGAAA | |
| | GTAAAAGATGCTGAAGATCAGTTGGGTGCACGAGTGGGTTACATCGAACTGGATCTCAA | |
| | CAGCGGTAAGATCCTTGAGAGTTTTCGCCCCGAAGAACGTTTTCCAATGATGAGCACTTT | |
| | TAAAGTTCTGCTATGTGGCGCGGTATTATCCCGTATTGACGCCGGGCAAGAGCAACTCGG | |
| | TCGCCGCATACACTATTCTCAGAATGACTTGGTTGAGTACTCACCAGTCACAGAAAAGCA | |
| | TCTTACGGATGGCATGACAGTAAGAGAATTATGCAGTGCTGCCATAACCATGAGTGATAA | |
| | CACTGCGGCCAACTTACTTCTGACAACGATCGGAGGACCGAAGGAGCTAACCGCTTTTTT | |
| | GCACAACATGGGGGATCATGTAACTCGCCTTGATCGTTGGGAACCGGAGCTGAATGAAG | |
| | CCATACCAAACGACGAGCGTGACACCACGATGCCTGTAGCAATGGCAACAACGTTGCGC | |
| | AAACTATTAACTGGCGAACTACTTACTCTAGCTTCCCGGCAACAATTAATAGACTGGATG | |
| | GAGGCGGATAAAGTTGCAGGACCACTTCTGCGCTCGGCCCTTCCGGCTGGCTGGTTTATT | |
| | GCTGATAAATCTGGAGCCGGTGAGCGTGGGTCTCGCGGTATCATTGCAGCACTGGGGCC | |
| | AGATGGTAAGCCCTCCCGTATCGTAGTTATCTACACGACGGGGAGTCAGGCAACTATGG | |
| | ATGAACGAAATAGACAGATCGCTGAGATAGGTGCCTCACTGATTAAGCATTGGTAACTGT | |
| | CAGACCAAGTTTACTCATATATACTTTAGATTGATTTAAAACTTCATTTTTAATTTAAAAG | |
| | GATCTAGGTGAAGATCCTTTTTGATAATCTCATGACCAAAATCCCTTAACGTGAGTTTTCG | |
| | TTCCACTGAGCGTCAGACCCCGTAGAAAAGATCAAAGGATCTTCTTGAGATCCTTTTTTTC | |
| | TGCGCGTAATCTGCTGCTTGCAAACAAAAAAACCACCGCTACCAGCGGTGGTTTGTTTGC | |
| | CGGATCAAGAGCTACCAACTCTTTTTCCGAAGGTAACTGGCTTCAGCAGAGCGCAGATAC | |
| | CAAATACTGTCCTTCTAGTGTAGCCGTAGTTAGGCCACCACTTCAAGAACTCTGTAGCAC | |
| | CGCCTACATACCTCGCTCTGCTAATCCTGTTACCAGTGGCTGCTGCCAGTGGCGATAAGT | |
| | CGTGTCTTACCGGGTTGGACTCAAGACGATAGTTACCGGATAAGGCGCAGCGGTCGGGC | |
| | TGAACGGGGGGTTCGTGCACACAGCCCAGCTTGGAGCGAACGACCTACACCGAACTGAG | |
| | ATACCTACAGCGTGAGCTATGAGAAAGCGCCACGCTTCCCGAAGGGAGAAAGGCGGACA | |
| | GGTATCCGGTAAGCGGCAGGGTCGGAACAGGAGAGCGCACGAGGGAGCTTCCAGGGGG | |
| | AAACGCCTGGTATCTTTATAGTCCTGTCGGGTTTCGCCACCTCTGACTTGAGCGTCGATTT | |
| | TTGTGATGCTCGTCAGGGGGGCGGAGCCTATGGAAAAACGCCAGCAACGCGGCCTTTTT | |
| | ACGGTTCCTGGCCTTTTGCTGGCCTTTTGCTCACATGTTCTTTCCTGCGTTATCCCCTGATT | |
| | CTGTGGATAACCGTATTACCGCCTTTGAGTGAGCTGATACCGCTCGCCGCAGCCGAACGA | |
| | CCGAGCGCAGCGAGTCAGTGAGCGAGGAAGCGGAAGAGCGCCCAATACGCAAACCGCC | |
| | TCTCCCCGCGCGTTGGCCGATTCATTAATGCAGCTGGCACGACAGGTTTCCCGACTGGAA | |
| | AGCGGGCAGTGAGCGCAACGCAATTAATGTGAGTTAGCTCACTCATTAGGCACCCCAGG | |
| | CTTTACACTTTATGCTTCCGGCTCGTATGTTGTGTGGAATTGTGAGCGGATAACAATTTCA | |
| | CACAGGAAACAGCTATGACCATGATTACGCCAAGCGCGCAATTAACCCTCACTAAAGGG | |
| | AACAAAAGCTGGAGCTGCA | |
| 67 | AGCTTAATGTAGTCTTATGCAATACTCTTGTAGTCTTGCAACATGGTAACGATGAGTTAG | CISC vector |
| | CAACATGCCTTACAAGGAGAGAAAAAGCACCGTGCATGCCGATTGGTGGAAGTAAGGTG | DNA |
| | GTACGATCGTGCCTTATTAGGAAGGCAACAGACGGGTCTGACATGGATTGGACGAACCA | |
| | CTGAATTGCCGCATTGCAGAGATATTGTATTTAAGTGCCTAGCTCGATACAATAAACGGG | |
| | TCTCTCTGGTTAGACCAGATCTGAGCCTGGGAGCTCTCTGGCTAACTAGGGAACCCACTG | |
| | CTTAAGCCTCAATAAAGCTTGCCTTGAGTGCTTCAAGTAGTGTGTGCCCGTCTGTTGTGTG | |
| | ACTCTGGTAACTAGAGATCCCTCAGACCCTTTTAGTCAGTGTGGAAAATCTCTAGCAGTG | |
| | GCGCCCGAACAGGGACTTGAAAGCGAAAGGGAAACCAGAGGAGCTCTCTCGACGCAGG | |
| | ACTCGGCTTGCTGAAGCGCGCACGGCAAGAGGCGAGGGGCGGCGACTGGTGAGTACGCC | |
| | AAAAATTTTGACTAGCGGAGGCTAGAAGGAGAGAGATGGGTGCGAGAGCGTCAGTATTA | |
| | AGCGGGGGAGAATTAGATCGCGATGGGAAAAAATTCGGTTAAGGCCAGGGGGAAAGAA | |
| | AAAATATAAATTAAAACATATAGTATGGGCAAGCAGGGAGCTAGAACGATTCGCAGTTA | |
| | ATCCTGGCCTGTTAGAAACATCAGAAGGCTGTAGACAAATACTGGGACAGCTACAACCA | |
| | TCCCTTCAGACAGGATCAGAAGAACTTAGATCATTATATAATACAGTAGCAACCCTCTAT | |
| | TGTGTGCATCAAAGGATAGAGATAAAAGACACCAAGGAAGCTTTAGACAAGATAGAGGA | |
| | AGAGCAAAACAAAAGTAAGACCACCGCACAGCAAGCGGCCGCTGATCTTCAGACCTGGA | |
| | GGAGGAGATATGAGGGACAATTGGAGAAGTGAATTATATAAATATAAAGTAGTAAAAAT | |
| | TGAACCATTAGGAGTAGCACCCACCAAGGCAAAGAGAAGAGTGGTGCAGAGAGAAAAA | |
| | AGAGCAGTGGGAATAGGAGCTTTGTTCCTTGGGTTCTTGGGAGCAGCAGGAAGCACTAT | |
| | GGGCGCAGCCTCAATGACGCTGACGGTACAGGCCAGACAATTATTGTCTGGTATAGTGC | |
| | AGCAGCAGAACAATTTGCTGAGGGCTATTGAGGCGCAACAGCATCTGTTGCAACTCACA | |
| | GTCTGGGGCATCAAGCAGCTCCAGGCAAGAATCCTGGCTGTGGAAAGATACCTAAAGGA | |
| | TCAACAGCTCCTGGGGATTTGGGGTTGCTCTGGAAAACTCATTTGCACCACTGCTGTGCC | |
| | TTGGAATGCTAGTTGGAGTAATAAATCTCTGGAACAGATTTGGAATCACACGACCTGGAT | |
| | GGAGTGGGACAGAGAAATTAACAATTACACAAGCTTAATACACTCCTTAATTGAAGAAT | |
| | CGCAAAACCAGCAAGAAAAGAATGAACAAGAATTATTGGAATTAGATAAATGGGCAAGT | |
| | TTGTGGAATTGGTTTAACATAACAAATTGGCTGTGGTATATAAAATTATTCATAATGATA | |
| | GTAGGAGGCTTGGTAGGTTTAAGAATAGTTTTTGCTGTACTTTCTATAGTGAATAGAGTT | |
| | AGGCAGGGATATTCACCATTATCGTTTCAGACCCACCTCCCAACCCCGAGGGGACCCGAC | |
| | AGGCCCGAAGGAATAGAAGAAGAAGGTGGAGAGAGAGACAGAGACAGATCCATTCGAT | |
| | TAGTGAACGGATCTCGACGGTATCGGTTAACTTTTAAAAGAAAAGGGGGGATTGGGGGG | |
| | TACAGTGCAGGGGAAAGAATAGTAGACATAATAGCAACAGACATACAAACTAAAGAATT | |
| | ACAAAAACAAATTACAAAAATTCAAAATTTTATCGATCACGAGACTAGCCTCGAGAAGC | |
| | TTGATATCGAATTCCCACGGGGTTGGACGCGTAGGAACAGAGAAACAGGAGAATATGGG | |
| | CCAAACAGGATATCTGTGGTAAGCAGTTCCTGCCCCGGCTCAGGGCCAAGAACAGTTGG | |
| | AACAGCAGAATATGGGCCAAACAGGATATCTGTGGTAAGCAGTTCCTGCCCCGGCTCAG | |
| | GGCCAAGAACAGATGGTCCCCAGATGCGGTCCCGCCCTCAGCAGTTTCTAGAGAACCAT | |
| | CAGATGTTTCCAGGGTGCCCCAAGGACCTGAAATGACCCTGTGCCTTATTTGAACTAACC | |
| | AATCAGTTCGCTTCTCGCTTCTGTTCGCGCGCTTCTGCTCCCCGAGCTCTATATAAGCAGA | |
| | GCTCGTTTAGTGAACCGTCAGATCGCTAGCACCGGTGCCGCCACCATGCCTCTGGGCCTG | |
| | CTGTGGCTGGGCCTGGCCCTGCTGGGCGCCCTGCACGCCCAGGCCGGCGTGCAGGTGGA | |
| | GACAATCTCCCCAGGCGACGGACGCACATTCCCTAAGCGGGGCCAGACCTGCGTGGTGC | |
| | ACTATACAGGCATGCTGGAGGATGGCAAGAAGTTTGACAGCTCCCGGGATAGAAACAAG | |
| | CCATTCAAGTTTATGCTGGGCAAGCAGGAAGTGATCAGAGGCTGGGAGGAGGGCGTGGC | |
| | CCAGATGTCTGTGGGCCAGAGGGCCAAGCTGACCATCAGCCCAGACTACGCCTATGGAG | |
| | CAACAGGCCACCCAGGAATCATCCCACCTCACGCCACCCTGGTGTTCGATGTGGAGCTGC | |
| | TGAAGCTGGGCGAGGGCGGTAGTCAGAACCTTGTGATACCATGGGCCCCAGAAAATCTC | |
| | ACACTTCATAAACTTTCCGAATCACAACTCGAACTCAACTGGAATAACCGGTTCCTGAAT | |
| | CACTGTCTTGAACACCTGGTACAATATCGGACCGACTGGGATCACTCATGGACAGAACA | |
| | ATCTGTGGACTATAGGCACAAATTCTCACTCCCAAGCGTAGACGGCCAAAAAAGATACA | |
| | CTTTTCGCGTACGATCCCGCTTTAATCCTCTCTGCGGCTCTGCTCAGCACTGGAGTGAATG | |
| | GTCCCATCCCATTCATTGGGGATCCAACACATCAAAAGAGAACCCCTTTCTGTTCGCATT | |
| | GGAGGCCGTAGTCATATCTGTTGGATCCATGGGACTTATTATCTCCCTGTTGTGTGTGTAC | |
| | TTCTGGCTGGAACGGACTATGCCCAGGATCCCCACGCTCAAGAATCTGGAAGATCTCGTC | |
| | ACAGAATACCATGGTAATTTCAGCGCCTGGAGCGGAGTCTCTAAGGGTCTGGCCGAATCC | |
| | CTCCAACCCGATTATTCTGAACGGTTGTGCCTCGTATCCGAAATACCACCAAAAGGCGGG | |
| | GCTCTGGGTGAGGGCCCAGGGGCGAGTCCGTGCAATCAACACAGCCCGTATTGGGCCCC | |
| | TCCTTGTTATACGTTGAAGCCCGAAACTGGAAGCGGAGCTACTAACTTCAGCCTGCTGAA | |
| | GCAGGCTGGAGACGTGGAGGAGAACCCTGGACCTATGGCACTGCCCGTGACCGCCCTGC | |
| | TGCTGCCTCTGGCCCTGCTGCTGCACGCAGCCCGGCCTATCCTGTGGCACGAGATGTGGC | |
| | ACGAGGGCCTGGAGGAGGCCAGCAGGCTGTATTTTGGCGAGCGCAACGTGAAGGGCATG | |
| | TTCGAGGTGCTGGAGCCTCTGCACGCCATGATGGAGAGAGGCCCACAGACCCTGAAGGA | |
| | GACATCCTTTAACCAGGCCTATGGACGGGACCTGATGGAGGCACAGGAGTGGTGCAGAA | |
| | AGTACATGAAGTCTGGCAATGTGAAGGACCTGCTGCAGGCCTGGGATCTGTACTATCACG | |
| | TGTTTCGGAGAATCTCCAAGGGAGGTTCAAAACCTTTTGAGAACCTTAGACTGATGGCGC | |
| | CCATCTCTCTGCAGGTAGTTCACGTTGAGACCCATAGATGCAATATAAGCTGGGAAATCT | |
| | CACAAGCCAGCCATTACTTTGAACGGCATTTGGAATTCGAGGCCCGAACACTTTCCCCCG | |
| | GTCATACGTGGGAAGAAGCTCCTCTCTTGACGCTGAAGCAGAAGCAGGAGTGGATTTGTC | |
| | TGGAGACTTTGACTCCTGATACTCAGTATGAGTTCCAAGTTCGGGTGAAACCACTCCAAG | |
| | GCGAGTTCACGACGTGGTCTCCGTGGAGTCAACCGTTGGCGTTCCGCACGAAGCCCGCTG | |
| | CCCTTGGCAAAGACACGATTCCGTGGCTTGGGCATCTGCTCGTTGGGCTGAGTGGTGCGT | |
| | TTGGTTTCATCATCTTGGTCTATCTCTTGATCAATTGCAGAAATACAGGCCCTTGGCTGAA | |
| | AAAAGTGCTCAAGTGTAATACCCCCGACCCAAGCAAGTTCTTCTCCCAGCTTTCTTCAGA | |
| | GCATGGAGGCGATGTGCAGAAATGGCTCTCTTCACCTTTTCCCTCCTCAAGCTTCTCCCCG | |
| | GGAGGGCTGGCGCCCGAGATTTCACCTCTTGAGGTACTTGAACGAGACAAGGTTACCCA | |
| | ACTTCTCCTTCAACAGGATAAGGTACCCGAACCTGCGAGCCTTAGCTCCAACCACTCTCT | |
| | TACGAGCTGCTTCACCAATCAGGGATACTTCTTTTTCCACCTTCCCGATGCGCTGGAAATC | |
| | GAAGCTTGTCAAGTTTACTTTACCTATGATCCATATAGCGAGGAAGATCCCGACGAAGGA | |
| | GTCGCCGGTGCGCCCACGGGTTCCTCACCCCAACCTCTCCAGCCTCTCTCAGGAGAAGAT | |
| | GATGCTTATTGCACTTTTCCCAGTAGAGACGATCTCCTCCTCTTTTCTCCATCTCTTTTGGG | |
| | GGGACCTTCCCCCCCTTCTACGGCACCTGGCGGGTCTGGTGCTGGCGAGGAGCGGATGCC | |
| | GCCGTCCCTCCAGGAGCGAGTACCACGAGATTGGGATCCCCAGCCACTTGGACCCCCCAC | |
| | CCCCGGCGTACCTGACCTTGTCGATTTTCAACCTCCCCCTGAATTGGTGCTGCGAGAGGC | |
| | TGGGGAGGAAGTTCCGGACGCTGGGCCGAGGGAGGGCGTGTCCTTTCCATGGAGTAGGC | |
| | CTCCAGGTCAAGGCGAGTTTAGGGCTCTCAACGCGCGGCTGCCGTTGAATACAGACGCTT | |
| | ATCTCTCACTGCAGGAACTGCAAGGTCAGGACCCAACACATCTTGTAGGATCTGGTGCTA | |
| | CTAATTTTTCTCTTTTGAAGCAAGCTGGAGATGTTGAAGAGAACCCTGGTCCAGTGAGCA | |
| | AGGGCGAGGAGCTGTTCACCGGGGTGGTGCCCATCCTGGTCGAGCTGGACGGCGACGTA | |
| | AACGGCCACAAGTTCAGCGTGTCCGGCGAGGGCGAGGGCGATGCCACCTACGGCAAGCT | |
| | GACCCTGAAGTTCATCTGCACCACCGGCAAGCTGCCCGTGCCCTGGCCCACCCTCGTGAC | |
| | CACCCTGACCTACGGCGTGCAGTGCTTCAGCCGCTACCCCGACCACATGAAGCAGCACG | |
| | ACTTCTTCAAGTCCGCCATGCCCGAAGGCTACGTCCAGGAGCGCACCATCTTCTTCAAGG | |
| | ACGACGGCAACTACAAGACCCGCGCCGAGGTGAAGTTCGAGGGCGACACCCTGGTGAAC | |
| | CGCATCGAGCTGAAGGGCATCGACTTCAAGGAGGACGGCAACATCCTGGGGCACAAGCT | |
| | GGAGTACAACTACAACAGCCACAACGTCTATATCATGGCCGACAAGCAGAAGAACGGCA | |
| | TCAAGGTGAACTTCAAGATCCGCCACAACATCGAGGACGGCAGCGTGCAGCTCGCCGAC | |
| | CACTACCAGCAGAACACCCCCATCGGCGACGGCCCCGTGCTGCTGCCCGACAACCACTA | |
| | CCTGAGCACCCAGTCCGCCCTGAGCAAAGACCCCAACGAGAAGCGCGATCACATGGTCC | |
| | TGCTGGAGTTCGTGACCGCCGCCGGGATCACTCTCGGCATGGACGAGCTGTACAAGTAA | |
| | ACTAGTGTCGACAATCAACCTCTGGATTACAAAATTTGTGAAAGATTGACTGGTATTCTT | |
| | AACTATGTTGCTCCTTTTACGCTATGTGGATACGCTGCTTTAATGCCTTTGTATCATGCTA | |
| | TTGCTTCCCGTATGGCTTTCATTTTCTCCTCCTTGTATAAATCCTGGTTGCTGTCTCTTTAT | |
| | GAGGAGTTGTGGCCCGTTGTCAGGCAACGTGGCGTGGTGTGCACTGTGTTTGCTGACGCA | |
| | ACCCCCACTGGTTGGGGCATTGCCACCACCTGTCAGCTCCTTTCCGGGACTTTCGCTTTCC | |
| | CCCTCCCTATTGCCACGGCGGAACTCATCGCCGCCTGCCTTGCCCGCTGCTGGACAGGGG | |
| | CTCGGCTGTTGGGCACTGACAATTCCGTGGTGTTGTCGGGGAAGCTGACGTCCTTTCCAT | |
| | GGCTGCTCGCCTGTGTTGCCACCTGGATTCTGCGCGGGACGTCCTTCTGCTACGTCCCTTC | |
| | GGCCCTCAATCCAGCGGACCTTCCTTCCCGCGGCCTGCTGCCGGCTCTGCGGCCTCTTCCG | |
| | CGTCTTCGCCTTCGCCCTCAGACGAGTCGGATCTCCCTTTGGGCCGCCTCCCCGCCTGGAA | |
| | TTCGAGCTCGGTACCTTTAAGACCAATGACTTACAAGGCAGCTGTAGATCTTAGCCACTT | |
| | TTTAAAAGAAAAGGGGGGACTGGAAGGGCTAATTCACTCCCAACGAAGACAAGATCTGC | |
| | TTTTTGCTTGTACTGGGTCTCTCTGGTTAGACCAGATCTGAGCCTGGGAGCTCTCTGGCTA | |
| | ACTAGGGAACCCACTGCTTAAGCCTCAATAAAGCTTGCCTTGAGTGCTTCAAGTAGTGTG | |
| | TGCCCGTCTGTTGTGTGACTCTGGTAACTAGAGATCCCTCAGACCCTTTTAGTCAGTGTGG | |
| | AAAATCTCTAGCAGTAGTAGTTCATGTCATCTTATTATTCAGTATTTATAACTTGCAAAGA | |
| | AATGAATATCAGAGAGTGAGAGGAACTTGTTTATTGCAGCTTATAATGGTTACAAATAAA | |
| | GCAATAGCATCACAAATTTCACAAATAAAGCATTTTTTTCACTGCATTCTAGTTGTGGTTT | |
| | GTCCAAACTCATCAATGTATCTTATCATGTCTGGCTCTAGCTATCCCGCCCCTAACTCCGC | |
| | CCAGTTCCGCCCATTCTCCGCCCCATGGCTGACTAATTTTTTTTATTTATGCAGAGGCCGA | |
| | GGCCGCCTCGGCCTCTGAGCTATTCCAGAAGTAGTGAGGAGGCTTTTTTGGAGGCCTAGG | |
| | CTTTTGCGTCGAGACGTACCCAATTCGCCCTATAGTGAGTCGTATTACGCGCGCTCACTG | |
| | GCCGTCGTTTTACAACGTCGTGACTGGGAAAACCCTGGCGTTACCCAACTTAATCGCCTT | |
| | GCAGCACATCCCCCTTTCGCCAGCTGGCGTAATAGCGAAGAGGCCCGCACCGATCGCCCT | |
| | TCCCAACAGTTGCGCAGCCTGAATGGCGAATGGCGCGACGCGCCCTGTAGCGGCGCATT | |
| | AAGCGCGGCGGGTGTGGTGGTTACGCGCAGCGTGACCGCTACACTTGCCAGCGCCCTAG | |
| | CGCCCGCTCCTTTCGCTTTCTTCCCTTCCTTTCTCGCCACGTTCGCCGGCTTTCCCCGTCAA | |
| | GCTCTAAATCGGGGGCTCCCTTTAGGGTTCCGATTTAGTGCTTTACGGCACCTCGACCCC | |
| | AAAAAACTTGATTAGGGTGATGGTTCACGTAGTGGGCCATCGCCCTGATAGACGGTTTTT | |
| | CGCCCTTTGACGTTGGAGTCCACGTTCTTTAATAGTGGACTCTTGTTCCAAACTGGAACAA | |
| | CACTCAACCCTATCTCGGTCTATTCTTTTGATTTATAAGGGATTTTGCCGATTTCGGCCTA | |
| | TTGGTTAAAAAATGAGCTGATTTAACAAAAATTTAACGCGAATTTTAACAAAATATTAAC | |
| | GTTTACAATTTCCCAGGTGGCACTTTTCGGGGAAATGTGCGCGGAACCCCTATTTGTTTAT | |
| | TTTTCTAAATACATTCAAATATGTATCCGCTCATGAGACAATAACCCTGATAAATGCTTCA | |
| | ATAATATTGAAAAAGGAAGAGTATGAGTATTCAACATTTCCGTGTCGCCCTTATTCCCTTT | |
| | TTTGCGGCATTTTGCCTTCCTGTTTTTGCTCACCCAGAAACGCTGGTGAAAGTAAAAGATG | |
| | CTGAAGATCAGTTGGGTGCACGAGTGGGTTACATCGAACTGGATCTCAACAGCGGTAAG | |
| | ATCCTTGAGAGTTTTCGCCCCGAAGAACGTTTTCCAATGATGAGCACTTTTAAAGTTCTGC | |
| | TATGTGGCGCGGTATTATCCCGTATTGACGCCGGGCAAGAGCAACTCGGTCGCCGCATAC | |
| | ACTATTCTCAGAATGACTTGGTTGAGTACTCACCAGTCACAGAAAAGCATCTTACGGATG | |
| | GCATGACAGTAAGAGAATTATGCAGTGCTGCCATAACCATGAGTGATAACACTGCGGCC | |
| | AACTTACTTCTGACAACGATCGGAGGACCGAAGGAGCTAACCGCTTTTTTGCACAACATG | |
| | GGGGATCATGTAACTCGCCTTGATCGTTGGGAACCGGAGCTGAATGAAGCCATACCAAA | |
| | CGACGAGCGTGACACCACGATGCCTGTAGCAATGGCAACAACGTTGCGCAAACTATTAA | |
| | CTGGCGAACTACTTACTCTAGCTTCCCGGCAACAATTAATAGACTGGATGGAGGCGGATA | |
| | AAGTTGCAGGACCACTTCTGCGCTCGGCCCTTCCGGCTGGCTGGTTTATTGCTGATAAAT | |
| | CTGGAGCCGGTGAGCGTGGGTCTCGCGGTATCATTGCAGCACTGGGGCCAGATGGTAAG | |
| | CCCTCCCGTATCGTAGTTATCTACACGACGGGGAGTCAGGCAACTATGGATGAACGAAAT | |
| | AGACAGATCGCTGAGATAGGTGCCTCACTGATTAAGCATTGGTAACTGTCAGACCAAGTT | |
| | TACTCATATATACTTTAGATTGATTTAAAACTTCATTTTTAATTTAAAAGGATCTAGGTGA | |
| | AGATCCTTTTTGATAATCTCATGACCAAAATCCCTTAACGTGAGTTTTCGTTCCACTGAGC | |
| | GTCAGACCCCGTAGAAAAGATCAAAGGATCTTCTTGAGATCCTTTTTTTCTGCGCGTAAT | |
| | CTGCTGCTTGCAAACAAAAAAACCACCGCTACCAGCGGTGGTTTGTTTGCCGGATCAAGA | |
| | GCTACCAACTCTTTTTCCGAAGGTAACTGGCTTCAGCAGAGCGCAGATACCAAATACTGT | |
| | CCTTCTAGTGTAGCCGTAGTTAGGCCACCACTTCAAGAACTCTGTAGCACCGCCTACATA | |
| | CCTCGCTCTGCTAATCCTGTTACCAGTGGCTGCTGCCAGTGGCGATAAGTCGTGTCTTACC | |
| | GGGTTGGACTCAAGACGATAGTTACCGGATAAGGCGCAGCGGTCGGGCTGAACGGGGGG | |
| | TTCGTGCACACAGCCCAGCTTGGAGCGAACGACCTACACCGAACTGAGATACCTACAGC | |
| | GTGAGCTATGAGAAAGCGCCACGCTTCCCGAAGGGAGAAAGGCGGACAGGTATCCGGTA | |
| | AGCGGCAGGGTCGGAACAGGAGAGCGCACGAGGGAGCTTCCAGGGGGAAACGCCTGGT | |
| | ATCTTTATAGTCCTGTCGGGTTTCGCCACCTCTGACTTGAGCGTCGATTTTTGTGATGCTC | |
| | GTCAGGGGGGCGGAGCCTATGGAAAAACGCCAGCAACGCGGCCTTTTTACGGTTCCTGG | |
| | CCTTTTGCTGGCCTTTTGCTCACATGTTCTTTCCTGCGTTATCCCCTGATTCTGTGGATAAC | |
| | CGTATTACCGCCTTTGAGTGAGCTGATACCGCTCGCCGCAGCCGAACGACCGAGCGCAGC | |
| | GAGTCAGTGAGCGAGGAAGCGGAAGAGCGCCCAATACGCAAACCGCCTCTCCCCGCGCG | |
| | TTGGCCGATTCATTAATGCAGCTGGCACGACAGGTTTCCCGACTGGAAAGCGGGCAGTG | |
| | AGCGCAACGCAATTAATGTGAGTTAGCTCACTCATTAGGCACCCCAGGCTTTACACTTTA | |
| | TGCTTCCGGCTCGTATGTTGTGTGGAATTGTGAGCGGATAACAATTTCACACAGGAAACA | |
| | GCTATGACCATGATTACGCCAAGCGCGCAATTAACCCTCACTAAAGGGAACAAAAGCTG | |
| | GAGCTGCA | |
| 68 | AGCTTAATGTAGTCTTATGCAATACTCTTGTAGTCTTGCAACATGGTAACGATGAGTTAG | CISC vector |
| | CAACATGCCTTACAAGGAGAGAAAAAGCACCGTGCATGCCGATTGGTGGAAGTAAGGTG | DNA |
| | GTACGATCGTGCCTTATTAGGAAGGCAACAGACGGGTCTGACATGGATTGGACGAACCA | |
| | CTGAATTGCCGCATTGCAGAGATATTGTATTTAAGTGCCTAGCTCGATACAATAAACGGG | |
| | TCTCTCTGGTTAGACCAGATCTGAGCCTGGGAGCTCTCTGGCTAACTAGGGAACCCACTG | |
| | CTTAAGCCTCAATAAAGCTTGCCTTGAGTGCTTCAAGTAGTGTGTGCCCGTCTGTTGTGTG | |
| | ACTCTGGTAACTAGAGATCCCTCAGACCCTTTTAGTCAGTGTGGAAAATCTCTAGCAGTG | |
| | GCGCCCGAACAGGGACTTGAAAGCGAAAGGGAAACCAGAGGAGCTCTCTCGACGCAGG | |
| | ACTCGGCTTGCTGAAGCGCGCACGGCAAGAGGCGAGGGGCGGCGACTGGTGAGTACGCC | |
| | AAAAATTTTGACTAGCGGAGGCTAGAAGGAGAGAGATGGGTGCGAGAGCGTCAGTATTA | |
| | AGCGGGGGAGAATTAGATCGCGATGGGAAAAAATTCGGTTAAGGCCAGGGGGAAAGAA | |
| | AAAATATAAATTAAAACATATAGTATGGGCAAGCAGGGAGCTAGAACGATTCGCAGTTA | |
| | ATCCTGGCCTGTTAGAAACATCAGAAGGCTGTAGACAAATACTGGGACAGCTACAACCA | |
| | TCCCTTCAGACAGGATCAGAAGAACTTAGATCATTATATAATACAGTAGCAACCCTCTAT | |
| | TGTGTGCATCAAAGGATAGAGATAAAAGACACCAAGGAAGCTTTAGACAAGATAGAGGA | |
| | AGAGCAAAACAAAAGTAAGACCACCGCACAGCAAGCGGCCGCTGATCTTCAGACCTGGA | |
| | GGAGGAGATATGAGGGACAATTGGAGAAGTGAATTATATAAATATAAAGTAGTAAAAAT | |
| | TGAACCATTAGGAGTAGCACCCACCAAGGCAAAGAGAAGAGTGGTGCAGAGAGAAAAA | |
| | AGAGCAGTGGGAATAGGAGCTTTGTTCCTTGGGTTCTTGGGAGCAGCAGGAAGCACTAT | |
| | GGGCGCAGCCTCAATGACGCTGACGGTACAGGCCAGACAATTATTGTCTGGTATAGTGC | |
| | AGCAGCAGAACAATTTGCTGAGGGCTATTGAGGCGCAACAGCATCTGTTGCAACTCACA | |
| | GTCTGGGGCATCAAGCAGCTCCAGGCAAGAATCCTGGCTGTGGAAAGATACCTAAAGGA | |
| | TCAACAGCTCCTGGGGATTTGGGGTTGCTCTGGAAAACTCATTTGCACCACTGCTGTGCC | |
| | TTGGAATGCTAGTTGGAGTAATAAATCTCTGGAACAGATTTGGAATCACACGACCTGGAT | |
| | GGAGTGGGACAGAGAAATTAACAATTACACAAGCTTAATACACTCCTTAATTGAAGAAT | |
| | CGCAAAACCAGCAAGAAAAGAATGAACAAGAATTATTGGAATTAGATAAATGGGCAAGT | |
| | TTGTGGAATTGGTTTAACATAACAAATTGGCTGTGGTATATAAAATTATTCATAATGATA | |
| | GTAGGAGGCTTGGTAGGTTTAAGAATAGTTTTTGCTGTACTTTCTATAGTGAATAGAGTT | |
| | AGGCAGGGATATTCACCATTATCGTTTCAGACCCACCTCCCAACCCCGAGGGGACCCGAC | |
| | AGGCCCGAAGGAATAGAAGAAGAAGGTGGAGAGAGAGACAGAGACAGATCCATTCGAT | |
| | TAGTGAACGGATCTCGACGGTATCGGTTAACTTTTAAAAGAAAAGGGGGGATTGGGGGG | |
| | TACAGTGCAGGGGAAAGAATAGTAGACATAATAGCAACAGACATACAAACTAAAGAATT | |
| | ACAAAAACAAATTACAAAAATTCAAAATTTTATCGATCACGAGACTAGCCTCGAGAAGC | |
| | TTGATATCGAATTCCCACGGGGTTGGACGCGTAGGAACAGAGAAACAGGAGAATATGGG | |
| | CCAAACAGGATATCTGTGGTAAGCAGTTCCTGCCCCGGCTCAGGGCCAAGAACAGTTGG | |
| | AACAGCAGAATATGGGCCAAACAGGATATCTGTGGTAAGCAGTTCCTGCCCCGGCTCAG | |
| | GGCCAAGAACAGATGGTCCCCAGATGCGGTCCCGCCCTCAGCAGTTTCTAGAGAACCAT | |
| | CAGATGTTTCCAGGGTGCCCCAAGGACCTGAAATGACCCTGTGCCTTATTTGAACTAACC | |
| | AATCAGTTCGCTTCTCGCTTCTGTTCGCGCGCTTCTGCTCCCCGAGCTCTATATAAGCAGA | |
| | GCTCGTTTAGTGAACCGTCAGATCGCTAGCACCGGTGCCGCCACCATGCCTCTGGGCCTG | |
| | CTGTGGCTGGGCCTGGCCCTGCTGGGCGCCCTGCACGCCCAGGCCGGCGTGCAGGTGGA | |
| | GACAATCTCCCCAGGCGACGGACGCACATTCCCTAAGCGGGGCCAGACCTGCGTGGTGC | |
| | ACTATACAGGCATGCTGGAGGATGGCAAGAAGTTTGACAGCTCCCGGGATAGAAACAAG | |
| | CCATTCAAGTTTATGCTGGGCAAGCAGGAAGTGATCAGAGGCTGGGAGGAGGGCGTGGC | |
| | CCAGATGTCTGTGGGCCAGAGGGCCAAGCTGACCATCAGCCCAGACTACGCCTATGGAG | |
| | CAACAGGCCACCCAGGAATCATCCCACCTCACGCCACCCTGGTGTTCGATGTGGAGCTGC | |
| | TGAAGCTGGGCGAGCAAAACTTGGTGATTCCTTGGGCCCCAGAAAATCTCACGCTTCACA | |
| | AGTTGTCCGAATCCCAGCTCGAGCTCAACTGGAATAATAGATTTCTTAATCATTGTTTGG | |
| | AACACCTGGTTCAATATAGAACGGATTGGGACCACTCATGGACCGAGCAGTCAGTTGAC | |
| | TACCGCCACAAATTTTCACTTCCCAGCGTAGATGGGCAGAAGAGGTACACATTTAGGGTC | |
| | AGATCCAGGTTTAATCCTCTGTGTGGTTCTGCTCAACACTGGTCTGAGTGGAGCCATCCG | |
| | ATCCACTGGGGCTCAAATACCTCTAAAGAAAATCCGTTCCTCTTTGCGCTCGAAGCCGTT | |
| | GTTATCAGCGTCGGAAGCATGGGACTTATCATTTCCCTTCTCTGCGTGTACTTCTGGCTGG | |
| | AGCGGACGATGCCGCGGATTCCGACGCTCAAAAACCTGGAGGACCTTGTAACAGAATAT | |
| | CACGGTAATTTCTCCGCTTGGAGTGGCGTATCAAAGGGGCTTGCTGAGTCCCTTCAACCG | |
| | GATTACTCTGAGCGCCTCTGCTTGGTGTCCGAGATACCTCCCAAAGGAGGTGCACTTGGG | |
| | GAGGGGCCAGGCGCGTCCCCTTGCAATCAGCATAGTCCGTATTGGGCGCCCCCCTGTTAT | |
| | ACCCTCAAACCGGAAACGGGAAGCGGAGCTACTAACTTCAGCCTGCTGAAGCAGGCTGG | |
| | AGACGTGGAGGAGAACCCTGGACCTATGGCACTGCCCGTGACCGCCCTGCTGCTGCCTCT | |
| | GGCCCTGCTGCTGCACGCAGCCCGGCCTATCCTGTGGCACGAGATGTGGCACGAGGGCCT | |
| | GGAGGAGGCCAGCAGGCTGTATTTTGGCGAGCGCAACGTGAAGGGCATGTTCGAGGTGC | |
| | TGGAGCCTCTGCACGCCATGATGGAGAGAGGCCCACAGACCCTGAAGGAGACATCCTTT | |
| | AACCAGGCCTATGGACGGGACCTGATGGAGGCACAGGAGTGGTGCAGAAAGTACATGA | |
| | AGTCTGGCAATGTGAAGGACCTGCTGCAGGCCTGGGATCTGTACTATCACGTGTTTCGGA | |
| | GAATCTCCAAGAAACCTTTTGAGAACCTTAGACTGATGGCGCCCATCTCTCTGCAGGTAG | |
| | TTCACGTTGAGACCCATAGATGCAATATAAGCTGGGAAATCTCACAAGCCAGCCATTACT | |
| | TTGAACGGCATTTGGAATTCGAGGCCCGAACACTTTCCCCCGGTCATACGTGGGAAGAAG | |
| | CTCCTCTCTTGACGCTGAAGCAGAAGCAGGAGTGGATTTGTCTGGAGACTTTGACTCCTG | |
| | ATACTCAGTATGAGTTCCAAGTTCGGGTGAAACCACTCCAAGGCGAGTTCACGACGTGGT | |
| | CTCCGTGGAGTCAACCGTTGGCGTTCCGCACGAAGCCCGCTGCCCTTGGCAAAGACACGA | |
| | TTCCGTGGCTTGGGCATCTGCTCGTTGGGCTGAGTGGTGCGTTTGGTTTCATCATCTTGGT | |
| | CTATCTCTTGATCAATTGCAGAAATACAGGCCCTTGGCTGAAAAAAGTGCTCAAGTGTAA | |
| | TACCCCCGACCCAAGCAAGTTCTTCTCCCAGCTTTCTTCAGAGCATGGAGGCGATGTGCA | |
| | GAAATGGCTCTCTTCACCTTTTCCCTCCTCAAGCTTCTCCCCGGGAGGGCTGGCGCCCGA | |
| | GATTTCACCTCTTGAGGTACTTGAACGAGACAAGGTTACCCAACTTCTCCTTCAACAGGA | |
| | TAAGGTACCCGAACCTGCGAGCCTTAGCTCCAACCACTCTCTTACGAGCTGCTTCACCAA | |
| | TCAGGGATACTTCTTTTTCCACCTTCCCGATGCGCTGGAAATCGAAGCTTGTCAAGTTTAC | |
| | TTTACCTATGATCCATATAGCGAGGAAGATCCCGACGAAGGAGTCGCCGGTGCGCCCAC | |
| | GGGTTCCTCACCCCAACCTCTCCAGCCTCTCTCAGGAGAAGATGATGCTTATTGCACTTTT | |
| | CCCAGTAGAGACGATCTCCTCCTCTTTTCTCCATCTCTTTTGGGGGGACCTTCCCCCCCTT | |
| | CTACGGCACCTGGCGGGTCTGGTGCTGGCGAGGAGCGGATGCCGCCGTCCCTCCAGGAG | |
| | CGAGTACCACGAGATTGGGATCCCCAGCCACTTGGACCCCCCACCCCCGGCGTACCTGAC | |
| | CTTGTCGATTTTCAACCTCCCCCTGAATTGGTGCTGCGAGAGGCTGGGGAGGAAGTTCCG | |
| | GACGCTGGGCCGAGGGAGGGCGTGTCCTTTCCATGGAGTAGGCCTCCAGGTCAAGGCGA | |
| | GTTTAGGGCTCTCAACGCGCGGCTGCCGTTGAATACAGACGCTTATCTCTCACTGCAGGA | |
| | ACTGCAAGGTCAGGACCCAACACATCTTGTAGGATCTGGTGCTACTAATTTTTCTCTTTTG | |
| | AAGCAAGCTGGAGATGTTGAAGAGAACCCTGGTCCAGTGAGCAAGGGCGAGGAGCTGTT | |
| | CACCGGGGTGGTGCCCATCCTGGTCGAGCTGGACGGCGACGTAAACGGCCACAAGTTCA | |
| | GCGTGTCCGGCGAGGGCGAGGGCGATGCCACCTACGGCAAGCTGACCCTGAAGTTCATC | |
| | TGCACCACCGGCAAGCTGCCCGTGCCCTGGCCCACCCTCGTGACCACCCTGACCTACGGC | |
| | GTGCAGTGCTTCAGCCGCTACCCCGACCACATGAAGCAGCACGACTTCTTCAAGTCCGCC | |
| | ATGCCCGAAGGCTACGTCCAGGAGCGCACCATCTTCTTCAAGGACGACGGCAACTACAA | |
| | GACCCGCGCCGAGGTGAAGTTCGAGGGCGACACCCTGGTGAACCGCATCGAGCTGAAGG | |
| | GCATCGACTTCAAGGAGGACGGCAACATCCTGGGGCACAAGCTGGAGTACAACTACAAC | |
| | AGCCACAACGTCTATATCATGGCCGACAAGCAGAAGAACGGCATCAAGGTGAACTTCAA | |
| | GATCCGCCACAACATCGAGGACGGCAGCGTGCAGCTCGCCGACCACTACCAGCAGAACA | |
| | CCCCCATCGGCGACGGCCCCGTGCTGCTGCCCGACAACCACTACCTGAGCACCCAGTCCG | |
| | CCCTGAGCAAAGACCCCAACGAGAAGCGCGATCACATGGTCCTGCTGGAGTTCGTGACC | |
| | GCCGCCGGGATCACTCTCGGCATGGACGAGCTGTACAAGTAAACTAGTGTCGACAATCA | |
| | ACCTCTGGATTACAAAATTTGTGAAAGATTGACTGGTATTCTTAACTATGTTGCTCCTTTT | |
| | ACGCTATGTGGATACGCTGCTTTAATGCCTTTGTATCATGCTATTGCTTCCCGTATGGCTT | |
| | TCATTTTCTCCTCCTTGTATAAATCCTGGTTGCTGTCTCTTTATGAGGAGTTGTGGCCCGTT | |
| | GTCAGGCAACGTGGCGTGGTGTGCACTGTGTTTGCTGACGCAACCCCCACTGGTTGGGGC | |
| | ATTGCCACCACCTGTCAGCTCCTTTCCGGGACTTTCGCTTTCCCCCTCCCTATTGCCACGG | |
| | CGGAACTCATCGCCGCCTGCCTTGCCCGCTGCTGGACAGGGGCTCGGCTGTTGGGCACTG | |
| | ACAATTCCGTGGTGTTGTCGGGGAAGCTGACGTCCTTTCCATGGCTGCTCGCCTGTGTTGC | |
| | CACCTGGATTCTGCGCGGGACGTCCTTCTGCTACGTCCCTTCGGCCCTCAATCCAGCGGA | |
| | CCTTCCTTCCCGCGGCCTGCTGCCGGCTCTGCGGCCTCTTCCGCGTCTTCGCCTTCGCCCT | |
| | CAGACGAGTCGGATCTCCCTTTGGGCCGCCTCCCCGCCTGGAATTCGAGCTCGGTACCTT | |
| | TAAGACCAATGACTTACAAGGCAGCTGTAGATCTTAGCCACTTTTTAAAAGAAAAGGGG | |
| | GGACTGGAAGGGCTAATTCACTCCCAACGAAGACAAGATCTGCTTTTTGCTTGTACTGGG | |
| | TCTCTCTGGTTAGACCAGATCTGAGCCTGGGAGCTCTCTGGCTAACTAGGGAACCCACTG | |
| | CTTAAGCCTCAATAAAGCTTGCCTTGAGTGCTTCAAGTAGTGTGTGCCCGTCTGTTGTGTG | |
| | ACTCTGGTAACTAGAGATCCCTCAGACCCTTTTAGTCAGTGTGGAAAATCTCTAGCAGTA | |
| | GTAGTTCATGTCATCTTATTATTCAGTATTTATAACTTGCAAAGAAATGAATATCAGAGA | |
| | GTGAGAGGAACTTGTTTATTGCAGCTTATAATGGTTACAAATAAAGCAATAGCATCACAA | |
| | ATTTCACAAATAAAGCATTTTTTTCACTGCATTCTAGTTGTGGTTTGTCCAAACTCATCAA | |
| | TGTATCTTATCATGTCTGGCTCTAGCTATCCCGCCCCTAACTCCGCCCAGTTCCGCCCATT | |
| | CTCCGCCCCATGGCTGACTAATTTTTTTTATTTATGCAGAGGCCGAGGCCGCCTCGGCCTC | |
| | TGAGCTATTCCAGAAGTAGTGAGGAGGCTTTTTTGGAGGCCTAGGCTTTTGCGTCGAGAC | |
| | GTACCCAATTCGCCCTATAGTGAGTCGTATTACGCGCGCTCACTGGCCGTCGTTTTACAA | |
| | CGTCGTGACTGGGAAAACCCTGGCGTTACCCAACTTAATCGCCTTGCAGCACATCCCCCT | |
| | TTCGCCAGCTGGCGTAATAGCGAAGAGGCCCGCACCGATCGCCCTTCCCAACAGTTGCGC | |
| | AGCCTGAATGGCGAATGGCGCGACGCGCCCTGTAGCGGCGCATTAAGCGCGGCGGGTGT | |
| | GGTGGTTACGCGCAGCGTGACCGCTACACTTGCCAGCGCCCTAGCGCCCGCTCCTTTCGC | |
| | TTTCTTCCCTTCCTTTCTCGCCACGTTCGCCGGCTTTCCCCGTCAAGCTCTAAATCGGGGG | |
| | CTCCCTTTAGGGTTCCGATTTAGTGCTTTACGGCACCTCGACCCCAAAAAACTTGATTAG | |
| | GGTGATGGTTCACGTAGTGGGCCATCGCCCTGATAGACGGTTTTTCGCCCTTTGACGTTG | |
| | GAGTCCACGTTCTTTAATAGTGGACTCTTGTTCCAAACTGGAACAACACTCAACCCTATC | |
| | TCGGTCTATTCTTTTGATTTATAAGGGATTTTGCCGATTTCGGCCTATTGGTTAAAAAATG | |
| | AGCTGATTTAACAAAAATTTAACGCGAATTTTAACAAAATATTAACGTTTACAATTTCCC | |
| | AGGTGGCACTTTTCGGGGAAATGTGCGCGGAACCCCTATTTGTTTATTTTTCTAAATACAT | |
| | TCAAATATGTATCCGCTCATGAGACAATAACCCTGATAAATGCTTCAATAATATTGAAAA | |
| | AGGAAGAGTATGAGTATTCAACATTTCCGTGTCGCCCTTATTCCCTTTTTTGCGGCATTTT | |
| | GCCTTCCTGTTTTTGCTCACCCAGAAACGCTGGTGAAAGTAAAAGATGCTGAAGATCAGT | |
| | TGGGTGCACGAGTGGGTTACATCGAACTGGATCTCAACAGCGGTAAGATCCTTGAGAGTT | |
| | TTCGCCCCGAAGAACGTTTTCCAATGATGAGCACTTTTAAAGTTCTGCTATGTGGCGCGG | |
| | TATTATCCCGTATTGACGCCGGGCAAGAGCAACTCGGTCGCCGCATACACTATTCTCAGA | |
| | ATGACTTGGTTGAGTACTCACCAGTCACAGAAAAGCATCTTACGGATGGCATGACAGTAA | |
| | GAGAATTATGCAGTGCTGCCATAACCATGAGTGATAACACTGCGGCCAACTTACTTCTGA | |
| | CAACGATCGGAGGACCGAAGGAGCTAACCGCTTTTTTGCACAACATGGGGGATCATGTA | |
| | ACTCGCCTTGATCGTTGGGAACCGGAGCTGAATGAAGCCATACCAAACGACGAGCGTGA | |
| | CACCACGATGCCTGTAGCAATGGCAACAACGTTGCGCAAACTATTAACTGGCGAACTACT | |
| | TACTCTAGCTTCCCGGCAACAATTAATAGACTGGATGGAGGCGGATAAAGTTGCAGGAC | |
| | CACTTCTGCGCTCGGCCCTTCCGGCTGGCTGGTTTATTGCTGATAAATCTGGAGCCGGTG | |
| | AGCGTGGGTCTCGCGGTATCATTGCAGCACTGGGGCCAGATGGTAAGCCCTCCCGTATCG | |
| | TAGTTATCTACACGACGGGGAGTCAGGCAACTATGGATGAACGAAATAGACAGATCGCT | |
| | GAGATAGGTGCCTCACTGATTAAGCATTGGTAACTGTCAGACCAAGTTTACTCATATATA | |
| | CTTTAGATTGATTTAAAACTTCATTTTTAATTTAAAAGGATCTAGGTGAAGATCCTTTTTG | |
| | ATAATCTCATGACCAAAATCCCTTAACGTGAGTTTTCGTTCCACTGAGCGTCAGACCCCG | |
| | TAGAAAAGATCAAAGGATCTTCTTGAGATCCTTTTTTTCTGCGCGTAATCTGCTGCTTGCA | |
| | AACAAAAAAACCACCGCTACCAGCGGTGGTTTGTTTGCCGGATCAAGAGCTACCAACTCT | |
| | TTTTCCGAAGGTAACTGGCTTCAGCAGAGCGCAGATACCAAATACTGTCCTTCTAGTGTA | |
| | GCCGTAGTTAGGCCACCACTTCAAGAACTCTGTAGCACCGCCTACATACCTCGCTCTGCT | |
| | AATCCTGTTACCAGTGGCTGCTGCCAGTGGCGATAAGTCGTGTCTTACCGGGTTGGACTC | |
| | AAGACGATAGTTACCGGATAAGGCGCAGCGGTCGGGCTGAACGGGGGGTTCGTGCACAC | |
| | AGCCCAGCTTGGAGCGAACGACCTACACCGAACTGAGATACCTACAGCGTGAGCTATGA | |
| | GAAAGCGCCACGCTTCCCGAAGGGAGAAAGGCGGACAGGTATCCGGTAAGCGGCAGGG | |
| | TCGGAACAGGAGAGCGCACGAGGGAGCTTCCAGGGGGAAACGCCTGGTATCTTTATAGT | |
| | CCTGTCGGGTTTCGCCACCTCTGACTTGAGCGTCGATTTTTGTGATGCTCGTCAGGGGGGC | |
| | GGAGCCTATGGAAAAACGCCAGCAACGCGGCCTTTTTACGGTTCCTGGCCTTTTGCTGGC | |
| | CTTTTGCTCACATGTTCTTTCCTGCGTTATCCCCTGATTCTGTGGATAACCGTATTACCGCC | |
| | TTTGAGTGAGCTGATACCGCTCGCCGCAGCCGAACGACCGAGCGCAGCGAGTCAGTGAG | |
| | CGAGGAAGCGGAAGAGCGCCCAATACGCAAACCGCCTCTCCCCGCGCGTTGGCCGATTC | |
| | ATTAATGCAGCTGGCACGACAGGTTTCCCGACTGGAAAGCGGGCAGTGAGCGCAACGCA | |
| | ATTAATGTGAGTTAGCTCACTCATTAGGCACCCCAGGCTTTACACTTTATGCTTCCGGCTC | |
| | GTATGTTGTGTGGAATTGTGAGCGGATAACAATTTCACACAGGAAACAGCTATGACCATG | |
| | ATTACGCCAAGCGCGCAATTAACCCTCACTAAAGGGAACAAAAGCTGGAGCTGCA | |
| 69 | PAAL | Linker/spacer |
| | | polypeptide |
| 70 | GAGATGTGGCATGAGGGTCTGGAAGAAGCGTCTCGACTGTACTTTGGTGAGCGCAATGT | Naked FRB |
| | GAAGGGCATGTTTGAAGTCCTCGAACCCCTTCATGCCATGATGGAACGCGGACCCCAGA | domain |
| | CCTTGAAGGAGACAAGTTTTAACCAAGCTTACGGAAGAGACCTGATGGAAGCCCAGGAA | nucleic acid |
| | TGGTGCAGGAAATACATGAAAAGCGGGAATGTGAAGGACTTGACCCAAGCGTGGGACCT | sequence |
| | GTACTATCATGTCTTTAGGCGCATTAGTAAG | |
| 71 | GAACAGAGAAACAGGAGAATATGGGCCAAACAGGATATCTGTGGTAAGCAGTTCCTGCC | MND |
| | CCGGCTCAGGGCCAAGAACAGTTGGAACAGCAGAATATGGGCCAAACAGGATATCTGTG | promoter |
| | GTAAGCAGTTCCTGCCCCGGCTCAGGGCCAAGAACAGATGGTCCCCAGATGCGGTCCCG | |
| | CCCTCAGCAGTTTCTAGAGAACCATCAGATGTTTCCAGGGTGCCCCAAGGACCTGAAATG | |
| | ACCCTGTGCCTTATTTGAACTAACCAATCAGTTCGCTTCTCGCTTCTGTTCGCGCGCTTCT | |
| | GCTCCCCGAGCTCTATATAAGCAGAGCTCGTTTAGTGAACCGTCAGATCGCTAGC | |
| 72 | GCCACCATGCCTAATCCTCGGCCTGGAAAGCCTAGCGCTCCTTCTCTTGCTCTGGGACCTT | FOXP3cDN |
| | CTCCTGGCGCCTCTCCATCTTGGAGAGCCGCTCCTAAAGCCAGCGATCTGCTGGGAGCTA | A-P2A- |
| | GAGGACCTGGCGGCACATTTCAGGGCAGAGATCTTAGAGGCGGAGCCCACGCTAGCTCC | LNGFR |
| | TCCAGCCTTAATCCTATGCCTCCTAGCCAGCTCCAGCTGCCTACACTGCCTCTGGTTATGG | (kozak-start |
| | TGGCTCCTAGCGGAGCTAGACTGGGCCCTCTGCCTCATCTGCAAGCTCTGCTGCAGGACA | codon- |
| | GACCCCACTTCATGCACCAGCTGAGCACCGTGGATGCCCACGCAAGAACACCTGTGCTGC | FOXP3cDN |
| | AGGTTCACCCTCTGGAATCCCCAGCCATGATCAGCCTGACACCTCCAACAACAGCCACCG | A-P2A- |
| | GCGTGTTCAGCCTGAAAGCCAGACCTGGACTGCCTCCTGGCATCAATGTGGCCAGCCTGG | LNGFR-stop |
| | AATGGGTGTCCAGAGAACCTGCTCTGCTGTGCACATTCCCCAATCCAAGCGCTCCCAGAA | codon) |
| | AGGACAGCACACTGTCTGCCGTGCCTCAGAGCAGCTATCCCCTGCTTGCTAACGGCGTGT | |
| | GCAAGTGGCCTGGATGCGAGAAGGTGTTCGAGGAACCCGAGGACTTCCTGAAGCACTGC | |
| | CAGGCCGATCATCTGCTGGACGAGAAAGGCAGAGCCCAGTGTCTGCTCCAGCGCGAGAT | |
| | GGTGCAGTCTCTGGAACAGCAGCTGGTCCTGGAAAAAGAAAAGCTGAGCGCCATGCAGG | |
| | CCCACCTGGCCGGAAAAATGGCCCTGACAAAGGCCAGCAGCGTGGCCTCTTCTGATAAG | |
| | GGCAGCTGCTGCATTGTGGCCGCTGGATCTCAGGGACCTGTGGTTCCTGCTTGGAGCGGA | |
| | CCTAGAGAGGCCCCTGATTCTCTGTTTGCCGTGCGGAGACACCTGTGGGGCTCTCACGGC | |
| | AACTCTACTTTCCCCGAGTTCCTGCACAACATGGACTACTTCAAGTTCCACAACATGCGG | |
| | CCTCCATTCACCTACGCCACACTGATCAGATGGGCCATTCTGGAAGCCCCTGAGAAGCAG | |
| | AGAACCCTGAACGAGATCTACCACTGGTTTACCCGGATGTTCGCCTTCTTCCGGAATCAC | |
| | CCTGCCACCTGGAAGAACGCCATCCGGCACAATCTGAGCCTGCACAAGTGCTTCGTGCGC | |
| | GTGGAATCTGAGAAAGGCGCCGTGTGGACAGTGGACGAGCTGGAATTCAGAAAGAAGA | |
| | GAAGCCAGCGGCCTAGCCGGTGCAGCAATCCTACACCTGGACCTGGAAGCGGAGCGACT | |
| | AACTTCAGCCTGCTGAAGCAGGCCGGAGATGTGGAGGAAAACCCTGGACCGATGGGGGC | |
| | AGGTGCCACCGGACGAGCCATGGACGGGCCGCGCCTGCTGCTGTTGCTGCTTCTGGGGGT | |
| | GTCCCTTGGAGGTGCCAAGGAGGCATGCCCCACAGGCCTGTACACACACAGCGGTGAGT | |
| | GCTGCAAAGCCTGCAACCTGGGCGAGGGTGTGGCCCAGCCTTGTGGAGCCAACCAGACC | |
| | GTGTGTGAGCCCTGCCTGGACAGCGTGACGTTCTCCGACGTGGTGAGCGCGACCGAGCC | |
| | GTGCAAGCCGTGCACCGAGTGCGTGGGGCTCCAGAGCATGTCGGCGCCGTGCGTGGAGG | |
| | CCGACGACGCCGTGTGCCGCTGCGCCTACGGCTACTACCAGGATGAGACGACTGGGCGC | |
| | TGCGAGGCGTGCCGCGTGTGCGAGGCGGGCTCGGGCCTCGTGTTCTCCTGCCAGGACAA | |
| | GCAGAACACCGTGTGCGAGGAGTGCCCCGACGGCACGTATTCCGACGAGGCCAACCACG | |
| | TGGACCCGTGCCTGCCCTGCACCGTGTGCGAGGACACCGAGCGCCAGCTCCGCGAGTGC | |
| | ACACGCTGGGCCGACGCCGAGTGCGAGGAGATCCCTGGCCGTTGGATTACACGGTCCAC | |
| | ACCCCCAGAGGGCTCGGACAGCACAGCCCCCAGCACCCAGGAGCCTGAGGCACCTCCAG | |
| | AACAAGACCTCATAGCCAGCACGGTGGCAGGTGTGGTGACCACAGTGATGGGCAGCTCC | |
| | CAGCCCGTGGTGACCCGAGGCACCACCGACAACCTCATCCCTGTCTATTGCTCCATCCTG | |
| | GCTGCTGTGGTTGTGGGTCTTGTGGCCTACATAGCCTTCAAGAGGTGA | |
| 73 | GCCACCATGGGGGCAGGTGCCACCGGACGAGCCATGGACGGGCCGCGCCTGCTGCTGTT | LNGFR- |
| | GCTGCTTCTGGGGGTGTCCCTTGGAGGTGCCAAGGAGGCATGCCCCACAGGCCTGTACAC | P2A- |
| | ACACAGCGGTGAGTGCTGCAAAGCCTGCAACCTGGGCGAGGGTGTGGCCCAGCCTTGTG | FOXP3cDN |
| | GAGCCAACCAGACCGTGTGTGAGCCCTGCCTGGACAGCGTGACGTTCTCCGACGTGGTG | A (kozak- |
| | AGCGCGACCGAGCCGTGCAAGCCGTGCACCGAGTGCGTGGGGCTCCAGAGCATGTCGGC | start codon- |
| | GCCGTGCGTGGAGGCCGACGACGCCGTGTGCCGCTGCGCCTACGGCTACTACCAGGATG | LNGFR- |
| | AGACGACTGGGCGCTGCGAGGCGTGCCGCGTGTGCGAGGCGGGCTCGGGCCTCGTGTTC | P2A- |
| | TCCTGCCAGGACAAGCAGAACACCGTGTGCGAGGAGTGCCCCGACGGCACGTATTCCGA | FOXP3cDN |
| | CGAGGCCAACCACGTGGACCCGTGCCTGCCCTGCACCGTGTGCGAGGACACCGAGCGCC | A-stop |
| | AGCTCCGCGAGTGCACACGCTGGGCCGACGCCGAGTGCGAGGAGATCCCTGGCCGTTGG | codon) |
| | ATTACACGGTCCACACCCCCAGAGGGCTCGGACAGCACAGCCCCCAGCACCCAGGAGCC | |
| | TGAGGCACCTCCAGAACAAGACCTCATAGCCAGCACGGTGGCAGGTGTGGTGACCACAG | |
| | TGATGGGCAGCTCCCAGCCCGTGGTGACCCGAGGCACCACCGACAACCTCATCCCTGTCT | |
| | ATTGCTCCATCCTGGCTGCTGTGGTTGTGGGTCTTGTGGCCTACATAGCCTTCAAGAGGG | |
| | GAAGCGGAGCGACTAACTTCAGCCTGCTGAAGCAGGCCGGAGATGTGGAGGAAAACCCT | |
| | GGACCGATGCCTAATCCTCGGCCTGGAAAGCCTAGCGCTCCTTCTCTTGCTCTGGGACCT | |
| | TCTCCTGGCGCCTCTCCATCTTGGAGAGCCGCTCCTAAAGCCAGCGATCTGCTGGGAGCT | |
| | AGAGGACCTGGCGGCACATTTCAGGGCAGAGATCTTAGAGGCGGAGCCCACGCTAGCTC | |
| | CTCCAGCCTTAATCCTATGCCTCCTAGCCAGCTCCAGCTGCCTACACTGCCTCTGGTTATG | |
| | GTGGCTCCTAGCGGAGCTAGACTGGGCCCTCTGCCTCATCTGCAAGCTCTGCTGCAGGAC | |
| | AGACCCCACTTCATGCACCAGCTGAGCACCGTGGATGCCCACGCAAGAACACCTGTGCT | |
| | GCAGGTTCACCCTCTGGAATCCCCAGCCATGATCAGCCTGACACCTCCAACAACAGCCAC | |
| | CGGCGTGTTCAGCCTGAAAGCCAGACCTGGACTGCCTCCTGGCATCAATGTGGCCAGCCT | |
| | GGAATGGGTGTCCAGAGAACCTGCTCTGCTGTGCACATTCCCCAATCCAAGCGCTCCCAG | |
| | AAAGGACAGCACACTGTCTGCCGTGCCTCAGAGCAGCTATCCCCTGCTTGCTAACGGCGT | |
| | GTGCAAGTGGCCTGGATGCGAGAAGGTGTTCGAGGAACCCGAGGACTTCCTGAAGCACT | |
| | GCCAGGCCGATCATCTGCTGGACGAGAAAGGCAGAGCCCAGTGTCTGCTCCAGCGCGAG | |
| | ATGGTGCAGTCTCTGGAACAGCAGCTGGTCCTGGAAAAAGAAAAGCTGAGCGCCATGCA | |
| | GGCCCACCTGGCCGGAAAAATGGCCCTGACAAAGGCCAGCAGCGTGGCCTCTTCTGATA | |
| | AGGGCAGCTGCTGCATTGTGGCCGCTGGATCTCAGGGACCTGTGGTTCCTGCTTGGAGCG | |
| | GACCTAGAGAGGCCCCTGATTCTCTGTTTGCCGTGCGGAGACACCTGTGGGGCTCTCACG | |
| | GCAACTCTACTTTCCCCGAGTTCCTGCACAACATGGACTACTTCAAGTTCCACAACATGC | |
| | GGCCTCCATTCACCTACGCCACACTGATCAGATGGGCCATTCTGGAAGCCCCTGAGAAGC | |
| | AGAGAACCCTGAACGAGATCTACCACTGGTTTACCCGGATGTTCGCCTTCTTCCGGAATC | |
| | ACCCTGCCACCTGGAAGAACGCCATCCGGCACAATCTGAGCCTGCACAAGTGCTTCGTGC | |
| | GCGTGGAATCTGAGAAAGGCGCCGTGTGGACAGTGGACGAGCTGGAATTCAGAAAGAAG | |
| | AGAAGCCAGCGGCCTAGCCGGTGCAGCAATCCTACACCTGGACCTTGA | |
| 74 | ATGCCTAATCCTCGGCCTGGAAAGCCTAGCGCTCCTTCTCTTGCTCTGGGACCTTCTCCTG | FOXP3cDN |
| | GCGCCTCTCCATCTTGGAGAGCCGCTCCTAAAGCCAGCGATCTGCTGGGAGCTAGAGGAC | A-μDISC |
| | CTGGCGGCACATTTCAGGGCAGAGATCTTAGAGGCGGAGCCCACGCTAGCTCCTCCAGC | nucleotide |
| | CTTAATCCTATGCCTCCTAGCCAGCTCCAGCTGCCTACACTGCCTCTGGTTATGGTGGCTC | sequence |
| | CTAGCGGAGCTAGACTGGGCCCTCTGCCTCATCTGCAAGCTCTGCTGCAGGACAGACCCC | (coding |
| | ACTTCATGCACCAGCTGAGCACCGTGGATGCCCACGCAAGAACACCTGTGCTGCAGGTTC | sequence |
| | ACCCTCTGGAATCCCCAGCCATGATCAGCCTGACACCTCCAACAACAGCCACCGGCGTGT | only; codon- |
| | TCAGCCTGAAAGCCAGACCTGGACTGCCTCCTGGCATCAATGTGGCCAGCCTGGAATGG | optimized; |
| | GTGTCCAGAGAACCTGCTCTGCTGTGCACATTCCCCAATCCAAGCGCTCCCAGAAAGGAC | our DISC |
| | AGCACACTGTCTGCCGTGCCTCAGAGCAGCTATCCCCTGCTTGCTAACGGCGTGTGCAAG | architecture |
| | TGGCCTGGATGCGAGAAGGTGTTCGAGGAACCCGAGGACTTCCTGAAGCACTGCCAGGC | version 6) |
| | CGATCATCTGCTGGACGAGAAAGGCAGAGCCCAGTGTCTGCTCCAGCGCGAGATGGTGC | |
| | AGTCTCTGGAACAGCAGCTGGTCCTGGAAAAAGAAAAGCTGAGCGCCATGCAGGCCCAC | |
| | CTGGCCGGAAAAATGGCCCTGACAAAGGCCAGCAGCGTGGCCTCTTCTGATAAGGGCAG | |
| | CTGCTGCATTGTGGCCGCTGGATCTCAGGGACCTGTGGTTCCTGCTTGGAGCGGACCTAG | |
| | AGAGGCCCCTGATTCTCTGTTTGCCGTGCGGAGACACCTGTGGGGCTCTCACGGCAACTC | |
| | TACTTTCCCCGAGTTCCTGCACAACATGGACTACTTCAAGTTCCACAACATGCGGCCTCC | |
| | ATTCACCTACGCCACACTGATCAGATGGGCCATTCTGGAAGCCCCTGAGAAGCAGAGAA | |
| | CCCTGAACGAGATCTACCACTGGTTTACCCGGATGTTCGCCTTCTTCCGGAATCACCCTGC | |
| | CACCTGGAAGAACGCCATCCGGCACAATCTGAGCCTGCACAAGTGCTTCGTGCGCGTGG | |
| | AATCTGAGAAAGGCGCCGTGTGGACAGTGGACGAGCTGGAATTCAGAAAGAAGAGAAG | |
| | CCAGCGGCCTAGCCGGTGCAGCAATCCTACACCTGGACCTGGAAGCGGAGCGACTAACT | |
| | TCAGCCTGCTTAAGCAGGCCGGAGATGTGGAGGAAAACCCTGGACCGATGCCTCTGGGC | |
| | CTGCTGTGGCTGGGCCTGGCCCTGCTGGGCGCCCTGCACGCCCAGGCCGGCGTGCAGGTG | |
| | GAGACAATCTCCCCAGGCGACGGACGCACATTCCCTAAGCGGGGCCAGACCTGCGTGGT | |
| | GCACTATACAGGCATGCTGGAGGATGGCAAGAAGTTTGACAGCTCCCGGGATAGAAACA | |
| | AGCCATTCAAGTTTATGCTGGGCAAGCAGGAAGTGATCAGAGGCTGGGAGGAGGGCGTG | |
| | GCCCAGATGTCTGTGGGCCAGAGGGCCAAGCTGACCATCAGCCCAGACTACGCCTATGG | |
| | AGCAACAGGCCACCCAGGAATCATCCCACCTCACGCCACCCTGGTGTTCGATGTGGAGCT | |
| | GCTGAAGCTGGGCGAGGGAGGGTCACCTGGATCCAACACATCAAAAGAGAACCCCTTTC | |
| | TGTTCGCATTGGAGGCCGTAGTCATATCTGTTGGATCCATGGGACTTATTATCTCCCTGTT | |
| | GTGTGTGTACTTCTGGCTGGAACGGACTATGCCCAGGATCCCCACGCTCAAGAATCTGGA | |
| | AGATCTCGTCACAGAATACCATGGTAATTTCAGCGCCTGGAGCGGAGTCTCTAAGGGTCT | |
| | GGCCGAATCCCTCCAACCCGATTATTCTGAACGGTTGTGCCTCGTATCCGAAATACCACC | |
| | AAAAGGCGGGGCTCTGGGTGAGGGCCCAGGGGCGAGTCCGTGCAATCAACACAGCCCGT | |
| | ATTGGGCCCCTCCTTGTTATACGTTGAAGCCCGAAACTGGAAGCGGAGCTACTAACTTCA | |
| | GCCTGCTGAAGCAGGCTGGAGACGTGGAGGAGAACCCTGGACCTATGGCACTGCCCGTG | |
| | ACCGCCCTGCTGCTGCCTCTGGCCCTGCTGCTGCACGCAGCCCGGCCTATCCTGTGGCAC | |
| | GAGATGTGGCACGAGGGCCTGGAGGAGGCCAGCAGGCTGTATTTTGGCGAGCGCAACGT | |
| | GAAGGGCATGTTCGAGGTGCTGGAGCCTCTGCACGCCATGATGGAGAGAGGCCCACAGA | |
| | CCCTGAAGGAGACATCCTTTAACCAGGCCTATGGACGGGACCTGATGGAGGCACAGGAG | |
| | TGGTGCAGAAAGTACATGAAGTCTGGCAATGTGAAGGACCTGCTGCAGGCCTGGGATCT | |
| | GTACTATCACGTGTTTCGGAGAATCTCCAAGCCAGCAGCTCTCGGCAAAGACACGATTCC | |
| | GTGGCTTGGGCATCTGCTCGTTGGGCTGAGCGGTGCGTTTGGTTTCATCATCTTGGTCTAT | |
| | CTCTTGATCAATTGCAGAAATACAGGCCCTTGGCTGAAAAAAGTGCTCAAGTGTAATACC | |
| | CCCGACCCAAGCAAGTTCTTCTCCCAGCTTTCTTCAGAGCATGGAGGCGATGTGCAGAAA | |
| | TGGCTCTCTTCACCTTTTCCCTCCTCAAGCTTCTCCCCGGGAGGGCTGGCGCCCGAGATTT | |
| | CACCTCTTGAGGTACTTGAACGAGACAAGGTTACCCAACTTCTCCTTCAACAGGATAAGG | |
| | TACCCGAACCTGCGAGCCTTAGCTTGAATACAGACGCTTATCTCTCACTGCAGGAACTGC | |
| | AAGGATCTGGTGCTACTAATTTTTCTCTTTTGAAGCAAGCTGGAGATGTTGAAGAGAACC | |
| | CCGGTCCGGAGATGTGGCATGAGGGTCTGGAAGAAGCGTCTCGACTGTACTTTGGTGAGC | |
| | GCAATGTGAAGGGCATGTTTGAAGTCCTCGAACCCCTTCATGCCATGATGGAACGCGGAC | |
| | CCCAGACCTTGAAGGAGACAAGTTTTAACCAAGCTTACGGAAGAGACCTGATGGAAGCC | |
| | CAGGAATGGTGCAGGAAATACATGAAAAGCGGGAATGTGAAGGACTTGCTCCAAGCGTG | |
| | GGACCTGTACTATCATGTCTTTAGGCGCATTAGTAAGTGA | |
| 75 | GAACAGAGAAACAGGAGAATATGGGCCAAACAGGATATCTGTGGTAAGCAGTTCCTGCC | MND- |
| | CCGGCTCAGGGCCAAGAACAGTTGGAACAGCAGAATATGGGCCAAACAGGATATCTGTG | FOXP3cDN |
| | GTAAGCAGTTCCTGCCCCGGCTCAGGGCCAAGAACAGATGGTCCCCAGATGCGGTCCCG | A-μDISC- |
| | CCCTCAGCAGTTTCTAGAGAACCATCAGATGTTTCCAGGGTGCCCCAAGGACCTGAAATG | SV40 polyA |
| | ACCCTGTGCCTTATTTGAACTAACCAATCAGTTCGCTTCTCGCTTCTGTTCGCGCGCTTCT | nucleotide |
| | GCTCCCCGAGCTCTATATAAGCAGAGCTCGTTTAGTGAACCGTCAGATCGCCTGGAGACG | sequence |
| | CCATCCACGCTGTTTTGACTTCCATAGAAGGATCTCGAGGCCACCATGCCTAATCCTCGG | (codon |
| | CCTGGAAAGCCTAGCGCTCCTTCTCTTGCTCTGGGACCTTCTCCTGGCGCCTCTCCATCTT | optimized; |
| | GGAGAGCCGCTCCTAAAGCCAGCGATCTGCTGGGAGCTAGAGGACCTGGCGGCACATTT | this is our |
| | CAGGGCAGAGATCTTAGAGGCGGAGCCCACGCTAGCTCCTCCAGCCTTAATCCTATGCCT | expression |
| | CCTAGCCAGCTCCAGCTGCCTACACTGCCTCTGGTTATGGTGGCTCCTAGCGGAGCTAGA | cassette part |
| | CTGGGCCCTCTGCCTCATCTGCAAGCTCTGCTGCAGGACAGACCCCACTTCATGCACCAG | of the donor |
| | CTGAGCACCGTGGATGCCCACGCAAGAACACCTGTGCTGCAGGTTCACCCTCTGGAATCC | template; |
| | CCAGCCATGATCAGCCTGACACCTCCAACAACAGCCACCGGCGTGTTCAGCCTGAAAGC | does not |
| | CAGACCTGGACTGCCTCCTGGCATCAATGTGGCCAGCCTGGAATGGGTGTCCAGAGAAC | include |
| | CTGCTCTGCTGTGCACATTCCCCAATCCAAGCGCTCCCAGAAAGGACAGCACACTGTCTG | homology |
| | CCGTGCCTCAGAGCAGCTATCCCCTGCTTGCTAACGGCGTGTGCAAGTGGCCTGGATGCG | arms (e.g. |
| | AGAAGGTGTTCGAGGAACCCGAGGACTTCCTGAAGCACTGCCAGGCCGATCATCTGCTG | targeting to |
| | GACGAGAAAGGCAGAGCCCAGTGTCTGCTCCAGCGCGAGATGGTGCAGTCTCTGGAACA | FoxP3, |
| | GCAGCTGGTCCTGGAAAAAGAAAAGCTGAGCGCCATGCAGGCCCACCTGGCCGGAAAAA | AAVS1, etc) |
| | TGGCCCTGACAAAGGCCAGCAGCGTGGCCTCTTCTGATAAGGGCAGCTGCTGCATTGTGG | nor AAV |
| | CCGCTGGATCTCAGGGACCTGTGGTTCCTGCTTGGAGCGGACCTAGAGAGGCCCCTGATT | vector |
| | CTCTGTTTGCCGTGCGGAGACACCTGTGGGGCTCTCACGGCAACTCTACTTTCCCCGAGTT | sequences) |
| | CCTGCACAACATGGACTACTTCAAGTTCCACAACATGCGGCCTCCATTCACCTACGCCAC | |
| | ACTGATCAGATGGGCCATTCTGGAAGCCCCTGAGAAGCAGAGAACCCTGAACGAGATCT | |
| | ACCACTGGTTTACCCGGATGTTCGCCTTCTTCCGGAATCACCCTGCCACCTGGAAGAACG | |
| | CCATCCGGCACAATCTGAGCCTGCACAAGTGCTTCGTGCGCGTGGAATCTGAGAAAGGC | |
| | GCCGTGTGGACAGTGGACGAGCTGGAATTCAGAAAGAAGAGAAGCCAGCGGCCTAGCC | |
| | GGTGCAGCAATCCTACACCTGGACCTGGAAGCGGAGCGACTAACTTCAGCCTGCTTAAG | |
| | CAGGCCGGAGATGTGGAGGAAAACCCTGGACCGATGCCTCTGGGCCTGCTGTGGCTGGG | |
| | CCTGGCCCTGCTGGGCGCCCTGCACGCCCAGGCCGGCGTGCAGGTGGAGACAATCTCCCC | |
| | AGGCGACGGACGCACATTCCCTAAGCGGGGCCAGACCTGCGTGGTGCACTATACAGGCA | |
| | TGCTGGAGGATGGCAAGAAGTTTGACAGCTCCCGGGATAGAAACAAGCCATTCAAGTTT | |
| | ATGCTGGGCAAGCAGGAAGTGATCAGAGGCTGGGAGGAGGGCGTGGCCCAGATGTCTGT | |
| | GGGCCAGAGGGCCAAGCTGACCATCAGCCCAGACTACGCCTATGGAGCAACAGGCCACC | |
| | CAGGAATCATCCCACCTCACGCCACCCTGGTGTTCGATGTGGAGCTGCTGAAGCTGGGCG | |
| | AGGGAGGGTCACCTGGATCCAACACATCAAAAGAGAACCCCTTTCTGTTCGCATTGGAG | |
| | GCCGTAGTCATATCTGTTGGATCCATGGGACTTATTATCTCCCTGTTGTGTGTGTACTTCT | |
| | GGCTGGAACGGACTATGCCCAGGATCCCCACGCTCAAGAATCTGGAAGATCTCGTCACA | |
| | GAATACCATGGTAATTTCAGCGCCTGGAGCGGAGTCTCTAAGGGTCTGGCCGAATCCCTC | |
| | CAACCCGATTATTCTGAACGGTTGTGCCTCGTATCCGAAATACCACCAAAAGGCGGGGCT | |
| | CTGGGTGAGGGCCCAGGGGCGAGTCCGTGCAATCAACACAGCCCGTATTGGGCCCCTCC | |
| | TTGTTATACGTTGAAGCCCGAAACTGGAAGCGGAGCTACTAACTTCAGCCTGCTGAAGCA | |
| | GGCTGGAGACGTGGAGGAGAACCCTGGACCTATGGCACTGCCCGTGACCGCCCTGCTGC | |
| | TGCCTCTGGCCCTGCTGCTGCACGCAGCCCGGCCTATCCTGTGGCACGAGATGTGGCACG | |
| | AGGGCCTGGAGGAGGCCAGCAGGCTGTATTTTGGCGAGCGCAACGTGAAGGGCATGTTC | |
| | GAGGTGCTGGAGCCTCTGCACGCCATGATGGAGAGAGGCCCACAGACCCTGAAGGAGAC | |
| | ATCCTTTAACCAGGCCTATGGACGGGACCTGATGGAGGCACAGGAGTGGTGCAGAAAGT | |
| | ACATGAAGTCTGGCAATGTGAAGGACCTGCTGCAGGCCTGGGATCTGTACTATCACGTGT | |
| | TTCGGAGAATCTCCAAGCCAGCAGCTCTCGGCAAAGACACGATTCCGTGGCTTGGGCATC | |
| | TGCTCGTTGGGCTGAGCGGTGCGTTTGGTTTCATCATCTTGGTCTATCTCTTGATCAATTG | |
| | CAGAAATACAGGCCCTTGGCTGAAAAAAGTGCTCAAGTGTAATACCCCCGACCCAAGCA | |
| | AGTTCTTCTCCCAGCTTTCTTCAGAGCATGGAGGCGATGTGCAGAAATGGCTCTCTTCAC | |
| | CTTTTCCCTCCTCAAGCTTCTCCCCGGGAGGGCTGGCGCCCGAGATTTCACCTCTTGAGGT | |
| | ACTTGAACGAGACAAGGTTACCCAACTTCTCCTTCAACAGGATAAGGTACCCGAACCTGC | |
| | GAGCCTTAGCTTGAATACAGACGCTTATCTCTCACTGCAGGAACTGCAAGGATCTGGTGC | |
| | TACTAATTTTTCTCTTTTGAAGCAAGCTGGAGATGTTGAAGAGAACCCCGGTCCGGAGAT | |
| | GTGGCATGAGGGTCTGGAAGAAGCGTCTCGACTGTACTTTGGTGAGCGCAATGTGAAGG | |
| | GCATGTTTGAAGTCCTCGAACCCCTTCATGCCATGATGGAACGCGGACCCCAGACCTTGA | |
| | AGGAGACAAGTTTTAACCAAGCTTACGGAAGAGACCTGATGGAAGCCCAGGAATGGTGC | |
| | AGGAAATACATGAAAAGCGGGAATGTGAAGGACTTGCTCCAAGCGTGGGACCTGTACTA | |
| | TCATGTCTTTAGGCGCATTAGTAAGTGAGTCGACTGCTTTATTTGTGAAATTTGTGATGCT | |
| | ATTGCTTTATTTGTAACCATTATAAGCTGCAATAAACAAGTTAACAACAACAATTGCATT | |
| | CATTTTATGTTTCAGGTTCAGGGGGAGATGTGGGAGGTTTTTTAAAGC | |
| 76 | MPNPRPGKPSAPSLALGPSPGASPSWRAAPKASDLLGARGPGGTFQGRDLRGGAHASSSSLN | FOXP3cDN |
| | PMPPSQLQLPTLPLVMVAPSGARLGPLPHLQALLQDRPHFMHQLSTVDAHARTPVLQVHPLE | A-μDISC |
| | SPAMISLTPPTTATGVFSLKARPGLPPGINVASLEWVSREPALLCTFPNPSAPRKDSTLSAVPQS | amino acid |
| | SYPLLANGVCKWPGCEKVFEEPEDFLKHCQADHLLDEKGRAQCLLQREMVQSLEQQLVLEK | sequence |
| | EKLSAMQAHLAGKMALTKASSVASSDKGSCCIVAAGSQGPVVPAWSGPREAPDSLFAVRRH | |
| | LWGSHGNSTFPEFLHNMDYFKFHNMRPPFTYATLIRWAILEAPEKQRTLNEIYHWFTRMFAF | |
| | FRNHPATWKNAIRHNLSLHKCFVRVESEKGAVWTVDELEFRKKRSQRPSRCSNPTPGPGSGA | |
| | TNFSLLKQAGDVEENPGPMPLGLLWLGLALLGALHAQAGVQVETISPGDGRTFPKRGQTCV | |
| | VHYTGMLEDGKKFDSSRDRNKPFKFMLGKQEVIRGWEEGVAQMSVGQRAKLTISPDYAYG | |
| | ATGHPGIIPPHATLVFDVELLKLGEGGSPGSNTSKENPFLFALEAVVISVGSMGLIISLLCVYFW | |
| | LERTMPRIPTLKNLEDLVTEYHGNFSAWSGVSKGLAESLQPDYSERLCLVSEIPPKGGALGEG | |
| | PGASPCNQHSPYWAPPCYTLKPETGSGATNFSLLKQAGDVEENPGPMALPVTALLLPLALLL | |
| | HAARPILWHEMWHEGLEEASRLYFGERNVKGMFEVLEPLHAMMERGPQTLKETSFNQAYG | |
| | RDLMEAQEWCRKYMKSGNVKDLLQAWDLYYHVFRRISKPAALGKDTIPWLGHLLVGLSGA | |
| | FGFIILVYLLINCRNTGPWLKKVLKCNTPDPSKFFSQLSSEHGGDVQKWLSSPFPSSSFSPGGL | |
| | APEISPLEVLERDKVTQLLLQQDKVPEPASLSLNTDAYLSLQELQGSGATNFSLLKQAGDVEE | |
| | NPGPEMWHEGLEEASRLYFGERNVKGMFEVLEPLHAMMERGPQTLKETSFNQAYGRDLME | |
| | AQEWCRKYMKSGNVKDLLQAWDLYYHVFRRISK* | |
| 77 | ATGCCTAATCCTCGGCCTGGAAAGCCTAGCGCTCCTTCTCTTGCTCTGGGACCTTCTCCTG | FOXP3cDN |
| | GCGCCTCTCCATCTTGGAGAGCCGCTCCTAAAGCCAGCGATCTGCTGGGAGCTAGAGGAC | A-LNGFRe- |
| | CTGGCGGCACATTTCAGGGCAGAGATCTTAGAGGCGGAGCCCACGCTAGCTCCTCCAGC | μDISC |
| | CTTAATCCTATGCCTCCTAGCCAGCTCCAGCTGCCTACACTGCCTCTGGTTATGGTGGCTC | nucleotide |
| | CTAGCGGAGCTAGACTGGGCCCTCTGCCTCATCTGCAAGCTCTGCTGCAGGACAGACCCC | sequence |
| | ACTTCATGCACCAGCTGAGCACCGTGGATGCCCACGCAAGAACACCTGTGCTGCAGGTTC | (coding |
| | ACCCTCTGGAATCCCCAGCCATGATCAGCCTGACACCTCCAACAACAGCCACCGGCGTGT | sequence |
| | TCAGCCTGAAAGCCAGACCTGGACTGCCTCCTGGCATCAATGTGGCCAGCCTGGAATGG | only; codon- |
| | GTGTCCAGAGAACCTGCTCTGCTGTGCACATTCCCCAATCCAAGCGCTCCCAGAAAGGAC | optimized; |
| | AGCACACTGTCTGCCGTGCCTCAGAGCAGCTATCCCCTGCTTGCTAACGGCGTGTGCAAG | our DISC |
| | TGGCCTGGATGCGAGAAGGTGTTCGAGGAACCCGAGGACTTCCTGAAGCACTGCCAGGC | architecture |
| | CGATCATCTGCTGGACGAGAAAGGCAGAGCCCAGTGTCTGCTCCAGCGCGAGATGGTGC | version 6) |
| | AGTCTCTGGAACAGCAGCTGGTCCTGGAAAAAGAAAAGCTGAGCGCCATGCAGGCCCAC | |
| | CTGGCCGGAAAAATGGCCCTGACAAAGGCCAGCAGCGTGGCCTCTTCTGATAAGGGCAG | |
| | CTGCTGCATTGTGGCCGCTGGATCTCAGGGACCTGTGGTTCCTGCTTGGAGCGGACCTAG | |
| | AGAGGCCCCTGATTCTCTGTTTGCCGTGCGGAGACACCTGTGGGGCTCTCACGGCAACTC | |
| | TACTTTCCCCGAGTTCCTGCACAACATGGACTACTTCAAGTTCCACAACATGCGGCCTCC | |
| | ATTCACCTACGCCACACTGATCAGATGGGCCATTCTGGAAGCCCCTGAGAAGCAGAGAA | |
| | CCCTGAACGAGATCTACCACTGGTTTACCCGGATGTTCGCCTTCTTCCGGAATCACCCTGC | |
| | CACCTGGAAGAACGCCATCCGGCACAATCTGAGCCTGCACAAGTGCTTCGTGCGCGTGG | |
| | AATCTGAGAAAGGCGCCGTGTGGACAGTGGACGAGCTGGAATTCAGAAAGAAGAGAAG | |
| | CCAGCGGCCTAGCCGGTGCAGCAATCCTACACCTGGACCTGGAAGCGGAGCGACTAACT | |
| | TCAGCCTGCTTAAGCAGGCCGGAGATGTGGAGGAAAACCCTGGACCGATGCCTCTGGGC | |
| | CTGCTGTGGCTGGGCCTGGCCCTGCTGGGCGCCCTGCACGCCCAGGCCATGGGGGCAGGT | |
| | GCCACCGGACGAGCCATGGACGGGCCGCGCCTGCTGCTGTTGCTGCTTCTGGGGGTGTCC | |
| | CTTGGAGGTGCCAAGGAGGCATGCCCCACAGGCCTGTACACACACAGCGGTGAGTGCTG | |
| | CAAAGCCTGCAACCTGGGCGAGGGTGTGGCCCAGCCTTGTGGAGCCAACCAGACCGTGT | |
| | GTGAGCCCTGCCTGGACAGCGTGACGTTCTCCGACGTGGTGAGCGCGACCGAGCCGTGC | |
| | AAGCCGTGCACCGAGTGCGTGGGGCTCCAGAGCATGTCGGCGCCGTGCGTGGAGGCCGA | |
| | CGACGCCGTGTGCCGCTGCGCCTACGGCTACTACCAGGATGAGACGACTGGGCGCTGCG | |
| | AGGCGTGCCGCGTGTGCGAGGCGGGCTCGGGCCTCGTGTTCTCCTGCCAGGACAAGCAG | |
| | AACACCGTGTGCGAGGAGTGCCCCGACGGCACGTATTCCGACGAGGCCAACCACGTGGA | |
| | CCCGTGCCTGCCCTGCACCGTGTGCGAGGACACCGAGCGCCAGCTCCGCGAGTGCACAC | |
| | GCTGGGCCGACGCCGAGTGCGAGGAGATCCCTGGCCGTTGGATTACACGGTCCACACCC | |
| | CCAGAGGGCTCGGACAGCACAGCCCCCAGCACCCAGGAGCCTGAGGCACCTCCAGAACA | |
| | AGACCTCATAGCCAGCACGGTGGCAGGTGTGGTGACCACAGTGATGGGCAGCTCCCAGC | |
| | CCGTGGTGACCCGAGGCACCACCGACAACCTCATCCCTGTCTATTGCTCCATCCTGGCTG | |
| | CTGTGGTTGTGGGTCTTGTGGCCTACATAGCCTTCAAGAGGGGCGTGCAGGTGGAGACAA | |
| | TCTCCCCAGGCGACGGACGCACATTCCCTAAGCGGGGCCAGACCTGCGTGGTGCACTATA | |
| | CAGGCATGCTGGAGGATGGCAAGAAGTTTGACAGCTCCCGGGATAGAAACAAGCCATTC | |
| | AAGTTTATGCTGGGCAAGCAGGAAGTGATCAGAGGCTGGGAGGAGGGCGTGGCCCAGAT | |
| | GTCTGTGGGCCAGAGGGCCAAGCTGACCATCAGCCCAGACTACGCCTATGGAGCAACAG | |
| | GCCACCCAGGAATCATCCCACCTCACGCCACCCTGGTGTTCGATGTGGAGCTGCTGAAGC | |
| | TGGGCGAGGGAGGGTCACCTGGATCCAACACATCAAAAGAGAACCCCTTTCTGTTCGCA | |
| | TTGGAGGCCGTAGTCATATCTGTTGGATCCATGGGACTTATTATCTCCCTGTTGTGTGTGT | |
| | ACTTCTGGCTGGAACGGACTATGCCCAGGATCCCCACGCTCAAGAATCTGGAAGATCTCG | |
| | TCACAGAATACCATGGTAATTTCAGCGCCTGGAGCGGAGTCTCTAAGGGTCTGGCCGAAT | |
| | CCCTCCAACCCGATTATTCTGAACGGTTGTGCCTCGTATCCGAAATACCACCAAAAGGCG | |
| | GGGCTCTGGGTGAGGGCCCAGGGGCGAGTCCGTGCAATCAACACAGCCCGTATTGGGCC | |
| | CCTCCTTGTTATACGTTGAAGCCCGAAACTGGAAGCGGAGCTACTAACTTCAGCCTGCTG | |
| | AAGCAGGCTGGAGACGTGGAGGAGAACCCTGGACCTATGGCACTGCCCGTGACCGCCCT | |
| | GCTGCTGCCTCTGGCCCTGCTGCTGCACGCAGCCCGGCCTATCCTGTGGCACGAGATGTG | |
| | GCACGAGGGCCTGGAGGAGGCCAGCAGGCTGTATTTTGGCGAGCGCAACGTGAAGGGCA | |
| | TGTTCGAGGTGCTGGAGCCTCTGCACGCCATGATGGAGAGAGGCCCACAGACCCTGAAG | |
| | GAGACATCCTTTAACCAGGCCTATGGACGGGACCTGATGGAGGCACAGGAGTGGTGCAG | |
| | AAAGTACATGAAGTCTGGCAATGTGAAGGACCTGCTGCAGGCCTGGGATCTGTACTATCA | |
| | CGTGTTTCGGAGAATCTCCAAGCCAGCAGCTCTCGGCAAAGACACGATTCCGTGGCTTGG | |
| | GCATCTGCTCGTTGGGCTGAGCGGTGCGTTTGGTTTCATCATCTTGGTCTATCTCTTGATC | |
| | AATTGCAGAAATACAGGCCCTTGGCTGAAAAAAGTGCTCAAGTGTAATACCCCCGACCC | |
| | AAGCAAGTTCTTCTCCCAGCTTTCTTCAGAGCATGGAGGCGATGTGCAGAAATGGCTCTC | |
| | TTCACCTTTTCCCTCCTCAAGCTTCTCCCCGGGAGGGCTGGCGCCCGAGATTTCACCTCTT | |
| | GAGGTACTTGAACGAGACAAGGTTACCCAACTTCTCCTTCAACAGGATAAGGTACCCGA | |
| | ACCTGCGAGCCTTAGCTTGAATACAGACGCTTATCTCTCACTGCAGGAACTGCAAGGATC | |
| | TGGTGCTACTAATTTTTCTCTTTTGAAGCAAGCTGGAGATGTTGAAGAGAACCCCGGTCC | |
| | GGAGATGTGGCATGAGGGTCTGGAAGAAGCGTCTCGACTGTACTTTGGTGAGCGCAATG | |
| | TGAAGGGCATGTTTGAAGTCCTCGAACCCCTTCATGCCATGATGGAACGCGGACCCCAGA | |
| | CCTTGAAGGAGACAAGTTTTAACCAAGCTTACGGAAGAGACCTGATGGAAGCCCAGGAA | |
| | TGGTGCAGGAAATACATGAAAAGCGGGAATGTGAAGGACTTGCTCCAAGCGTGGGACCT | |
| | GTACTATCATGTCTTTAGGCGCATTAGTAAG | |
| 78 | SEQ ID NO: 78: FOXP3cDNA-LNGFRe-μDISC amino acid sequence: | |
| | MPNPRPGKPSAPSLALGPSPGASPSWRAAPKASDLLGARGPGGTFQGRDLRGGAHASSSSLN | |
| | PMPPSQLQLPTLPLVMVAPSGARLGPLPHLQALLQDRPHFMHQLSTVDAHARTPVLQVHPLE | |
| | SPAMISLTPPTTATGVFSLKARPGLPPGINVASLEWVSREPALLCTFPNPSAPRKDSTLSAVPQS | |
| | SYPLLANGVCKWPGCEKVFEEPEDFLKHCQADHLLDEKGRAQCLLQREMVQSLEQQLVLEK | |
| | EKLSAMQAHLAGKMALTKASSVASSDKGSCCIVAAGSQGPVVPAWSGPREAPDSLFAVRRH | |
| | LWGSHGNSTFPEFLHNMDYFKFHNMRPPFTYATLIRWAILEAPEKQRTLNEIYHWFTRMFAF | |
| | FRNHPATWKNAIRHNLSLHKCFVRVESEKGAVWTVDELEFRKKRSQRPSRCSNPTPGPGSGA | |
| | TNFSLLKQAGDVEENPGPMPLGLLWLGLALLGALHAQAMGAGATGRAMDGPRLLLLLLLG | |
| | VSLGGAKEACPTGLYTHSGECCKACNLGEGVAQPCGANQTVCEPCLDSVTFSDVVSATEPC | |
| | KPCTECVGLQSMSAPCVEADDAVCRCAYGYYQDETTGRCEACRVCEAGSGLVFSCQDKQN | |
| | TVCEECPDGTYSDEANHVDPCLPCTVCEDTERQLRECTRWADAECEEIPGRWITRSTPPEGSD | |
| | STAPSTQEPEAPPEQDLIASTVAGVVTTVMGSSQPVVTRGTTDNUPVYCSILAAVVVGLVAYI | |
| | AFKRGVQVETISPGDGRTFPKRGQTCVVHYTGMLEDGKKFDSSRDRNKPFKFMLGKQEVIR | |
| | GWEEGVAQMSVGQRAKLTISPDYAYGATGHPGIIPPHATLVFDVELLKLGEGGSPGSNTSKE | |
| | NPFLFALEAVVISVGSMGLIISLLCVYFWLERTMPRIPTLKNLEDLVTEYHGNFSAWSGVSKG | |
| | LAESLQPDYSERLCLVSEIPPKGGALGEGPGASPCNQHSPYWAPPCYTLKPETGSGATNFSLL | |
| | KQAGDVEENPGPMALPVTALLLPLALLLHAARPILWHEMWHEGLEEASRLYFGERNVKGMF | |
| | EVLEPLHAMMERGPQTLKETSFNQAYGRDLMEAQEWCRKYMKSGNVKDLLQAWDLYYHV | |
| | FRRISKPAALGKDTIPWLGHLLVGLSGAFGFIILVYLLINCRNTGPWLKKVLKCNTPDPSKFFS | |
| | QLSSEHGGDVQKWLSSPFPSSSFSPGGLAPEISPLEVLERDKVTQLLLQQDKVPEPASLSLNTD | |
| | AYLSLQELQGSGATNFSLLKQAGDVEENPGPEMWHEGLEEASRLYFGERNVKGMFEVLEPL | |
| | HAMMERGPQTLKETSFNQAYGRDLMEAQEWCRKYMKSGNVKDLLQAWDLYYHVFRRISK | |
| 79 | ATGCCTCTGGGCCTGCTGTGGCTGGGCCTGGCCCTGCTGGGCGCCCTGCACGCCCAGGCC | μDISC- |
| | GGCGTGCAGGTGGAGACAATCTCCCCAGGCGACGGACGCACATTCCCTAAGCGGGGCCA | FOXP3cDN |
| | GACCTGCGTGGTGCACTATACAGGCATGCTGGAGGATGGCAAGAAGTTTGACAGCTCCC | A nucleotide |
| | GGGATAGAAACAAGCCATTCAAGTTTATGCTGGGCAAGCAGGAAGTGATCAGAGGCTGG | sequence |
| | GAGGAGGGCGTGGCCCAGATGTCTGTGGGCCAGAGGGCCAAGCTGACCATCAGCCCAGA | (coding |
| | CTACGCCTATGGAGCAACAGGCCACCCAGGAATCATCCCACCTCACGCCACCCTGGTGTT | sequence |
| | CGATGTGGAGCTGCTGAAGCTGGGCGAGGGAGGGTCACCTGGATCCAACACATCAAAAG | only; codon- |
| | AGAACCCCTTTCTGTTCGCATTGGAGGCCGTAGTCATATCTGTTGGATCCATGGGACTTAT | optimized; |
| | TATCTCCCTGTTGTGTGTGTACTTCTGGCTGGAACGGACTATGCCCAGGATCCCCACGCTC | our DISC |
| | AAGAATCTGGAAGATCTCGTCACAGAATACCATGGTAATTTCAGCGCCTGGAGCGGAGT | architecture |
| | CTCTAAGGGTCTGGCCGAATCCCTCCAACCCGATTATTCTGAACGGTTGTGCCTCGTATCC | version 6) |
| | GAAATACCACCAAAAGGCGGGGCTCTGGGTGAGGGCCCAGGGGCGAGTCCGTGCAATCA | |
| | ACACAGCCCGTATTGGGCCCCTCCTTGTTATACGTTGAAGCCCGAAACTGGAAGCGGAGC | |
| | TACTAACTTCAGCCTGCTGAAGCAGGCTGGAGACGTGGAGGAGAACCCTGGACCTATGG | |
| | CACTGCCCGTGACCGCCCTGCTGCTGCCTCTGGCCCTGCTGCTGCACGCAGCCCGGCCTA | |
| | TCCTGTGGCACGAGATGTGGCACGAGGGCCTGGAGGAGGCCAGCAGGCTGTATTTTGGC | |
| | GAGCGCAACGTGAAGGGCATGTTCGAGGTGCTGGAGCCTCTGCACGCCATGATGGAGAG | |
| | AGGCCCACAGACCCTGAAGGAGACATCCTTTAACCAGGCCTATGGACGGGACCTGATGG | |
| | AGGCACAGGAGTGGTGCAGAAAGTACATGAAGTCTGGCAATGTGAAGGACCTGCTGCAG | |
| | GCCTGGGATCTGTACTATCACGTGTTTCGGAGAATCTCCAAGCCAGCAGCTCTCGGCAAA | |
| | GACACGATTCCGTGGCTTGGGCATCTGCTCGTTGGGCTGAGCGGTGCGTTTGGTTTCATC | |
| | ATCTTGGTCTATCTCTTGATCAATTGCAGAAATACAGGCCCTTGGCTGAAAAAAGTGCTC | |
| | AAGTGTAATACCCCCGACCCAAGCAAGTTCTTCTCCCAGCTTTCTTCAGAGCATGGAGGC | |
| | GATGTGCAGAAATGGCTCTCTTCACCTTTTCCCTCCTCAAGCTTCTCCCCGGGAGGGCTGG | |
| | CGCCCGAGATTTCACCTCTTGAGGTACTTGAACGAGACAAGGTTACCCAACTTCTCCTTC | |
| | AACAGGATAAGGTACCCGAACCTGCGAGCCTTAGCTTGAATACAGACGCTTATCTCTCAC | |
| | TGCAGGAACTGCAAGGATCTGGTGCTACTAATTTTTCTCTTTTGAAGCAAGCTGGAGATG | |
| | TTGAAGAGAACCCCGGTCCGGAGATGTGGCATGAGGGTCTGGAAGAAGCGTCTCGACTG | |
| | TACTTTGGTGAGCGCAATGTGAAGGGCATGTTTGAAGTCCTCGAACCCCTTCATGCCATG | |
| | ATGGAACGCGGACCCCAGACCTTGAAGGAGACAAGTTTTAACCAAGCTTACGGAAGAGA | |
| | CCTGATGGAAGCCCAGGAATGGTGCAGGAAATACATGAAAAGCGGGAATGTGAAGGACT | |
| | TGCTCCAAGCGTGGGACCTGTACTATCATGTCTTTAGGCGCATTAGTAAGGGAAGCGGAG | |
| | CGACTAACTTCAGCCTGCTTAAGCAGGCCGGAGATGTGGAGGAAAACCCTGGACCGATG | |
| | CCTAATCCTCGGCCTGGAAAGCCTAGCGCTCCTTCTCTTGCTCTGGGACCTTCTCCTGGCG | |
| | CCTCTCCATCTTGGAGAGCCGCTCCTAAAGCCAGCGATCTGCTGGGAGCTAGAGGACCTG | |
| | GCGGCACATTTCAGGGCAGAGATCTTAGAGGCGGAGCCCACGCTAGCTCCTCCAGCCTTA | |
| | ATCCTATGCCTCCTAGCCAGCTCCAGCTGCCTACACTGCCTCTGGTTATGGTGGCTCCTAG | |
| | CGGAGCTAGACTGGGCCCTCTGCCTCATCTGCAAGCTCTGCTGCAGGACAGACCCCACTT | |
| | CATGCACCAGCTGAGCACCGTGGATGCCCACGCAAGAACACCTGTGCTGCAGGTTCACC | |
| | CTCTGGAATCCCCAGCCATGATCAGCCTGACACCTCCAACAACAGCCACCGGCGTGTTCA | |
| | GCCTGAAAGCCAGACCTGGACTGCCTCCTGGCATCAATGTGGCCAGCCTGGAATGGGTGT | |
| | CCAGAGAACCTGCTCTGCTGTGCACATTCCCCAATCCAAGCGCTCCCAGAAAGGACAGC | |
| | ACACTGTCTGCCGTGCCTCAGAGCAGCTATCCCCTGCTTGCTAACGGCGTGTGCAAGTGG | |
| | CCTGGATGCGAGAAGGTGTTCGAGGAACCCGAGGACTTCCTGAAGCACTGCCAGGCCGA | |
| | TCATCTGCTGGACGAGAAAGGCAGAGCCCAGTGTCTGCTCCAGCGCGAGATGGTGCAGT | |
| | CTCTGGAACAGCAGCTGGTCCTGGAAAAAGAAAAGCTGAGCGCCATGCAGGCCCACCTG | |
| | GCCGGAAAAATGGCCCTGACAAAGGCCAGCAGCGTGGCCTCTTCTGATAAGGGCAGCTG | |
| | CTGCATTGTGGCCGCTGGATCTCAGGGACCTGTGGTTCCTGCTTGGAGCGGACCTAGAGA | |
| | GGCCCCTGATTCTCTGTTTGCCGTGCGGAGACACCTGTGGGGCTCTCACGGCAACTCTAC | |
| | TTTCCCCGAGTTCCTGCACAACATGGACTACTTCAAGTTCCACAACATGCGGCCTCCATTC | |
| | ACCTACGCCACACTGATCAGATGGGCCATTCTGGAAGCCCCTGAGAAGCAGAGAACCCT | |
| | GAACGAGATCTACCACTGGTTTACCCGGATGTTCGCCTTCTTCCGGAATCACCCTGCCAC | |
| | CTGGAAGAACGCCATCCGGCACAATCTGAGCCTGCACAAGTGCTTCGTGCGCGTGGAAT | |
| | CTGAGAAAGGCGCCGTGTGGACAGTGGACGAGCTGGAATTCAGAAAGAAGAGAAGCCA | |
| | GCGGCCTAGCCGGTGCAGCAATCCTACACCTGGACCT | |
| 80 | MPLGLLWLGLALLGALHAQAGVQVETISPGDGRTFPKRGQTCVVHYTGMLEDGKKFDSSRD | μDISC- |
| | RNKPFKFMLGKQEVIRGWEEGVAQMSVGQRAKLTISPDYAYGATGHPGIIPPHATLVFDVEL | FOXP3cDN |
| | LKLGEGGSPGSNTSKENPFLFALEAVVISVGSMGLIISLLCVYFWLERTMPRIPTLKNLEDLVT | A amino acid |
| | EYHGNFSAWSGVSKGLAESLQPDYSERLCLVSEIPPKGGALGEGPGASPCNQHSPYWAPPCY | sequence |
| | TLKPETGSGATNFSLLKQAGDVEENPGPMALPVTALLLPLALLLHAARPILWHEMWHEGLEE | |
| | ASRLYFGERNVKGMFEVLEPLHAMMERGPQTLKETSFNQAYGRDLMEAQEWCRKYMKSG | |
| | NVKDLLQAWDLYYHVFRRISKPAALGKDTIPWLGHLLVGLSGAFGFIILVYLLINCRNTGPWL | |
| | KKVLKCNTPDPSKFFSQLSSEHGGDVQKWLSSPFPSSSFSPGGLAPEISPLEVLERDKVTQLLL | |
| | QQDKVPEPASLSLNTDAYLSLQELQGSGATNFSLLKQAGDVEENPGPEMWHEGLEEASRLYF | |
| | GERNVKGMFEVLEPLHAMMERGPQTLKETSFNQAYGRDLMEAQEWCRKYMKSGNVKDLL | |
| | QAWDLYYHVFRRISKGSGATNFSLLKQAGDVEENPGPMPNPRPGKPSAPSLALGPSPGASPS | |
| | WRAAPKASDLLGARGPGGTFQGRDLRGGAHASSSSLNPMPPSQLQLPTLPLVMVAPSGARL | |
| | GPLPHLQALLQDRPHFMHQLSTVDAHARTPVLQVHPLESPAMISLTPPTTATGVFSLKARPGL | |
| | PPGINVASLEWVSREPALLCTFPNPSAPRKDSTLSAVPQSSYPLLANGVCKWPGCEKVFEEPE | |
| | DFLKHCQADHLLDEKGRAQCLLQREMVQSLEQQLVLEKEKLSAMQAHLAGKMALTKASSV | |
| | ASSDKGSCCIVAAGSQGPVVPAWSGPREAPDSLFAVRRHLWGSHGNSTFPEFLHNMDYFKFH | |
| | NMRPPFTYATLIRWAILEAPEKQRTLNEIYHWFTRMFAFFRNHPATWKNAIRHNLSLHKCFV | |
| | RVESEKGAVWTVDELEFRKKRSQRPSRCSNPTPGP | |
| 81 | ATGCCTCTGGGCCTGCTGTGGCTGGGCCTGGCCCTGCTGGGCGCCCTGCACGCCCAGGCC | LNGFRe- |
| | ATGGGGGCAGGTGCCACCGGACGAGCCATGGACGGGCCGCGCCTGCTGCTGTTGCTGCT | μDISC - |
| | TCTGGGGGTGTCCCTTGGAGGTGCCAAGGAGGCATGCCCCACAGGCCTGTACACACACA | FOXP3cDN |
| | GCGGTGAGTGCTGCAAAGCCTGCAACCTGGGCGAGGGTGTGGCCCAGCCTTGTGGAGCC | A nucleotide |
| | AACCAGACCGTGTGTGAGCCCTGCCTGGACAGCGTGACGTTCTCCGACGTGGTGAGCGC | sequence |
| | GACCGAGCCGTGCAAGCCGTGCACCGAGTGCGTGGGGCTCCAGAGCATGTCGGCGCCGT | (coding |
| | GCGTGGAGGCCGACGACGCCGTGTGCCGCTGCGCCTACGGCTACTACCAGGATGAGACG | sequence |
| | ACTGGGCGCTGCGAGGCGTGCCGCGTGTGCGAGGCGGGCTCGGGCCTCGTGTTCTCCTGC | only; codon- |
| | CAGGACAAGCAGAACACCGTGTGCGAGGAGTGCCCCGACGGCACGTATTCCGACGAGGC | optimized; |
| | CAACCACGTGGACCCGTGCCTGCCCTGCACCGTGTGCGAGGACACCGAGCGCCAGCTCC | our DISC |
| | GCGAGTGCACACGCTGGGCCGACGCCGAGTGCGAGGAGATCCCTGGCCGTTGGATTACA | architecture |
| | CGGTCCACACCCCCAGAGGGCTCGGACAGCACAGCCCCCAGCACCCAGGAGCCTGAGGC | version 6) |
| | ACCTCCAGAACAAGACCTCATAGCCAGCACGGTGGCAGGTGTGGTGACCACAGTGATGG | |
| | GCAGCTCCCAGCCCGTGGTGACCCGAGGCACCACCGACAACCTCATCCCTGTCTATTGCT | |
| | CCATCCTGGCTGCTGTGGTTGTGGGTCTTGTGGCCTACATAGCCTTCAAGAGGGGCGTGC | |
| | AGGTGGAGACAATCTCCCCAGGCGACGGACGCACATTCCCTAAGCGGGGCCAGACCTGC | |
| | GTGGTGCACTATACAGGCATGCTGGAGGATGGCAAGAAGTTTGACAGCTCCCGGGATAG | |
| | AAACAAGCCATTCAAGTTTATGCTGGGCAAGCAGGAAGTGATCAGAGGCTGGGAGGAGG | |
| | GCGTGGCCCAGATGTCTGTGGGCCAGAGGGCCAAGCTGACCATCAGCCCAGACTACGCC | |
| | TATGGAGCAACAGGCCACCCAGGAATCATCCCACCTCACGCCACCCTGGTGTTCGATGTG | |
| | GAGCTGCTGAAGCTGGGCGAGGGAGGGTCACCTGGATCCAACACATCAAAAGAGAACCC | |
| | CTTTCTGTTCGCATTGGAGGCCGTAGTCATATCTGTTGGATCCATGGGACTTATTATCTCC | |
| | CTGTTGTGTGTGTACTTCTGGCTGGAACGGACTATGCCCAGGATCCCCACGCTCAAGAAT | |
| | CTGGAAGATCTCGTCACAGAATACCATGGTAATTTCAGCGCCTGGAGCGGAGTCTCTAAG | |
| | GGTCTGGCCGAATCCCTCCAACCCGATTATTCTGAACGGTTGTGCCTCGTATCCGAAATA | |
| | CCACCAAAAGGCGGGGCTCTGGGTGAGGGCCCAGGGGCGAGTCCGTGCAATCAACACAG | |
| | CCCGTATTGGGCCCCTCCTTGTTATACGTTGAAGCCCGAAACTGGAAGCGGAGCTACTAA | |
| | CTTCAGCCTGCTGAAGCAGGCTGGAGACGTGGAGGAGAACCCTGGACCTATGGCACTGC | |
| | CCGTGACCGCCCTGCTGCTGCCTCTGGCCCTGCTGCTGCACGCAGCCCGGCCTATCCTGT | |
| | GGCACGAGATGTGGCACGAGGGCCTGGAGGAGGCCAGCAGGCTGTATTTTGGCGAGCGC | |
| | AACGTGAAGGGCATGTTCGAGGTGCTGGAGCCTCTGCACGCCATGATGGAGAGAGGCCC | |
| | ACAGACCCTGAAGGAGACATCCTTTAACCAGGCCTATGGACGGGACCTGATGGAGGCAC | |
| | AGGAGTGGTGCAGAAAGTACATGAAGTCTGGCAATGTGAAGGACCTGCTGCAGGCCTGG | |
| | GATCTGTACTATCACGTGTTTCGGAGAATCTCCAAGCCAGCAGCTCTCGGCAAAGACACG | |
| | ATTCCGTGGCTTGGGCATCTGCTCGTTGGGCTGAGCGGTGCGTTTGGTTTCATCATCTTGG | |
| | TCTATCTCTTGATCAATTGCAGAAATACAGGCCCTTGGCTGAAAAAAGTGCTCAAGTGTA | |
| | ATACCCCCGACCCAAGCAAGTTCTTCTCCCAGCTTTCTTCAGAGCATGGAGGCGATGTGC | |
| | AGAAATGGCTCTCTTCACCTTTTCCCTCCTCAAGCTTCTCCCCGGGAGGGCTGGCGCCCG | |
| | AGATTTCACCTCTTGAGGTACTTGAACGAGACAAGGTTACCCAACTTCTCCTTCAACAGG | |
| | ATAAGGTACCCGAACCTGCGAGCCTTAGCTTGAATACAGACGCTTATCTCTCACTGCAGG | |
| | AACTGCAAGGATCTGGTGCTACTAATTTTTCTCTTTTGAAGCAAGCTGGAGATGTTGAAG | |
| | AGAACCCCGGTCCGGAGATGTGGCATGAGGGTCTGGAAGAAGCGTCTCGACTGTACTTT | |
| | GGTGAGCGCAATGTGAAGGGCATGTTTGAAGTCCTCGAACCCCTTCATGCCATGATGGAA | |
| | CGCGGACCCCAGACCTTGAAGGAGACAAGTTTTAACCAAGCTTACGGAAGAGACCTGAT | |
| | GGAAGCCCAGGAATGGTGCAGGAAATACATGAAAAGCGGGAATGTGAAGGACTTGCTCC | |
| | AAGCGTGGGACCTGTACTATCATGTCTTTAGGCGCATTAGTAAGGGAAGCGGAGCGACT | |
| | AACTTCAGCCTGCTTAAGCAGGCCGGAGATGTGGAGGAAAACCCTGGACCGATGCCTAA | |
| | TCCTCGGCCTGGAAAGCCTAGCGCTCCTTCTCTTGCTCTGGGACCTTCTCCTGGCGCCTCT | |
| | CCATCTTGGAGAGCCGCTCCTAAAGCCAGCGATCTGCTGGGAGCTAGAGGACCTGGCGG | |
| | CACATTTCAGGGCAGAGATCTTAGAGGCGGAGCCCACGCTAGCTCCTCCAGCCTTAATCC | |
| | TATGCCTCCTAGCCAGCTCCAGCTGCCTACACTGCCTCTGGTTATGGTGGCTCCTAGCGG | |
| | AGCTAGACTGGGCCCTCTGCCTCATCTGCAAGCTCTGCTGCAGGACAGACCCCACTTCAT | |
| | GCACCAGCTGAGCACCGTGGATGCCCACGCAAGAACACCTGTGCTGCAGGTTCACCCTCT | |
| | GGAATCCCCAGCCATGATCAGCCTGACACCTCCAACAACAGCCACCGGCGTGTTCAGCCT | |
| | GAAAGCCAGACCTGGACTGCCTCCTGGCATCAATGTGGCCAGCCTGGAATGGGTGTCCA | |
| | GAGAACCTGCTCTGCTGTGCACATTCCCCAATCCAAGCGCTCCCAGAAAGGACAGCACA | |
| | CTGTCTGCCGTGCCTCAGAGCAGCTATCCCCTGCTTGCTAACGGCGTGTGCAAGTGGCCT | |
| | GGATGCGAGAAGGTGTTCGAGGAACCCGAGGACTTCCTGAAGCACTGCCAGGCCGATCA | |
| | TCTGCTGGACGAGAAAGGCAGAGCCCAGTGTCTGCTCCAGCGCGAGATGGTGCAGTCTC | |
| | TGGAACAGCAGCTGGTCCTGGAAAAAGAAAAGCTGAGCGCCATGCAGGCCCACCTGGCC | |
| | GGAAAAATGGCCCTGACAAAGGCCAGCAGCGTGGCCTCTTCTGATAAGGGCAGCTGCTG | |
| | CATTGTGGCCGCTGGATCTCAGGGACCTGTGGTTCCTGCTTGGAGCGGACCTAGAGAGGC | |
| | CCCTGATTCTCTGTTTGCCGTGCGGAGACACCTGTGGGGCTCTCACGGCAACTCTACTTTC | |
| | CCCGAGTTCCTGCACAACATGGACTACTTCAAGTTCCACAACATGCGGCCTCCATTCACC | |
| | TACGCCACACTGATCAGATGGGCCATTCTGGAAGCCCCTGAGAAGCAGAGAACCCTGAA | |
| | CGAGATCTACCACTGGTTTACCCGGATGTTCGCCTTCTTCCGGAATCACCCTGCCACCTGG | |
| | AAGAACGCCATCCGGCACAATCTGAGCCTGCACAAGTGCTTCGTGCGCGTGGAATCTGA | |
| | GAAAGGCGCCGTGTGGACAGTGGACGAGCTGGAATTCAGAAAGAAGAGAAGCCAGCGG | |
| | CCTAGCCGGTGCAGCAATCCTACACCTGGACCTTGA | |
| 82 | MPLGLLWLGLALLGALHAQAMGAGATGRAMDGPRLLLLLLLGVSLGGAKEACPTGLYTHS | LNGFRe- |
| | GECCKACNLGEGVAQPCGANQTVCEPCLDSVTFSDVVSATEPCKPCTECVGLQSMSAPCVE | μDISC- |
| | ADDAVCRCAYGYYQDETTGRCEACRVCEAGSGLVFSCQDKQNTVCEECPDGTYSDEANHV | FOXP3cDN |
| | DPCLPCTVCEDTERQLRECTRWADAECEEIPGRWITRSTPPEGSDSTAPSTQEPEAPPEQDLIA | A amino acid |
| | STVAGVVTTVMGSSQPVVTRGTTDNLIPVYCSILAAVVVGLVAYIAFKRGVQVETISPGDGRT | sequence |
| | FPKRGQTCVVHYTGMLEDGKKFDSSRDRNKPFKFMLGKQEVIRGWEEGVAQMSVGQRAKL | |
| | TISPDYAYGATGHPGIIPPHATLVFDVELLKLGEGGSPGSNTSKENPFLFALEAVVISVGSMGLI | |
| | ISLLCVYFWLERTMPRIPTLKNLEDLVTEYHGNFSAWSGVSKGLAESLQPDYSERLCLVSEIPP | |
| | KGGALGEGPGASPCNQHSPYWAPPCYTLKPETGSGATNFSLLKQAGDVEENPGPMALPVTA | |
| | LLLPLALLLHAARPILWHEMWHEGLEEASRLYFGERNVKGMFEVLEPLHAMMERGPQTLKE | |
| | TSFNQAYGRDLMEAQEWCRKYMKSGNVKDLLQAWDLYYHVFRRISKPAALGKDTIPWLGH | |
| | LLVGLSGAFGFIILVYLLINCRNTGPWLKKVLKCNTPDPSKFFSQLSSEHGGDVQKWLSSPFPS | |
| | SSFSPGGLAPEISPLEVLERDKVTQLLLQQDKVPEPASLSLNTDAYLSLQELQGSGATNFSLLK | |
| | QAGDVEENPGPEMWHEGLEEASRLYFGERNVKGMFEVLEPLHAMMERGPQTLKETSFNQA | |
| | YGRDLMEAQEWCRKYMKSGNVKDLLQAWDLYYHVFRRISKGSGATNFSLLKQAGDVEENP | |
| | GPMPNPRPGKPSAPSLALGPSPGASPSWRAAPKASDLLGARGPGGTFQGRDLRGGAHASSSS | |
| | LNPMPPSQLQLPTLPLVMVAPSGARLGPLPHLQALLQDRPHFMHQLSTVDAHARTPVLQVHP | |
| | LESPAMISLTPPTTATGVFSLKARPGLPPGINVASLEWVSREPALLCTFPNPSAPRKDSTLSAVP | |
| | QSSYPLLANGVCKWPGCEKVFEEPEDFLKHCQADHLLDEKGRAQCLLQREMVQSLEQQLVL | |
| | EKEKLSAMQAHLAGKMALTKASSVASSDKGSCCIVAAGSQGPVVPAWSGPREAPDSLFAVR | |
| | RHLWGSHGNSTFPEFLHNMDYFKFHNMRPPFTYATLIRWAILEAPEKQRTLNEIYHWFTRMF | |
| | AFFRNHPATWKNAIRHNLSLHKCFVRVESEKGAVWTVDELEFRKKRSQRPSRCSNPTPGP* | |
| 83 | ATGCCTCTGGGCCTGCTGTGGCTGGGCCTGGCCCTGCTGGGCGCCCTGCACGCCCAGGCC | DISC |
| | GGCGTGCAGGTGGAGACAATCTCCCCAGGCGACGGACGCACATTCCCTAAGCGGGGCCA | nucleotide |
| | GACCTGCGTGGTGCACTATACAGGCATGCTGGAGGATGGCAAGAAGTTTGACAGCTCCC | sequence |
| | GGGATAGAAACAAGCCATTCAAGTTTATGCTGGGCAAGCAGGAAGTGATCAGAGGCTGG | (coding |
| | GAGGAGGGCGTGGCCCAGATGTCTGTGGGCCAGAGGGCCAAGCTGACCATCAGCCCAGA | sequence |
| | CTACGCCTATGGAGCAACAGGCCACCCAGGAATCATCCCACCTCACGCCACCCTGGTGTT | only; codon- |
| | CGATGTGGAGCTGCTGAAGCTGGGCGAGGGAGGGTCACCTGGATCCAACACATCAAAAG | optimized; |
| | AGAACCCCTTTCTGTTCGCATTGGAGGCCGTAGTCATATCTGTTGGATCCATGGGACTTAT | our DISC |
| | TATCTCCCTGTTGTGTGTGTACTTCTGGCTGGAACGGACTATGCCCAGGATCCCCACGCTC | architecture |
| | AAGAATCTGGAAGATCTCGTCACAGAATACCATGGTAATTTCAGCGCCTGGAGCGGAGT | version 6) |
| | CTCTAAGGGTCTGGCCGAATCCCTCCAACCCGATTATTCTGAACGGTTGTGCCTCGTATCC | |
| | GAAATACCACCAAAAGGCGGGGCTCTGGGTGAGGGCCCAGGGGCGAGTCCGTGCAATCA | |
| | ACACAGCCCGTATTGGGCCCCTCCTTGTTATACGTTGAAGCCCGAAACTGGAAGCGGAGC | |
| | TACTAACTTCAGCCTGCTGAAGCAGGCTGGAGACGTGGAGGAGAACCCTGGACCTATGG | |
| | CACTGCCCGTGACCGCCCTGCTGCTGCCTCTGGCCCTGCTGCTGCACGCAGCCCGGCCTA | |
| | TCCTGTGGCACGAGATGTGGCACGAGGGCCTGGAGGAGGCCAGCAGGCTGTATTTTGGC | |
| | GAGCGCAACGTGAAGGGCATGTTCGAGGTGCTGGAGCCTCTGCACGCCATGATGGAGAG | |
| | AGGCCCACAGACCCTGAAGGAGACATCCTTTAACCAGGCCTATGGACGGGACCTGATGG | |
| | AGGCACAGGAGTGGTGCAGAAAGTACATGAAGTCTGGCAATGTGAAGGACCTGCTGCAG | |
| | GCCTGGGATCTGTACTATCACGTGTTTCGGAGAATCTCCAAGCCAGCAGCTCTCGGCAAA | |
| | GACACGATTCCGTGGCTTGGGCATCTGCTCGTTGGGCTGAGCGGTGCGTTTGGTTTCATC | |
| | ATCTTGGTCTATCTCTTGATCAATTGCAGAAATACAGGCCCTTGGCTGAAAAAAGTGCTC | |
| | AAGTGTAATACCCCCGACCCAAGCAAGTTCTTCTCCCAGCTTTCTTCAGAGCATGGAGGC | |
| | GATGTGCAGAAATGGCTCTCTTCACCTTTTCCCTCCTCAAGCTTCTCCCCGGGAGGGCTGG | |
| | CGCCCGAGATTTCACCTCTTGAGGTACTTGAACGAGACAAGGTTACCCAACTTCTCCTTC | |
| | AACAGGATAAGGTACCCGAACCTGCGAGCCTTAGCTCCAACCACTCTCTTACGAGCTGCT | |
| | TCACCAATCAGGGATACTTCTTTTTCCACCTTCCCGATGCGCTGGAAATCGAAGCTTGTCA | |
| | AGTTTACTTTACCTATGATCCATATAGCGAGGAAGATCCCGACGAAGGAGTCGCCGGTGC | |
| | GCCCACGGGTTCCTCACCCCAACCTCTCCAGCCTCTCTCAGGAGAAGATGATGCTTATTG | |
| | CACTTTTCCCAGTAGAGACGATCTCCTCCTCTTTTCTCCATCTCTTTTGGGGGGACCTTCC | |
| | CCCCCTTCTACGGCACCTGGCGGGTCTGGTGCTGGCGAGGAGCGGATGCCGCCGTCCCTC | |
| | CAGGAGCGAGTACCACGAGATTGGGATCCCCAGCCACTTGGACCCCCCACCCCCGGCGT | |
| | ACCTGACCTTGTCGATTTTCAACCTCCCCCTGAATTGGTGCTGCGAGAGGCTGGGGAGGA | |
| | AGTTCCGGACGCTGGGCCGAGGGAGGGCGTGTCCTTTCCATGGAGTAGGCCTCCAGGTC | |
| | AAGGCGAGTTTAGGGCTCTCAACGCGCGGCTGCCGTTGAATACAGACGCTTATCTCTCAC | |
| | TGCAGGAACTGCAAGGTCAGGACCCAACACATCTTGTAGGATCTGGTGCTACTAATTTTT | |
| | CTCTTTTGAAGCAAGCTGGAGATGTTGAAGAGAACCCCGGTCCGGAGATGTGGCATGAG | |
| | GGTCTGGAAGAAGCGTCTCGACTGTACTTTGGTGAGCGCAATGTGAAGGGCATGTTTGAA | |
| | GTCCTCGAACCCCTTCATGCCATGATGGAACGCGGACCCCAGACCTTGAAGGAGACAAG | |
| | TTTTAACCAAGCTTACGGAAGAGACCTGATGGAAGCCCAGGAATGGTGCAGGAAATACA | |
| | TGAAAAGCGGGAATGTGAAGGACTTGCTCCAAGCGTGGGACCTGTACTATCATGTCTTTA | |
| | GGCGCATTAGTAAG | |
| 84 | MPLGLLWLGLALLGALHAQAGVQVETISPGDGRTFPKRGQTCVVHYTGMLEDGKKFDSSRD | DISC amino |
| | RNKPFKFMLGKQEVIRGWEEGVAQMSVGQRAKLTISPDYAYGATGHPGIIPPHATLVFDVEL | acid |
| | LKLGEGGSPGSNTSKENPFLFALEAVVISVGSMGLIISLLCVYFWLERTMPRIPTLKNLEDLVT | sequence |
| | EYHGNFSAWSGVSKGLAESLQPDYSERLCLVSEIPPKGGALGEGPGASPCNQHSPYWAPPCY | |
| | TLKPETGSGATNFSLLKQAGDVEENPGPMALPVTALLLPLALLLHAARPILWHEMWHEGLEE | |
| | ASRLYFGERNVKGMFEVLEPLHAMMERGPQTLKETSFNQAYGRDLMEAQEWCRKYMKSG | |
| | NVKDLLQAWDLYYHVFRRISKPAALGKDTIPWLGHLLVGLSGAFGFIILVYLLINCRNTGPWL | |
| | KKVLKCNTPDPSKFFSQLSSEHGGDVQKWLSSPFPSSSFSPGGLAPEISPLEVLERDKVTQLLL | |
| | QQDKVPEPASLSSNHSLTSCFTNQGYFFFHLPDALEIEACQVYFTYDPYSEEDPDEGVAGAPT | |
| | GSSPQPLQPLSGEDDAYCTFPSRDDLLLFSPSLLGGPSPPSTAPGGSGAGEERMPPSLQERVPR | |
| | DWDPQPLGPPTPGVPDLVDFQPPPELVLREAGEEVPDAGPREGVSFPWSRPPGQGEFRALNA | |
| | RLPLNTDAYLSLQELQGQDPTHLVGSGATNFSLLKQAGDVEENPGPEMWHEGLEEASRLYF | |
| | GERNVKGMFEVLEPLHAMMERGPQTLKETSFNQAYGRDLMEAQEWCRKYMKSGNVKDLL | |
| | QAWDLYYHVFRRISK | |
| 85 | ATGCCTCTGGGCCTGCTGTGGCTGGGCCTGGCCCTGCTGGGCGCCCTGCACGCCCAGGCC | μDISC |
| | GGCGTGCAGGTGGAGACAATCTCCCCAGGCGACGGACGCACATTCCCTAAGCGGGGCCA | nucleotide |
| | GACCTGCGTGGTGCACTATACAGGCATGCTGGAGGATGGCAAGAAGTTTGACAGCTCCC | sequence |
| | GGGATAGAAACAAGCCATTCAAGTTTATGCTGGGCAAGCAGGAAGTGATCAGAGGCTGG | (coding |
| | GAGGAGGGCGTGGCCCAGATGTCTGTGGGCCAGAGGGCCAAGCTGACCATCAGCCCAGA | sequence |
| | CTACGCCTATGGAGCAACAGGCCACCCAGGAATCATCCCACCTCACGCCACCCTGGTGTT | only; codon- |
| | CGATGTGGAGCTGCTGAAGCTGGGCGAGGGAGGGTCACCTGGATCCAACACATCAAAAG | optimized; |
| | AGAACCCCTTTCTGTTCGCATTGGAGGCCGTAGTCATATCTGTTGGATCCATGGGACTTAT | our DISC |
| | TATCTCCCTGTTGTGTGTGTACTTCTGGCTGGAACGGACTATGCCCAGGATCCCCACGCTC | architecture |
| | AAGAATCTGGAAGATCTCGTCACAGAATACCATGGTAATTTCAGCGCCTGGAGCGGAGT | version 6) |
| | CTCTAAGGGTCTGGCCGAATCCCTCCAACCCGATTATTCTGAACGGTTGTGCCTCGTATCC | |
| | GAAATACCACCAAAAGGCGGGGCTCTGGGTGAGGGCCCAGGGGCGAGTCCGTGCAATCA | |
| | ACACAGCCCGTATTGGGCCCCTCCTTGTTATACGTTGAAGCCCGAAACTGGAAGCGGAGC | |
| | TACTAACTTCAGCCTGCTGAAGCAGGCTGGAGACGTGGAGGAGAACCCTGGACCTATGG | |
| | CACTGCCCGTGACCGCCCTGCTGCTGCCTCTGGCCCTGCTGCTGCACGCAGCCCGGCCTA | |
| | TCCTGTGGCACGAGATGTGGCACGAGGGCCTGGAGGAGGCCAGCAGGCTGTATTTTGGC | |
| | GAGCGCAACGTGAAGGGCATGTTCGAGGTGCTGGAGCCTCTGCACGCCATGATGGAGAG | |
| | AGGCCCACAGACCCTGAAGGAGACATCCTTTAACCAGGCCTATGGACGGGACCTGATGG | |
| | AGGCACAGGAGTGGTGCAGAAAGTACATGAAGTCTGGCAATGTGAAGGACCTGCTGCAG | |
| | GCCTGGGATCTGTACTATCACGTGTTTCGGAGAATCTCCAAGCCAGCAGCTCTCGGCAAA | |
| | GACACGATTCCGTGGCTTGGGCATCTGCTCGTTGGGCTGAGCGGTGCGTTTGGTTTCATC | |
| | ATCTTGGTCTATCTCTTGATCAATTGCAGAAATACAGGCCCTTGGCTGAAAAAAGTGCTC | |
| | AAGTGTAATACCCCCGACCCAAGCAAGTTCTTCTCCCAGCTTTCTTCAGAGCATGGAGGC | |
| | GATGTGCAGAAATGGCTCTCTTCACCTTTTCCCTCCTCAAGCTTCTCCCCGGGAGGGCTGG | |
| | CGCCCGAGATTTCACCTCTTGAGGTACTTGAACGAGACAAGGTTACCCAACTTCTCCTTC | |
| | AACAGGATAAGGTACCCGAACCTGCGAGCCTTAGCTTGAATACAGACGCTTATCTCTCAC | |
| | TGCAGGAACTGCAAGGATCTGGTGCTACTAATTTTTCTCTTTTGAAGCAAGCTGGAGATG | |
| | TTGAAGAGAACCCCGGTCCGGAGATGTGGCATGAGGGTCTGGAAGAAGCGTCTCGACTG | |
| | TACTTTGGTGAGCGCAATGTGAAGGGCATGTTTGAAGTCCTCGAACCCCTTCATGCCATG | |
| | ATGGAACGCGGACCCCAGACCTTGAAGGAGACAAGTTTTAACCAAGCTTACGGAAGAGA | |
| | CCTGATGGAAGCCCAGGAATGGTGCAGGAAATACATGAAAAGCGGGAATGTGAAGGACT | |
| | TGCTCCAAGCGTGGGACCTGTACTATCATGTCTTTAGGCGCATTAGTAAG | |
| 86 | MPLGLLWLGLALLGALHAQAGVQVETISPGDGRTFPKRGQTCVVHYTGMLEDGKKFDSSRD | μDISC |
| | RNKPFKFMLGKQEVIRGWEEGVAQMSVGQRAKLTISPDYAYGATGHPGIIPPHATLVFDVEL | amino acid |
| | LKLGEGGSPGSNTSKENPFLFALEAVVISVGSMGLIISLLCVYFWLERTMPRIPTLKNLEDLVT | sequence |
| | EYHGNFSAWSGVSKGLAESLQPDYSERLCLVSEIPPKGGALGEGPGASPCNQHSPYWAPPCY | |
| | TLKPETGSGATNFSLLKQAGDVEENPGPMALPVTALLLPLALLLHAARPILWHEMWHEGLEE | |
| | ASRLYFGERNVKGMFEVLEPLHAMMERGPQTLKETSFNQAYGRDLMEAQEWCRKYMKSG | |
| | NVKDLLQAWDLYYHVFRRISKPAALGKDTIPWLGHLLVGLSGAFGFIILVYLLINCRNTGPWL | |
| | KKVLKCNTPDPSKFFSQLSSEHGGDVQKWLSSPFPSSSFSPGGLAPEISPLEVLERDKVTQLLL | |
| | QQDKVPEPASLSLNTDAYLSLQELQGSGATNFSLLKQAGDVEENPGPEMWHEGLEEASRLYF | |
| | GERNVKGMFEVLEPLHAMMERGPQTLKETSFNQAYGRDLMEAQEWCRKYMKSGNVKDLL | |
| | QAWDLYYHVFRRISK | |
| 87 | ATGGCACTGCCCGTGACCGCCCTGCTGCTGCCTCTGGCCCTGCTGCTGCACGCAGCCCGG | CISCβ-DN |
| | CCTATCCTGTGGCACGAGATGTGGCACGAGGGCCTGGAGGAGGCCAGCAGGCTGTATTT | nucleotide |
| | TGGCGAGCGCAACGTGAAGGGCATGTTCGAGGTGCTGGAGCCTCTGCACGCCATGATGG | sequence |
| | AGAGAGGCCCACAGACCCTGAAGGAGACATCCTTTAACCAGGCCTATGGACGGGACCTG | (coding |
| | ATGGAGGCACAGGAGTGGTGCAGAAAGTACATGAAGTCTGGCAATGTGAAGGACCTGCT | sequence |
| | GCAGGCCTGGGATCTGTACTATCACGTGTTTCGGAGAATCTCCAAGCCAGCAGCTCTCGG | only; codon- |
| | CAAAGACACGATTCCGTGGCTTGGGCATCTGCTCGTTGGGCTGAGCGGTGCGTTTGGTTT | optimized; |
| | CATCATCTTGGTCTATCTCTTGATCAATTGCAGAAATACAGGCCCTTGGCTGAAAAAAGT | our DISC |
| | GCTCAAGTGTAATACCCCCGACCCAAGCAAGTTCTTCTCCCAGCTTTCTTCAGAGCATGG | architecture |
| | AGGCGATGTGCAGAAATGGCTCTCTTCACCTTTTCCCTCCTCAAGCTTCTCCCCGGGAGG | version 6 |
| | GCTGGCGCCCGAGATTTCACCTCTTGAGGTACTTGAACGAGACAAGGTTACCCAACTTCT | |
| | CCTTCAACAGGATAAGGTACCCGAACCTGCGAGCCTTAGCTCCAACCACTCTCTTACGAG | |
| | CTGCTTCACCAATCAGGGATACTTCTTTTTCCACCTTCCCGATGCGCTGGAAATCGAAGCT | |
| | TGTCAAGTTTACTTTACCTATGATCCATATAGCGAGGAAGATCCCGACGAAGGAGTCGCC | |
| | GGTGCGCCCACGGGTTCCTCACCCCAACCTCTCCAGCCTCTCTCAGGAGAAGATGATGCT | |
| | TATTGCACTTTTCCCAGTAGAGACGATCTCCTCCTCTTTTCTCCATCTCTTTTGGGGGGAC | |
| | CTTCCCCCCCTTCTACGGCACCTGGCGGGTCTGGTGCTGGCGAGGAGCGGATGCCGCCGT | |
| | CCCTCCAGGAGCGAGTACCACGAGATTGGGATCCCCAGCCACTTGGACCCCCCACCCCCG | |
| | GCGTACCTGACCTTGTCGATTTTCAACCTCCCCCTGAATTGGTGCTGCGAGAGGCTGGGG | |
| | AGGAAGTTCCGGACGCTGGGCCGAGGGAGGGCGTGTCCTTTCCATGGAGTAGGCCTCCA | |
| | GGTCAAGGCGAGTTTAGGGCTCTCAACGCGCGGCTGCCGTTGAATACAGACGCTTATCTC | |
| | TCACTGCAGGAACTGCAAGGTCAGGACCCAACACATCTTGTAGGATCTGGTGCTACTAAT | |
| | TTTTCTCTTTTGAAGCAAGCTGGAGATGTTGAAGAGAACCCCGGTCCGGAGATGTGGCAT | |
| | GAGGGTCTGGAAGAAGCGTCTCGACTGTACTTTGGTGAGCGCAATGTGAAGGGCATGTTT | |
| | GAAGTCCTCGAACCCCTTCATGCCATGATGGAACGCGGACCCCAGACCTTGAAGGAGAC | |
| | AAGTTTTAACCAAGCTTACGGAAGAGACCTGATGGAAGCCCAGGAATGGTGCAGGAAAT | |
| | ACATGAAAAGCGGGAATGTGAAGGACTTGCTCCAAGCGTGGGACCTGTACTATCATGTCT | |
| | TTAGGCGCATTAGTAAG | |
| 88 | MALPVTALLLPLALLLHAARPILWHEMWHEGLEEASRLYFGERNVKGMFEVLEPLHAMMER | CISCβ-DN |
| | GPQTLKETSFNQAYGRDLMEAQEWCRKYMKSGNVKDLLQAWDLYYHVFRRISKPAALGKD | amino acid |
| | TIPWLGHLLVGLSGAFGFIILVYLLINCRNTGPWLKKVLKCNTPDPSKFFSQLSSEHGGDVQK | sequence |
| | WLSSPFPSSSFSPGGLAPEISPLEVLERDKVTQLLLQQDKVPEPASLSSNHSLTSCFTNQGYFFF | |
| | HLPDALEIEACQVYFTYDPYSEEDPDEGVAGAPTGSSPQPLQPLSGEDDAYCTFPSRDDLLLFS | |
| | PSLLGGPSPPSTAPGGSGAGEERMPPSLQERVPRDWDPQPLGPPTPGVPDLVDFQPPPELVLRE | |
| | AGEEVPDAGPREGVSFPWSRPPGQGEFRALNARLPLNTDAYLSLQELQGQDPTHLVGSGATN | |
| | FSLLKQAGDVEENPGPEMWHEGLEEASRLYFGERNVKGMFEVLEPLHAMMERGPQTLKETS | |
| | FNQAYGRDLMEAQEWCRKYMKSGNVKDLLQAWDLYYHVFRRISK | |
| 89 | ATGCCTCTGGGCCTGCTGTGGCTGGGCCTGGCCCTGCTGGGCGCCCTGCACGCCCAGGCC | CISCγ- |
| | GGCGTGCAGGTGGAGACAATCTCCCCAGGCGACGGACGCACATTCCCTAAGCGGGGCCA | FOXP3 |
| | GACCTGCGTGGTGCACTATACAGGCATGCTGGAGGATGGCAAGAAGTTTGACAGCTCCC | cDNA- |
| | GGGATAGAAACAAGCCATTCAAGTTTATGCTGGGCAAGCAGGAAGTGATCAGAGGCTGG | LNGFR |
| | GAGGAGGGCGTGGCCCAGATGTCTGTGGGCCAGAGGGCCAAGCTGACCATCAGCCCAGA | nucleotide |
| | CTACGCCTATGGAGCAACAGGCCACCCAGGAATCATCCCACCTCACGCCACCCTGGTGTT | sequence |
| | CGATGTGGAGCTGCTGAAGCTGGGCGAGGGAGGGTCACCTGGATCCAACACATCAAAAG | (coding |
| | AGAACCCCTTTCTGTTCGCATTGGAGGCCGTAGTCATATCTGTTGGATCCATGGGACTTAT | sequence |
| | TATCTCCCTGTTGTGTGTGTACTTCTGGCTGGAACGGACTATGCCCAGGATCCCCACGCTC | only; codon- |
| | AAGAATCTGGAAGATCTCGTCACAGAATACCATGGTAATTTCAGCGCCTGGAGCGGAGT | optimized; |
| | CTCTAAGGGTCTGGCCGAATCCCTCCAACCCGATTATTCTGAACGGTTGTGCCTCGTATCC | our DISC |
| | GAAATACCACCAAAAGGCGGGGCTCTGGGTGAGGGCCCAGGGGCGAGTCCGTGCAATCA | architecture |
| | ACACAGCCCGTATTGGGCCCCTCCTTGTTATACGTTGAAGCCCGAAACTGGAAGCGGAGC | version 6) |
| | GACTAACTTCAGCCTGCTTAAGCAGGCCGGAGATGTGGAGGAAAACCCTGGACCGATGC | |
| | CTAATCCTCGGCCTGGAAAGCCTAGCGCTCCTTCTCTTGCTCTGGGACCTTCTCCTGGCGC | |
| | CTCTCCATCTTGGAGAGCCGCTCCTAAAGCCAGCGATCTGCTGGGAGCTAGAGGACCTGG | |
| | CGGCACATTTCAGGGCAGAGATCTTAGAGGCGGAGCCCACGCTAGCTCCTCCAGCCTTAA | |
| | TCCTATGCCTCCTAGCCAGCTCCAGCTGCCTACACTGCCTCTGGTTATGGTGGCTCCTAGC | |
| | GGAGCTAGACTGGGCCCTCTGCCTCATCTGCAAGCTCTGCTGCAGGACAGACCCCACTTC | |
| | ATGCACCAGCTGAGCACCGTGGATGCCCACGCAAGAACACCTGTGCTGCAGGTTCACCCT | |
| | CTGGAATCCCCAGCCATGATCAGCCTGACACCTCCAACAACAGCCACCGGCGTGTTCAGC | |
| | CTGAAAGCCAGACCTGGACTGCCTCCTGGCATCAATGTGGCCAGCCTGGAATGGGTGTCC | |
| | AGAGAACCTGCTCTGCTGTGCACATTCCCCAATCCAAGCGCTCCCAGAAAGGACAGCAC | |
| | ACTGTCTGCCGTGCCTCAGAGCAGCTATCCCCTGCTTGCTAACGGCGTGTGCAAGTGGCC | |
| | TGGATGCGAGAAGGTGTTCGAGGAACCCGAGGACTTCCTGAAGCACTGCCAGGCCGATC | |
| | ATCTGCTGGACGAGAAAGGCAGAGCCCAGTGTCTGCTCCAGCGCGAGATGGTGCAGTCT | |
| | CTGGAACAGCAGCTGGTCCTGGAAAAAGAAAAGCTGAGCGCCATGCAGGCCCACCTGGC | |
| | CGGAAAAATGGCCCTGACAAAGGCCAGCAGCGTGGCCTCTTCTGATAAGGGCAGCTGCT | |
| | GCATTGTGGCCGCTGGATCTCAGGGACCTGTGGTTCCTGCTTGGAGCGGACCTAGAGAGG | |
| | CCCCTGATTCTCTGTTTGCCGTGCGGAGACACCTGTGGGGCTCTCACGGCAACTCTACTTT | |
| | CCCCGAGTTCCTGCACAACATGGACTACTTCAAGTTCCACAACATGCGGCCTCCATTCAC | |
| | CTACGCCACACTGATCAGATGGGCCATTCTGGAAGCCCCTGAGAAGCAGAGAACCCTGA | |
| | ACGAGATCTACCACTGGTTTACCCGGATGTTCGCCTTCTTCCGGAATCACCCTGCCACCTG | |
| | GAAGAACGCCATCCGGCACAATCTGAGCCTGCACAAGTGCTTCGTGCGCGTGGAATCTG | |
| | AGAAAGGCGCCGTGTGGACAGTGGACGAGCTGGAATTCAGAAAGAAGAGAAGCCAGCG | |
| | GCCTAGCCGGTGCAGCAATCCTACACCTGGACCTGGAAGCGGAGCGACTAACTTCAGCC | |
| | TGCTGAAGCAGGCCGGAGATGTGGAGGAAAACCCTGGACCGATGGGGGCAGGTGCCACC | |
| | GGACGAGCCATGGACGGGCCGCGCCTGCTGCTGTTGCTGCTTCTGGGGGTGTCCCTTGGA | |
| | GGTGCCAAGGAGGCATGCCCCACAGGCCTGTACACACACAGCGGTGAGTGCTGCAAAGC | |
| | CTGCAACCTGGGCGAGGGTGTGGCCCAGCCTTGTGGAGCCAACCAGACCGTGTGTGAGC | |
| | CCTGCCTGGACAGCGTGACGTTCTCCGACGTGGTGAGCGCGACCGAGCCGTGCAAGCCG | |
| | TGCACCGAGTGCGTGGGGCTCCAGAGCATGTCGGCGCCGTGCGTGGAGGCCGACGACGC | |
| | CGTGTGCCGCTGCGCCTACGGCTACTACCAGGATGAGACGACTGGGCGCTGCGAGGCGT | |
| | GCCGCGTGTGCGAGGCGGGCTCGGGCCTCGTGTTCTCCTGCCAGGACAAGCAGAACACC | |
| | GTGTGCGAGGAGTGCCCCGACGGCACGTATTCCGACGAGGCCAACCACGTGGACCCGTG | |
| | CCTGCCCTGCACCGTGTGCGAGGACACCGAGCGCCAGCTCCGCGAGTGCACACGCTGGG | |
| | CCGACGCCGAGTGCGAGGAGATCCCTGGCCGTTGGATTACACGGTCCACACCCCCAGAG | |
| | GGCTCGGACAGCACAGCCCCCAGCACCCAGGAGCCTGAGGCACCTCCAGAACAAGACCT | |
| | CATAGCCAGCACGGTGGCAGGTGTGGTGACCACAGTGATGGGCAGCTCCCAGCCCGTGG | |
| | TGACCCGAGGCACCACCGACAACCTCATCCCTGTCTATTGCTCCATCCTGGCTGCTGTGG | |
| | TTGTGGGTCTTGTGGCCTACATAGCCTTCAAGAGGTGA | |
| 90 | MPLGLLWLGLALLGALHAQAGVQVETISPGDGRTFPKRGQTCVVHYTGMLEDGKKFDSSRD | CISCγ- |
| | RNKPFKFMLGKQEVIRGWEEGVAQMSVGQRAKLTISPDYAYGATGHPGIIPPHATLVFDVEL | FOXP3 |
| | LKLGEGGSPGSNTSKENPFLFALEAVVISVGSMGLIISLLCVYFWLERTMPRIPTLKNLEDLVT | cDNA- |
| | EYHGNFSAWSGVSKGLAESLQPDYSERLCLVSEIPPKGGALGEGPGASPCNQHSPYWAPPCY | LNGFR |
| | TLKPETGSGATNFSLLKQAGDVEENPGPMPNPRPGKPSAPSLALGPSPGASPSWRAAPKASDL | amino acid |
| | LGARGPGGTFQGRDLRGGAHASSSSLNPMPPSQLQLPTLPLVMVAPSGARLGPLPHLQALLQ | sequence |
| | DRPHFMHQLSTVDAHARTPVLQVHPLESPAMISLTPPTTATGVFSLKARPGLPPGINVASLEW | |
| | VSREPALLCTFPNPSAPRKDSTLSAVPQSSYPLLANGVCKWPGCEKVFEEPEDFLKHCQADHL | |
| | LDEKGRAQCLLQREMVQSLEQQLVLEKEKLSAMQAHLAGKMALTKASSVASSDKGSCCIVA | |
| | AGSQGPVVPAWSGPREAPDSLFAVRRHLWGSHGNSTFPEFLHNMDYFKFHNMRPPFTYATLI | |
| | RWAILEAPEKQRTLNEIYHWFTRMFAFFRNHPATWKNAIRHNLSLHKCFVRVESEKGAVWT | |
| | VDELEFRKKRSQRPSRCSNPTPGPGSGATNFSLLKQAGDVEENPGPMGAGATGRAMDGPRLL | |
| | LLLLLGVSLGGAKEACPTGLYTHSGECCKACNLGEGVAQPCGANQTVCEPCLDSVTFSDVVS | |
| | ATEPCKPCTECVGLQSMSAPCVEADDAVCRCAYGYYQDETTGRCEACRVCEAGSGLVFSCQ | |
| | DKQNTVCEECPDGTYSDEANHVDPCLPCTVCEDTERQLRECTRWADAECEEIPGRWITRSTP | |
| | PEGSDSTAPSTQEPEAPPEQDLIASTVAGVVTTVMGSSQPVVTRGTTDNLIPVYCSILAAVVV | |
| | GLVAYIAFKR* | |
| 91 | ATGCCTCTGGGCCTGCTGTGGCTGGGCCTGGCCCTGCTGGGCGCCCTGCACGCCCAGGCC | CISCγ- |
| | GGCGTGCAGGTGGAGACAATCTCCCCAGGCGACGGACGCACATTCCCTAAGCGGGGCCA | LNGFR- |
| | GACCTGCGTGGTGCACTATACAGGCATGCTGGAGGATGGCAAGAAGTTTGACAGCTCCC | FOXP3 |
| | GGGATAGAAACAAGCCATTCAAGTTTATGCTGGGCAAGCAGGAAGTGATCAGAGGCTGG | cDNA |
| | GAGGAGGGCGTGGCCCAGATGTCTGTGGGCCAGAGGGCCAAGCTGACCATCAGCCCAGA | nucleotide |
| | CTACGCCTATGGAGCAACAGGCCACCCAGGAATCATCCCACCTCACGCCACCCTGGTGTT | sequence |
| | CGATGTGGAGCTGCTGAAGCTGGGCGAGGGAGGGTCACCTGGATCCAACACATCAAAAG | (coding |
| | AGAACCCCTTTCTGTTCGCATTGGAGGCCGTAGTCATATCTGTTGGATCCATGGGACTTAT | sequence |
| | TATCTCCCTGTTGTGTGTGTACTTCTGGCTGGAACGGACTATGCCCAGGATCCCCACGCTC | only; codon- |
| | AAGAATCTGGAAGATCTCGTCACAGAATACCATGGTAATTTCAGCGCCTGGAGCGGAGT | optimized; |
| | CTCTAAGGGTCTGGCCGAATCCCTCCAACCCGATTATTCTGAACGGTTGTGCCTCGTATCC | our DISC |
| | GAAATACCACCAAAAGGCGGGGCTCTGGGTGAGGGCCCAGGGGCGAGTCCGTGCAATCA | architecture |
| | ACACAGCCCGTATTGGGCCCCTCCTTGTTATACGTTGAAGCCCGAAACTGGAAGCGGAGC | version 6) |
| | GACTAACTTCAGCCTGCTTAAGCAGGCCGGAGATGTGGAGGAAAACCCTGGACCGATGG | |
| | GGGCAGGTGCCACCGGACGAGCCATGGACGGGCCGCGCCTGCTGCTGTTGCTGCTTCTGG | |
| | GGGTGTCCCTTGGAGGTGCCAAGGAGGCATGCCCCACAGGCCTGTACACACACAGCGGT | |
| | GAGTGCTGCAAAGCCTGCAACCTGGGCGAGGGTGTGGCCCAGCCTTGTGGAGCCAACCA | |
| | GACCGTGTGTGAGCCCTGCCTGGACAGCGTGACGTTCTCCGACGTGGTGAGCGCGACCG | |
| | AGCCGTGCAAGCCGTGCACCGAGTGCGTGGGGCTCCAGAGCATGTCGGCGCCGTGCGTG | |
| | GAGGCCGACGACGCCGTGTGCCGCTGCGCCTACGGCTACTACCAGGATGAGACGACTGG | |
| | GCGCTGCGAGGCGTGCCGCGTGTGCGAGGCGGGCTCGGGCCTCGTGTTCTCCTGCCAGGA | |
| | CAAGCAGAACACCGTGTGCGAGGAGTGCCCCGACGGCACGTATTCCGACGAGGCCAACC | |
| | ACGTGGACCCGTGCCTGCCCTGCACCGTGTGCGAGGACACCGAGCGCCAGCTCCGCGAG | |
| | TGCACACGCTGGGCCGACGCCGAGTGCGAGGAGATCCCTGGCCGTTGGATTACACGGTC | |
| | CACACCCCCAGAGGGCTCGGACAGCACAGCCCCCAGCACCCAGGAGCCTGAGGCACCTC | |
| | CAGAACAAGACCTCATAGCCAGCACGGTGGCAGGTGTGGTGACCACAGTGATGGGCAGC | |
| | TCCCAGCCCGTGGTGACCCGAGGCACCACCGACAACCTCATCCCTGTCTATTGCTCCATC | |
| | CTGGCTGCTGTGGTTGTGGGTCTTGTGGCCTACATAGCCTTCAAGAGGGGAAGCGGAGCG | |
| | ACTAACTTCAGCCTGCTGAAGCAGGCCGGAGATGTGGAGGAAAACCCTGGACCGATGCC | |
| | TAATCCTCGGCCTGGAAAGCCTAGCGCTCCTTCTCTTGCTCTGGGACCTTCTCCTGGCGCC | |
| | TCTCCATCTTGGAGAGCCGCTCCTAAAGCCAGCGATCTGCTGGGAGCTAGAGGACCTGGC | |
| | GGCACATTTCAGGGCAGAGATCTTAGAGGCGGAGCCCACGCTAGCTCCTCCAGCCTTAAT | |
| | CCTATGCCTCCTAGCCAGCTCCAGCTGCCTACACTGCCTCTGGTTATGGTGGCTCCTAGCG | |
| | GAGCTAGACTGGGCCCTCTGCCTCATCTGCAAGCTCTGCTGCAGGACAGACCCCACTTCA | |
| | TGCACCAGCTGAGCACCGTGGATGCCCACGCAAGAACACCTGTGCTGCAGGTTCACCCTC | |
| | TGGAATCCCCAGCCATGATCAGCCTGACACCTCCAACAACAGCCACCGGCGTGTTCAGCC | |
| | TGAAAGCCAGACCTGGACTGCCTCCTGGCATCAATGTGGCCAGCCTGGAATGGGTGTCCA | |
| | GAGAACCTGCTCTGCTGTGCACATTCCCCAATCCAAGCGCTCCCAGAAAGGACAGCACA | |
| | CTGTCTGCCGTGCCTCAGAGCAGCTATCCCCTGCTTGCTAACGGCGTGTGCAAGTGGCCT | |
| | GGATGCGAGAAGGTGTTCGAGGAACCCGAGGACTTCCTGAAGCACTGCCAGGCCGATCA | |
| | TCTGCTGGACGAGAAAGGCAGAGCCCAGTGTCTGCTCCAGCGCGAGATGGTGCAGTCTC | |
| | TGGAACAGCAGCTGGTCCTGGAAAAAGAAAAGCTGAGCGCCATGCAGGCCCACCTGGCC | |
| | GGAAAAATGGCCCTGACAAAGGCCAGCAGCGTGGCCTCTTCTGATAAGGGCAGCTGCTG | |
| | CATTGTGGCCGCTGGATCTCAGGGACCTGTGGTTCCTGCTTGGAGCGGACCTAGAGAGGC | |
| | CCCTGATTCTCTGTTTGCCGTGCGGAGACACCTGTGGGGCTCTCACGGCAACTCTACTTTC | |
| | CCCGAGTTCCTGCACAACATGGACTACTTCAAGTTCCACAACATGCGGCCTCCATTCACC | |
| | TACGCCACACTGATCAGATGGGCCATTCTGGAAGCCCCTGAGAAGCAGAGAACCCTGAA | |
| | CGAGATCTACCACTGGTTTACCCGGATGTTCGCCTTCTTCCGGAATCACCCTGCCACCTGG | |
| | AAGAACGCCATCCGGCACAATCTGAGCCTGCACAAGTGCTTCGTGCGCGTGGAATCTGA | |
| | GAAAGGCGCCGTGTGGACAGTGGACGAGCTGGAATTCAGAAAGAAGAGAAGCCAGCGG | |
| | CCTAGCCGGTGCAGCAATCCTACACCTGGACCTTGA | |
| 92 | ATGCCTCTGGGCCTGCTGTGGCTGGGCCTGGCCCTGCTGGGCGCCCTGCACGCCCAGGCC | CISCγ- |
| | GGCGTGCAGGTGGAGACAATCTCCCCAGGCGACGGACGCACATTCCCTAAGCGGGGCCA | LNGFR- |
| | GACCTGCGTGGTGCACTATACAGGCATGCTGGAGGATGGCAAGAAGTTTGACAGCTCCC | FOXP3 |
| | GGGATAGAAACAAGCCATTCAAGTTTATGCTGGGCAAGCAGGAAGTGATCAGAGGCTGG | cDNA |
| | GAGGAGGGCGTGGCCCAGATGTCTGTGGGCCAGAGGGCCAAGCTGACCATCAGCCCAGA | |
| | CTACGCCTATGGAGCAACAGGCCACCCAGGAATCATCCCACCTCACGCCACCCTGGTGTT | |
| | CGATGTGGAGCTGCTGAAGCTGGGCGAGGGAGGGTCACCTGGATCCAACACATCAAAAG | |
| | AGAACCCCTTTCTGTTCGCATTGGAGGCCGTAGTCATATCTGTTGGATCCATGGGACTTAT | |
| | TATCTCCCTGTTGTGTGTGTACTTCTGGCTGGAACGGACTATGCCCAGGATCCCCACGCTC | |
| | AAGAATCTGGAAGATCTCGTCACAGAATACCATGGTAATTTCAGCGCCTGGAGCGGAGT | |
| | CTCTAAGGGTCTGGCCGAATCCCTCCAACCCGATTATTCTGAACGGTTGTGCCTCGTATCC | |
| | GAAATACCACCAAAAGGCGGGGCTCTGGGTGAGGGCCCAGGGGCGAGTCCGTGCAATCA | |
| | ACACAGCCCGTATTGGGCCCCTCCTTGTTATACGTTGAAGCCCGAAACTGGAAGCGGAGC | |
| | GACTAACTTCAGCCTGCTTAAGCAGGCCGGAGATGTGGAGGAAAACCCTGGACCGATGG | |
| | GGGCAGGTGCCACCGGACGAGCCATGGACGGGCCGCGCCTGCTGCTGTTGCTGCTTCTGG | |
| | GGGTGTCCCTTGGAGGTGCCAAGGAGGCATGCCCCACAGGCCTGTACACACACAGCGGT | |
| | GAGTGCTGCAAAGCCTGCAACCTGGGCGAGGGTGTGGCCCAGCCTTGTGGAGCCAACCA | |
| | GACCGTGTGTGAGCCCTGCCTGGACAGCGTGACGTTCTCCGACGTGGTGAGCGCGACCG | |
| | AGCCGTGCAAGCCGTGCACCGAGTGCGTGGGGCTCCAGAGCATGTCGGCGCCGTGCGTG | |
| | GAGGCCGACGACGCCGTGTGCCGCTGCGCCTACGGCTACTACCAGGATGAGACGACTGG | |
| | GCGCTGCGAGGCGTGCCGCGTGTGCGAGGCGGGCTCGGGCCTCGTGTTCTCCTGCCAGGA | |
| | CAAGCAGAACACCGTGTGCGAGGAGTGCCCCGACGGCACGTATTCCGACGAGGCCAACC | |
| | ACGTGGACCCGTGCCTGCCCTGCACCGTGTGCGAGGACACCGAGCGCCAGCTCCGCGAG | |
| | TGCACACGCTGGGCCGACGCCGAGTGCGAGGAGATCCCTGGCCGTTGGATTACACGGTC | |
| | CACACCCCCAGAGGGCTCGGACAGCACAGCCCCCAGCACCCAGGAGCCTGAGGCACCTC | |
| | CAGAACAAGACCTCATAGCCAGCACGGTGGCAGGTGTGGTGACCACAGTGATGGGCAGC | |
| | TCCCAGCCCGTGGTGACCCGAGGCACCACCGACAACCTCATCCCTGTCTATTGCTCCATC | |
| | CTGGCTGCTGTGGTTGTGGGTCTTGTGGCCTACATAGCCTTCAAGAGGGGAAGCGGAGCG | |
| | ACTAACTTCAGCCTGCTGAAGCAGGCCGGAGATGTGGAGGAAAACCCTGGACCGATGCC | |
| | TAATCCTCGGCCTGGAAAGCCTAGCGCTCCTTCTCTTGCTCTGGGACCTTCTCCTGGCGCC | |
| | TCTCCATCTTGGAGAGCCGCTCCTAAAGCCAGCGATCTGCTGGGAGCTAGAGGACCTGGC | |
| | GGCACATTTCAGGGCAGAGATCTTAGAGGCGGAGCCCACGCTAGCTCCTCCAGCCTTAAT | |
| | CCTATGCCTCCTAGCCAGCTCCAGCTGCCTACACTGCCTCTGGTTATGGTGGCTCCTAGCG | |
| | GAGCTAGACTGGGCCCTCTGCCTCATCTGCAAGCTCTGCTGCAGGACAGACCCCACTTCA | |
| | TGCACCAGCTGAGCACCGTGGATGCCCACGCAAGAACACCTGTGCTGCAGGTTCACCCTC | |
| | TGGAATCCCCAGCCATGATCAGCCTGACACCTCCAACAACAGCCACCGGCGTGTTCAGCC | |
| | TGAAAGCCAGACCTGGACTGCCTCCTGGCATCAATGTGGCCAGCCTGGAATGGGTGTCCA | |
| | GAGAACCTGCTCTGCTGTGCACATTCCCCAATCCAAGCGCTCCCAGAAAGGACAGCACA | |
| | CTGTCTGCCGTGCCTCAGAGCAGCTATCCCCTGCTTGCTAACGGCGTGTGCAAGTGGCCT | |
| | GGATGCGAGAAGGTGTTCGAGGAACCCGAGGACTTCCTGAAGCACTGCCAGGCCGATCA | |
| | TCTGCTGGACGAGAAAGGCAGAGCCCAGTGTCTGCTCCAGCGCGAGATGGTGCAGTCTC | |
| | TGGAACAGCAGCTGGTCCTGGAAAAAGAAAAGCTGAGCGCCATGCAGGCCCACCTGGCC | |
| | GGAAAAATGGCCCTGACAAAGGCCAGCAGCGTGGCCTCTTCTGATAAGGGCAGCTGCTG | |
| | CATTGTGGCCGCTGGATCTCAGGGACCTGTGGTTCCTGCTTGGAGCGGACCTAGAGAGGC | |
| | CCCTGATTCTCTGTTTGCCGTGCGGAGACACCTGTGGGGCTCTCACGGCAACTCTACTTTC | |
| | CCCGAGTTCCTGCACAACATGGACTACTTCAAGTTCCACAACATGCGGCCTCCATTCACC | |
| | TACGCCACACTGATCAGATGGGCCATTCTGGAAGCCCCTGAGAAGCAGAGAACCCTGAA | |
| | CGAGATCTACCACTGGTTTACCCGGATGTTCGCCTTCTTCCGGAATCACCCTGCCACCTGG | |
| | AAGAACGCCATCCGGCACAATCTGAGCCTGCACAAGTGCTTCGTGCGCGTGGAATCTGA | |
| | GAAAGGCGCCGTGTGGACAGTGGACGAGCTGGAATTCAGAAAGAAGAGAAGCCAGCGG | |
| | CCTAGCCGGTGCAGCAATCCTACACCTGGACCTTGA | |
| 93 | MPLGLLWLGLALLGALHAQAGVQVETISPGDGRTFPKRGQTCVVHYTGMLEDGKKFDSSRD | IL2Rγ-CISC |
| | RNKPFKFMLGKQEVIRGWEEGVAQMSVGQRAKLTISPDYAYGATGHPGIIPPHATLVFDVEL | amino acid |
| | LKLGEGSNTSKENPFLFALEAVVISVGSMGLIISLLCVYFWLERTMPRIPTLKNLEDLVTEYHG | sequence |
| | NFSAWSGVSKGLAESLQPDYSERLCLVSEIPPKGGALGEGPGASPCNQHSPYWAPPCYTLKPE | |
| | T | |
| 94 | (MALPVTALLLPLALLLHAARPILWHEMWHEGLEEASRLYFGERNVKGMFEVLEPLHAMME | IL2Rβ-CISC |
| | RGPQTLKETSFNQAYGRDLMEAQEWCRKYMKSGNVKDLLQAWDLYYHVFRRISKGKDTIP | |
| | WLGHLLVGLSGAFGFIILVYLLINCRNTGPWLKKVLKCNTPDPSKFFSQLSSEHGGDVQKWL | |
| | SSPFPSSSFSPGGLAPEISPLEVLERDKVTQLLLQQDKVPEPASLSSNHSLTSCFTNQGYFFFHLP | |
| | DALEIEACQVYFTYDPYSEEDPDEGVAGAPTGSSPQPLQPLSGEDDAYCTFPSRDDLLLFSPSL | |
| | LGGPSPPSTAPGGSGAGEERMPPSLQERVPRDWDPQPLGPPTPGVPDLVDFQPPPELVLREAG | |
| | EEVPDAGPREGVSFPWSRPPGQGEFRALNARLPLNTDAYLSLQELQGQDPTHLV | |
| 95 | (MPLGLLWLGLALLGALHAQAGVQVETISPGDGRTFPKRGQTCVVHYTGMLEDGKKFDSSR | IL2Rγ-CISC |
| | DRNKPFKFMLGKQEVIRGWEEGVAQMSVGQRAKLTISPDYAYGATGHPGIIPPHATLVFDVE | |
| | LLKLEGGGSQNLVIPWAPENLTLHKLSESQLELNWNNRFLNHCLEHLVQYRTDWDHSWTEQ | |
| | SVDYRHKFSLPSVDGQKRYTFRVRSRFNPLCGSAQHWSEWSHPIHWGSNTSKENPFLFALEA | |
| | VVISVGSMGLIISLLCVYFWLERTMPRIPTLKNLEDLVTEYHGNFSAWSGVSKGLAESLQPDY | |
| | SERLCLVSEIPPKGGALGEGPGASPCNQHSPYWAPPCYTLKPET | |
| 96 | (MALPVTALLLPLALLLHAARPILWHEMWHEGLEEASRLYFGERNVKGMFEVLEPLHAMME | IL2Rβ-CISC |
| | RGPQTLKETSFNQAYGRDLMEAQEWCRKYMKSGNVKDLLQAWDLYYHVFRRISKGGSKPF | |
| | ENLRLMAPISLQVVHVETHRCNISWEISQASHYFERHLEFEARTLSPGHTWEEAPLLTLKQKQ | |
| | EWICLETLTPDTQYEFQVRVKPLQGEFTTWSPWSQPLAFRTKPAALGKDTIPWLGHLLVGLS | |
| | GAFGFIILVYLLINCRNTGPWLKKVLKCNTPDPSKFFQLSSEHGGDVQKWLSSPFPSSSFSPGG | |
| | LAPEISPLEVLERDKVTQLLLQQDKVPEPASLSSNHSLTSCFTNQGYFFFHLPDALEIEACQVY | |
| | FTYDPYSEEDPDEGVAGAPTGSSPQPLQPLSGEDDAYCTFPSRDDLLLFSPSLLGGPSPPSTAP | |
| | GGSGAGEERMPPSLQERVPRDWDPQPLGPPTPGVPDLVDFQPPPELVLREAGEEVPDAGPRE | |
| | GVSFPWSRPPGQGEFRALNARLPLNTDAYLSLQELQGQDPTHLV | |
| 97 | (MPLGLLWLGLALLGALHAQAGVQVETISPGDGRTFPKRGQTCVVHYTGMLEDGKKFDSSR | IL2Rγ-CISC |
| | DRNKPFKFMLGKQEVIRGWEEGVAQMSVGQRAKLTISPDYAYGATGHPGIIPPHATLVFDVE | |
| | LLKLEGQNLVIPWAPENLTLHKLSESQLELNWNNRFLNHCLEHLVQYRTDWDHSWTEQSVD | |
| | YRHKFSLPSVDGQKRYTFRVRSRFNPLCGSAQHWSEWSHPIHWGSNTSKENPFLFALEAVVIS | |
| | VGSMGLIISLLCVYFWLERTMPRIPTLKNLEDLVTEYHGNFSAWSGVSKGLAESLQPDYSERL | |
| | CLVSEIPPKGGALGEGPGASPCNQHSPYWAPPCYTLKPET | |
| 98 | (MALPVTALLLPLALLLHAARPILWHEMWHEGLEEASRLYFGERNVKGMFEVLEPLHAMME | IL2Rβ-CISC |
| | RGPQTLKETSFNQAYGRDLMEAQEWCRKYMKSGNVKDLLQAWDLYYHVFRRISKKPFENL | |
| | RLMAPISLQVVHVETHRCNISWEISQASHYFERHLEFEARTLSPGHTWEEAPLLTLKQKQEWI | |
| | CLETLTPDTQYEFQVRVKPLQGEFTTWSPWSQPLAFRTKPAALGKDTIPWLGHLLVGLSGAF | |
| | GFIILVYLLINCRNTGPWLKKVLKCNTPDPSKFFSQLSSEHGGDVQKWLSSPFPSSSFSPGGLA | |
| | PEISPLEVLERDKVTQLLLQQDKVPEPASLSSNHSLTSCFTNQGYFFFHLPDALEIEACQVYFT | |
| | YDPYSEEDPDEGVAGAPTGSSPQPLQPLSGEDDAYCTFPSRDDLLLFSPSLLGGPSPPSTAPGG | |
| | SGAGEERMPPSLQERVPRDWDPQPLGPPTPGVPDLVDFQPPPELVLREAGEEVPDAGPREGVS | |
| | FPWSRPPGQGEFRALNARLPLNTDAYLSLQELQGQDPTHLV | |
| 99 | (MPLGLLWLGLALLGALHAQAGVQVETISPGDGRTFPKRGQTCVVHYTGMLEDGKKFDSSR | IL2Rγ-CISC |
| | DRNKPFKFMLGKQEVIRGWEEGVAQMSVGQRAKLTISPDYAYGATGHPGIIPPHATLVFDVE | |
| | LLKLEGGSNTSKENPFLFALEAVVISVGSMGLIISLLCVYFWLERTMPRIPTLKNLEDLVTEYH | |
| | GNFSAWSGVSKGLAESLQPDYSERLCLVSEIPPKGGALGEGPGASPCNQHSPYWAPPCYTLK | |
| | PET | |
| 100 | (MALPVTALLLPLALLLHAARPILWHEMWHEGLEEASRLYFGERNVKGMFEVLEPLHAMME | IL2Rβ-CISC |
| | RGPQTLKETSWLGHLLVGLSGAFGFIILVYLLINCRNTGPWLKKVLKCNTPDPSKFFSQLSSEH | |
| | GGDVQKWLSSPFPSSSFSPGGLAPEISPLEVLERDKVTQLLLQQDKVPEPASLSSNHSLTSCFT | |
| | NQGYFFFHLPDALEIEACQVYFTYDPYSEEDPDEGVAGAPTGSSPQPLQPLSGEDDAYCTFPS | |
| | RDDLLLFSPSLLGGPSPPSTAPGGSGAGEERMPPSLQERVPRDWDPQPLGPPTPGVPDLVDFQ | |
| | PPPELVLREAGEEVPDAGPREGVSFPWSRPPGQGEFRALNARLPLNTDAYLSLQELQGQDPTH | |
| | LV | |
| 101 | (MALPVTALLLPLALLLHAARPILWHEMWHEGLEEASRLYFGERNVKGMFEVLEPLHAMME | IL7Rα-CISC |
| | RGPQTLKETSFNQAYGRDLMEAQEWCRKYMKSGNVKDLLQAWDLYYHVFRRISKGEINNS | |
| | SGEMDPILLTISILSFFSVALLVILACVLWKKRIKPIVWPSLPDHKKTLEHLCKKPRKNLNVSFN | |
| | PESFLDCQIHRVDDIQARDEVEGFLQDTFPQQLEESEKQRLGGDVQSPNCPSEDVVITPESFGR | |
| | DSSLTCLAGNVSACDAPILSSSRSLDCRESGKNGPHVYQDLLLSLGTTNSTLPPPFSLQSGILTL | |
| | NPVAQGQPILTSLGSNQEEAYVTMSSFYQNQ | |
| 102 | (MPLGLLWLGLALLGALHAQAGVQVETISPGDGRTFPKRGQTCVVHYTGMLEDGKKVDSSR | IL2Rβ-CISC |
| | DRNKPFKFMLGKQEVIRGWEEGVAQMSVGQRAKLTISPDYAYGATGHPGIIPPHATLVFDVE | |
| | LLKLEGGKDTIPWLGHLLVGLSGAFGFIILVYLLINCRNTGPWLKKVLKCNTPDPSKFFSQLSS | |
| | EHGGDVQKWLSSPFPSSSFSPGGLAPEISPLEVLERDKVTQLLLQQDKVPEPASLSSNHSLTSC | |
| | FTNQGYFFFHLPDALEIEACQVYFTYDPYSEEDPDEGVAGAPTGSSPQPLQPLSGEDDAYCTF | |
| | PSRDDLLLFSPSLLGGPSPPSTAPGGSGAGEERMPPSLQERVPRDWDPQPLGPPTPGVPDLVDF | |
| | QPPPELVLREAGEEVPDAGPREGVSFPWSRPPGQGEFRALNARLPLNTDAYLSLQELQGQDPT | |
| | HLV | |
| 103 | (MPLGLLWLGLALLGALHAQAGVQVETISPGDGRTFPKRGQTCVVHYTGMLEDGKKVDSSR | IL2Rγ-CISC |
| | DRNKPFKFMLGKQEVIRGWEEGVAQMSVGQRAKLTISPDYAYGATGHPGIIPPHATLVFDVE | |
| | LLKLEGGSNTSKENPFLFALEAVVISVGSMGLIISLLCVYFWLERTMPRIPTLKNLEDLVTEYH | |
| | GNFSAWSGVSKGLAESLQPDYSERLCLVSEIPPKGGALGEGPGASPCNQHSPYWAPPCYTLK | |
| | PET | |
| 104 | (MPLGLLWLGLALLGALHAQAGVQVETISPGDGRTFPKRGQTCVVHYTGMLEDGKKVDSSR | IL2Rα-CISC |
| | DRNKPFKFMLGKQEVIRGWEEGVAQMSVGQRAKLTISPDYAYGATGHPGIIPPHATLVFDVE | |
| | LLKLEGEINNSSGEMDPILLTISILSFFSVALLVILACVLWKKRIKPIVWPSLPDHKKTLEHLCK | |
| | KPRKNLNVSFNPESFLDCQIHRVDDIQARDEVEGFLQDTFPQQLEESEKQRLGGDVQSPNCPS | |
| | EDVVITPESFGRDSSLTCLAGNVSACDAPILSSSRSLDCRESGKNGPHVYQDLLLSLGTTNSTL | |
| | PPPFSLQSGILTLNPVAQGQPILTSLGSNQEEAYVTMSSFYQNQ | |
| 105 | (MPLGLLWLGLALLGALHAQAGVQVETISPGDGRTFPKRGQTCVVHYTGMLEDGKKVDSSR | IL7Rα-CISC |
| | DRNKPFKFMLGKQEVIRGWEEGVAQMSVGQRAKLTISPDYAYGATGHPGIIPPHATLVFDVE | |
| | LLKLEGEINNSSGEMDPILLTISILSFFSVALLVILACVLWKKRIKPIVWPSLPDHKKTLEHLCK | |
| | KPRKNLNVSFNPESFLDCQIHRVDDIQARDEVEGFLQDTFPQQLEESEKQRLGGDVQSPNCPS | |
| | EDVVITPESFGRDSSLTCLAGNVSACDAPILSSSRSLDCRESGKNGPHVYQDLLLSLGTTNSTL | |
| | PPPFSLQSGILTLNPVAQGQPILTSLGSNQEEAYVTMSSFYQNQ | |
| 106 | ((MPLGLLWLGLALLGALHAQAGVQVETISPGDGRTFPKRGQTCVVHYTGMLEDGKKVDSSR | MPL-CISC |
| | DRNKPFKFMLGKQEVIRGWEEGVAQMSVGQRAKLTISPDYAYGATGHPGIIPPHATLVFDVE | |
| | LLKLGEETAWISLVTALHLVLGLSAVLGLLLLRWQFPAHYRRLRHALWPSLPDLHRVLGQYL | |
| | RDTAALSPPKATVSDTCEEVEPSLLEILPKSSERTPLPLCSSQAQMDYRRLQPSCLGTMPLSVC | |
| | PPMAESGSCCTTHIANHSYLPLSYWQQP | |
| 107 | ((AGCTTAATGTAGTCTTATGCAATACTCTTGTAGTCTTGCAACATGGTAACGATGAGTTA | Expression |
| | GCAACATGCCTTACAAGGAGAGAAAAAGCACCGTGCATGCCGATTGGTGGAAGTAAGGT | vector |
| | GGTACGATCGTGCCTTATTAGGAAGGCAACAGACGGGTCTGACATGGATTGGACGAACC | |
| | ACTGAATTGCCGCATTGCAGAGATATTGTATTTAAGTGCCTAGCTCGATACAATAAACGG | |
| | GTCTCTCTGGTTAGACCAGATCTGAGCCTGGGAGCTCTCTGGCTAACTAGGGAACCCACT | |
| | GCTTAAGCCTCAATAAAGCTTGCCTTGAGTGCTTCAAGTAGTGTGTGCCCGTCTGTTGTGT | |
| | GACTCTGGTAACTAGAGATCCCTCAGACCCTTTTAGTCAGTGTGGAAAATCTCTAGCAGT | |
| | GGCGCCCGAACAGGGACTTGAAAGCGAAAGGGAAACCAGAGGAGCTCTCTCGACGCAG | |
| | GACTCGGCTTGCTGAAGCGCGCACGGCAAGAGGCGAGGGGCGGCGACTGGTGAGTACGC | |
| | CAAAAATTTTGACTAGCGGAGGCTAGAAGGAGAGAGATGGGTGCGAGAGCGTCAGTATT | |
| | AAGCGGGGGAGAATTAGATCGCGATGGGAAAAAATTCGGTTAAGGCCAGGGGGAAAGA | |
| | AAAAATATAAATTAAAACATATAGTATGGGCAAGCAGGGAGCTAGAACGATTCGCAGTT | |
| | AATCCTGGCCTGTTAGAAACATCAGAAGGCTGTAGACAAATACTGGGACAGCTACAACC | |
| | ATCCCTTCAGACAGGATCAGAAGAACTTAGATCATTATATAATACAGTAGCAACCCTCTA | |
| | TTGTGTGCATCAAAGGATAGAGATAAAAGACACCAAGGAAGCTTTAGACAAGATAGAGG | |
| | AAGAGCAAAACAAAAGTAAGACCACCGCACAGCAAGCGGCCGCTGATCTTCAGACCTGG | |
| | AGGAGGAGATATGAGGGACAATTGGAGAAGTGAATTATATAAATATAAAGTAGTAAAAA | |
| | TTGAACCATTAGGAGTAGCACCCACCAAGGCAAAGAGAAGAGTGGTGCAGAGAGAAAA | |
| | AAGAGCAGTGGGAATAGGAGCTTTGTTCCTTGGGTTCTTGGGAGCAGCAGGAAGCACTA | |
| | TGGGCGCAGCCTCAATGACGCTGACGGTACAGGCCAGACAATTATTGTCTGGTATAGTGC | |
| | AGCAGCAGAACAATTTGCTGAGGGCTATTGAGGCGCAACAGCATCTGTTGCAACTCACA | |
| | GTCTGGGGCATCAAGCAGCTCCAGGCAAGAATCCTGGCTGTGGAAAGATACCTAAAGGA | |
| | TCAACAGCTCCTGGGGATTTGGGGTTGCTCTGGAAAACTCATTTGCACCACTGCTGTGCC | |
| | TTGGAATGCTAGTTGGAGTAATAAATCTCTGGAACAGATTTGGAATCACACGACCTGGAT | |
| | GGAGTGGGACAGAGAAATTAACAATTACACAAGCTTAATACACTCCTTAATTGAAGAAT | |
| | CGCAAAACCAGCAAGAAAAGAATGAACAAGAATTATTGGAATTAGATAAATGGGCAAGT | |
| | TTGTGGAATTGGTTTAACATAACAAATTGGCTGTGGTATATAAAATTATTCATAATGATA | |
| | GTAGGAGGCTTGGTAGGTTTAAGAATAGTTTTTGCTGTACTTTCTATAGTGAATAGAGTT | |
| | AGGCAGGGATATTCACCATTATCGTTTCAGACCCACCTCCCAACCCCGAGGGGACCCGAC | |
| | AGGCCCGAAGGAATAGAAGAAGAAGGTGGAGAGAGAGACAGAGACAGATCCATTCGAT | |
| | TAGTGAACGGATCTCGACGGTATCGGTTAACTTTTAAAAGAAAAGGGGGGATTGGGGGG | |
| | TACAGTGCAGGGGAAAGAATAGTAGACATAATAGCAACAGACATACAAACTAAAGAATT | |
| | ACAAAAACAAATTACAAAAATTCAAAATTTTATCGATCACGAGACTAGCCTCGAGAAGC | |
| | TTGATATCGAATTCCCACGGGGTTGGACGCGTAGGAACAGAGAAACAGGAGAATATGGG | |
| | CCAAACAGGATATCTGTGGTAAGCAGTTCCTGCCCCGGCTCAGGGCCAAGAACAGTTGG | |
| | AACAGCAGAATATGGGCCAAACAGGATATCTGTGGTAAGCAGTTCCTGCCCCGGCTCAG | |
| | GGCCAAGAACAGATGGTCCCCAGATGCGGTCCCGCCCTCAGCAGTTTCTAGAGAACCAT | |
| | CAGATGTTTCCAGGGTGCCCCAAGGACCTGAAATGACCCTGTGCCTTATTTGAACTAACC | |
| | AATCAGTTCGCTTCTCGCTTCTGTTCGCGCGCTTCTGCTCCCCGAGCTCTATATAAGCAGA | |
| | GCTCGTTTAGTGAACCGTCAGATCGCTAGCACCGGTGCCGCCACCATGCCTCTGGGCCTG | |
| | CTGTGGCTGGGCCTGGCCCTGCTGGGCGCCCTGCACGCCCAGGCCGGCGTGCAGGTGGA | |
| | GACAATCTCCCCAGGCGACGGACGCACATTCCCTAAGCGGGGCCAGACCTGCGTGGTGC | |
| | ACTATACAGGCATGCTGGAGGATGGCAAGAAGTTTGACAGCTCCCGGGATAGAAACAAG | |
| | CCATTCAAGTTTATGCTGGGCAAGCAGGAAGTGATCAGAGGCTGGGAGGAGGGCGTGGC | |
| | CCAGATGTCTGTGGGCCAGAGGGCCAAGCTGACCATCAGCCCAGACTACGCCTATGGAG | |
| | CAACAGGCCACCCAGGAATCATCCCACCTCACGCCACCCTGGTGTTCGATGTGGAGCTGC | |
| | TGAAGCTGGGCGAGGGCGGTAGTCAGAACCTTGTGATACCATGGGCCCCAGAAAATCTC | |
| | ACACTTCATAAACTTTCCGAATCACAACTCGAACTCAACTGGAATAACCGGTTCCTGAAT | |
| | CACTGTCTTGAACACCTGGTACAATATCGGACCGACTGGGATCACTCATGGACAGAACA | |
| | ATCTGTGGACTATAGGCACAAATTCTCACTCCCAAGCGTAGACGGCCAAAAAAGATACA | |
| | CTTTTCGCGTACGATCCCGCTTTAATCCTCTCTGCGGCTCTGCTCAGCACTGGAGTGAATG | |
| | GTCCCATCCCATTCATTGGGGATCCAACACATCAAAAGAGAACCCCTTTCTGTTCGCATT | |
| | GGAGGCCGTAGTCATATCTGTTGGATCCATGGGACTTATTATCTCCCTGTTGTGTGTGTAC | |
| | TTCTGGCTGGAACGGACTATGCCCAGGATCCCCACGCTCAAGAATCTGGAAGATCTCGTC | |
| | ACAGAATACCATGGTAATTTCAGCGCCTGGAGCGGAGTCTCTAAGGGTCTGGCCGAATCC | |
| | CTCCAACCCGATTATTCTGAACGGTTGTGCCTCGTATCCGAAATACCACCAAAAGGCGGG | |
| | GCTCTGGGTGAGGGCCCAGGGGCGAGTCCGTGCAATCAACACAGCCCGTATTGGGCCCC | |
| | TCCTTGTTATACGTTGAAGCCCGAAACTGGAAGCGGAGCTACTAACTTCAGCCTGCTGAA | |
| | GCAGGCTGGAGACGTGGAGGAGAACCCTGGACCTATGGCACTGCCCGTGACCGCCCTGC | |
| | TGCTGCCTCTGGCCCTGCTGCTGCACGCAGCCCGGCCTATCCTGTGGCACGAGATGTGGC | |
| | ACGAGGGCCTGGAGGAGGCCAGCAGGCTGTATTTTGGCGAGCGCAACGTGAAGGGCATG | |
| | TTCGAGGTGCTGGAGCCTCTGCACGCCATGATGGAGAGAGGCCCACAGACCCTGAAGGA | |
| | GACATCCTTTAACCAGGCCTATGGACGGGACCTGATGGAGGCACAGGAGTGGTGCAGAA | |
| | AGTACATGAAGTCTGGCAATGTGAAGGACCTGCTGCAGGCCTGGGATCTGTACTATCACG | |
| | TGTTTCGGAGAATCTCCAAGGGAGGTTCAAAACCTTTTGAGAACCTTAGACTGATGGCGC | |
| | CCATCTCTCTGCAGGTAGTTCACGTTGAGACCCATAGATGCAATATAAGCTGGGAAATCT | |
| | CACAAGCCAGCCATTACTTTGAACGGCATTTGGAATTCGAGGCCCGAACACTTTCCCCCG | |
| | GTCATACGTGGGAAGAAGCTCCTCTCTTGACGCTGAAGCAGAAGCAGGAGTGGATTTGTC | |
| | TGGAGACTTTGACTCCTGATACTCAGTATGAGTTCCAAGTTCGGGTGAAACCACTCCAAG | |
| | GCGAGTTCACGACGTGGTCTCCGTGGAGTCAACCGTTGGCGTTCCGCACGAAGCCCGCTG | |
| | CCCTTGGCAAAGACACGATTCCGTGGCTTGGGCATCTGCTCGTTGGGCTGAGTGGTGCGT | |
| | TTGGTTTCATCATCTTGGTCTATCTCTTGATCAATTGCAGAAATACAGGCCCTTGGCTGAA | |
| | AAAAGTGCTCAAGTGTAATACCCCCGACCCAAGCAAGTTCTTCTCCCAGCTTTCTTCAGA | |
| | GCATGGAGGCGATGTGCAGAAATGGCTCTCTTCACCTTTTCCCTCCTCAAGCTTCTCCCCG | |
| | GGAGGGCTGGCGCCCGAGATTTCACCTCTTGAGGTACTTGAACGAGACAAGGTTACCCA | |
| | ACTTCTCCTTCAACAGGATAAGGTACCCGAACCTGCGAGCCTTAGCTCCAACCACTCTCT | |
| | TACGAGCTGCTTCACCAATCAGGGATACTTCTTTTTCCACCTTCCCGATGCGCTGGAAATC | |
| | GAAGCTTGTCAAGTTTACTTTACCTATGATCCATATAGCGAGGAAGATCCCGACGAAGGA | |
| | GTCGCCGGTGCGCCCACGGGTTCCTCACCCCAACCTCTCCAGCCTCTCTCAGGAGAAGAT | |
| | GATGCTTATTGCACTTTTCCCAGTAGAGACGATCTCCTCCTCTTTTCTCCATCTCTTTTGGG | |
| | GGGACCTTCCCCCCCTTCTACGGCACCTGGCGGGTCTGGTGCTGGCGAGGAGCGGATGCC | |
| | GCCGTCCCTCCAGGAGCGAGTACCACGAGATTGGGATCCCCAGCCACTTGGACCCCCCAC | |
| | CCCCGGCGTACCTGACCTTGTCGATTTTCAACCTCCCCCTGAATTGGTGCTGCGAGAGGC | |
| | TGGGGAGGAAGTTCCGGACGCTGGGCCGAGGGAGGGCGTGTCCTTTCCATGGAGTAGGC | |
| | CTCCAGGTCAAGGCGAGTTTAGGGCTCTCAACGCGCGGCTGCCGTTGAATACAGACGCTT | |
| | ATCTCTCACTGCAGGAACTGCAAGGTCAGGACCCAACACATCTTGTAGGATCTGGTGCTA | |
| | CTAATTTTTCTCTTTTGAAGCAAGCTGGAGATGTTGAAGAGAACCCTGGTCCAGTGAGCA | |
| | AGGGCGAGGAGCTGTTCACCGGGGTGGTGCCCATCCTGGTCGAGCTGGACGGCGACGTA | |
| | AACGGCCACAAGTTCAGCGTGTCCGGCGAGGGCGAGGGCGATGCCACCTACGGCAAGCT | |
| | GACCCTGAAGTTCATCTGCACCACCGGCAAGCTGCCCGTGCCCTGGCCCACCCTCGTGAC | |
| | CACCCTGACCTACGGCGTGCAGTGCTTCAGCCGCTACCCCGACCACATGAAGCAGCACG | |
| | ACTTCTTCAAGTCCGCCATGCCCGAAGGCTACGTCCAGGAGCGCACCATCTTCTTCAAGG | |
| | ACGACGGCAACTACAAGACCCGCGCCGAGGTGAAGTTCGAGGGCGACACCCTGGTGAAC | |
| | CGCATCGAGCTGAAGGGCATCGACTTCAAGGAGGACGGCAACATCCTGGGGCACAAGCT | |
| | GGAGTACAACTACAACAGCCACAACGTCTATATCATGGCCGACAAGCAGAAGAACGGCA | |
| | TCAAGGTGAACTTCAAGATCCGCCACAACATCGAGGACGGCAGCGTGCAGCTCGCCGAC | |
| | CACTACCAGCAGAACACCCCCATCGGCGACGGCCCCGTGCTGCTGCCCGACAACCACTA | |
| | CCTGAGCACCCAGTCCGCCCTGAGCAAAGACCCCAACGAGAAGCGCGATCACATGGTCC | |
| | TGCTGGAGTTCGTGACCGCCGCCGGGATCACTCTCGGCATGGACGAGCTGTACAAGTAA | |
| | ACTAGTGTCGACAATCAACCTCTGGATTACAAAATTTGTGAAAGATTGACTGGTATTCTT | |
| | AACTATGTTGCTCCTTTTACGCTATGTGGATACGCTGCTTTAATGCCTTTGTATCATGCTA | |
| | TTGCTTCCCGTATGGCTTTCATTTTCTCCTCCTTGTATAAATCCTGGTTGCTGTCTCTTTAT | |
| | GAGGAGTTGTGGCCCGTTGTCAGGCAACGTGGCGTGGTGTGCACTGTGTTTGCTGACGCA | |
| | ACCCCCACTGGTTGGGGCATTGCCACCACCTGTCAGCTCCTTTCCGGGACTTTCGCTTTCC | |
| | CCCTCCCTATTGCCACGGCGGAACTCATCGCCGCCTGCCTTGCCCGCTGCTGGACAGGGG | |
| | CTCGGCTGTTGGGCACTGACAATTCCGTGGTGTTGTCGGGGAAGCTGACGTCCTTTCCAT | |
| | GGCTGCTCGCCTGTGTTGCCACCTGGATTCTGCGCGGGACGTCCTTCTGCTACGTCCCTTC | |
| | GGCCCTCAATCCAGCGGACCTTCCTTCCCGCGGCCTGCTGCCGGCTCTGCGGCCTCTTCCG | |
| | CGTCTTCGCCTTCGCCCTCAGACGAGTCGGATCTCCCTTTGGGCCGCCTCCCCGCCTGGAA | |
| | TTCGAGCTCGGTACCTTTAAGACCAATGACTTACAAGGCAGCTGTAGATCTTAGCCACTT | |
| | TTTAAAAGAAAAGGGGGGACTGGAAGGGCTAATTCACTCCCAACGAAGACAAGATCTGC | |
| | TTTTTGCTTGTACTGGGTCTCTCTGGTTAGACCAGATCTGAGCCTGGGAGCTCTCTGGCTA | |
| | ACTAGGGAACCCACTGCTTAAGCCTCAATAAAGCTTGCCTTGAGTGCTTCAAGTAGTGTG | |
| | TGCCCGTCTGTTGTGTGACTCTGGTAACTAGAGATCCCTCAGACCCTTTTAGTCAGTGTGG | |
| | AAAATCTCTAGCAGTAGTAGTTCATGTCATCTTATTATTCAGTATTTATAACTTGCAAAGA | |
| | AATGAATATCAGAGAGTGAGAGGAACTTGTTTATTGCAGCTTATAATGGTTACAAATAAA | |
| | GCAATAGCATCACAAATTTCACAAATAAAGCATTTTTTTCACTGCATTCTAGTTGTGGTTT | |
| | GTCCAAACTCATCAATGTATCTTATCATGTCTGGCTCTAGCTATCCCGCCCCTAACTCCGC | |
| | CCAGTTCCGCCCATTCTCCGCCCCATGGCTGACTAATTTTTTTTATTTATGCAGAGGCCGA | |
| | GGCCGCCTCGGCCTCTGAGCTATTCCAGAAGTAGTGAGGAGGCTTTTTTGGAGGCCTAGG | |
| | CTTTTGCGTCGAGACGTACCCAATTCGCCCTATAGTGAGTCGTATTACGCGCGCTCACTG | |
| | GCCGTCGTTTTACAACGTCGTGACTGGGAAAACCCTGGCGTTACCCAACTTAATCGCCTT | |
| | GCAGCACATCCCCCTTTCGCCAGCTGGCGTAATAGCGAAGAGGCCCGCACCGATCGCCCT | |
| | TCCCAACAGTTGCGCAGCCTGAATGGCGAATGGCGCGACGCGCCCTGTAGCGGCGCATT | |
| | AAGCGCGGCGGGTGTGGTGGTTACGCGCAGCGTGACCGCTACACTTGCCAGCGCCCTAG | |
| | CGCCCGCTCCTTTCGCTTTCTTCCCTTCCTTTCTCGCCACGTTCGCCGGCTTTCCCCGTCAA | |
| | GCTCTAAATCGGGGGCTCCCTTTAGGGTTCCGATTTAGTGCTTTACGGCACCTCGACCCC | |
| | AAAAAACTTGATTAGGGTGATGGTTCACGTAGTGGGCCATCGCCCTGATAGACGGTTTTT | |
| | CGCCCTTTGACGTTGGAGTCCACGTTCTTTAATAGTGGACTCTTGTTCCAAACTGGAACAA | |
| | CACTCAACCCTATCTCGGTCTATTCTTTTGATTTATAAGGGATTTTGCCGATTTCGGCCTA | |
| | TTGGTTAAAAAATGAGCTGATTTAACAAAAATTTAACGCGAATTTTAACAAAATATTAAC | |
| | GTTTACAATTTCCCAGGTGGCACTTTTCGGGGAAATGTGCGCGGAACCCCTATTTGTTTAT | |
| | TTTTCTAAATACATTCAAATATGTATCCGCTCATGAGACAATAACCCTGATAAATGCTTCA | |
| | ATAATATTGAAAAAGGAAGAGTATGAGTATTCAACATTTCCGTGTCGCCCTTATTCCCTTT | |
| | TTTGCGGCATTTTGCCTTCCTGTTTTTGCTCACCCAGAAACGCTGGTGAAAGTAAAAGATG | |
| | CTGAAGATCAGTTGGGTGCACGAGTGGGTTACATCGAACTGGATCTCAACAGCGGTAAG | |
| | ATCCTTGAGAGTTTTCGCCCCGAAGAACGTTTTCCAATGATGAGCACTTTTAAAGTTCTGC | |
| | TATGTGGCGCGGTATTATCCCGTATTGACGCCGGGCAAGAGCAACTCGGTCGCCGCATAC | |
| | ACTATTCTCAGAATGACTTGGTTGAGTACTCACCAGTCACAGAAAAGCATCTTACGGATG | |
| | GCATGACAGTAAGAGAATTATGCAGTGCTGCCATAACCATGAGTGATAACACTGCGGCC | |
| | AACTTACTTCTGACAACGATCGGAGGACCGAAGGAGCTAACCGCTTTTTTGCACAACATG | |
| | GGGGATCATGTAACTCGCCTTGATCGTTGGGAACCGGAGCTGAATGAAGCCATACCAAA | |
| | CGACGAGCGTGACACCACGATGCCTGTAGCAATGGCAACAACGTTGCGCAAACTATTAA | |
| | CTGGCGAACTACTTACTCTAGCTTCCCGGCAACAATTAATAGACTGGATGGAGGCGGATA | |
| | AAGTTGCAGGACCACTTCTGCGCTCGGCCCTTCCGGCTGGCTGGTTTATTGCTGATAAAT | |
| | CTGGAGCCGGTGAGCGTGGGTCTCGCGGTATCATTGCAGCACTGGGGCCAGATGGTAAG | |
| | CCCTCCCGTATCGTAGTTATCTACACGACGGGGAGTCAGGCAACTATGGATGAACGAAAT | |
| | AGACAGATCGCTGAGATAGGTGCCTCACTGATTAAGCATTGGTAACTGTCAGACCAAGTT | |
| | TACTCATATATACTTTAGATTGATTTAAAACTTCATTTTTAATTTAAAAGGATCTAGGTGA | |
| | AGATCCTTTTTGATAATCTCATGACCAAAATCCCTTAACGTGAGTTTTCGTTCCACTGAGC | |
| | GTCAGACCCCGTAGAAAAGATCAAAGGATCTTCTTGAGATCCTTTTTTTCTGCGCGTAAT | |
| | CTGCTGCTTGCAAACAAAAAAACCACCGCTACCAGCGGTGGTTTGTTTGCCGGATCAAGA | |
| | GCTACCAACTCTTTTTCCGAAGGTAACTGGCTTCAGCAGAGCGCAGATACCAAATACTGT | |
| | CCTTCTAGTGTAGCCGTAGTTAGGCCACCACTTCAAGAACTCTGTAGCACCGCCTACATA | |
| | CCTCGCTCTGCTAATCCTGTTACCAGTGGCTGCTGCCAGTGGCGATAAGTCGTGTCTTACC | |
| | GGGTTGGACTCAAGACGATAGTTACCGGATAAGGCGCAGCGGTCGGGCTGAACGGGGGG | |
| | TTCGTGCACACAGCCCAGCTTGGAGCGAACGACCTACACCGAACTGAGATACCTACAGC | |
| | GTGAGCTATGAGAAAGCGCCACGCTTCCCGAAGGGAGAAAGGCGGACAGGTATCCGGTA | |
| | AGCGGCAGGGTCGGAACAGGAGAGCGCACGAGGGAGCTTCCAGGGGGAAACGCCTGGT | |
| | ATCTTTATAGTCCTGTCGGGTTTCGCCACCTCTGACTTGAGCGTCGATTTTTGTGATGCTC | |
| | GTCAGGGGGGCGGAGCCTATGGAAAAACGCCAGCAACGCGGCCTTTTTACGGTTCCTGG | |
| | CCTTTTGCTGGCCTTTTGCTCACATGTTCTTTCCTGCGTTATCCCCTGATTCTGTGGATAAC | |
| | CGTATTACCGCCTTTGAGTGAGCTGATACCGCTCGCCGCAGCCGAACGACCGAGCGCAGC | |
| | GAGTCAGTGAGCGAGGAAGCGGAAGAGCGCCCAATACGCAAACCGCCTCTCCCCGCGCG | |
| | TTGGCCGATTCATTAATGCAGCTGGCACGACAGGTTTCCCGACTGGAAAGCGGGCAGTG | |
| | AGCGCAACGCAATTAATGTGAGTTAGCTCACTCATTAGGCACCCCAGGCTTTACACTTTA | |
| | TGCTTCCGGCTCGTATGTTGTGTGGAATTGTGAGCGGATAACAATTTCACACAGGAAACA | |
| | GCTATGACCATGATTACGCCAAGCGCGCAATTAACCCTCACTAAAGGGAACAAAAGCTG | |
| | GAGCTGCA | |
| 108 | (AGCTTAATGTAGTCTTATGCAATACTCTTGTAGTCTTGCAACATGGTAACGATGAGTTAG | Expression |
| | CAACATGCCTTACAAGGAGAGAAAAAGCACCGTGCATGCCGATTGGTGGAAGTAAGGTG | vector |
| | GTACGATCGTGCCTTATTAGGAAGGCAACAGACGGGTCTGACATGGATTGGACGAACCA | |
| | CTGAATTGCCGCATTGCAGAGATATTGTATTTAAGTGCCTAGCTCGATACAATAAACGGG | |
| | TCTCTCTGGTTAGACCAGATCTGAGCCTGGGAGCTCTCTGGCTAACTAGGGAACCCACTG | |
| | CTTAAGCCTCAATAAAGCTTGCCTTGAGTGCTTCAAGTAGTGTGTGCCCGTCTGTTGTGTG | |
| | ACTCTGGTAACTAGAGATCCCTCAGACCCTTTTAGTCAGTGTGGAAAATCTCTAGCAGTG | |
| | GCGCCCGAACAGGGACTTGAAAGCGAAAGGGAAACCAGAGGAGCTCTCTCGACGCAGG | |
| | ACTCGGCTTGCTGAAGCGCGCACGGCAAGAGGCGAGGGGCGGCGACTGGTGAGTACGCC | |
| | AAAAATTTTGACTAGCGGAGGCTAGAAGGAGAGAGATGGGTGCGAGAGCGTCAGTATTA | |
| | AGCGGGGGAGAATTAGATCGCGATGGGAAAAAATTCGGTTAAGGCCAGGGGGAAAGAA | |
| | AAAATATAAATTAAAACATATAGTATGGGCAAGCAGGGAGCTAGAACGATTCGCAGTTA | |
| | ATCCTGGCCTGTTAGAAACATCAGAAGGCTGTAGACAAATACTGGGACAGCTACAACCA | |
| | TCCCTTCAGACAGGATCAGAAGAACTTAGATCATTATATAATACAGTAGCAACCCTCTAT | |
| | TGTGTGCATCAAAGGATAGAGATAAAAGACACCAAGGAAGCTTTAGACAAGATAGAGGA | |
| | AGAGCAAAACAAAAGTAAGACCACCGCACAGCAAGCGGCCGCTGATCTTCAGACCTGGA | |
| | GGAGGAGATATGAGGGACAATTGGAGAAGTGAATTATATAAATATAAAGTAGTAAAAAT | |
| | TGAACCATTAGGAGTAGCACCCACCAAGGCAAAGAGAAGAGTGGTGCAGAGAGAAAAA | |
| | AGAGCAGTGGGAATAGGAGCTTTGTTCCTTGGGTTCTTGGGAGCAGCAGGAAGCACTAT | |
| | GGGCGCAGCCTCAATGACGCTGACGGTACAGGCCAGACAATTATTGTCTGGTATAGTGC | |
| | AGCAGCAGAACAATTTGCTGAGGGCTATTGAGGCGCAACAGCATCTGTTGCAACTCACA | |
| | GTCTGGGGCATCAAGCAGCTCCAGGCAAGAATCCTGGCTGTGGAAAGATACCTAAAGGA | |
| | TCAACAGCTCCTGGGGATTTGGGGTTGCTCTGGAAAACTCATTTGCACCACTGCTGTGCC | |
| | TTGGAATGCTAGTTGGAGTAATAAATCTCTGGAACAGATTTGGAATCACACGACCTGGAT | |
| | GGAGTGGGACAGAGAAATTAACAATTACACAAGCTTAATACACTCCTTAATTGAAGAAT | |
| | CGCAAAACCAGCAAGAAAAGAATGAACAAGAATTATTGGAATTAGATAAATGGGCAAGT | |
| | TTGTGGAATTGGTTTAACATAACAAATTGGCTGTGGTATATAAAATTATTCATAATGATA | |
| | GTAGGAGGCTTGGTAGGTTTAAGAATAGTTTTTGCTGTACTTTCTATAGTGAATAGAGTT | |
| | AGGCAGGGATATTCACCATTATCGTTTCAGACCCACCTCCCAACCCCGAGGGGACCCGAC | |
| | AGGCCCGAAGGAATAGAAGAAGAAGGTGGAGAGAGAGACAGAGACAGATCCATTCGAT | |
| | TAGTGAACGGATCTCGACGGTATCGGTTAACTTTTAAAAGAAAAGGGGGGATTGGGGGG | |
| | TACAGTGCAGGGGAAAGAATAGTAGACATAATAGCAACAGACATACAAACTAAAGAATT | |
| | ACAAAAACAAATTACAAAAATTCAAAATTTTATCGATCACGAGACTAGCCTCGAGAAGC | |
| | TTGATATCGAATTCCCACGGGGTTGGACGCGTAGGAACAGAGAAACAGGAGAATATGGG | |
| | CCAAACAGGATATCTGTGGTAAGCAGTTCCTGCCCCGGCTCAGGGCCAAGAACAGTTGG | |
| | AACAGCAGAATATGGGCCAAACAGGATATCTGTGGTAAGCAGTTCCTGCCCCGGCTCAG | |
| | GGCCAAGAACAGATGGTCCCCAGATGCGGTCCCGCCCTCAGCAGTTTCTAGAGAACCAT | |
| | CAGATGTTTCCAGGGTGCCCCAAGGACCTGAAATGACCCTGTGCCTTATTTGAACTAACC | |
| | AATCAGTTCGCTTCTCGCTTCTGTTCGCGCGCTTCTGCTCCCCGAGCTCTATATAAGCAGA | |
| | GCTCGTTTAGTGAACCGTCAGATCGCTAGCACCGGTGCCGCCACCATGCCTCTGGGCCTG | |
| | CTGTGGCTGGGCCTGGCCCTGCTGGGCGCCCTGCACGCCCAGGCCGGCGTGCAGGTGGA | |
| | GACAATCTCCCCAGGCGACGGACGCACATTCCCTAAGCGGGGCCAGACCTGCGTGGTGC | |
| | ACTATACAGGCATGCTGGAGGATGGCAAGAAGTTTGACAGCTCCCGGGATAGAAACAAG | |
| | CCATTCAAGTTTATGCTGGGCAAGCAGGAAGTGATCAGAGGCTGGGAGGAGGGCGTGGC | |
| | CCAGATGTCTGTGGGCCAGAGGGCCAAGCTGACCATCAGCCCAGACTACGCCTATGGAG | |
| | CAACAGGCCACCCAGGAATCATCCCACCTCACGCCACCCTGGTGTTCGATGTGGAGCTGC | |
| | TGAAGCTGGGCGAGCAAAACTTGGTGATTCCTTGGGCCCCAGAAAATCTCACGCTTCACA | |
| | AGTTGTCCGAATCCCAGCTCGAGCTCAACTGGAATAATAGATTTCTTAATCATTGTTTGG | |
| | AACACCTGGTTCAATATAGAACGGATTGGGACCACTCATGGACCGAGCAGTCAGTTGAC | |
| | TACCGCCACAAATTTTCACTTCCCAGCGTAGATGGGCAGAAGAGGTACACATTTAGGGTC | |
| | AGATCCAGGTTTAATCCTCTGTGTGGTTCTGCTCAACACTGGTCTGAGTGGAGCCATCCG | |
| | ATCCACTGGGGCTCAAATACCTCTAAAGAAAATCCGTTCCTCTTTGCGCTCGAAGCCGTT | |
| | GTTATCAGCGTCGGAAGCATGGGACTTATCATTTCCCTTCTCTGCGTGTACTTCTGGCTGG | |
| | AGCGGACGATGCCGCGGATTCCGACGCTCAAAAACCTGGAGGACCTTGTAACAGAATAT | |
| | CACGGTAATTTCTCCGCTTGGAGTGGCGTATCAAAGGGGCTTGCTGAGTCCCTTCAACCG | |
| | GATTACTCTGAGCGCCTCTGCTTGGTGTCCGAGATACCTCCCAAAGGAGGTGCACTTGGG | |
| | GAGGGGCCAGGCGCGTCCCCTTGCAATCAGCATAGTCCGTATTGGGCGCCCCCCTGTTAT | |
| | ACCCTCAAACCGGAAACGGGAAGCGGAGCTACTAACTTCAGCCTGCTGAAGCAGGCTGG | |
| | AGACGTGGAGGAGAACCCTGGACCTATGGCACTGCCCGTGACCGCCCTGCTGCTGCCTCT | |
| | GGCCCTGCTGCTGCACGCAGCCCGGCCTATCCTGTGGCACGAGATGTGGCACGAGGGCCT | |
| | GGAGGAGGCCAGCAGGCTGTATTTTGGCGAGCGCAACGTGAAGGGCATGTTCGAGGTGC | |
| | TGGAGCCTCTGCACGCCATGATGGAGAGAGGCCCACAGACCCTGAAGGAGACATCCTTT | |
| | AACCAGGCCTATGGACGGGACCTGATGGAGGCACAGGAGTGGTGCAGAAAGTACATGA | |
| | AGTCTGGCAATGTGAAGGACCTGCTGCAGGCCTGGGATCTGTACTATCACGTGTTTCGGA | |
| | GAATCTCCAAGAAACCTTTTGAGAACCTTAGACTGATGGCGCCCATCTCTCTGCAGGTAG | |
| | TTCACGTTGAGACCCATAGATGCAATATAAGCTGGGAAATCTCACAAGCCAGCCATTACT | |
| | TTGAACGGCATTTGGAATTCGAGGCCCGAACACTTTCCCCCGGTCATACGTGGGAAGAAG | |
| | CTCCTCTCTTGACGCTGAAGCAGAAGCAGGAGTGGATTTGTCTGGAGACTTTGACTCCTG | |
| | ATACTCAGTATGAGTTCCAAGTTCGGGTGAAACCACTCCAAGGCGAGTTCACGACGTGGT | |
| | CTCCGTGGAGTCAACCGTTGGCGTTCCGCACGAAGCCCGCTGCCCTTGGCAAAGACACGA | |
| | TTCCGTGGCTTGGGCATCTGCTCGTTGGGCTGAGTGGTGCGTTTGGTTTCATCATCTTGGT | |
| | CTATCTCTTGATCAATTGCAGAAATACAGGCCCTTGGCTGAAAAAAGTGCTCAAGTGTAA | |
| | TACCCCCGACCCAAGCAAGTTCTTCTCCCAGCTTTCTTCAGAGCATGGAGGCGATGTGCA | |
| | GAAATGGCTCTCTTCACCTTTTCCCTCCTCAAGCTTCTCCCCGGGAGGGCTGGCGCCCGA | |
| | GATTTCACCTCTTGAGGTACTTGAACGAGACAAGGTTACCCAACTTCTCCTTCAACAGGA | |
| | TAAGGTACCCGAACCTGCGAGCCTTAGCTCCAACCACTCTCTTACGAGCTGCTTCACCAA | |
| | TCAGGGATACTTCTTTTTCCACCTTCCCGATGCGCTGGAAATCGAAGCTTGTCAAGTTTAC | |
| | TTTACCTATGATCCATATAGCGAGGAAGATCCCGACGAAGGAGTCGCCGGTGCGCCCAC | |
| | GGGTTCCTCACCCCAACCTCTCCAGCCTCTCTCAGGAGAAGATGATGCTTATTGCACTTTT | |
| | CCCAGTAGAGACGATCTCCTCCTCTTTTCTCCATCTCTTTTGGGGGGACCTTCCCCCCCTT | |
| | CTACGGCACCTGGCGGGTCTGGTGCTGGCGAGGAGCGGATGCCGCCGTCCCTCCAGGAG | |
| | CGAGTACCACGAGATTGGGATCCCCAGCCACTTGGACCCCCCACCCCCGGCGTACCTGAC | |
| | CTTGTCGATTTTCAACCTCCCCCTGAATTGGTGCTGCGAGAGGCTGGGGAGGAAGTTCCG | |
| | GACGCTGGGCCGAGGGAGGGCGTGTCCTTTCCATGGAGTAGGCCTCCAGGTCAAGGCGA | |
| | GTTTAGGGCTCTCAACGCGCGGCTGCCGTTGAATACAGACGCTTATCTCTCACTGCAGGA | |
| | ACTGCAAGGTCAGGACCCAACACATCTTGTAGGATCTGGTGCTACTAATTTTTCTCTTTTG | |
| | AAGCAAGCTGGAGATGTTGAAGAGAACCCTGGTCCAGTGAGCAAGGGCGAGGAGCTGTT | |
| | CACCGGGGTGGTGCCCATCCTGGTCGAGCTGGACGGCGACGTAAACGGCCACAAGTTCA | |
| | GCGTGTCCGGCGAGGGCGAGGGCGATGCCACCTACGGCAAGCTGACCCTGAAGTTCATC | |
| | TGCACCACCGGCAAGCTGCCCGTGCCCTGGCCCACCCTCGTGACCACCCTGACCTACGGC | |
| | GTGCAGTGCTTCAGCCGCTACCCCGACCACATGAAGCAGCACGACTTCTTCAAGTCCGCC | |
| | ATGCCCGAAGGCTACGTCCAGGAGCGCACCATCTTCTTCAAGGACGACGGCAACTACAA | |
| | GACCCGCGCCGAGGTGAAGTTCGAGGGCGACACCCTGGTGAACCGCATCGAGCTGAAGG | |
| | GCATCGACTTCAAGGAGGACGGCAACATCCTGGGGCACAAGCTGGAGTACAACTACAAC | |
| | AGCCACAACGTCTATATCATGGCCGACAAGCAGAAGAACGGCATCAAGGTGAACTTCAA | |
| | GATCCGCCACAACATCGAGGACGGCAGCGTGCAGCTCGCCGACCACTACCAGCAGAACA | |
| | CCCCCATCGGCGACGGCCCCGTGCTGCTGCCCGACAACCACTACCTGAGCACCCAGTCCG | |
| | CCCTGAGCAAAGACCCCAACGAGAAGCGCGATCACATGGTCCTGCTGGAGTTCGTGACC | |
| | GCCGCCGGGATCACTCTCGGCATGGACGAGCTGTACAAGTAAACTAGTGTCGACAATCA | |
| | ACCTCTGGATTACAAAATTTGTGAAAGATTGACTGGTATTCTTAACTATGTTGCTCCTTTT | |
| | ACGCTATGTGGATACGCTGCTTTAATGCCTTTGTATCATGCTATTGCTTCCCGTATGGCTT | |
| | TCATTTTCTCCTCCTTGTATAAATCCTGGTTGCTGTCTCTTTATGAGGAGTTGTGGCCCGTT | |
| | GTCAGGCAACGTGGCGTGGTGTGCACTGTGTTTGCTGACGCAACCCCCACTGGTTGGGGC | |
| | ATTGCCACCACCTGTCAGCTCCTTTCCGGGACTTTCGCTTTCCCCCTCCCTATTGCCACGG | |
| | CGGAACTCATCGCCGCCTGCCTTGCCCGCTGCTGGACAGGGGCTCGGCTGTTGGGCACTG | |
| | ACAATTCCGTGGTGTTGTCGGGGAAGCTGACGTCCTTTCCATGGCTGCTCGCCTGTGTTGC | |
| | CACCTGGATTCTGCGCGGGACGTCCTTCTGCTACGTCCCTTCGGCCCTCAATCCAGCGGA | |
| | CCTTCCTTCCCGCGGCCTGCTGCCGGCTCTGCGGCCTCTTCCGCGTCTTCGCCTTCGCCCT | |
| | CAGACGAGTCGGATCTCCCTTTGGGCCGCCTCCCCGCCTGGAATTCGAGCTCGGTACCTT | |
| | TAAGACCAATGACTTACAAGGCAGCTGTAGATCTTAGCCACTTTTTAAAAGAAAAGGGG | |
| | GGACTGGAAGGGCTAATTCACTCCCAACGAAGACAAGATCTGCTTTTTGCTTGTACTGGG | |
| | TCTCTCTGGTTAGACCAGATCTGAGCCTGGGAGCTCTCTGGCTAACTAGGGAACCCACTG | |
| | CTTAAGCCTCAATAAAGCTTGCCTTGAGTGCTTCAAGTAGTGTGTGCCCGTCTGTTGTGTG | |
| | ACTCTGGTAACTAGAGATCCCTCAGACCCTTTTAGTCAGTGTGGAAAATCTCTAGCAGTA | |
| | GTAGTTCATGTCATCTTATTATTCAGTATTTATAACTTGCAAAGAAATGAATATCAGAGA | |
| | GTGAGAGGAACTTGTTTATTGCAGCTTATAATGGTTACAAATAAAGCAATAGCATCACAA | |
| | ATTTCACAAATAAAGCATTTTTTTCACTGCATTCTAGTTGTGGTTTGTCCAAACTCATCAA | |
| | TGTATCTTATCATGTCTGGCTCTAGCTATCCCGCCCCTAACTCCGCCCAGTTCCGCCCATT | |
| | CTCCGCCCCATGGCTGACTAATTTTTTTTATTTATGCAGAGGCCGAGGCCGCCTCGGCCTC | |
| | TGAGCTATTCCAGAAGTAGTGAGGAGGCTTTTTTGGAGGCCTAGGCTTTTGCGTCGAGAC | |
| | GTACCCAATTCGCCCTATAGTGAGTCGTATTACGCGCGCTCACTGGCCGTCGTTTTACAA | |
| | CGTCGTGACTGGGAAAACCCTGGCGTTACCCAACTTAATCGCCTTGCAGCACATCCCCCT | |
| | TTCGCCAGCTGGCGTAATAGCGAAGAGGCCCGCACCGATCGCCCTTCCCAACAGTTGCGC | |
| | AGCCTGAATGGCGAATGGCGCGACGCGCCCTGTAGCGGCGCATTAAGCGCGGCGGGTGT | |
| | GGTGGTTACGCGCAGCGTGACCGCTACACTTGCCAGCGCCCTAGCGCCCGCTCCTTTCGC | |
| | TTTCTTCCCTTCCTTTCTCGCCACGTTCGCCGGCTTTCCCCGTCAAGCTCTAAATCGGGGG | |
| | CTCCCTTTAGGGTTCCGATTTAGTGCTTTACGGCACCTCGACCCCAAAAAACTTGATTAG | |
| | GGTGATGGTTCACGTAGTGGGCCATCGCCCTGATAGACGGTTTTTCGCCCTTTGACGTTG | |
| | GAGTCCACGTTCTTTAATAGTGGACTCTTGTTCCAAACTGGAACAACACTCAACCCTATC | |
| | TCGGTCTATTCTTTTGATTTATAAGGGATTTTGCCGATTTCGGCCTATTGGTTAAAAAATG | |
| | AGCTGATTTAACAAAAATTTAACGCGAATTTTAACAAAATATTAACGTTTACAATTTCCC | |
| | AGGTGGCACTTTTCGGGGAAATGTGCGCGGAACCCCTATTTGTTTATTTTTCTAAATACAT | |
| | TCAAATATGTATCCGCTCATGAGACAATAACCCTGATAAATGCTTCAATAATATTGAAAA | |
| | AGGAAGAGTATGAGTATTCAACATTTCCGTGTCGCCCTTATTCCCTTTTTTGCGGCATTTT | |
| | GCCTTCCTGTTTTTGCTCACCCAGAAACGCTGGTGAAAGTAAAAGATGCTGAAGATCAGT | |
| | TGGGTGCACGAGTGGGTTACATCGAACTGGATCTCAACAGCGGTAAGATCCTTGAGAGTT | |
| | TTCGCCCCGAAGAACGTTTTCCAATGATGAGCACTTTTAAAGTTCTGCTATGTGGCGCGG | |
| | TATTATCCCGTATTGACGCCGGGCAAGAGCAACTCGGTCGCCGCATACACTATTCTCAGA | |
| | ATGACTTGGTTGAGTACTCACCAGTCACAGAAAAGCATCTTACGGATGGCATGACAGTAA | |
| | GAGAATTATGCAGTGCTGCCATAACCATGAGTGATAACACTGCGGCCAACTTACTTCTGA | |
| | CAACGATCGGAGGACCGAAGGAGCTAACCGCTTTTTTGCACAACATGGGGGATCATGTA | |
| | ACTCGCCTTGATCGTTGGGAACCGGAGCTGAATGAAGCCATACCAAACGACGAGCGTGA | |
| | CACCACGATGCCTGTAGCAATGGCAACAACGTTGCGCAAACTATTAACTGGCGAACTACT | |
| | TACTCTAGCTTCCCGGCAACAATTAATAGACTGGATGGAGGCGGATAAAGTTGCAGGAC | |
| | CACTTCTGCGCTCGGCCCTTCCGGCTGGCTGGTTTATTGCTGATAAATCTGGAGCCGGTG | |
| | AGCGTGGGTCTCGCGGTATCATTGCAGCACTGGGGCCAGATGGTAAGCCCTCCCGTATCG | |
| | TAGTTATCTACACGACGGGGAGTCAGGCAACTATGGATGAACGAAATAGACAGATCGCT | |
| | GAGATAGGTGCCTCACTGATTAAGCATTGGTAACTGTCAGACCAAGTTTACTCATATATA | |
| | CTTTAGATTGATTTAAAACTTCATTTTTAATTTAAAAGGATCTAGGTGAAGATCCTTTTTG | |
| | ATAATCTCATGACCAAAATCCCTTAACGTGAGTTTTCGTTCCACTGAGCGTCAGACCCCG | |
| | TAGAAAAGATCAAAGGATCTTCTTGAGATCCTTTTTTTCTGCGCGTAATCTGCTGCTTGCA | |
| | AACAAAAAAACCACCGCTACCAGCGGTGGTTTGTTTGCCGGATCAAGAGCTACCAACTCT | |
| | TTTTCCGAAGGTAACTGGCTTCAGCAGAGCGCAGATACCAAATACTGTCCTTCTAGTGTA | |
| | GCCGTAGTTAGGCCACCACTTCAAGAACTCTGTAGCACCGCCTACATACCTCGCTCTGCT | |
| | AATCCTGTTACCAGTGGCTGCTGCCAGTGGCGATAAGTCGTGTCTTACCGGGTTGGACTC | |
| | AAGACGATAGTTACCGGATAAGGCGCAGCGGTCGGGCTGAACGGGGGGTTCGTGCACAC | |
| | AGCCCAGCTTGGAGCGAACGACCTACACCGAACTGAGATACCTACAGCGTGAGCTATGA | |
| | GAAAGCGCCACGCTTCCCGAAGGGAGAAAGGCGGACAGGTATCCGGTAAGCGGCAGGG | |
| | TCGGAACAGGAGAGCGCACGAGGGAGCTTCCAGGGGGAAACGCCTGGTATCTTTATAGT | |
| | CCTGTCGGGTTTCGCCACCTCTGACTTGAGCGTCGATTTTTGTGATGCTCGTCAGGGGGGC | |
| | GGAGCCTATGGAAAAACGCCAGCAACGCGGCCTTTTTACGGTTCCTGGCCTTTTGCTGGC | |
| | CTTTTGCTCACATGTTCTTTCCTGCGTTATCCCCTGATTCTGTGGATAACCGTATTACCGCC | |
| | TTTGAGTGAGCTGATACCGCTCGCCGCAGCCGAACGACCGAGCGCAGCGAGTCAGTGAG | |
| | CGAGGAAGCGGAAGAGCGCCCAATACGCAAACCGCCTCTCCCCGCGCGTTGGCCGATTC | |
| | ATTAATGCAGCTGGCACGACAGGTTTCCCGACTGGAAAGCGGGCAGTGAGCGCAACGCA | |
| | ATTAATGTGAGTTAGCTCACTCATTAGGCACCCCAGGCTTTACACTTTATGCTTCCGGCTC | |
| | GTATGTTGTGTGGAATTGTGAGCGGATAACAATTTCACACAGGAAACAGCTATGACCATG | |
| | ATTACGCCAAGCGCGCAATTAACCCTCACTAAAGGGAACAAAAGCTGGAGCTGCA | |
| 109 | (AGCTTAATGTAGTCTTATGCAATACTCTTGTAGTCTTGCAACATGGTAACGATGAGTTAG | Expression |
| | CAACATGCCTTACAAGGAGAGAAAAAGCACCGTGCATGCCGATTGGTGGAAGTAAGGTG | vector |
| | GTACGATCGTGCCTTATTAGGAAGGCAACAGACGGGTCTGACATGGATTGGACGAACCA | |
| | CTGAATTGCCGCATTGCAGAGATATTGTATTTAAGTGCCTAGCTCGATACAATAAACGGG | |
| | TCTCTCTGGTTAGACCAGATCTGAGCCTGGGAGCTCTCTGGCTAACTAGGGAACCCACTG | |
| | CTTAAGCCTCAATAAAGCTTGCCTTGAGTGCTTCAAGTAGTGTGTGCCCGTCTGTTGTGTG | |
| | ACTCTGGTAACTAGAGATCCCTCAGACCCTTTTAGTCAGTGTGGAAAATCTCTAGCAGTG | |
| | GCGCCCGAACAGGGACTTGAAAGCGAAAGGGAAACCAGAGGAGCTCTCTCGACGCAGG | |
| | ACTCGGCTTGCTGAAGCGCGCACGGCAAGAGGCGAGGGGCGGCGACTGGTGAGTACGCC | |
| | AAAAATTTTGACTAGCGGAGGCTAGAAGGAGAGAGATGGGTGCGAGAGCGTCAGTATTA | |
| | AGCGGGGGAGAATTAGATCGCGATGGGAAAAAATTCGGTTAAGGCCAGGGGGAAAGAA | |
| | AAAATATAAATTAAAACATATAGTATGGGCAAGCAGGGAGCTAGAACGATTCGCAGTTA | |
| | ATCCTGGCCTGTTAGAAACATCAGAAGGCTGTAGACAAATACTGGGACAGCTACAACCA | |
| | TCCCTTCAGACAGGATCAGAAGAACTTAGATCATTATATAATACAGTAGCAACCCTCTAT | |
| | TGTGTGCATCAAAGGATAGAGATAAAAGACACCAAGGAAGCTTTAGACAAGATAGAGGA | |
| | AGAGCAAAACAAAAGTAAGACCACCGCACAGCAAGCGGCCGCTGATCTTCAGACCTGGA | |
| | GGAGGAGATATGAGGGACAATTGGAGAAGTGAATTATATAAATATAAAGTAGTAAAAAT | |
| | TGAACCATTAGGAGTAGCACCCACCAAGGCAAAGAGAAGAGTGGTGCAGAGAGAAAAA | |
| | AGAGCAGTGGGAATAGGAGCTTTGTTCCTTGGGTTCTTGGGAGCAGCAGGAAGCACTAT | |
| | GGGCGCAGCCTCAATGACGCTGACGGTACAGGCCAGACAATTATTGTCTGGTATAGTGC | |
| | AGCAGCAGAACAATTTGCTGAGGGCTATTGAGGCGCAACAGCATCTGTTGCAACTCACA | |
| | GTCTGGGGCATCAAGCAGCTCCAGGCAAGAATCCTGGCTGTGGAAAGATACCTAAAGGA | |
| | TCAACAGCTCCTGGGGATTTGGGGTTGCTCTGGAAAACTCATTTGCACCACTGCTGTGCC | |
| | TTGGAATGCTAGTTGGAGTAATAAATCTCTGGAACAGATTTGGAATCACACGACCTGGAT | |
| | GGAGTGGGACAGAGAAATTAACAATTACACAAGCTTAATACACTCCTTAATTGAAGAAT | |
| | CGCAAAACCAGCAAGAAAAGAATGAACAAGAATTATTGGAATTAGATAAATGGGCAAGT | |
| | TTGTGGAATTGGTTTAACATAACAAATTGGCTGTGGTATATAAAATTATTCATAATGATA | |
| | GTAGGAGGCTTGGTAGGTTTAAGAATAGTTTTTGCTGTACTTTCTATAGTGAATAGAGTT | |
| | AGGCAGGGATATTCACCATTATCGTTTCAGACCCACCTCCCAACCCCGAGGGGACCCGAC | |
| | AGGCCCGAAGGAATAGAAGAAGAAGGTGGAGAGAGAGACAGAGACAGATCCATTCGAT | |
| | TAGTGAACGGATCTCGACGGTATCGGTTAACTTTTAAAAGAAAAGGGGGGATTGGGGGG | |
| | TACAGTGCAGGGGAAAGAATAGTAGACATAATAGCAACAGACATACAAACTAAAGAATT | |
| | ACAAAAACAAATTACAAAAATTCAAAATTTTATCGATCACGAGACTAGCCTCGAGAAGC | |
| | TTGATATCGAATTCCCACGGGGTTGGACGCGTAGGAACAGAGAAACAGGAGAATATGGG | |
| | CCAAACAGGATATCTGTGGTAAGCAGTTCCTGCCCCGGCTCAGGGCCAAGAACAGTTGG | |
| | AACAGCAGAATATGGGCCAAACAGGATATCTGTGGTAAGCAGTTCCTGCCCCGGCTCAG | |
| | GGCCAAGAACAGATGGTCCCCAGATGCGGTCCCGCCCTCAGCAGTTTCTAGAGAACCAT | |
| | CAGATGTTTCCAGGGTGCCCCAAGGACCTGAAATGACCCTGTGCCTTATTTGAACTAACC | |
| | AATCAGTTCGCTTCTCGCTTCTGTTCGCGCGCTTCTGCTCCCCGAGCTCTATATAAGCAGA | |
| | GCTCGTTTAGTGAACCGTCAGATCGCTAGCACCGGTGCCGCCACCATGCCTCTGGGCCTG | |
| | CTGTGGCTGGGCCTGGCCCTGCTGGGCGCCCTGCACGCCCAGGCCGGCGTGCAGGTGGA | |
| | GACAATCTCCCCAGGCGACGGACGCACATTCCCTAAGCGGGGCCAGACCTGCGTGGTGC | |
| | ACTATACAGGCATGCTGGAGGATGGCAAGAAGTTTGACAGCTCCCGGGATAGAAACAAG | |
| | CCATTCAAGTTTATGCTGGGCAAGCAGGAAGTGATCAGAGGCTGGGAGGAGGGCGTGGC | |
| | CCAGATGTCTGTGGGCCAGAGGGCCAAGCTGACCATCAGCCCAGACTACGCCTATGGAG | |
| | CAACAGGCCACCCAGGAATCATCCCACCTCACGCCACCCTGGTGTTCGATGTGGAGCTGC | |
| | TGAAGCTGGGCGAGGGATCCAACACATCAAAAGAGAACCCCTTTCTGTTCGCATTGGAG | |
| | GCCGTAGTCATATCTGTTGGATCCATGGGACTTATTATCTCCCTGTTGTGTGTGTACTTCT | |
| | GGCTGGAACGGACTATGCCCAGGATCCCCACGCTCAAGAATCTGGAAGATCTCGTCACA | |
| | GAATACCATGGTAATTTCAGCGCCTGGAGCGGAGTCTCTAAGGGTCTGGCCGAATCCCTC | |
| | CAACCCGATTATTCTGAACGGTTGTGCCTCGTATCCGAAATACCACCAAAAGGCGGGGCT | |
| | CTGGGTGAGGGCCCAGGGGCGAGTCCGTGCAATCAACACAGCCCGTATTGGGCCCCTCC | |
| | TTGTTATACGTTGAAGCCCGAAACTGGAAGCGGAGCTACTAACTTCAGCCTGCTGAAGCA | |
| | GGCTGGAGACGTGGAGGAGAACCCTGGACCTATGGCACTGCCCGTGACCGCCCTGCTGC | |
| | TGCCTCTGGCCCTGCTGCTGCACGCAGCCCGGCCTATCCTGTGGCACGAGATGTGGCACG | |
| | AGGGCCTGGAGGAGGCCAGCAGGCTGTATTTTGGCGAGCGCAACGTGAAGGGCATGTTC | |
| | GAGGTGCTGGAGCCTCTGCACGCCATGATGGAGAGAGGCCCACAGACCCTGAAGGAGAC | |
| | ATCCTTTAACCAGGCCTATGGACGGGACCTGATGGAGGCACAGGAGTGGTGCAGAAAGT | |
| | ACATGAAGTCTGGCAATGTGAAGGACCTGCTGCAGGCCTGGGATCTGTACTATCACGTGT | |
| | TTCGGAGAATCTCCAAGGGCAAAGACACGATTCCGTGGCTTGGGCATCTGCTCGTTGGGC | |
| | TGAGTGGTGCGTTTGGTTTCATCATCTTGGTCTATCTCTTGATCAATTGCAGAAATACAGG | |
| | CCCTTGGCTGAAAAAAGTGCTCAAGTGTAATACCCCCGACCCAAGCAAGTTCTTCTCCCA | |
| | GCTTTCTTCAGAGCATGGAGGCGATGTGCAGAAATGGCTCTCTTCACCTTTTCCCTCCTCA | |
| | AGCTTCTCCCCGGGAGGGCTGGCGCCCGAGATTTCACCTCTTGAGGTACTTGAACGAGAC | |
| | AAGGTTACCCAACTTCTCCTTCAACAGGATAAGGTACCCGAACCTGCGAGCCTTAGCTCC | |
| | AACCACTCTCTTACGAGCTGCTTCACCAATCAGGGATACTTCTTTTTCCACCTTCCCGATG | |
| | CGCTGGAAATCGAAGCTTGTCAAGTTTACTTTACCTATGATCCATATAGCGAGGAAGATC | |
| | CCGACGAAGGAGTCGCCGGTGCGCCCACGGGTTCCTCACCCCAACCTCTCCAGCCTCTCT | |
| | CAGGAGAAGATGATGCTTATTGCACTTTTCCCAGTAGAGACGATCTCCTCCTCTTTTCTCC | |
| | ATCTCTTTTGGGGGGACCTTCCCCCCCTTCTACGGCACCTGGCGGGTCTGGTGCTGGCGA | |
| | GGAGCGGATGCCGCCGTCCCTCCAGGAGCGAGTACCACGAGATTGGGATCCCCAGCCAC | |
| | TTGGACCCCCCACCCCCGGCGTACCTGACCTTGTCGATTTTCAACCTCCCCCTGAATTGGT | |
| | GCTGCGAGAGGCTGGGGAGGAAGTTCCGGACGCTGGGCCGAGGGAGGGCGTGTCCTTTC | |
| | CATGGAGTAGGCCTCCAGGTCAAGGCGAGTTTAGGGCTCTCAACGCGCGGCTGCCGTTG | |
| | AATACAGACGCTTATCTCTCACTGCAGGAACTGCAAGGTCAGGACCCAACACATCTTGTA | |
| | GGATCTGGTGCTACTAATTTTTCTCTTTTGAAGCAAGCTGGAGATGTTGAAGAGAACCCT | |
| | GGTCCAGTGAGCAAGGGCGAGGAGCTGTTCACCGGGGTGGTGCCCATCCTGGTCGAGCT | |
| | GGACGGCGACGTAAACGGCCACAAGTTCAGCGTGTCCGGCGAGGGCGAGGGCGATGCCA | |
| | CCTACGGCAAGCTGACCCTGAAGTTCATCTGCACCACCGGCAAGCTGCCCGTGCCCTGGC | |
| | CCACCCTCGTGACCACCCTGACCTACGGCGTGCAGTGCTTCAGCCGCTACCCCGACCACA | |
| | TGAAGCAGCACGACTTCTTCAAGTCCGCCATGCCCGAAGGCTACGTCCAGGAGCGCACC | |
| | ATCTTCTTCAAGGACGACGGCAACTACAAGACCCGCGCCGAGGTGAAGTTCGAGGGCGA | |
| | CACCCTGGTGAACCGCATCGAGCTGAAGGGCATCGACTTCAAGGAGGACGGCAACATCC | |
| | TGGGGCACAAGCTGGAGTACAACTACAACAGCCACAACGTCTATATCATGGCCGACAAG | |
| | CAGAAGAACGGCATCAAGGTGAACTTCAAGATCCGCCACAACATCGAGGACGGCAGCGT | |
| | GCAGCTCGCCGACCACTACCAGCAGAACACCCCCATCGGCGACGGCCCCGTGCTGCTGC | |
| | CCGACAACCACTACCTGAGCACCCAGTCCGCCCTGAGCAAAGACCCCAACGAGAAGCGC | |
| | GATCACATGGTCCTGCTGGAGTTCGTGACCGCCGCCGGGATCACTCTCGGCATGGACGAG | |
| | CTGTACAAGTAAACTAGTGTCGACAATCAACCTCTGGATTACAAAATTTGTGAAAGATTG | |
| | ACTGGTATTCTTAACTATGTTGCTCCTTTTACGCTATGTGGATACGCTGCTTTAATGCCTTT | |
| | GTATCATGCTATTGCTTCCCGTATGGCTTTCATTTTCTCCTCCTTGTATAAATCCTGGTTGC | |
| | TGTCTCTTTATGAGGAGTTGTGGCCCGTTGTCAGGCAACGTGGCGTGGTGTGCACTGTGTT | |
| | TGCTGACGCAACCCCCACTGGTTGGGGCATTGCCACCACCTGTCAGCTCCTTTCCGGGAC | |
| | TTTCGCTTTCCCCCTCCCTATTGCCACGGCGGAACTCATCGCCGCCTGCCTTGCCCGCTGC | |
| | TGGACAGGGGCTCGGCTGTTGGGCACTGACAATTCCGTGGTGTTGTCGGGGAAGCTGACG | |
| | TCCTTTCCATGGCTGCTCGCCTGTGTTGCCACCTGGATTCTGCGCGGGACGTCCTTCTGCT | |
| | ACGTCCCTTCGGCCCTCAATCCAGCGGACCTTCCTTCCCGCGGCCTGCTGCCGGCTCTGCG | |
| | GCCTCTTCCGCGTCTTCGCCTTCGCCCTCAGACGAGTCGGATCTCCCTTTGGGCCGCCTCC | |
| | CCGCCTGGAATTCGAGCTCGGTACCTTTAAGACCAATGACTTACAAGGCAGCTGTAGATC | |
| | TTAGCCACTTTTTAAAAGAAAAGGGGGGACTGGAAGGGCTAATTCACTCCCAACGAAGA | |
| | CAAGATCTGCTTTTTGCTTGTACTGGGTCTCTCTGGTTAGACCAGATCTGAGCCTGGGAGC | |
| | TCTCTGGCTAACTAGGGAACCCACTGCTTAAGCCTCAATAAAGCTTGCCTTGAGTGCTTC | |
| | AAGTAGTGTGTGCCCGTCTGTTGTGTGACTCTGGTAACTAGAGATCCCTCAGACCCTTTTA | |
| | GTCAGTGTGGAAAATCTCTAGCAGTAGTAGTTCATGTCATCTTATTATTCAGTATTTATAA | |
| | CTTGCAAAGAAATGAATATCAGAGAGTGAGAGGAACTTGTTTATTGCAGCTTATAATGGT | |
| | TACAAATAAAGCAATAGCATCACAAATTTCACAAATAAAGCATTTTTTTCACTGCATTCT | |
| | AGTTGTGGTTTGTCCAAACTCATCAATGTATCTTATCATGTCTGGCTCTAGCTATCCCGCC | |
| | CCTAACTCCGCCCAGTTCCGCCCATTCTCCGCCCCATGGCTGACTAATTTTTTTTATTTAT | |
| | GCAGAGGCCGAGGCCGCCTCGGCCTCTGAGCTATTCCAGAAGTAGTGAGGAGGCTTTTTT | |
| | GGAGGCCTAGGCTTTTGCGTCGAGACGTACCCAATTCGCCCTATAGTGAGTCGTATTACG | |
| | CGCGCTCACTGGCCGTCGTTTTACAACGTCGTGACTGGGAAAACCCTGGCGTTACCCAAC | |
| | TTAATCGCCTTGCAGCACATCCCCCTTTCGCCAGCTGGCGTAATAGCGAAGAGGCCCGCA | |
| | CCGATCGCCCTTCCCAACAGTTGCGCAGCCTGAATGGCGAATGGCGCGACGCGCCCTGTA | |
| | GCGGCGCATTAAGCGCGGCGGGTGTGGTGGTTACGCGCAGCGTGACCGCTACACTTGCC | |
| | AGCGCCCTAGCGCCCGCTCCTTTCGCTTTCTTCCCTTCCTTTCTCGCCACGTTCGCCGGCTT | |
| | TCCCCGTCAAGCTCTAAATCGGGGGCTCCCTTTAGGGTTCCGATTTAGTGCTTTACGGCAC | |
| | CTCGACCCCAAAAAACTTGATTAGGGTGATGGTTCACGTAGTGGGCCATCGCCCTGATAG | |
| | ACGGTTTTTCGCCCTTTGACGTTGGAGTCCACGTTCTTTAATAGTGGACTCTTGTTCCAAA | |
| | CTGGAACAACACTCAACCCTATCTCGGTCTATTCTTTTGATTTATAAGGGATTTTGCCGAT | |
| | TTCGGCCTATTGGTTAAAAAATGAGCTGATTTAACAAAAATTTAACGCGAATTTTAACAA | |
| | AATATTAACGTTTACAATTTCCCAGGTGGCACTTTTCGGGGAAATGTGCGCGGAACCCCT | |
| | ATTTGTTTATTTTTCTAAATACATTCAAATATGTATCCGCTCATGAGACAATAACCCTGAT | |
| | AAATGCTTCAATAATATTGAAAAAGGAAGAGTATGAGTATTCAACATTTCCGTGTCGCCC | |
| | TTATTCCCTTTTTTGCGGCATTTTGCCTTCCTGTTTTTGCTCACCCAGAAACGCTGGTGAAA | |
| | GTAAAAGATGCTGAAGATCAGTTGGGTGCACGAGTGGGTTACATCGAACTGGATCTCAA | |
| | CAGCGGTAAGATCCTTGAGAGTTTTCGCCCCGAAGAACGTTTTCCAATGATGAGCACTTT | |
| | TAAAGTTCTGCTATGTGGCGCGGTATTATCCCGTATTGACGCCGGGCAAGAGCAACTCGG | |
| | TCGCCGCATACACTATTCTCAGAATGACTTGGTTGAGTACTCACCAGTCACAGAAAAGCA | |
| | TCTTACGGATGGCATGACAGTAAGAGAATTATGCAGTGCTGCCATAACCATGAGTGATAA | |
| | CACTGCGGCCAACTTACTTCTGACAACGATCGGAGGACCGAAGGAGCTAACCGCTTTTTT | |
| | GCACAACATGGGGGATCATGTAACTCGCCTTGATCGTTGGGAACCGGAGCTGAATGAAG | |
| | CCATACCAAACGACGAGCGTGACACCACGATGCCTGTAGCAATGGCAACAACGTTGCGC | |
| | AAACTATTAACTGGCGAACTACTTACTCTAGCTTCCCGGCAACAATTAATAGACTGGATG | |
| | GAGGCGGATAAAGTTGCAGGACCACTTCTGCGCTCGGCCCTTCCGGCTGGCTGGTTTATT | |
| | GCTGATAAATCTGGAGCCGGTGAGCGTGGGTCTCGCGGTATCATTGCAGCACTGGGGCC | |
| | AGATGGTAAGCCCTCCCGTATCGTAGTTATCTACACGACGGGGAGTCAGGCAACTATGG | |
| | ATGAACGAAATAGACAGATCGCTGAGATAGGTGCCTCACTGATTAAGCATTGGTAACTGT | |
| | CAGACCAAGTTTACTCATATATACTTTAGATTGATTTAAAACTTCATTTTTAATTTAAAAG | |
| | GATCTAGGTGAAGATCCTTTTTGATAATCTCATGACCAAAATCCCTTAACGTGAGTTTTCG | |
| | TTCCACTGAGCGTCAGACCCCGTAGAAAAGATCAAAGGATCTTCTTGAGATCCTTTTTTTC | |
| | TGCGCGTAATCTGCTGCTTGCAAACAAAAAAACCACCGCTACCAGCGGTGGTTTGTTTGC | |
| | CGGATCAAGAGCTACCAACTCTTTTTCCGAAGGTAACTGGCTTCAGCAGAGCGCAGATAC | |
| | CAAATACTGTCCTTCTAGTGTAGCCGTAGTTAGGCCACCACTTCAAGAACTCTGTAGCAC | |
| | CGCCTACATACCTCGCTCTGCTAATCCTGTTACCAGTGGCTGCTGCCAGTGGCGATAAGT | |
| | CGTGTCTTACCGGGTTGGACTCAAGACGATAGTTACCGGATAAGGCGCAGCGGTCGGGC | |
| | TGAACGGGGGGTTCGTGCACACAGCCCAGCTTGGAGCGAACGACCTACACCGAACTGAG | |
| | ATACCTACAGCGTGAGCTATGAGAAAGCGCCACGCTTCCCGAAGGGAGAAAGGCGGACA | |
| | GGTATCCGGTAAGCGGCAGGGTCGGAACAGGAGAGCGCACGAGGGAGCTTCCAGGGGG | |
| | AAACGCCTGGTATCTTTATAGTCCTGTCGGGTTTCGCCACCTCTGACTTGAGCGTCGATTT | |
| | TTGTGATGCTCGTCAGGGGGGCGGAGCCTATGGAAAAACGCCAGCAACGCGGCCTTTTT | |
| | ACGGTTCCTGGCCTTTTGCTGGCCTTTTGCTCACATGTTCTTTCCTGCGTTATCCCCTGATT | |
| | CTGTGGATAACCGTATTACCGCCTTTGAGTGAGCTGATACCGCTCGCCGCAGCCGAACGA | |
| | CCGAGCGCAGCGAGTCAGTGAGCGAGGAAGCGGAAGAGCGCCCAATACGCAAACCGCC | |
| | TCTCCCCGCGCGTTGGCCGATTCATTAATGCAGCTGGCACGACAGGTTTCCCGACTGGAA | |
| | AGCGGGCAGTGAGCGCAACGCAATTAATGTGAGTTAGCTCACTCATTAGGCACCCCAGG | |
| | CTTTACACTTTATGCTTCCGGCTCGTATGTTGTGTGGAATTGTGAGCGGATAACAATTTCA | |
| | CACAGGAAACAGCTATGACCATGATTACGCCAAGCGCGCAATTAACCCTCACTAAAGGG | |
| | AACAAAAGCTGGAGCTGCA | |
| 110 | (ATGCCTAATCCTCGGCCTGGAAAGCCTAGCGCTCCTTCTCTTGCTCTGGGACCTTCTCCT | Codon- |
| | GGCGCCTCTCCATCTTGGAGAGCCGCTCCTAAAGCCAGCGATCTGCTGGGAGCTAGAGG | optimized |
| | ACCTGGCGGCACATTTCAGGGCAGAGATCTTAGAGGCGGAGCCCACGCTAGCTCCTCCA | human |
| | GCCTTAATCCTATGCCTCCTAGCCAGCTCCAGCTGCCTACACTGCCTCTGGTTATGGTGGC | FOXP3 |
| | TCCTAGCGGAGCTAGACTGGGCCCTCTGCCTCATCTGCAAGCTCTGCTGCAGGACAGACC | cDNA, |
| | CCACTTCATGCACCAGCTGAGCACCGTGGATGCCCACGCAAGAACACCTGTGCTGCAGGT | Without stop |
| | TCACCCTCTGGAATCCCCAGCCATGATCAGCCTGACACCTCCAACAACAGCCACCGGCGT | codon |
| | GTTCAGCCTGAAAGCCAGACCTGGACTGCCTCCTGGCATCAATGTGGCCAGCCTGGAATG | |
| | GGTGTCCAGAGAACCTGCTCTGCTGTGCACATTCCCCAATCCAAGCGCTCCCAGAAAGGA | |
| | CAGCACACTGTCTGCCGTGCCTCAGAGCAGCTATCCCCTGCTTGCTAACGGCGTGTGCAA | |
| | GTGGCCTGGATGCGAGAAGGTGTTCGAGGAACCCGAGGACTTCCTGAAGCACTGCCAGG | |
| | CCGATCATCTGCTGGACGAGAAAGGCAGAGCCCAGTGTCTGCTCCAGCGCGAGATGGTG | |
| | CAGTCTCTGGAACAGCAGCTGGTCCTGGAAAAAGAAAAGCTGAGCGCCATGCAGGCCCA | |
| | CCTGGCCGGAAAAATGGCCCTGACAAAGGCCAGCAGCGTGGCCTCTTCTGATAAGGGCA | |
| | GCTGCTGCATTGTGGCCGCTGGATCTCAGGGACCTGTGGTTCCTGCTTGGAGCGGACCTA | |
| | GAGAGGCCCCTGATTCTCTGTTTGCCGTGCGGAGACACCTGTGGGGCTCTCACGGCAACT | |
| | CTACTTTCCCCGAGTTCCTGCACAACATGGACTACTTCAAGTTCCACAACATGCGGCCTC | |
| | CATTCACCTACGCCACACTGATCAGATGGGCCATTCTGGAAGCCCCTGAGAAGCAGAGA | |
| | ACCCTGAACGAGATCTACCACTGGTTTACCCGGATGTTCGCCTTCTTCCGGAATCACCCT | |
| | GCCACCTGGAAGAACGCCATCCGGCACAATCTGAGCCTGCACAAGTGCTTCGTGCGCGT | |
| | GGAATCTGAGAAAGGCGCCGTGTGGACAGTGGACGAGCTGGAATTCAGAAAGAAGAGA | |
| | AGCCAGCGGCCTAGCCGGTGCAGCAATCCTACACCTGGACCT) | |
| 111 | (ATGCCTAATCCTCGGCCTGGAAAGCCTAGCGCTCCTTCTCTTGCTCTGGGACCTTCTCCT | Codon- |
| | GGCGCCTCTCCATCTTGGAGAGCCGCTCCTAAAGCCAGCGATCTGCTGGGAGCTAGAGG | optimized |
| | ACCTGGCGGCACATTTCAGGGCAGAGATCTTAGAGGCGGAGCCCACGCTAGCTCCTCCA | human |
| | GCCTTAATCCTATGCCTCCTAGCCAGCTCCAGCTGCCTACACTGCCTCTGGTTATGGTGGC | FOXP3 |
| | TCCTAGCGGAGCTAGACTGGGCCCTCTGCCTCATCTGCAAGCTCTGCTGCAGGACAGACC | cDNA, With |
| | CCACTTCATGCACCAGCTGAGCACCGTGGATGCCCACGCAAGAACACCTGTGCTGCAGGT | stop codon |
| | TCACCCTCTGGAATCCCCAGCCATGATCAGCCTGACACCTCCAACAACAGCCACCGGCGT | |
| | GTTCAGCCTGAAAGCCAGACCTGGACTGCCTCCTGGCATCAATGTGGCCAGCCTGGAATG | |
| | GGTGTCCAGAGAACCTGCTCTGCTGTGCACATTCCCCAATCCAAGCGCTCCCAGAAAGGA | |
| | CAGCACACTGTCTGCCGTGCCTCAGAGCAGCTATCCCCTGCTTGCTAACGGCGTGTGCAA | |
| | GTGGCCTGGATGCGAGAAGGTGTTCGAGGAACCCGAGGACTTCCTGAAGCACTGCCAGG | |
| | CCGATCATCTGCTGGACGAGAAAGGCAGAGCCCAGTGTCTGCTCCAGCGCGAGATGGTG | |
| | CAGTCTCTGGAACAGCAGCTGGTCCTGGAAAAAGAAAAGCTGAGCGCCATGCAGGCCCA | |
| | CCTGGCCGGAAAAATGGCCCTGACAAAGGCCAGCAGCGTGGCCTCTTCTGATAAGGGCA | |
| | GCTGCTGCATTGTGGCCGCTGGATCTCAGGGACCTGTGGTTCCTGCTTGGAGCGGACCTA | |
| | GAGAGGCCCCTGATTCTCTGTTTGCCGTGCGGAGACACCTGTGGGGCTCTCACGGCAACT | |
| | CTACTTTCCCCGAGTTCCTGCACAACATGGACTACTTCAAGTTCCACAACATGCGGCCTC | |
| | CATTCACCTACGCCACACTGATCAGATGGGCCATTCTGGAAGCCCCTGAGAAGCAGAGA | |
| | ACCCTGAACGAGATCTACCACTGGTTTACCCGGATGTTCGCCTTCTTCCGGAATCACCCT | |
| | GCCACCTGGAAGAACGCCATCCGGCACAATCTGAGCCTGCACAAGTGCTTCGTGCGCGT | |
| | GGAATCTGAGAAAGGCGCCGTGTGGACAGTGGACGAGCTGGAATTCAGAAAGAAGAGA | |
| | AGCCAGCGGCCTAGCCGGTGCAGCAATCCTACACCTGGACCTTGA | |
| 112 | MEMWHEGLEEASRLYFGERNVKGMFEVLEPLHAMMERGPQTLKETSFNQAYGRDLMEAQE | Naked FRB |
| | WCRKYMKSGNVKDLTQAWDLYYHVFRRISK | domain |
| 113 | MEMWHEGLEEASRLYFGERNVKGMFEVLEPLHAMMERGPQTLKETSFNQAYGRDLMEAQE | Naked FRB |
| | WCRKYMKSGNVKDLLQAWDLYYHVFRRISK | domain |
| 114 | ATGGCACTGCCCGTGACCGCCCTGCTGCTGCCTCTGGCCCTGCTGCTGCACGCAGCCCGG | CISCβ: FRB- |
| | CCTATCCTGTGGCACGAGATGTGGCACGAGGGCCTGGAGGAGGCCAGCAGGCTGTATTT | IL2Rβ; |
| | TGGCGAGCGCAACGTGAAGGGCATGTTCGAGGTGCTGGAGCCTCTGCACGCCATGATGG | nucleotide |
| | AGAGAGGCCCACAGACCCTGAAGGAGACATCCTTTAACCAGGCCTATGGACGGGACCTG | sequence |
| | ATGGAGGCACAGGAGTGGTGCAGAAAGTACATGAAGTCTGGCAATGTGAAGGACCTGCT | |
| | GCAGGCCTGGGATCTGTACTATCACGTGTTTCGGAGAATCTCCAAGCCAGCAGCTCTCGG | |
| | CAAAGACACGATTCCGTGGCTTGGGCATCTGCTCGTTGGGCTGAGCGGTGCGTTTGGTTT | |
| | CATCATCTTGGTCTATCTCTTGATCAATTGCAGAAATACAGGCCCTTGGCTGAAAAAAGT | |
| | GCTCAAGTGTAATACCCCCGACCCAAGCAAGTTCTTCTCCCAGCTTTCTTCAGAGCATGG | |
| | AGGCGATGTGCAGAAATGGCTCTCTTCACCTTTTCCCTCCTCAAGCTTCTCCCCGGGAGG | |
| | GCTGGCGCCCGAGATTTCACCTCTTGAGGTACTTGAACGAGACAAGGTTACCCAACTTCT | |
| | CCTTCAACAGGATAAGGTACCCGAACCTGCGAGCCTTAGCTCCAACCACTCTCTTACGAG | |
| | CTGCTTCACCAATCAGGGATACTTCTTTTTCCACCTTCCCGATGCGCTGGAAATCGAAGCT | |
| | TGTCAAGTTTACTTTACCTATGATCCATATAGCGAGGAAGATCCCGACGAAGGAGTCGCC | |
| | GGTGCGCCCACGGGTTCCTCACCCCAACCTCTCCAGCCTCTCTCAGGAGAAGATGATGCT | |
| | TATTGCACTTTTCCCAGTAGAGACGATCTCCTCCTCTTTTCTCCATCTCTTTTGGGGGGAC | |
| | CTTCCCCCCCTTCTACGGCACCTGGCGGGTCTGGTGCTGGCGAGGAGCGGATGCCGCCGT | |
| | CCCTCCAGGAGCGAGTACCACGAGATTGGGATCCCCAGCCACTTGGACCCCCCACCCCCG | |
| | GCGTACCTGACCTTGTCGATTTTCAACCTCCCCCTGAATTGGTGCTGCGAGAGGCTGGGG | |
| | AGGAAGTTCCGGACGCTGGGCCGAGGGAGGGCGTGTCCTTTCCATGGAGTAGGCCTCCA | |
| | GGTCAAGGCGAGTTTAGGGCTCTCAACGCGCGGCTGCCGTTGAATACAGACGCTTATCTC | |
| | TCACTGCAGGAACTGCAAGGTCAGGACCCAACACATCTTGTA | |
| 115 | MALPVTALLLPLALLLHAARPILWHEMWHEGLEEASRLYFGERNVKGMFEVLEPLHAMMER | CISCβ: FRB- |
| | GPQTLKETSFNQAYGRDLMEAQEWCRKYMKSGNVKDLLQAWDLYYHVFRRISKPAALGKD | IL2Rβ amino |
| | TIPWLGHLLVGLSGAFGFIILVYLLINCRNTGPWLKKVLKCNTPDPSKFFSQLSSEHGGDVQK | acid |
| | WLSSPFPSSSFSPGGLAPEISPLEVLERDKVTQLLLQQDKVPEPASLSSNHSLTSCFTNQGYFFF | sequence |
| | HLPDALEIEACQVYFTYDPYSEEDPDEGVAGAPTGSSPQPLQPLSGEDDAYCTFPSRDDLLLFS | |
| | PSLLGGPSPPSTAPGGSGAGEERMPPSLQERVPRDWDPQPLGPPTPGVPDLVDFQPPPELVLRE | |
| | AGEEVPDAGPREGVSFPWSRPPGQGEFRALNARLPLNTDAYLSLQELQGQDPTHLV | |
| 116 | ATGCCTCTGGGCCTGCTGTGGCTGGGCCTGGCCCTGCTGGGCGCCCTGCACGCCCAGGCC | CISCγ: |
| | GGCGTGCAGGTGGAGACAATCTCCCCAGGCGACGGACGCACATTCCCTAAGCGGGGCCA | FKBP- |
| | GACCTGCGTGGTGCACTATACAGGCATGCTGGAGGATGGCAAGAAGTTTGACAGCTCCC | IL2Rγ; |
| | GGGATAGAAACAAGCCATTCAAGTTTATGCTGGGCAAGCAGGAAGTGATCAGAGGCTGG | nucleotide |
| | GAGGAGGGCGTGGCCCAGATGTCTGTGGGCCAGAGGGCCAAGCTGACCATCAGCCCAGA | sequence |
| | CTACGCCTATGGAGCAACAGGCCACCCAGGAATCATCCCACCTCACGCCACCCTGGTGTT | |
| | CGATGTGGAGCTGCTGAAGCTGGGCGAGGGAGGGTCACCTGGATCCAACACATCAAAAG | |
| | AGAACCCCTTTCTGTTCGCATTGGAGGCCGTAGTCATATCTGTTGGATCCATGGGACTTAT | |
| | TATCTCCCTGTTGTGTGTGTACTTCTGGCTGGAACGGACTATGCCCAGGATCCCCACGCTC | |
| | AAGAATCTGGAAGATCTCGTCACAGAATACCATGGTAATTTCAGCGCCTGGAGCGGAGT | |
| | CTCTAAGGGTCTGGCCGAATCCCTCCAACCCGATTATTCTGAACGGTTGTGCCTCGTATCC | |
| | GAAATACCACCAAAAGGCGGGGCTCTGGGTGAGGGCCCAGGGGCGAGTCCGTGCAATCA | |
| | ACACAGCCCGTATTGGGCCCCTCCTTGTTATACGTTGAAGCCCGAAACT | |
| 117 | MPLGLLWLGLALLGALHAQAGVQVETISPGDGRTFPKRGQTCVVHYTGMLEDGKKFDSSRD | CISCγ: |
| | RNKPFKFMLGKQEVIRGWEEGVAQMSVGQRAKLTISPDYAYGATGHPGIIPPHATLVFDVEL | FKBP-IL2Rγ |
| | LKLGEGGSPGSNTSKENPFLFALEAVVISVGSMGLIISLLCVYFWLERTMPRIPTLKNLEDLVT | amino acid |
| | EYHGNFSAWSGVSKGLAESLQPDYSERLCLVSEIPPKGGALGEGPGASPCNQHSPYWAPPCY | sequence |
| | TLKPET | |
| 118 | ATGCCTCTGGGCCTGCTGTGGCTGGGCCTGGCCCTGCTGGGCGCCCTGCACGCCCAGGCC | DISC: CISC- |
| | GGCGTGCAGGTGGAGACAATCTCCCCAGGCGACGGACGCACATTCCCTAAGCGGGGCCA | FRB; μDISC: |
| | GACCTGCGTGGTGCACTATACAGGCATGCTGGAGGATGGCAAGAAGTTTGACAGCTCCC | μCISC-FRB |
| | GGGATAGAAACAAGCCATTCAAGTTTATGCTGGGCAAGCAGGAAGTGATCAGAGGCTGG | DISC: CISC- |
| | GAGGAGGGCGTGGCCCAGATGTCTGTGGGCCAGAGGGCCAAGCTGACCATCAGCCCAGA | FRB; |
| | CTACGCCTATGGAGCAACAGGCCACCCAGGAATCATCCCACCTCACGCCACCCTGGTGTT | nucleotide |
| | CGATGTGGAGCTGCTGAAGCTGGGCGAGGGAGGGTCACCTGGATCCAACACATCAAAAG | sequence |
| | AGAACCCCTTTCTGTTCGCATTGGAGGCCGTAGTCATATCTGTTGGATCCATGGGACTTAT | |
| | TATCTCCCTGTTGTGTGTGTACTTCTGGCTGGAACGGACTATGCCCAGGATCCCCACGCTC | |
| | AAGAATCTGGAAGATCTCGTCACAGAATACCATGGTAATTTCAGCGCCTGGAGCGGAGT | |
| | CTCTAAGGGTCTGGCCGAATCCCTCCAACCCGATTATTCTGAACGGTTGTGCCTCGTATCC | |
| | GAAATACCACCAAAAGGCGGGGCTCTGGGTGAGGGCCCAGGGGCGAGTCCGTGCAATCA | |
| | ACACAGCCCGTATTGGGCCCCTCCTTGTTATACGTTGAAGCCCGAAACTGGAAGCGGAGC | |
| | TACTAACTTCAGCCTGCTGAAGCAGGCTGGAGACGTGGAGGAGAACCCTGGACCTATGG | |
| | CACTGCCCGTGACCGCCCTGCTGCTGCCTCTGGCCCTGCTGCTGCACGCAGCCCGGCCTA | |
| | TCCTGTGGCACGAGATGTGGCACGAGGGCCTGGAGGAGGCCAGCAGGCTGTATTTTGGC | |
| | GAGCGCAACGTGAAGGGCATGTTCGAGGTGCTGGAGCCTCTGCACGCCATGATGGAGAG | |
| | AGGCCCACAGACCCTGAAGGAGACATCCTTTAACCAGGCCTATGGACGGGACCTGATGG | |
| | AGGCACAGGAGTGGTGCAGAAAGTACATGAAGTCTGGCAATGTGAAGGACCTGCTGCAG | |
| | GCCTGGGATCTGTACTATCACGTGTTTCGGAGAATCTCCAAGCCAGCAGCTCTCGGCAAA | |
| | GACACGATTCCGTGGCTTGGGCATCTGCTCGTTGGGCTGAGCGGTGCGTTTGGTTTCATC | |
| | ATCTTGGTCTATCTCTTGATCAATTGCAGAAATACAGGCCCTTGGCTGAAAAAAGTGCTC | |
| | AAGTGTAATACCCCCGACCCAAGCAAGTTCTTCTCCCAGCTTTCTTCAGAGCATGGAGGC | |
| | GATGTGCAGAAATGGCTCTCTTCACCTTTTCCCTCCTCAAGCTTCTCCCCGGGAGGGCTGG | |
| | CGCCCGAGATTTCACCTCTTGAGGTACTTGAACGAGACAAGGTTACCCAACTTCTCCTTC | |
| | AACAGGATAAGGTACCCGAACCTGCGAGCCTTAGCTCCAACCACTCTCTTACGAGCTGCT | |
| | TCACCAATCAGGGATACTTCTTTTTCCACCTTCCCGATGCGCTGGAAATCGAAGCTTGTCA | |
| | AGTTTACTTTACCTATGATCCATATAGCGAGGAAGATCCCGACGAAGGAGTCGCCGGTGC | |
| | GCCCACGGGTTCCTCACCCCAACCTCTCCAGCCTCTCTCAGGAGAAGATGATGCTTATTG | |
| | CACTTTTCCCAGTAGAGACGATCTCCTCCTCTTTTCTCCATCTCTTTTGGGGGGACCTTCC | |
| | CCCCCTTCTACGGCACCTGGCGGGTCTGGTGCTGGCGAGGAGCGGATGCCGCCGTCCCTC | |
| | CAGGAGCGAGTACCACGAGATTGGGATCCCCAGCCACTTGGACCCCCCACCCCCGGCGT | |
| | ACCTGACCTTGTCGATTTTCAACCTCCCCCTGAATTGGTGCTGCGAGAGGCTGGGGAGGA | |
| | AGTTCCGGACGCTGGGCCGAGGGAGGGCGTGTCCTTTCCATGGAGTAGGCCTCCAGGTC | |
| | AAGGCGAGTTTAGGGCTCTCAACGCGCGGCTGCCGTTGAATACAGACGCTTATCTCTCAC | |
| | TGCAGGAACTGCAAGGTCAGGACCCAACACATCTTGTAGGATCTGGTGCTACTAATTTTT | |
| | CTCTTTTGAAGCAAGCTGGAGATGTTGAAGAGAACCCCGGTCCGGAGATGTGGCATGAG | |
| | GGTCTGGAAGAAGCGTCTCGACTGTACTTTGGTGAGCGCAATGTGAAGGGCATGTTTGAA | |
| | GTCCTCGAACCCCTTCATGCCATGATGGAACGCGGACCCCAGACCTTGAAGGAGACAAG | |
| | TTTTAACCAAGCTTACGGAAGAGACCTGATGGAAGCCCAGGAATGGTGCAGGAAATACA | |
| | TGAAAAGCGGGAATGTGAAGGACTTGCTCCAAGCGTGGGACCTGTACTATCATGTCTTTA | |
| | GGCGCATTAGTAAG | |
| 119 | MPLGLLWLGLALLGALHAQAGVQVETISPGDGRTFPKRGQTCVVHYTGMLEDGKKFDSSRD | DISC: CISC- |
| | RNKPFKFMLGKQEVIRGWEEGVAQMSVGQRAKLTISPDYAYGATGHPGIIPPHATLVFDVEL | FRB; μDISC |
| | LKLGEGGSPGSNTSKENPFLFALEAVVISVGSMGLIISLLCVYFWLERTMPRIPTLKNLEDLVT | amino acid |
| | EYHGNFSAWSGVSKGLAESLQPDYSERLCLVSEIPPKGGALGEGPGASPCNQHSPYWAPPCY | sequence |
| | TLKPETGSGATNFSLLKQAGDVEENPGPMALPVTALLLPLALLLHAARPILWHEMWHEGLEE | |
| | ASRLYFGERNVKGMFEVLEPLHAMMERGPQTLKETSFNQAYGRDLMEAQEWCRKYMKSG | |
| | NVKDLLQAWDLYYHVFRRISKPAALGKDTIPWLGHLLVGLSGAFGFIILVYLLINCRNTGPWL | |
| | KKVLKCNTPDPSKFFSQLSSEHGGDVQKWLSSPFPSSSFSPGGLAPEISPLEVLERDKVTQLLL | |
| | QQDKVPEPASLSSNHSLTSCFTNQGYFFFHLPDALEIEACQVYFTYDPYSEEDPDEGVAGAPT | |
| | GSSPQPLQPLSGEDDAYCTFPSRDDLLLFSPSLLGGPSPPSTAPGGSGAGEERMPPSLQERVPR | |
| | DWDPQPLGPPTPGVPDLVDFQPPPELVLREAGEEVPDAGPREGVSFPWSRPPGQGEFRALNA | |
| | RLPLNTDAYLSLQELQGQDPTHLVGSGATNFSLLKQAGDVEENPGPEMWHEGLEEASRLYF | |
| | GERNVKGMFEVLEPLHAMMERGPQTLKETSFNQAYGRDLMEAQEWCRKYMKSGNVKDLL | |
| | QAWDLYYHVFRRISK | |
| 120 | GAGATGTGGCATGAGGGTCTGGAAGAAGCGTCTCGACTGTACTTTGGTGAGCGCAATGT | FRB: express |
| | GAAGGGCATGTTTGAAGTCCTCGAACCCCTTCATGCCATGATGGAACGCGGACCCCAGA | intracellularly |
| | CCTTGAAGGAGACAAGTTTTAACCAAGCTTACGGAAGAGACCTGATGGAAGCCCAGGAA | to function |
| | TGGTGCAGGAAATACATGAAAAGCGGGAATGTGAAGGACTTGACCCAAGCGTGGGACCT | as a decoy |
| | GTACTATCATGTCTTTAGGCGCATTAGTAAG | for |
| | | rapamycin: |
| | | FRB; |
| | | nucleotide |
| | | sequence |
| 121 | EMWHEGLEEASRLYFGERNVKGMFEVLEPLHAMMERGPQTLKETSFNQAYGRDLMEAQE | FRB amino |
| | WCRKYMKSGNVKDLTQAWDLYYHVFRRISK | acid |
| | | sequence |
| 122 | ATGGGGGCAGGTGCCACCGGACGAGCCATGGACGGGCCGCGCCTGCTGCTGTTGCTGCT | LNGFR |
| | TCTGGGGGTGTCCCTTGGAGGTGCCAAGGAGGCATGCCCCACAGGCCTGTACACACACA | coding |
| | GCGGTGAGTGCTGCAAAGCCTGCAACCTGGGCGAGGGTGTGGCCCAGCCTTGTGGAGCC | sequence |
| | AACCAGACCGTGTGTGAGCCCTGCCTGGACAGCGTGACGTTCTCCGACGTGGTGAGCGC | with stop |
| | GACCGAGCCGTGCAAGCCGTGCACCGAGTGCGTGGGGCTCCAGAGCATGTCGGCGCCGT | codon |
| | GCGTGGAGGCCGACGACGCCGTGTGCCGCTGCGCCTACGGCTACTACCAGGATGAGACG | |
| | ACTGGGCGCTGCGAGGCGTGCCGCGTGTGCGAGGCGGGCTCGGGCCTCGTGTTCTCCTGC | |
| | CAGGACAAGCAGAACACCGTGTGCGAGGAGTGCCCCGACGGCACGTATTCCGACGAGGC | |
| | CAACCACGTGGACCCGTGCCTGCCCTGCACCGTGTGCGAGGACACCGAGCGCCAGCTCC | |
| | GCGAGTGCACACGCTGGGCCGACGCCGAGTGCGAGGAGATCCCTGGCCGTTGGATTACA | |
| | CGGTCCACACCCCCAGAGGGCTCGGACAGCACAGCCCCCAGCACCCAGGAGCCTGAGGC | |
| | ACCTCCAGAACAAGACCTCATAGCCAGCACGGTGGCAGGTGTGGTGACCACAGTGATGG | |
| | GCAGCTCCCAGCCCGTGGTGACCCGAGGCACCACCGACAACCTCATCCCTGTCTATTGCT | |
| | CCATCCTGGCTGCTGTGGTTGTGGGTCTTGTGGCCTACATAGCCTTCAAGAGGTGA | |
| 123 | GGAAGCGGAGCGACTAACTTCAGCCTGCTGAAGCAGGCCGGAGATGTGGAGGAAAACCC | LNGFRe: |
| | TGGACCG | LNGFR |
| | | epitope |
| | | coding |
| | | sequence |
| | | 2A: P2A self- |
| | | cleaving |
| | | peptide |
| 124 | TGCTAGCGTGGGCAGGCAAGCCAGGTGCTGGACCTCTGCACGTGGGGCATGTGTGGGTA | 0.25 kb |
| | TGTACATGTACCTGTGTTCTTGGTGTGTGTGTGTGTGTGTGTGTGTGTGTGTGTCTAGAGC | human |
| | TGGGGTGCAACTATGGGGCCCCTCGGGACATGTCCCAGCCAATGCCTGCTTTGACCAGAG | FOXP3 |
| | GAGTGTCCACGTGGCTCAGGTGGTCGAGTATCTCATACCGCCCTAGCACACGTGTGACTC | 5′HA |
| | CTTTCCCCTATTGTCTAC | designed for |
| | | both TALEN |
| | | and Cas9 |
| | | approache |
| 125 | CATGTGTGGGTATGTACATGTACCTGTGTTCTTGGTGTGTGTGTGTGTGTGTGTGTGTGTG | 0.3 kb human |
| | TGTGTCTAGAGCTGGGGTGCAACTATGGGGCCCCTCGGGACATGTCCCAGCCAATGCCTG | FOXP3 |
| | CTTTGACCAGAGGAGTGTCCACGTGGCTCAGGTGGTCGAGTATCTCATACCGCCCTAGCA | 5′HA for |
| | CACGTGTGACTCCTTTCCCCTATTGTCTACGCAGCCTGCCCTTGGACAAGGACCCGATGC | Cas9-T9 |
| | CCAACCCCAGGCCTGGCAAGCCCTCGGCCCCTTCCTTGGCCCTTGGCCCATCCCC | |
| 126 | AGCCTGTGCAGGGTGCAGGGAGGGCTAGAGGCCTGAGGCTTGAAACAGCTCTCAAGTGG | 0.45 kb |
| | AGGGGGAAACAACCATTGCCCTCATAGAGGACACATCCACACCAGGGCTGTGCTAGCGT | human |
| | GGGCAGGCAAGCCAGGTGCTGGACCTCTGCACGTGGGGCATGTGTGGGTATGTACATGT | FOXP3 |
| | ACCTGTGTTCTTGGTGTGTGTGTGTGTGTGTGTGTGTGTGTGTGTCTAGAGCTGGGGTGCA | 5′HA for |
| | ACTATGGGGCCCCTCGGGACATGTCCCAGCCAATGCCTGCTTTGACCAGAGGAGTGTCCA | Cas9-T9 |
| | CGTGGCTCAGGTGGTCGAGTATCTCATACCGCCCTAGCACACGTGTGACTCCTTTCCCCT | |
| | ATTGTCTACGCAGCCTGCCCTTGGACAAGGACCCGATGCCCAACCCCAGGCCTGGCAAGC | |
| | CCTCGGCCCCTTCCTTGGCCCTTGGCCCATCCCC | |
| 127 | ATCACTTGCCAGGACTGTTACAATAGCCTCCTCACTAGCCCCACTCACAGCAGCCAGATG | 0.6 kb human |
| | AATCTTTTGAGTCCATGCCTAGTCACTGGGGCAAAATAGGACTCCGAGGAGAAAGTCCG | FOXP3 |
| | AGACCAGCTCCGGCAAGATGAGCAAACACAGCCTGTGCAGGGTGCAGGGAGGGCTAGA | 5′HA for |
| | GGCCTGAGGCTTGAAACAGCTCTCAAGTGGAGGGGGAAACAACCATTGCCCTCATAGAG | Cas9-T9 |
| | GACACATCCACACCAGGGCTGTGCTAGCGTGGGCAGGCAAGCCAGGTGCTGGACCTCTG | |
| | CACGTGGGGCATGTGTGGGTATGTACATGTACCTGTGTTCTTGGTGTGTGTGTGTGTGTGT | |
| | GTGTGTGTGTGTGTCTAGAGCTGGGGTGCAACTATGGGGCCCCTCGGGACATGTCCCAGC | |
| | CAATGCCTGCTTTGACCAGAGGAGTGTCCACGTGGCTCAGGTGGTCGAGTATCTCATACC | |
| | GCCCTAGCACACGTGTGACTCCTTTCCCCTATTGTCTACGCAGCCTGCCCTTGGACAAGG | |
| | ACCCGATGCCCAACCCCAGGCCTGGCAAGCCCTCGGCCCCTTCCTTGGCCCTTGGCCCAT | |
| | CCCC | |
| 128 | ATCTCAGGTAATGTCAGCTCGGTCCTTCCAGCTGCTCAAGCTAAAACCCATGTCACTTTG | 0.8 kb human |
| | ACTCTCCCTCTTGCCCACTACATCCAAGCTGCTAGCACTGCTCCTGATCCAGCTTCAGATT | FOXP3 |
| | AAGTCTCAGAATCTACCCACTTCTCGCCTTCTCCACTGCCACCAGCCCATTCTGTGCCAGC | 5′HA for |
| | ATCATCACTTGCCAGGACTGTTACAATAGCCTCCTCACTAGCCCCACTCACAGCAGCCAG | Cas9-T9 |
| | ATGAATCTTTTGAGTCCATGCCTAGTCACTGGGGCAAAATAGGACTCCGAGGAGAAAGTC | |
| | CGAGACCAGCTCCGGCAAGATGAGCAAACACAGCCTGTGCAGGGTGCAGGGAGGGCTA | |
| | GAGGCCTGAGGCTTGAAACAGCTCTCAAGTGGAGGGGGAAACAACCATTGCCCTCATAG | |
| | AGGACACATCCACACCAGGGCTGTGCTAGCGTGGGCAGGCAAGCCAGGTGCTGGACCTC | |
| | TGCACGTGGGGCATGTGTGGGTATGTACATGTACCTGTGTTCTTGGTGTGTGTGTGTGTGT | |
| | GTGTGTGTGTGTGTGTCTAGAGCTGGGGTGCAACTATGGGGCCCCTCGGGACATGTCCCA | |
| | GCCAATGCCTGCTTTGACCAGAGGAGTGTCCACGTGGCTCAGGTGGTCGAGTATCTCATA | |
| | CCGCCCTAGCACACGTGTGACTCCTTTCCCCTATTGTCTACGCAGCCTGCCCTTGGACAAG | |
| | GACCCGATGCCCAACCCCAGGCCTGGCAAGCCCTCGGCCCCTTCCTTGGCCCTTGGCCCA | |
| | TCCCC | |
| 129 | GACATGTCCCAGCCAATGCCTGCTTTGACCAGAGGAGTGTCCACGTGGCTCAGGTGGTCG | 0.3 kb human |
| | AGTATCTCATACCGCCCTAGCACACGTGTGACTCCTTTCCCCTATTGTCTACGCAGCCTGC | FOXP3 |
| | CCTTGGACAAGGACCCGATGCCCAACCCCAGGCCTGGCAAGCCCTCGGCCCCTTCCTTGG | 5′HA for |
| | CCCTTGGCCCATCCCCAGGAGCCTCGCCCAGCTGGAGGGCTGCACCCAAAGCCTCAGACC | Cas9-T3 |
| | TGCTGGGGGCCCGGGGCCCAGGGGGAACCTTCCA | |
| 130 | CATAGAGGACACATCCACACCAGGGCTGTGCTAGCGTGGGCAGGCAAGCCAGGTGCTGG | 0.45 kb |
| | ACCTCTGCACGTGGGGCATGTGTGGGTATGTACATGTACCTGTGTTCTTGGTGTGTGTGTG | human |
| | TGTGTGTGTGTGTGTGTGTGTCTAGAGCTGGGGTGCAACTATGGGGCCCCTCGGGACATG | FOXP3 |
| | TCCCAGCCAATGCCTGCTTTGACCAGAGGAGTGTCCACGTGGCTCAGGTGGTCGAGTATC | 5′HA for |
| | TCATACCGCCCTAGCACACGTGTGACTCCTTTCCCCTATTGTCTACGCAGCCTGCCCTTGG | Cas9-T3 |
| | ACAAGGACCCGATGCCCAACCCCAGGCCTGGCAAGCCCTCGGCCCCTTCCTTGGCCCTTG | |
| | GCCCATCCCCAGGAGCCTCGCCCAGCTGGAGGGCTGCACCCAAAGCCTCAGACCTGCTG | |
| | GGGGCCCGGGGCCCAGGGGGAACCTTCCA | |
| 131 | CTAGTCACTGGGGCAAAATAGGACTCCGAGGAGAAAGTCCGAGACCAGCTCCGGCAAGA | 0.6 kb human |
| | TGAGCAAACACAGCCTGTGCAGGGTGCAGGGAGGGCTAGAGGCCTGAGGCTTGAAACAG | FOXP3 |
| | CTCTCAAGTGGAGGGGGAAACAACCATTGCCCTCATAGAGGACACATCCACACCAGGGC | 5′HA for |
| | TGTGCTAGCGTGGGCAGGCAAGCCAGGTGCTGGACCTCTGCACGTGGGGCATGTGTGGG | Cas9-T3 |
| | TATGTACATGTACCTGTGTTCTTGGTGTGTGTGTGTGTGTGTGTGTGTGTGTGTGTCTAGA | |
| | GCTGGGGTGCAACTATGGGGCCCCTCGGGACATGTCCCAGCCAATGCCTGCTTTGACCAG | |
| | AGGAGTGTCCACGTGGCTCAGGTGGTCGAGTATCTCATACCGCCCTAGCACACGTGTGAC | |
| | TCCTTTCCCCTATTGTCTACGCAGCCTGCCCTTGGACAAGGACCCGATGCCCAACCCCAG | |
| | GCCTGGCAAGCCCTCGGCCCCTTCCTTGGCCCTTGGCCCATCCCCAGGAGCCTCGCCCAG | |
| | CTGGAGGGCTGCACCCAAAGCCTCAGACCTGCTGGGGGCCCGGGGCCCAGGGGGAACCT | |
| | TCCA | |
| 132 | GTGAGGCCCTGGGCCCAGGATGGGGCAGGCAGGGTGGGGTACCTGGACCTACAGGTGCC | 0.25 kb |
| | GACCTTTACTGTGGCACTGGGCGGGAGGGGGGCTGGCTGGGGCACAGGAAGTGGTTTCT | human |
| | GGGTCCCAGGCAAGTCTGTGACTTATGCAGATGTTGCAGGGCCAAGAAAATCCCCACCT | FOXP3 |
| | GCCAGGCCTCAGAGATTGGAGGCTCTCCCCGACCTCCCAATCCCTGTCTCAGGAGAGGAG | 3′HA |
| | GAGGCCGT | designed for |
| | | both TALEN |
| | | and Cas9 |
| | | approaches: |
| 133 | GCCTCGCCCAGCTGGAGGGCTGCACCCAAAGCCTCAGACCTGCTGGGGGCCCGGGGCCC | 0.3 kb human |
| | AGGGGGAACCTTCCAGGGCCGAGATCTTCGAGGCGGGGCCCATGCCTCCTCTTCTTCCTT | FOXP3 |
| | GAACCCCATGCCACCATCGCAGCTGCAGGTGAGGCCCTGGGCCCAGGATGGGGCAGGCA | 3′HA for |
| | GGGTGGGGTACCTGGACCTACAGGTGCCGACCTTTACTGTGGCACTGGGCGGGAGGGGG | Cas9-T9 |
| | GCTGGCTGGGGCACAGGAAGTGGTTTCTGGGTCCCAGGCAAGTCTGTGACTTATGCAGAT | |
| | GTT | |
| 134 | GCCTCGCCCAGCTGGAGGGCTGCACCCAAAGCCTCAGACCTGCTGGGGGCCCGGGGCCC | 0.45 kb |
| | AGGGGGAACCTTCCAGGGCCGAGATCTTCGAGGCGGGGCCCATGCCTCCTCTTCTTCCTT | human |
| | GAACCCCATGCCACCATCGCAGCTGCAGGTGAGGCCCTGGGCCCAGGATGGGGCAGGCA | FOXP3 |
| | GGGTGGGGTACCTGGACCTACAGGTGCCGACCTTTACTGTGGCACTGGGCGGGAGGGGG | 3′HA for |
| | GCTGGCTGGGGCACAGGAAGTGGTTTCTGGGTCCCAGGCAAGTCTGTGACTTATGCAGAT | Cas9-T9 |
| | GTTGCAGGGCCAAGAAAATCCCCACCTGCCAGGCCTCAGAGATTGGAGGCTCTCCCCGA | |
| | CCTCCCAATCCCTGTCTCAGGAGAGGAGGAGGCCGTATTGTAGTCCCATGAGCATAGCTA | |
| | TGTGTCCCCATCCCCATGTGACAAGAGAAGAGGA | |
| 135 | GCCTCGCCCAGCTGGAGGGCTGCACCCAAAGCCTCAGACCTGCTGGGGGCCCGGGGCCC | 0.6 kb human |
| | AGGGGGAACCTTCCAGGGCCGAGATCTTCGAGGCGGGGCCCATGCCTCCTCTTCTTCCTT | FOXF3 |
| | GAACCCCATGCCACCATCGCAGCTGCAGGTGAGGCCCTGGGCCCAGGATGGGGCAGGCA | 3′HA for |
| | GGGTGGGGTACCTGGACCTACAGGTGCCGACCTTTACTGTGGCACTGGGCGGGAGGGGG | Cas9-T9 |
| | GCTGGCTGGGGCACAGGAAGTGGTTTCTGGGTCCCAGGCAAGTCTGTGACTTATGCAGAT | |
| | GTTGCAGGGCCAAGAAAATCCCCACCTGCCAGGCCTCAGAGATTGGAGGCTCTCCCCGA | |
| | CCTCCCAATCCCTGTCTCAGGAGAGGAGGAGGCCGTATTGTAGTCCCATGAGCATAGCTA | |
| | TGTGTCCCCATCCCCATGTGACAAGAGAAGAGGACTGGGGCCAAGTAGGTGAGGTGACA | |
| | GGGCTGAGGCCAGCTCTGCAACTTATTAGCTGTTTGATCTTTAAAAAGTTACTCGATCTCC | |
| | ATGAGCCTCAGTTTCCATACGTGTAAAAGGGGGATGATCATAGCATCTACCATGTGGGCT | |
| | TGCA | |
| 136 | GCCTCGCCCAGCTGGAGGGCTGCACCCAAAGCCTCAGACCTGCTGGGGGCCCGGGGCCC | 0.8 kb human |
| | AGGGGGAACCTTCCAGGGCCGAGATCTTCGAGGCGGGGCCCATGCCTCCTCTTCTTCCTT | FOXF3 |
| | GAACCCCATGCCACCATCGCAGCTGCAGGTGAGGCCCTGGGCCCAGGATGGGGCAGGCA | 3′HA for |
| | GGGTGGGGTACCTGGACCTACAGGTGCCGACCTTTACTGTGGCACTGGGCGGGAGGGGG | Cas9-T9 |
| | GCTGGCTGGGGCACAGGAAGTGGTTTCTGGGTCCCAGGCAAGTCTGTGACTTATGCAGAT | |
| | GTTGCAGGGCCAAGAAAATCCCCACCTGCCAGGCCTCAGAGATTGGAGGCTCTCCCCGA | |
| | CCTCCCAATCCCTGTCTCAGGAGAGGAGGAGGCCGTATTGTAGTCCCATGAGCATAGCTA | |
| | TGTGTCCCCATCCCCATGTGACAAGAGAAGAGGACTGGGGCCAAGTAGGTGAGGTGACA | |
| | GGGCTGAGGCCAGCTCTGCAACTTATTAGCTGTTTGATCTTTAAAAAGTTACTCGATCTCC | |
| | ATGAGCCTCAGTTTCCATACGTGTAAAAGGGGGATGATCATAGCATCTACCATGTGGGCT | |
| | TGCAGTGCAGAGTATTTGAATTAGACACAGAACAGTGAGGATCAGGATGGCCTCTCACC | |
| | CACCTGCCTTTCTGCCCAGCTGCCCACACTGCCCCTAGTCATGGTGGCACCCTCCGGGGC | |
| | ACGGCTGGGCCCCTTGCCCCACTTACAGGCACTCCTCCAGGACAGGCCACATTTCATGCA | |
| | CCAGGTATGGACGGTGAAT | |
| 137 | CGAGATCTTCGAGGCGGGGCCCATGCCTCCTCTTCTTCCTTGAACCCCATGCCACCATCG | 0.3 kb human |
| | CAGCTGCAGGTGAGGCCCTGGGCCCAGGATGGGGCAGGCAGGGTGGGGTACCTGGACCT | FOXP3 |
| | ACAGGTGCCGACCTTTACTGTGGCACTGGGCGGGAGGGGGGCTGGCTGGGGCACAGGAA | 3′HA for |
| | GTGGTTTCTGGGTCCCAGGCAAGTCTGTGACTTATGCAGATGTTGCAGGGCCAAGAAAAT | Cas9-T3 |
| | CCCCACCTGCCAGGCCTCAGAGATTGGAGGCTCTCCCCGACCTCCCAATCCCTGTCTCAG | |
| | GA | |
| 138 | CGAGATCTTCGAGGCGGGGCCCATGCCTCCTCTTCTTCCTTGAACCCCATGCCACCATCG | 0.45 kb |
| | CAGCTGCAGGTGAGGCCCTGGGCCCAGGATGGGGCAGGCAGGGTGGGGTACCTGGACCT | human |
| | ACAGGTGCCGACCTTTACTGTGGCACTGGGCGGGAGGGGGGCTGGCTGGGGCACAGGAA | FOXP3 |
| | GTGGTTTCTGGGTCCCAGGCAAGTCTGTGACTTATGCAGATGTTGCAGGGCCAAGAAAAT | 3′HA for |
| | CCCCACCTGCCAGGCCTCAGAGATTGGAGGCTCTCCCCGACCTCCCAATCCCTGTCTCAG | Cas9-T3 |
| | GAGAGGAGGAGGCCGTATTGTAGTCCCATGAGCATAGCTATGTGTCCCCATCCCCATGTG | |
| | ACAAGAGAAGAGGACTGGGGCCAAGTAGGTGAGGTGACAGGGCTGAGGCCAGCTCTGC | |
| | AACTTATTAGCTGTTTGATCTTTAAAAAGTTACTC | |
| 139 | CGAGATCTTCGAGGCGGGGCCCATGCCTCCTCTTCTTCCTTGAACCCCATGCCACCATCG | 0.6 kb human |
| | CAGCTGCAGGTGAGGCCCTGGGCCCAGGATGGGGCAGGCAGGGTGGGGTACCTGGACCT | FOXP3 |
| | ACAGGTGCCGACCTTTACTGTGGCACTGGGCGGGAGGGGGGCTGGCTGGGGCACAGGAA | 3′HA for |
| | GTGGTTTCTGGGTCCCAGGCAAGTCTGTGACTTATGCAGATGTTGCAGGGCCAAGAAAAT | Cas9-T3 |
| | CCCCACCTGCCAGGCCTCAGAGATTGGAGGCTCTCCCCGACCTCCCAATCCCTGTCTCAG | |
| | GAGAGGAGGAGGCCGTATTGTAGTCCCATGAGCATAGCTATGTGTCCCCATCCCCATGTG | |
| | ACAAGAGAAGAGGACTGGGGCCAAGTAGGTGAGGTGACAGGGCTGAGGCCAGCTCTGC | |
| | AACTTATTAGCTGTTTGATCTTTAAAAAGTTACTCGATCTCCATGAGCCTCAGTTTCCATA | |
| | CGTGTAAAAGGGGGATGATCATAGCATCTACCATGTGGGCTTGCAGTGCAGAGTATTTGA | |
| | ATTAGACACAGAACAGTGAGGATCAGGATGGCCTCTCACCCACCTGCCTTTCTGCCCAGC | |
| | TGC | |
| 140 | TAGCCACCTCTCCATCCTCTTGCTTTCTTTGCCTGGACACCCCGTTCTCCTGTGGATTCGG | 0.25 kb |
| | GTCACCTCTCACTCCTTTCATTTGGGCAGCTCCCCTACCCCCCTTACCTCTCTAGTCTGTGC | AAVS1 |
| | TAGCTCTTCCAGCCCCCTGTCATGGCATCTTCCAGGGGTCCGAGAGCTCAGCTAGTCTTCT | 5′HA for |
| | TCCTCCAACCCGGGCCCCTATGTCCACTTCAGGACAGCATGTTTGCTGCCTCCAGGGATC | Cas9-P1 and |
| | CTGTGT | Cas9-N2 |
| 141 | AGGTTCCGTCTTCCTCCACTCCCTCTTCCCCTTGCTCTCTGCTGTGTTGCTGCCCAAGGAT | 0.6 kb |
| | GCTCTTTCCGGAGCACTTCCTTCTCGGCGCTGCACCACGTGATGTCCTCTGAGCGGATCCT | AAVS1 |
| | CCCCGTGTCTGGGTCCTCTCCGGGCATCTCTCCTCCCTCACCCAACCCCATGCCGTCTTCA | 5′HA for |
| | CTCGCTGGGTTCCCTTTTCCTTCTCCTTCTGGGGCCTGTGCCATCTCTCGTTTCTTAGGATG | Cas9-P1 and |
| | GCCTTCTCCGACGGATGTCTCCCTTGCGTCCCGCCTCCCCTTCTTGTAGGCCTGCATCATC | Cas9-N2 |
| | ACCGTTTTTCTGGACAACCCCAAAGTACCCCGTCTCCCTGGCTTTAGCCACCTCTCCATCC | |
| | TCTTGCTTTCTTTGCCTGGACACCCCGTTCTCCTGTGGATTCGGGTCACCTCTCACTCCTTT | |
| | CATTTGGGCAGCTCCCCTACCCCCCTTACCTCTCTAGTCTGTGCTAGCTCTTCCAGCCCCC | |
| | TGTCATGGCATCTTCCAGGGGTCCGAGAGCTCAGCTAGTCTTCTTCCTCCAACCCGGGCC | |
| | CCTATGTCCACTTCAGGACAGCATGTTTGCTGCCTCCAGGGATCCTGTGT | |
| 142 | CTCTGGTTCTGGGTACTTTTATCTGTCCCCTCCACCCCACAGTGGGGCCACTAGGGACAG | 0.25 kb |
| | GATTGGTGACAGAAAAGCCCCATCCTTAGGCCTCCTCCTTCCTAGTCTCCTGATATTGGGT | AAVS1 |
| | CTAACCCCCACCTCCTGTTAGGCAGATTCCTTATCTGGTGACACACCCCCATTTCCTGGAG | 3′HA for |
| | CCATCTCTCTCCTTGCCAGAACCTCTAAGGTTTGCTTACGATGGAGCCAGAGAGGATCCT | Cas9-P1 and |
| | GGGAGGGA | Cas9-N2 |
| 143 | CTCTGGTTCTGGGTACTTTTATCTGTCCCCTCCACCCCACAGTGGGGCCACTAGGGACAG | 0.6 kb |
| | GATTGGTGACAGAAAAGCCCCATCCTTAGGCCTCCTCCTTCCTAGTCTCCTGATATTGGGT | AAVS1 |
| | CTAACCCCCACCTCCTGTTAGGCAGATTCCTTATCTGGTGACACACCCCCATTTCCTGGAG | 3′HA for |
| | CCATCTCTCTCCTTGCCAGAACCTCTAAGGTTTGCTTACGATGGAGCCAGAGAGGATCCT | Cas9-P1 and |
| | GGGAGGGAGAGCTTGGCAGGGGGTGGGAGGGAAGGGGGGGATGCGTGACCTGCCCGGT | Cas9-N2 |
| | TCTCAGTGGCCACCCTGCGCTACCCTCTCCCAGAACCTGAGCTGCTCTGACGCGGCCGTC | |
| | TGGTGCGTTTCACTGATCCTGGTGCTGCAGCTTCCTTACACTTCCCAAGAGGAGAAGCAG | |
| | TTTGGAAAAACAAAATCAGAATAAGTTGGTCCTGAGTTCTAACTTTGGCTCTTCACCTTTC | |
| | TAGTCCCCAATTTATATTGTTCCTCCGTGCGTCAGTTTTACCTGTGAGATAAGGCCAGTAG | |
| | CCAGCCCCGTCCTGGCAGGGCTGTGGTGAGGAGGGGGGTGTCCGTGTGGAAAACTCCC | |
| 144 | MGAGATGRAMDGPRLLLLLLLGVSLGGAKEACPTGLYTHSGECCKACNLGEGVAQPCGAN | LNGFRt |
| | QTVCEPCLDSVTFSDVVSATEPCKPCTECVGLQSMSAPCVEADDAVCRCAYGYYQDETTGR | protein |
| | CEACRVCEAGSGLVFSCQDKQNTVCEECPDGTYSDEANHVDPCLPCTVCEDTERQLRECTR | sequence |
| | WADAECEEIPGRWITRSTPPEGSDSTAPSTQEPEAPPEQDLIASTVAGVVTTVMGSSQPVVTR | |
| | GTTDNLIPVYCSILAAVVVGLVAYIAFKR | |
| 145 | MGTSLLCWMALCLLGADHADACPYSNPSLCSGGGGSELPTQGTFSNVSTNVSPAKPTTTACP | RQR8 |
| | YSNPSLCSGGGGSPAPRPPTPAPTIASQPLSLRPEACRPAAGGAVHTRGLDFACDIYIWAPLAG | protein |
| | TCGVLLLSLVITLYCNHRNRRRVCKCPRPVV | sequence |
| 146 | MLLLVTSLLLCELPHPAFLLIPRKVCNGIGIGEFKDSLSINATNIKHFKNCTSISGDLHILPVAFR | EGFRt with |
| | GDSFTHTPPLDPQELDILKTVKEITGFLLIQAWPENRTDLHAFENLEIIRGRTKQHGQFSLAVVS | GM-CSFR |
| | LNITSLGLRSLKEISDGDVIISGNKNLCYANTINWKKLFGTSGQKTKIISNRGENSCKATGQVC | signal |
| | HALCSPEGCWGPEPRDCVSCRNVSRGRECVDKCNLLEGEPREFVENSECIQCHPECLPQAMNI | peptide |
| | TCTGRGPDNCIQCAHYIDGPHCVKTCPAGVMGENNTLVWKYADAGHVCHLCHPNCTYGCT | |
| | GPGLEGCPTNGPKIPSIATGMVGALLLLLVVALGIGLFM | |
| 147 | GAACAGAGAAACAGGAGAATATGGGCCAAACAGGATATCTGTGGTAAGCAGTTCCTGCC | MND |
| | CCGGCTCAGGGCCAAGAACAGTTGGAACAGCAGAATATGGGCCAAACAGGATATCTGTG | promoter |
| | GTAAGCAGTTCCTGCCCCGGCTCAGGGCCAAGAACAGATGGTCCCCAGATGCGGTCCCG | |
| | CCCTCAGCAGTTTCTAGAGAACCATCAGATGTTTCCAGGGTGCCCCAAGGACCTGAAATG | |
| | ACCCTGTGCCTTATTTGAACTAACCAATCAGTTCGCTTCTCGCTTCTGTTCGCGCGCTTCT | |
| | GCTCCCCGAGCTCTATATAAGCAGAGCTCGTTTAGTGAACCGTCAGATC | |
| 148 | CCACGGGGTTGGGGTTGCGCCTTTTCCAAGGCAGCCCTGGGTTTGCGCAGGGACGCGGCT | PGK |
| | GCTCTGGGCGTGGTTCCGGGAAACGCAGCGGCGCCGACCCTGGGTCTCGCACATTCTTCA | promoter |
| | CGTCCGTTCGCAGCGTCACCCGGATCTTCGCCGCTACCCTTGTGGGCCCCCCGGCGACGC | |
| | TTCCTGCTCCGCCCCTAAGTCGGGAAGGTTCCTTGCGGTTCGCGGCGTGCCGGACGTGAC | |
| | AAACGGAAGCCGCACGTCTCACTAGTACCCTCGCAGACGGACAGCGCCAGGGAGCAATG | |
| | GCAGCGCGCCGACCGCGATGGGCTGTGGCCAATAGCGGCTGCTCAGCGGGGCGCGCCGA | |
| | GAGCAGCGGCCGGGAAGGGGCGGTGCGGGAGGCGGGGTGTGGGGCGGTAGTGTGGGCC | |
| | CTGTTCCTGCCCGCGCGGTGTTCCGCATTCTGCAAGCCTCCGGAGCGCACGTCGGCAGTC | |
| | GGCTCCCTCGTTGACCGAATCACCGACCTCTCTCCCCAGGGGGATCC | |
| 149 | AGGCTCCGGTGCCCGTCAGTGGGCAGAGCGCACATCGCCCACAGTCCCCGAGAAGTTGG | EF1 |
| | GGGGAGGGGTCGGCAATTGAACCGGTGCCTAGAGAAGGTGGCGCGGGGTAAACTGGGA | promoter |
| | AAGTGATGTCGTGTACTGGCTCCGCCTTTTTCCCGAGGGTGGGGGAGAACCGTATATAAG | |
| | TGCAGTAGTCGCCGTGAACGTTCTTTTTCGCAACGGGTTTGCCGCCAGAACACA | |
| 150 | TGCTTTATTTGTGAAATTTGTGATGCTATTGCTTTATTTGTAACCATTATAAGCTGCAATA | SV40 poly A |
| | AACAAGTTAACAACAACAATTGCATTCATTTTATGTTTCAGGTTCAGGGGGAGATGTGGG | |
| | AGGTTTTTTAAAGC | |
| 151 | CCTCAAGATCAAGGAAAGGAGGATGGACGAACAGGGGCCAAACTGGTGGGAGGCAGAG | 3′UTR of |
| | GTGGTGGGGGCAGGGATGATAGGCCCTGGATGTGCCCACAGGGACCAAGAAGTGAGGTT | FOXP3 |
| | TCCACTGTCTTGCCTGCCAGGGCCCCTGTTCCCCCGCTGGCAGCCACCCCCTCCCCCATCA | |
| | TATCCTTTGCCCCAAGGCTGCTCAGAGGGGCCCCGGTCCTGGCCCCAGCCCCCACCTCCG | |
| | CCCCAGACACACCCCCCAGTCGAGCCCTGCAGCCAAACAGAGCCTTCACAACCAGCCAC | |
| | ACAGAGCCTGCCTCAGCTGCTCGCACAGATTACTTCAGGGCTGGAAAAGTCACACAGAC | |
| | ACACAAAATGTCACAATCCTGTCCCTCACTCAACACAAACCCCAAAACACAGAGAGCCT | |
| | GCCTCAGTACACTCAAACAACCTCAAAGCTGCATCATCACACAATCACACACAAGCACA | |
| | GCCCTGACAACCCACACACCCCAAGGCACGCACCCACAGCCAGCCTCAGGGCCCACAGG | |
| | GGCACTGTCAACACAGGGGTGTGCCCAGAGGCCTACACAGAAGCAGCGTCAGTACCCTC | |
| | AGGATCTGAGGTCCCAACACGTGCTCGCTCACACACACGGCCTGTTAGAATTCACCTGTG | |
| | TATCTCACGCATATGCACACGCACAGCCCCCCAGTGGGTCTCTTGAGTCCCGTGCAGACA | |
| | CACACAGCCACACACACTGCCTTGCCAAAAATACCCCGTGTCTCCCCTGCCACTCACCTC | |
| | ACTCCCATTCCCTGAGCCCTGATCCATGCCTCAGCTTAGACTGCAGAGGAACTACTCATT | |
| | TATTTGGGATCCAAGGCCCCCAACCCACAGTACCGTCCCCAATAAACTGCAGCCGAGCTC | |
| | CCCACA | |
| 152 | ATGGGGGCAGGTGCCACCGGACGAGCCATGGACGGGCCGCGCCTGCTGCTGTTGCTGCT | LNGFR |
| | TCTGGGGGTGTCCCTTGGAGGTGCCAAGGAGGCATGCCCCACAGGCCTGTACACACACA | coding |
| | GCGGTGAGTGCTGCAAAGCCTGCAACCTGGGCGAGGGTGTGGCCCAGCCTTGTGGAGCC | sequence |
| | AACCAGACCGTGTGTGAGCCCTGCCTGGACAGCGTGACGTTCTCCGACGTGGTGAGCGC | without stop |
| | GACCGAGCCGTGCAAGCCGTGCACCGAGTGCGTGGGGCTCCAGAGCATGTCGGCGCCGT | codon |
| | GCGTGGAGGCCGACGACGCCGTGTGCCGCTGCGCCTACGGCTACTACCAGGATGAGACG | |
| | ACTGGGCGCTGCGAGGCGTGCCGCGTGTGCGAGGCGGGCTCGGGCCTCGTGTTCTCCTGC | |
| | CAGGACAAGCAGAACACCGTGTGCGAGGAGTGCCCCGACGGCACGTATTCCGACGAGGC | |
| | CAACCACGTGGACCCGTGCCTGCCCTGCACCGTGTGCGAGGACACCGAGCGCCAGCTCC | |
| | GCGAGTGCACACGCTGGGCCGACGCCGAGTGCGAGGAGATCCCTGGCCGTTGGATTACA | |
| | CGGTCCACACCCCCAGAGGGCTCGGACAGCACAGCCCCCAGCACCCAGGAGCCTGAGGC | |
| | ACCTCCAGAACAAGACCTCATAGCCAGCACGGTGGCAGGTGTGGTGACCACAGTGATGG | |
| | GCAGCTCCCAGCCCGTGGTGACCCGAGGCACCACCGACAACCTCATCCCTGTCTATTGCT | |
| | CCATCCTGGCTGCTGTGGTTGTGGGTCTTGTGGCCTACATAGCCTTCAAGAGG | |
| 153 | ATGCCTCTGGGCCTGCTGTGGCTGGGCCTGGCCCTGCTGGGCGCCCTGCACGCCCAGGCC | μDISC: |
| | GGCGTGCAGGTGGAGACAATCTCCCCAGGCGACGGACGCACATTCCCTAAGCGGGGCCA | μCISC-FRB; |
| | GACCTGCGTGGTGCACTATACAGGCATGCTGGAGGATGGCAAGAAGTTTGACAGCTCCC | nucleotide |
| | GGGATAGAAACAAGCCATTCAAGTTTATGCTGGGCAAGCAGGAAGTGATCAGAGGCTGG | sequence |
| | GAGGAGGGCGTGGCCCAGATGTCTGTGGGCCAGAGGGCCAAGCTGACCATCAGCCCAGA | |
| | CTACGCCTATGGAGCAACAGGCCACCCAGGAATCATCCCACCTCACGCCACCCTGGTGTT | |
| | CGATGTGGAGCTGCTGAAGCTGGGCGAGGGAGGGTCACCTGGATCCAACACATCAAAAG | |
| | AGAACCCCTTTCTGTTCGCATTGGAGGCCGTAGTCATATCTGTTGGATCCATGGGACTTAT | |
| | TATCTCCCTGTTGTGTGTGTACTTCTGGCTGGAACGGACTATGCCCAGGATCCCCACGCTC | |
| | AAGAATCTGGAAGATCTCGTCACAGAATACCATGGTAATTTCAGCGCCTGGAGCGGAGT | |
| | CTCTAAGGGTCTGGCCGAATCCCTCCAACCCGATTATTCTGAACGGTTGTGCCTCGTATCC | |
| | GAAATACCACCAAAAGGCGGGGCTCTGGGTGAGGGCCCAGGGGCGAGTCCGTGCAATCA | |
| | ACACAGCCCGTATTGGGCCCCTCCTTGTTATACGTTGAAGCCCGAAACTGGAAGCGGAGC | |
| | TACTAACTTCAGCCTGCTGAAGCAGGCTGGAGACGTGGAGGAGAACCCTGGACCTATGG | |
| | CACTGCCCGTGACCGCCCTGCTGCTGCCTCTGGCCCTGCTGCTGCACGCAGCCCGGCCTA | |
| | TCCTGTGGCACGAGATGTGGCACGAGGGCCTGGAGGAGGCCAGCAGGCTGTATTTTGGC | |
| | GAGCGCAACGTGAAGGGCATGTTCGAGGTGCTGGAGCCTCTGCACGCCATGATGGAGAG | |
| | AGGCCCACAGACCCTGAAGGAGACATCCTTTAACCAGGCCTATGGACGGGACCTGATGG | |
| | AGGCACAGGAGTGGTGCAGAAAGTACATGAAGTCTGGCAATGTGAAGGACCTGCTGCAG | |
| | GCCTGGGATCTGTACTATCACGTGTTTCGGAGAATCTCCAAGCCAGCAGCTCTCGGCAAA | |
| | GACACGATTCCGTGGCTTGGGCATCTGCTCGTTGGGCTGAGCGGTGCGTTTGGTTTCATC | |
| | ATCTTGGTCTATCTCTTGATCAATTGCAGAAATACAGGCCCTTGGCTGAAAAAAGTGCTC | |
| | AAGTGTAATACCCCCGACCCAAGCAAGTTCTTCTCCCAGCTTTCTTCAGAGCATGGAGGC | |
| | GATGTGCAGAAATGGCTCTCTTCACCTTTTCCCTCCTCAAGCTTCTCCCCGGGAGGGCTGG | |
| | CGCCCGAGATTTCACCTCTTGAGGTACTTGAACGAGACAAGGTTACCCAACTTCTCCTTC | |
| | AACAGGATAAGGTACCCGAACCTGCGAGCCTTAGCTTGAATACAGACGCTTATCTCTCAC | |
| | TGCAGGAACTGCAAGGATCTGGTGCTACTAATTTTTCTCTTTTGAAGCAAGCTGGAGATG | |
| | TTGAAGAGAACCCCGGTCCGGAGATGTGGCATGAGGGTCTGGAAGAAGCGTCTCGACTG | |
| | TACTTTGGTGAGCGCAATGTGAAGGGCATGTTTGAAGTCCTCGAACCCCTTCATGCCATG | |
| | ATGGAACGCGGACCCCAGACCTTGAAGGAGACAAGTTTTAACCAAGCTTACGGAAGAGA | |
| | CCTGATGGAAGCCCAGGAATGGTGCAGGAAATACATGAAAAGCGGGAATGTGAAGGACT | |
| | TGCTCCAAGCGTGGGACCTGTACTATCATGTCTTTAGGCGCATTAGTAAG | |
| 154 | MPLGLLWLGLALLGALHAQAGVQVETISPGDGRTFPKRGQTCVVHYTGMLEDGKKFDSSRD | μDISC: |
| | RNKPFKFMLGKQEVIRGWEEGVAQMSVGQRAKLTISPDYAYGATGHPGIIPPHATLVFDVEL | μCISC-FRB |
| | LKLGEGGSPGSNTSKENPFLFALEAVVISVGSMGLIISLLCVYFWLERTMPRIPTLKNLEDLVT | amino acid |
| | EYHGNFSAWSGVSKGLAESLQPDYSERLCLVSEIPPKGGALGEGPGASPCNQHSPYWAPPCY | sequence |
| | TLKPETGSGATNFSLLKQAGDVEENPGPMALPVTALLLPLALLLHAARPILWHEMWHEGLEE | |
| | ASRLYFGERNVKGMFEVLEPLHAMMERGPQTLKETSFNQAYGRDLMEAQEWCRKYMKSG | |
| | NVKDLLQAWDLYYHVFRRISKPAALGKDTIPWLGHLLVGLSGAFGFIILVYLLINCRNTGPWL | |
| | KKVLKCNTPDPSKFFSQLSSEHGGDVQKWLSSPFPSSSFSPGGLAPEISPLEVLERDKVTQLLL | |
| | QQDKVPEPASLSLNTDAYLSLQELQGSGATNFSLLKQAGDVEENPGPEMWHEGLEEASRLYF | |
| | GERNVKGMFEVLEPLHAMMERGPQTLKETSFNQAYGRDLMEAQEWCRKYMKSGNVKDLL | |
| | QAWDLYYHVFRRISK | |
| 155 | CACGTGTGACTCCTTTCCC | NHEJ_F |
| 156 | CCCAGTGCCACAGTAAAGGT | NHEJ_R |
| 157 | AGGGCCGAGATCTTCGAGGC | FAM_NHEJ |
| | | probe |
| 158 | CGACACTTCACCCCTTTTCT | Control_F |
| 159 | CTCCCCAATGTGCCTATGAG | Control_R |
| 160 | GTGGCGGTGACTGGGATGGC | HEXControl |
| | | probe |
| 161 | GTAGAAAAGATCAAAGGATCTTCTTGAGATCCTTTTTTTCTGCGCGTAATCTGCTGCTTGC | #3232_pAAV. |
| | AAACAAAAAAACCACCGCTACCAGCGGTGGTTTGTTTGCCGGATCAAGAGCTACCAACT | FOXP3.0.8H |
| | CTTTTTCCGAAGGTAACTGGCTTCAGCAGAGCGCAGATACCAAATACTGTCCTTCTAGTG | A.ATG.FOX |
| | TAGCCGTAGTTAGGCCACCACTTCAAGAACTCTGTAGCACCGCCTACATACCTCGCTCTG | P3cDNA.WP |
| | CTAATCCTGTTACCAGTGGCTGCTGCCAGTGGCGATAAGTCGTGTCTTACCGGGTTGGAC | RE3.pA_T3 |
| | TCAAGACGATAGTTACCGGATAAGGCGCAGCGGTCGGGCTGAACGGGGGGTTCGTGCAC | specific |
| | ACAGCCCAGCTTGGAGCGAACGACCTACACCGAACTGAGATACCTACAGCGTGAGCTAT | |
| | GAGAAAGCGCCACGCTTCCCGAAGGGAGAAAGGCGGACAGGTATCCGGTAAGCGGCAG | |
| | GGTCGGAACAGGAGAGCGCACGAGGGAGCTTCCAGGGGGAAACGCCTGGTATCTTTATA | |
| | GTCCTGTCGGGTTTCGCCACCTCTGACTTGAGCGTCGATTTTTGTGATGCTCGTCAGGGGG | |
| | GCGGAGCCTATGGAAAAACGCCAGCAACGCGGCCTTTTTACGGTTCCTGGCCTTTTGCTG | |
| | GCCTTTTGCTCACATGTTCTTTCCTGCGTTATCCCCTGATTCTGTGGATAACCGTATTACCG | |
| | CCTTTGAGTGAGCTGATACCGCTCGCCGCAGCCGAACGACCGAGCGCAGCGAGTCAGTG | |
| | AGCGAGGAAGCGGAAGAGCGCCCAATACGCAAACCGCCTCTCCCCGCGCGTTGGCCGAT | |
| | TCATTAATGCAGCTGCGCGCTCGCTCGCTCACTGAGGCCGCCCGGGCAAAGCCCGGGCGT | |
| | CGGGCGACCTTTGGTCGCCCGGCCTCAGTGAGCGAGCGAGCGCGCAGAGAGGGAGTGGC | |
| | CAACTCCATCACTAGGGGTTCCTTGTAGTTAATGATTAACCCGCCATGCTACTTATCTACG | |
| | TAGCGGCCGCATTTAATGCCAGACTCTTCATGTCTATCTACACCTGCACTTTTGCACCCAA | |
| | TCCAACTCCCCGCCATGTCCCCCATCTCAGGTAATGTCAGCTCGGTCCTTCCAGCTGCTCA | |
| | AGCTAAAACCCATGTCACTTTGACTCTCCCTCTTGCCCACTACATCCAAGCTGCTAGCACT | |
| | GCTCCTGATCCAGCTTCAGATTAAGTCTCAGAATCTACCCACTTCTCGCCTTCTCCACTGC | |
| | CACCAGCCCATTCTGTGCCAGCATCATCACTTGCCAGGACTGTTACAATAGCCTCCTCAC | |
| | TAGCCCCACTCACAGCAGCCAGATGAATCTTTTGAGTCCATGCCTAGTCACTGGGGCAAA | |
| | ATAGGACTCCGAGGAGAAAGTCCGAGACCAGCTCCGGCAAGATGAGCAAACACAGCCTG | |
| | TGCAGGGTGCAGGGAGGGCTAGAGGCCTGAGGCTTGAAACAGCTCTCAAGTGGAGGGGG | |
| | AAACAACCATTGCCCTCATAGAGGACACATCCACACCAGGGCTGTGCTAGCGTGGGCAG | |
| | GCAAGCCAGGTGCTGGACCTCTGCACGTGGGGCATGTGTGGGTATGTACATGTACCTGTG | |
| | TTCTTGGTGTGTGTGTGTGTGTGTGTGTGTGTGTGTGTCTAGAGCTGGGGTGCAACTATGG | |
| | GGCCCCTCGGGACATGTCCCAGCCAATGCCTGCTTTGACCAGAGGAGTGTCCACGTGGCT | |
| | CAGGTGGTCGAGTATCTCATACCGCCCTAGCACACGTGTGACTCCTTTCCCCTATTGTCTA | |
| | CGCAGCCTGCCCTTGGACAAGGACCCGATGCCTAATCCTCGGCCTGGAAAGCCTAGCGCT | |
| | CCTTCTCTTGCTCTGGGACCTTCTCCTGGCGCCTCTCCATCTTGGAGAGCCGCTCCTAAAG | |
| | CCAGCGATCTGCTGGGAGCTAGAGGACCTGGCGGCACATTTCAGGGCAGAGATCTTAGA | |
| | GGCGGAGCCCACGCTAGCTCCTCCAGCCTTAATCCTATGCCTCCTAGCCAGCTCCAGCTG | |
| | CCTACACTGCCTCTGGTTATGGTGGCTCCTAGCGGAGCTAGACTGGGCCCTCTGCCTCAT | |
| | CTGCAAGCTCTGCTGCAGGACAGACCCCACTTCATGCACCAGCTGAGCACCGTGGATGCC | |
| | CACGCAAGAACACCTGTGCTGCAGGTTCACCCTCTGGAATCCCCAGCCATGATCAGCCTG | |
| | ACACCTCCAACAACAGCCACCGGCGTGTTCAGCCTGAAAGCCAGACCTGGACTGCCTCCT | |
| | GGCATCAATGTGGCCAGCCTGGAATGGGTGTCCAGAGAACCTGCTCTGCTGTGCACATTC | |
| | CCCAATCCAAGCGCTCCCAGAAAGGACAGCACACTGTCTGCCGTGCCTCAGAGCAGCTA | |
| | TCCCCTGCTTGCTAACGGCGTGTGCAAGTGGCCTGGATGCGAGAAGGTGTTCGAGGAACC | |
| | CGAGGACTTCCTGAAGCACTGCCAGGCCGATCATCTGCTGGACGAGAAAGGCAGAGCCC | |
| | AGTGTCTGCTCCAGCGCGAGATGGTGCAGTCTCTGGAACAGCAGCTGGTCCTGGAAAAA | |
| | GAAAAGCTGAGCGCCATGCAGGCCCACCTGGCCGGAAAAATGGCCCTGACAAAGGCCAG | |
| | CAGCGTGGCCTCTTCTGATAAGGGCAGCTGCTGCATTGTGGCCGCTGGATCTCAGGGACC | |
| | TGTGGTTCCTGCTTGGAGCGGACCTAGAGAGGCCCCTGATTCTCTGTTTGCCGTGCGGAG | |
| | ACACCTGTGGGGCTCTCACGGCAACTCTACTTTCCCCGAGTTCCTGCACAACATGGACTA | |
| | CTTCAAGTTCCACAACATGCGGCCTCCATTCACCTACGCCACACTGATCAGATGGGCCAT | |
| | TCTGGAAGCCCCTGAGAAGCAGAGAACCCTGAACGAGATCTACCACTGGTTTACCCGGA | |
| | TGTTCGCCTTCTTCCGGAATCACCCTGCCACCTGGAAGAACGCCATCCGGCACAATCTGA | |
| | GCCTGCACAAGTGCTTCGTGCGCGTGGAATCTGAGAAAGGCGCCGTGTGGACAGTGGAC | |
| | GAGCTGGAATTCAGAAAGAAGAGAAGCCAGCGGCCTAGCCGGTGCAGCAATCCTACACC | |
| | TGGACCTTGAAAGCTTGATAATCAACCTCTGGATTACAAAATTTGTGAAAGATTGACTGG | |
| | TATTCTTAACTATGTTGCTCCTTTTACGCTATGTGGATACGCTGCTTTAATGCCTTTGTATC | |
| | ATGCTATTGCTTCCCGTATGGCTTTCATTTTCTCCTCCTTGTATAAATCCTGGTTAGTTCTT | |
| | GCCACGGCGGAACTCATCGCCGCCTGCCTTGCCCGCTGCTGGACAGGGGCTCGGCTGTTG | |
| | GGCACTGACAATTCCGTGGGTCGACTGCTTTATTTGTGAAATTTGTGATGCTATTGCTTTA | |
| | TTTGTAACCATTATAAGCTGCAATAAACAAGTTAACAACAACAATTGCATTCATTTTATG | |
| | TTTCAGGTTCAGGGGGAGATGTGGGAGGTTTTTTAAAGCACTAGTCGAGATCTTCGAGGC | |
| | GGGGCCCATGCCTCCTCTTCTTCCTTGAACCCCATGCCACCATCGCAGCTGCAGGTGAGG | |
| | CCCTGGGCCCAGGATGGGGCAGGCAGGGTGGGGTACCTGGACCTACAGGTGCCGACCTT | |
| | TACTGTGGCACTGGGCGGGAGGGGGGCTGGCTGGGGCACAGGAAGTGGTTTCTGGGTCC | |
| | CAGGCAAGTCTGTGACTTATGCAGATGTTGCAGGGCCAAGAAAATCCCCACCTGCCAGG | |
| | CCTCAGAGATTGGAGGCTCTCCCCGACCTCCCAATCCCTGTCTCAGGAGAGGAGGAGGCC | |
| | GTATTGTAGTCCCATGAGCATAGCTATGTGTCCCCATCCCCATGTGACAAGAGAAGAGGA | |
| | CTGGGGCCAAGTAGGTGAGGTGACAGGGCTGAGGCCAGCTCTGCAACTTATTAGCTGTTT | |
| | GATCTTTAAAAAGTTACTCGATCTCCATGAGCCTCAGTTTCCATACGTGTAAAAGGGGGA | |
| | TGATCATAGCATCTACCATGTGGGCTTGCAGTGCAGAGTATTTGAATTAGACACAGAACA | |
| | GTGAGGATCAGGATGGCCTCTCACCCACCTGCCTTTCTGCCCAGCTGCCCACACTGCCCC | |
| | TAGTCATGGTGGCACCCTCCGGGGCACGGCTGGGCCCCTTGCCCCACTTACAGGCACTCC | |
| | TCCAGGACAGGCCACATTTCATGCACCAGGTATGGACGGTGAATGGGCAGGGAGGAGGG | |
| | AGCAGGTGGGAGAACTGTGGGGAGGGGCCCCGAGTCAGGCTGAACCGGATCCTACGTAG | |
| | ATAAGTAGCATGGCGGGTTAATCATTAACTACAAGGAACCCCTAGTGATGGAGTTGGCC | |
| | ACTCCCTCTCTGCGCGCTCGCTCGCTCACTGAGGCCGGGCGACCAAAGGTCGCCCGACGC | |
| | CCGGGCTTTGCCCGGGCGGCCTCAGTGAGCGAGCGAGCGCGCCAGCTGGCGTAATAGCG | |
| | AAGAGGCCCGCACCGATCGCCCTTCCCAACAGTTGCGCAGCCTGAATGGCGAATGGCGA | |
| | TTCCGTTGCAATGGCTGGCGGTAATATTGTTCTGGATATTACCAGCAAGGCCGATAGTTT | |
| | GAGTTCTTCTACTCAGGCAAGTGATGTTATTACTAATCAAAGAAGTATTGCGACAACGGT | |
| | TAATTTGCGTGATGGACAGACTCTTTTACTCGGTGGCCTCACTGATTATAAAAACACTTCT | |
| | CAGGATTCTGGCGTACCGTTCCTGTCTAAAATCCCTTTAATCGGCCTCCTGTTTAGCTCCC | |
| | GCTCTGATTCTAACGAGGAAAGCACGTTATACGTGCTCGTCAAAGCAACCATAGTACGCG | |
| | CCCTGTAGCGGCGCATTAAGCGCGGCGGGTGTGGTGGTTACGCGCAGCGTGACCGCTAC | |
| | ACTTGCCAGCGCCCTAGCGCCCGCTCCTTTCGCTTTCTTCCCTTCCTTTCTCGCCACGTTCG | |
| | CCGGCTTTCCCCGTCAAGCTCTAAATCGGGGGCTCCCTTTAGGGTTCCGATTTAGTGCTTT | |
| | ACGGCACCTCGACCCCAAAAAACTTGATTAGGGTGATGGTTCACGTAGTGGGCCATCGCC | |
| | CTGATAGACGGTTTTTCGCCCTTTGACGTTGGAGTCCACGTTCTTTAATAGTGGACTCTTG | |
| | TTCCAAACTGGAACAACACTCAACCCTATCTCGGTCTATTCTTTTGATTTATAAGGGATTT | |
| | TGCCGATTTCGGCCTATTGGTTAAAAAATGAGCTGATTTAACAAAAATTTAACGCGAATT | |
| | TTAACAAAATATTAACGTTTACAATTTAAATATTTGCTTATACAATCTTCCTGTTTTTGGG | |
| | GCTTTTCTGATTATCAACCGGGGTACATATGATTGACATGCTAGTTTTACGATTACCGTTC | |
| | ATCGATTCTCTTGTTTGCTCCAGACTCTCAGGCAATGACCTGATAGCCTTTGTAGAGACCT | |
| | CTCAAAAATAGCTACCCTCTCCGGCATGAATTTATCAGCTAGAACGGTTGAATATCATAT | |
| | TGATGGTGATTTGACTGTCTCCGGCCTTTCTCACCCGTTTGAATCTTTACCTACACATTAC | |
| | TCAGGCATTGCATTTAAAATATATGAGGGTTCTAAAAATTTTTATCCTTGCGTTGAAATAA | |
| | AGGCTTCTCCCGCAAAAGTATTACAGGGTCATAATGTTTTTGGTACAACCGATTTAGCTTT | |
| | ATGCTCTGAGGCTTTATTGCTTAATTTTGCTAATTCTTTGCCTTGCCTGTATGATTTATTGG | |
| | ATGTTGGAATCGCCTGATGCGGTATTTTCTCCTTACGCATCTGTGCGGTATTTCACACCGC | |
| | ATATGGTGCACTCTCAGTACAATCTGCTCTGATGCCGCATAGTTAAGCCAGCCCCGACAC | |
| | CCGCCAACACCCGCTGACGCGCCCTGACGGGCTTGTCTGCTCCCGGCATCCGCTTACAGA | |
| | CAAGCTGTGACCGTCTCCGGGAGCTGCATGTGTCAGAGGTTTTCACCGTCATCACCGAAA | |
| | CGCGCGAGACGAAAGGGCCTCGTGATACGCCTATTTTTATAGGTTAATGTCATGATAATA | |
| | ATGGTTTCTTAGACGTCAGGTGGCACTTTTCGGGGAAATGTGCGCGGAACCCCTATTTGT | |
| | TTATTTTTCTAAATACATTCAAATATGTATCCGCTCATGAGACAATAACCCTGATAAATGC | |
| | TTCAATAATATTGAAAAAGGAAGAGTATGAGTATTCAACATTTCCGTGTCGCCCTTATTC | |
| | CCTTTTTTGCGGCATTTTGCCTTCCTGTTTTTGCTCACCCAGAAACGCTGGTGAAAGTAAA | |
| | AGATGCTGAAGATCAGTTGGGTGCACGAGTGGGTTACATCGAACTGGATCTCAACAGCG | |
| | GTAAGATCCTTGAGAGTTTTCGCCCCGAAGAACGTTTTCCAATGATGAGCACTTTTAAAG | |
| | TTCTGCTATGTGGCGCGGTATTATCCCGTATTGACGCCGGGCAAGAGCAACTCGGTCGCC | |
| | GCATACACTATTCTCAGAATGACTTGGTTGAGTACTCACCAGTCACAGAAAAGCATCTTA | |
| | CGGATGGCATGACAGTAAGAGAATTATGCAGTGCTGCCATAACCATGAGTGATAACACT | |
| | GCGGCCAACTTACTTCTGACAACGATCGGAGGACCGAAGGAGCTAACCGCTTTTTTGCAC | |
| | AACATGGGGGATCATGTAACTCGCCTTGATCGTTGGGAACCGGAGCTGAATGAAGCCAT | |
| | ACCAAACGACGAGCGTGACACCACGATGCCTGTAGCAATGGCAACAACGTTGCGCAAAC | |
| | TATTAACTGGCGAACTACTTACTCTAGCTTCCCGGCAACAATTAATAGACTGGATGGAGG | |
| | CGGATAAAGTTGCAGGACCACTTCTGCGCTCGGCCCTTCCGGCTGGCTGGTTTATTGCTG | |
| | ATAAATCTGGAGCCGGTGAGCGTGGGTCTCGCGGTATCATTGCAGCACTGGGGCCAGAT | |
| | GGTAAGCCCTCCCGTATCGTAGTTATCTACACGACGGGGAGTCAGGCAACTATGGATGA | |
| | ACGAAATAGACAGATCGCTGAGATAGGTGCCTCACTGATTAAGCATTGGTAACTGTCAG | |
| | ACCAAGTTTACTCATATATACTTTAGATTGATTTAAAACTTCATTTTTAATTTAAAAGGAT | |
| | CTAGGTGAAGATCCTTTTTGATAATCTCATGACCAAAATCCCTTAACGTGAGTTTTCGTTC | |
| | CACTGAGCGTCAGACCCC | |

1. (canceled)

2. A CD34+ cell comprising a promoter operably linked to a FOXP3 cDNA sequence, wherein the promoter and the FOXP3 cDNA sequence are located in a FOXP3 gene, AAVS1 locus, or TRA gene in the CD34+ cell.

3. The CD34+ cell of claim 2, wherein the promoter is a constitutive promoter.

4. The CD34+ cell of claim 2, wherein the promoter is an MND promoter, PGK promoter, E2F promoter, or EF-1α promoter.

5. The CD34+ cell of claim 2, wherein the FOXP3 cDNA sequence encodes a wild-type human FOXP3.

6. The CD34+ cell of claim 2, wherein the FOXP3 cDNA sequence is codon-optimized for expression in the CD34+ cell.

7. The CD34+ cell of claim 2, wherein the FOXP3 cDNA sequence comprises at least 90% sequence identity to a nucleotide sequence of SEQ ID NO: 110.

8. The CD34+ cell of claim 2, wherein the promoter and FOXP3 cDNA sequence are in the FOXP3 gene.

9. The CD34+ cell of claim 2, wherein the promoter and FOXP3 cDNA sequence are in the AAVS1 locus.

10. The CD34+ cell of claim 2, wherein the promoter and FOXP3 cDNA sequence are in the TRA gene.

11. The CD34+ cell of claim 2, wherein the CD34+ cell further comprises a nucleic acid encoding a chimeric antigen receptor.

12. The CD34+ cell of claim 2, wherein the CD34+ cell further comprises a nucleic acid encoding a T cell receptor.

13. A CD34+ cell comprising an MND promoter operably linked to a FOXP3 cDNA sequence, wherein the MND promoter and the FOXP3 cDNA sequence are located in a FOXP3 gene, AAVS1 locus, or TRA gene in the CD34+ cell.

14. The CD34+ cell of claim 13, wherein the FOXP3 cDNA sequence encodes a wild-type human FOXP3.

15. The CD34+ cell of claim 13, wherein the FOXP3 cDNA sequence is codon-optimized for expression in the CD34+ cell.

16. The CD34+ cell of claim 13, wherein the FOXP3 cDNA sequence comprises at least 90% sequence identity to a nucleotide sequence of SEQ ID NO: 110.

17. The CD34+ cell of claim 13, wherein the MND promoter and FOXP3 cDNA sequence are in the FOXP3 gene.

18. The CD34+ cell of claim 13, wherein the MND promoter and FOXP3 cDNA sequence are in the AAVS1 locus.

19. The CD34+ cell of claim 13, wherein the MND promoter and FOXP3 cDNA sequence are in the TRA gene.

20. The CD34+ cell of claim 13, wherein the CD34+ cell further comprises a nucleic acid encoding a chimeric antigen receptor.

21. The CD34+ cell of claim 13, wherein the CD34+ cell further comprises a nucleic acid encoding a T cell receptor.
